(19) United States
(12) Patent Application Publication
(10) Pub. No.: US 2024/0376048 A1
(43) Pub. Date: Nov. 14, 2024

(54) Carboxylic Acid Containing Indanyl Compounds for the Treatment of Neurodegenerative Diseases

(71) Applicant: Celgene Corporation, Princeton, NJ (US)

(72) Inventors: Jean-Francois Brazeau, San Diego, CA (US); Rulin Ma, Winchester, MA (US); Jeffrey M. Schkeryantz, Summit, NJ (US); Karin Worm, Summit, NJ (US); Patrick W. Papa, Carlsbad, CA (US)

(73) Assignee: Celgene Corporation, Princeton, NJ (US)

(21) Appl. No.: 18/714,760

(22) PCT Filed: Dec. 7, 2022
(86) PCT No.: PCT/US2022/052142

§ 371 Date: May 30, 2024

Related U.S. Application Data
(60) Provisional application No. 63/286,749, filed on Dec. 7, 2021.

(51) Int. Cl. C07D 205/04; A61K 31/397; A61K 31/401; A61K 31/403; A61K 31/451; C07D 205/12; C07D 207/06; C07D 209/02; C07D 211/32

(52) U.S. Cl. CPC C07D 205/04; A61K 31/397; A61K 31/401; A61K 31/403; A61K 31/451; C07D 205/12; C07D 207/06; C07D 209/02; C07D 211/32

(57) ABSTRACT

Provided herein are compounds and compositions thereof for modulating S1P5. In some embodiments, the compounds and compositions are provided for treatment of neurological diseases. Formula (I).

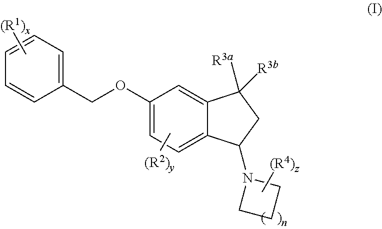

### CROSS-REFERENCE TO RELATED APPLICATIONS

This application claims priority to U.S. Provisional Application No. 63/286,749, filed Dec. 7, 2021, the disclosure of which is incorporated herein by reference in its entirety for any purpose.

### FIELD

The present disclosure relates generally to compounds, compositions, and methods for their preparation and use of the compounds and compositions for treating neurological diseases.

### BACKGROUND

Sphingosine-1-phosphate (S1P; (2S,3R,4E)-2-amino-3-hydroxyoctadec-4-enyl-1-phosphate) is a bioactive sphingolipid that is synthesized by metabolic turnover of sphingolipids in cells and by the extracellular action of a secreted sphingosine kinase. S1P binds to and stimulates members of the endothelial cell differentiation gene family (EDG receptors), which are plasma membrane-localized G protein-coupled receptors. The five members of this family of receptors are S1P1 (EDG-1), S1P2 (EDG-5), S1P3 (EDG-3), S1P4 (EDG-6), and S1P5 (EDG-8). S1P mediates a wide variety of cellular responses including proliferation, cytoskeletal organization and migration, adherence- and tight junction assembly, and morphogenesis.

S1P5 is primarily expressed in the central nervous system. Specifically, S1P5 is highly expressed in oligodendrocytes (oligodendroglia) and oligodendrocyte progenitor cells (Jaillard, C. et al., J. Neuroscience, 2005, 25(6), 1459-1469; Novgorodov, A. S. et al., FASEB J., 2007, 21, 1503-1514). Oligodendrocytes are glial cells that form myelin sheaths (myelin) by binding to the axons of nerve cells. Compounds that bind to S1P5 can modulate the function of S1P5 and may be useful for treating neurodegenerative diseases.

Accordingly, in one aspect, provided herein are compounds that modulate S1P5 for use in treating neurodegenerative diseases.

### SUMMARY

Described herein, in certain embodiments, are compounds and compositions thereof for modulating S1P5. In various embodiments, the compounds and compositions thereof may be used for treatment of neurodegenerative diseases.

The present embodiments can be understood more fully by reference to the detailed description and examples, which are intended to exemplify non-limiting embodiments.

Embodiment 1 is a compound of Formula (I):

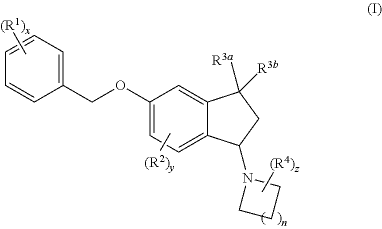

or a pharmaceutically acceptable salt thereof, wherein:
each R¹ is independently halo, —CN, C₁-C₆ alkyl, C₁-C₆ haloalkyl, C₁-C₆ alkoxy, or C₃-C₆ cycloalkyl;
each R² is independently halo, C₁-C₆ alkyl, or C₁-C₆ haloalkyl;
R^{3a} and R^{3b} are independently H, C₁-C₆ alkyl, halo, or C₁-C₆ haloalkyl;
x is 1-5;
y is 0-3;
z is 1-5;
n is 1, 2, or 3; and
each R⁴ is independently —CO₂H, halo, C₁-C₆ haloalkyl, or C₁-C₆ alkyl, or two R⁴ groups are taken together with the carbon atoms to which they are attached to form a fused, bridged, or spiro C₃-C₅ cycloalkyl optionally substituted by —CO₂H, provided that at least one R⁴ group is —CO₂H or contains a —CO₂H moiety.

Embodiment 2 is the compound of embodiment 1, or a pharmaceutically acceptable salt thereof, wherein:
each R¹ is independently halo, —CN, C₁-C₃ alkyl, C₁-C₃ haloalkyl, C₁-C₃ alkoxy, or C₃-C₆ cycloalkyl.

Embodiment 3 is the compound of embodiment 1 or 2, or a pharmaceutically acceptable salt thereof, wherein:
each R¹ is independently F, Cl, I, —CN, —CH₃, —CF₃, —OCH(CH₃)₂, or cyclopropyl.

Embodiment 4 is the compound of any one of embodiments 1-3, or a pharmaceutically acceptable salt thereof, wherein:
x is 1, 2, or 3.

Embodiment 5 is the compound of any one of embodiments 1-4, or a pharmaceutically acceptable salt thereof, wherein:

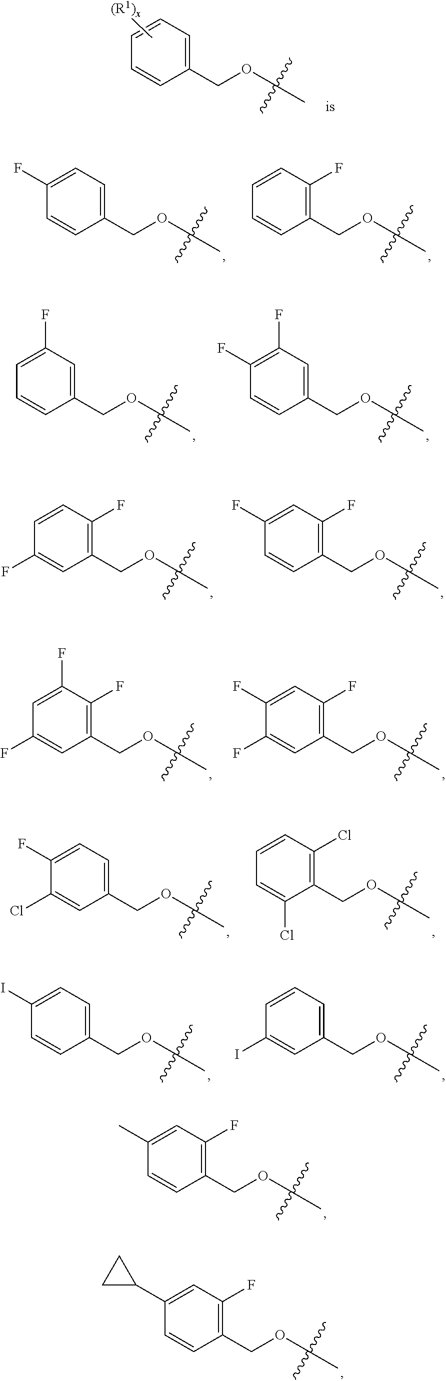

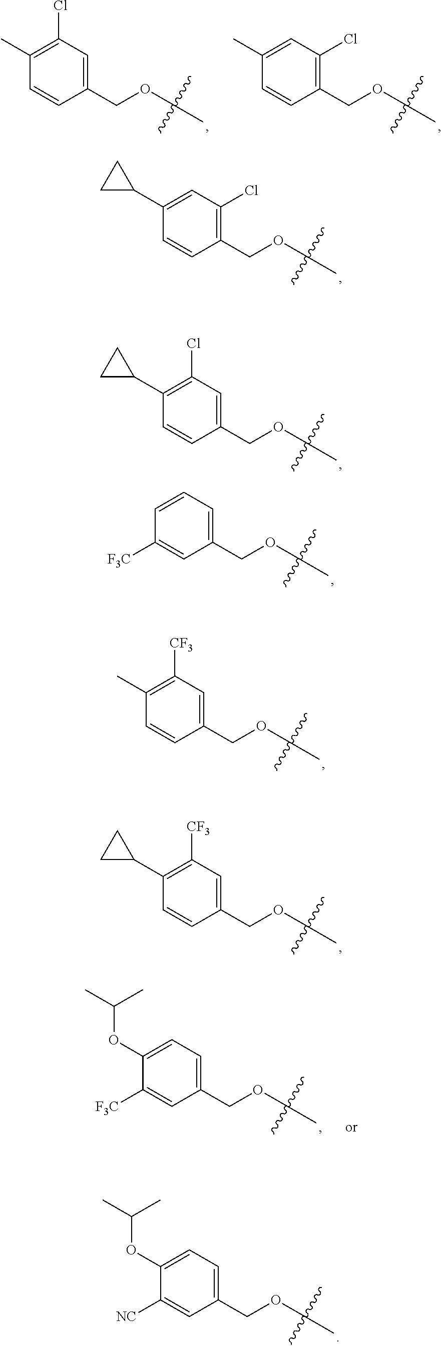

Embodiment 6 is the compound of any one of embodiments 1-5, or a pharmaceutically acceptable salt thereof, wherein:
each R² is independently halo, C₁-C₃ alkyl, or C₁-C₃ haloalkyl.

Embodiment 7 is the compound of embodiment 6, or a pharmaceutically acceptable salt thereof, wherein:
each R² is independently F or —CH₃.

Embodiment 8 is the compound of any one of embodiments 1-7, or a pharmaceutically acceptable salt thereof, wherein:
y is 1.

Embodiment 9 is the compound of any one of embodiments 1-5, or a pharmaceutically acceptable salt thereof, wherein:
y is 0.

Embodiment 10 is the compound of any one of embodiments 1-9, or a pharmaceutically acceptable salt thereof, wherein:

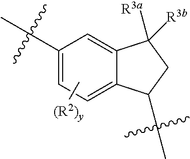

is

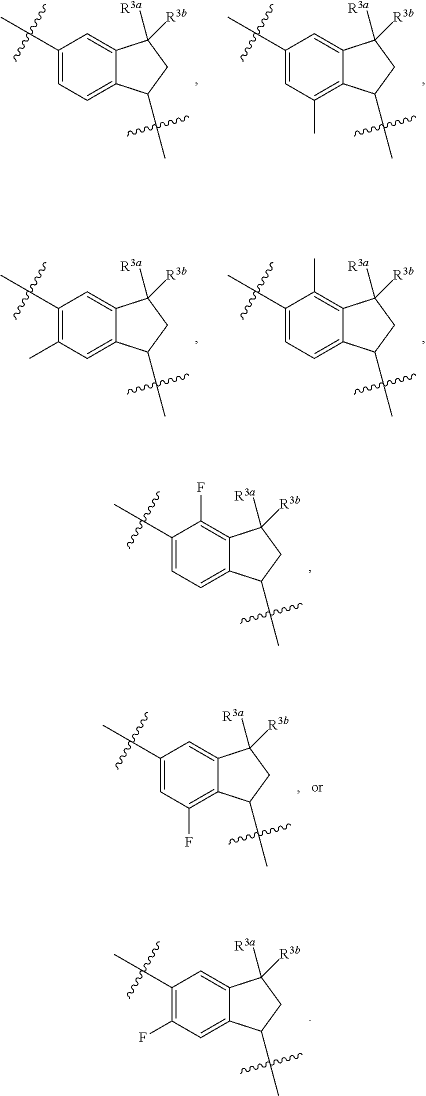

Embodiment 11 is the compound of any one of embodiments 1-10, or a pharmaceutically acceptable salt thereof, wherein:
R^{3a} and R^{3b} are independently H, C₁-C₃ alkyl, halo, or C₁-C₃ haloalkyl.

Embodiment 12 is the compound of embodiment 11, or a pharmaceutically acceptable salt thereof, wherein:
R^{3a} and R^{3b} are independently H or —CH₃.

Embodiment 13 is the compound of any one of embodiments 1-12, or a pharmaceutically acceptable salt thereof, wherein:

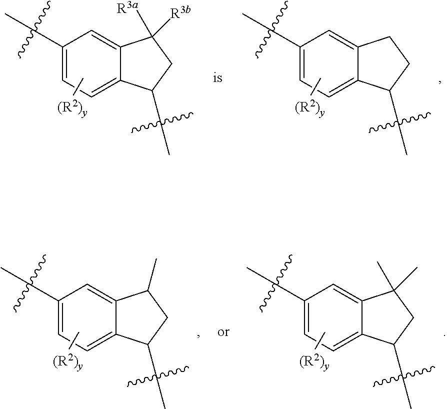

Embodiment 14 is the compound of any one of embodiments 1-13, or a pharmaceutically acceptable salt thereof, wherein:

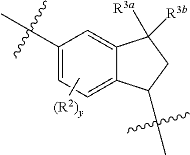

is

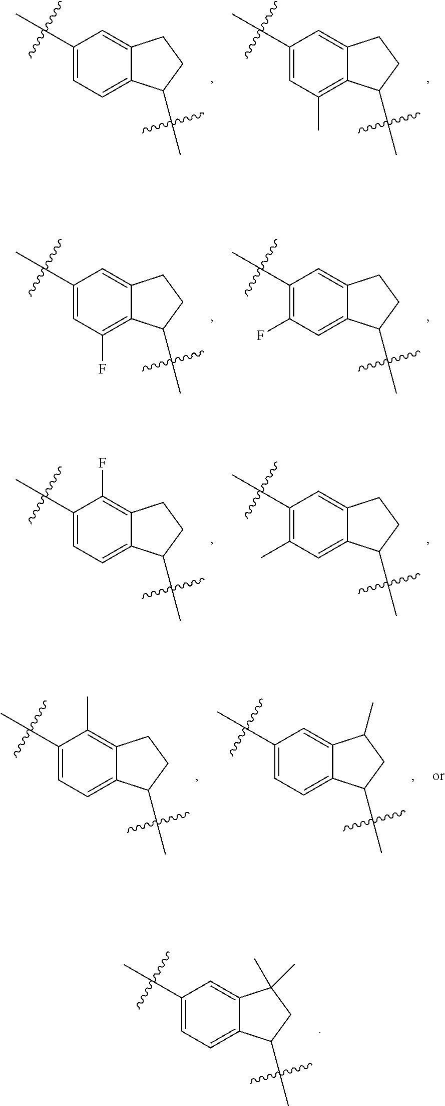

Embodiment 15 is the compound of any one of embodiments 1-14, or a pharmaceutically acceptable salt thereof, wherein:
n is 1.

Embodiment 16 is the compound of any one of embodiments 1-14, or a pharmaceutically acceptable salt thereof, wherein:
n is 2.

Embodiment 17 is the compound of any one of embodiments 1-14, or a pharmaceutically acceptable salt thereof, wherein:
n is 3.

Embodiment 18 is the compound of any one of embodiments 1-17, or a pharmaceutically acceptable salt thereof, wherein:
each R⁴ is independently —CO₂H, halo, C₁-C₃ haloalkyl, or C₁-C₃ alkyl, provided that at least one R⁴ group is —CO₂H.

Embodiment 19 is the compound of any one of embodiments 1-17, or a pharmaceutically acceptable salt thereof, wherein:
two R⁴ groups are taken together with the carbon atoms to which they are attached to form a fused or spiro C₃-C₅ cycloalkyl substituted by —CO₂H.

Embodiment 20 is the compound of any one of embodiments 1-19, or a pharmaceutically acceptable salt thereof, wherein:
z is 1-3.

Embodiment 21 is the compound of any one of embodiments 1-20, or a pharmaceutically acceptable salt thereof, wherein:

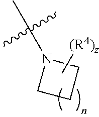

is

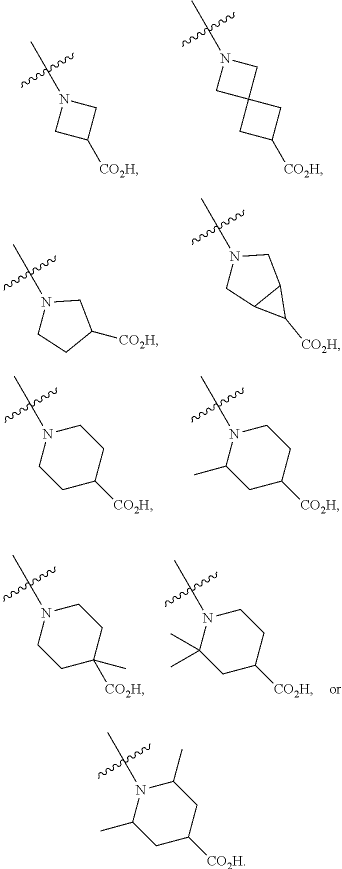

Embodiment 22 is a compound selected from the compounds of Table 1 or a pharmaceutically acceptable salt thereof.

Embodiment 23 is a pharmaceutical composition comprising the compound of any one of embodiments 1-22, or a pharmaceutically acceptable salt thereof, and a pharmaceutically acceptable excipient.

Embodiment 24 is a method of modulating sphingosine 1-phosphate receptor 5 (S1P5) comprising contacting S1P5 with an effective amount of the compound of any one of embodiments 1-22, or a pharmaceutically acceptable salt thereof, or the pharmaceutical composition of embodiment 23.

Embodiment 25 is a method of treating a neurological disease in a subject in need thereof, comprising administering to the subject an effective amount of the compound of any one of embodiments 1-22, or a pharmaceutically acceptable salt thereof, or the pharmaceutical composition of embodiment 23.

Embodiment 26 is the method of embodiment 25, wherein the neurological disease is Alzheimer's disease or multiple sclerosis.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C—CH, —C—C(CH₃), —C—C(CH₂CH₃), —CH₂C—CH, —CH₂C—C(CH₃) and —CH₂C—C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₅ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “alkoxy” group is —O-(alkyl), wherein alkyl is defined above.

A “carboxy” group is a radical of the formula: —C(O)OH.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

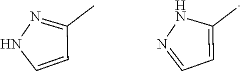

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an S1P5 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

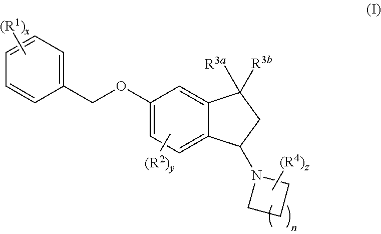

or a pharmaceutically acceptable salt thereof, wherein:
each R¹ is independently halo, —CN, C₁-C₆ alkyl, C₁-C₆ haloalkyl, C₁-C₆ alkoxy, or C₃-C₆ cycloalkyl;
each R² is independently halo, C₁-C₆ alkyl, or C₁-C₆ haloalkyl;
R^{3a} and R^{3b} are independently H, C₁-C₆ alkyl, halo, or C₁-C₆ haloalkyl;
x is 1-5;
y is 0-3;
z is 1-5;
n is 1, 2, or 3; and
each R⁴ is independently —CO₂H, halo, C₁-C₆ haloalkyl, or C₁-C₆ alkyl,
or two R⁴ groups are taken together with the carbon atoms to which they are attached to form a fused, bridged, or spiro C₃-C₅ cycloalkyl optionally substituted by —CO₂H, provided that at least one R⁴ group is —CO₂H or contains a —CO₂H moiety.

In some embodiments, each R¹ is independently halo, —CN, C₁-C₆ alkyl, C₁-C₆ haloalkyl, C₁-C₆ alkoxy, or C₃-C₆ cycloalkyl. In some embodiments, each R¹ is independently halo, —CN, C₁-C₃ alkyl, C₁-C₃ haloalkyl, C₁-C₃ alkoxy, or C₃-C₅ cycloalkyl. In some embodiments, each R¹ is independently F, Cl, I, —CN, methyl, —CF₃, —OCH(CH₃)₂, or cyclopropyl.

In some embodiments, R¹ is halo. In some embodiments, R¹ is F, Cl, Br, or I. In some embodiments, R¹ is F. In some embodiments, R¹ is Cl. In some embodiments, R¹ is Br. In some embodiments, R¹ is I.

In some embodiments, R¹ is —CN.

In some embodiments, R¹ is C₁-C₆ alkyl. In some embodiments, R¹ is C₁-C₃ alkyl. In some embodiments, R¹ is methyl, ethyl, n-propyl, or isopropyl. In some embodiments, R¹ is methyl. In some embodiments, R¹ is ethyl. In some embodiments, R¹ is n-propyl. In some embodiments, R¹ is isopropyl.

In some embodiments, R¹ is C₁-C₆ haloalkyl. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R¹ is —CF₃.

In some embodiments, R¹ is C₁-C₆ alkoxy. In some embodiments, R¹ is C₁-C₃ alkoxy. In some embodiments, R¹ is —OCH₃, —OCH₂CH₃, —OCH₂CH₂CH₃, or —OCH(CH₃)₂. In some embodiments, R¹ is —OCH(CH₃)₂.

In some embodiments, R¹ is C₃-C₆ cycloalkyl. In some embodiments, R¹ is C₃-C₅ cycloalkyl. In some embodiments, R¹ is cyclopropyl, cyclobutyl, cyclopentyl, or cyclohexyl. In some embodiments, R¹ is cyclopropyl. In some embodiments, R¹ is cyclobutyl.

In some embodiments, x is 1-5. In some embodiments, x is 1-3. In some embodiments, x is 1. In some embodiments, x is 2. In some embodiments, x is 3. In some embodiments, x is 4. In some embodiments, x is 5.

In some embodiments,

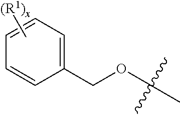

is:

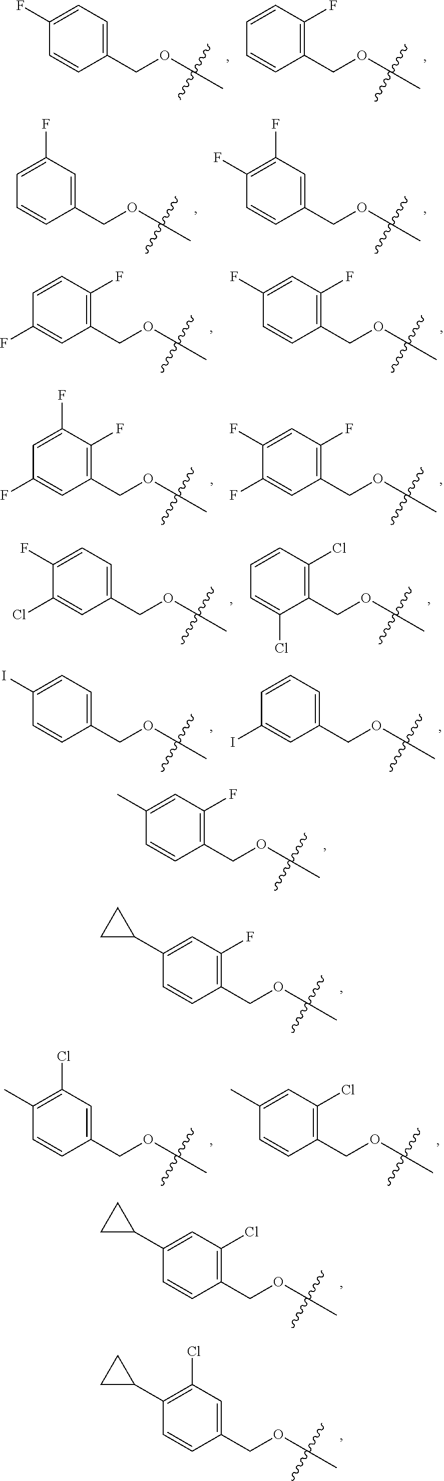

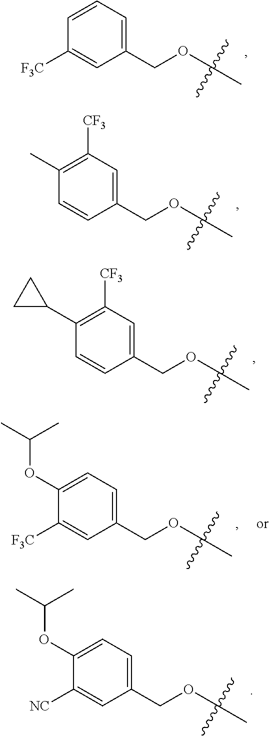 [SMILES: *OCc1cccc(C(F)(F)F)c1 ; *OCc1ccc(C)c(C(F)(F)F)c1 ; *OCc1ccc(C2CC2)c(C(F)(F)F)c1 ; *OCc1ccc(OC(C)C)c(C(F)(F)F)c1 ; *OCc1ccc(OC(C)C)c(C#N)c1]

In some embodiments, each R² is independently halo, C₁-C₆ alkyl, or C₁-C₆ haloalkyl. In some embodiments, each R² is independently halo, C₁-C₃ alkyl, or C₁-C₃ haloalkyl. In some embodiments, each R² is independently halo or C₁-C₃ alkyl. In some embodiments, each R² is independently F or —CH₃.

In some embodiments, R² is halo. In some embodiments, R² is F, Cl, Br, or I. In some embodiments, R² is F. In some embodiments, R² is Cl. In some embodiments, R² is Br. In some embodiments, R² is I.

In some embodiments, R² is C₁-C₆ alkyl. In some embodiments, R² is C₁-C₃ alkyl. In some embodiments, R² is methyl, ethyl, n-propyl, or isopropyl. In some embodiments, R² is methyl. In some embodiments, R² is ethyl. In some embodiments, R² is n-propyl. In some embodiments, R² is isopropyl.

In some embodiments, R² is C₁-C₆ haloalkyl. In some embodiments, R² is C₁-C₆ haloalkyl containing 1-13 halogen atoms. In some embodiments, R² is C₁-C₃ haloalkyl. In some embodiments, R² is C₁-C₃ haloalkyl containing 1-7 halogen atoms. In some embodiments, R² is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R² is —CF₃.

In some embodiments, y is 0-3. In some embodiments, y is 0. In some embodiments, y is 1 or 2. In some embodiments, y is 1. In some embodiments, y is 2. In some embodiments, y is 3.

In some embodiments,

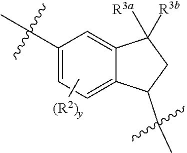

is:

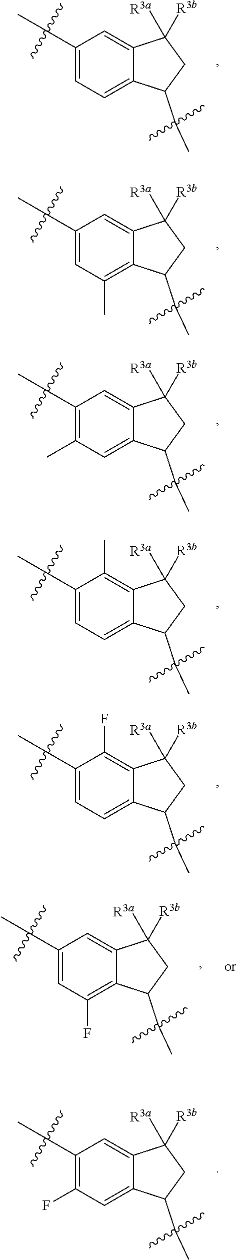

In some embodiments, R^{3a} and R^{3b} are independently H, C₁-C₆ alkyl, halo, or C₁-C₆ haloalkyl. In some embodiments, R^{3a} and R^{3b} are independently H, C₁-C₃ alkyl, halo, or C₁-C₃ haloalkyl. In some embodiments, R^{3a} and R^{3b} are independently H, C₃-C₆ cycloalkyl, or halo. In some embodiments, R^{3a} and R^{3b} are independently H or —CH₃.

In some embodiments, R^{3a} is H. In some embodiments, R^{3a} is C₁-C₆ alkyl. In some embodiments, R^{3a} is C₁-C₃ alkyl. In some embodiments, R^{3a} is methyl, ethyl, n-propyl, or isopropyl. In some embodiments, R^{3a} is methyl or ethyl. In some embodiments, R^{3a} is methyl. In some embodiments, R^{3a} is halo. In some embodiments, R^{3a} is F, Cl, or Br. In some embodiments, R^{3a} is C₁-C₆ haloalkyl. In some embodiments, R^{3a} is C₁-C₆ haloalkyl containing 1-13 halogen atoms. In some embodiments, R^{3a} is C₁-C₃ haloalkyl. In some embodiments, R^{3a} is C₁-C₃ haloalkyl containing 1-7 halogen atoms. In some embodiments, R^{3a} is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R^{3a} is —CF₃.

In some embodiments, R^{3b} is H. In some embodiments, R^{3b} is C₁-C₆ alkyl. In some embodiments, R^{3b} is C₁-C₃ alkyl. In some embodiments, R^{3b} is methyl, ethyl, n-propyl, or isopropyl. In some embodiments, R^{3b} is methyl or ethyl. In some embodiments, R^{3b} is methyl. In some embodiments, R^{3b} is halo. In some embodiments, R^{3b} is F, Cl, or Br. In some embodiments, R^{3b} is C₁-C₆ haloalkyl. In some embodiments, R^{3b} is C₁-C₆ haloalkyl containing 1-13 halogen atoms. In some embodiments, R^{3b} is C₁-C₃ haloalkyl. In some embodiments, R^{3b} is C₁-C₃ haloalkyl containing 1-7 halogen atoms. In some embodiments, R^{3b} is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R^{3b} is —CF₃.

In some embodiments, R^{3a} and R^{3b} are each H. In some embodiments, R^{3a} and R^{3b} are each C₁-C₆ alkyl. In some embodiments, R^{3a} and R^{3b} are each C₁-C₃ alkyl. In some embodiments, R^{3a} and R^{3b} are each methyl, ethyl, n-propyl, or isopropyl. In some embodiments, R^{3a} and R^{3b} are each methyl. In some embodiments, R^{3a} and R^{3b} are each halo. In some embodiments, R^{3a} and R^{3b} are each F, Cl, or Br. In some embodiments, R^{3a} and R^{3b} are each C₁-C₆ haloalkyl containing 1-13 halogen atoms. In some embodiments, R^{3a} and R^{3b} are each C₁-C₃ haloalkyl containing 1-7 halogen atoms. In some embodiments, R^{3a} and R^{3b} are each —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R^{3a} and R^{3b} are each —CF₃. In some embodiments, one of R^{3a} and R^{3b} is H and the other is C₁-C₆ alkyl. In some embodiments, one of R^{3a} and R^{3b} is H and the other is C₁-C₃ alkyl. In some embodiments, one of R^{3a} and R^{3b} is H and the other is methyl, ethyl, n-propyl, or isopropyl. In some embodiments, one of R^{3a} and R^{3b} is H and the other is methyl. In some embodiments, one of R^{3a} and R^{3b} is H and the other is halo. In some embodiments, one of R^{3a} and R^{3b} is H and the other is F, Cl, or Br. In some embodiments, one of R^{3a} and R^{3b} is H and the other is C₁-C₆ haloalkyl containing 1-13 halogen atoms. In some embodiments, one of R^{3a} and R^{3b} is H and the other is C₁-C₃ haloalkyl containing 1-7 halogen atoms. In some embodiments, one of R^{3a} and R^{3b} is H and the other is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, one of R^{3a} and R^{3b} is H and the other is —CF₃. In some embodiments, one of R^{3a} and R^{3b} is C₁-C₆ alkyl and the other is halo. In some embodiments, one of R^{3a} and R^{3b} is C₁-C₃ alkyl and the other is halo. In some embodiments, one of R^{3a} and R^{3b} is methyl, ethyl, n-propyl, or isopropyl, and the other is F, Cl, or Br. In some embodiments, one of R^{3a} and R^{3b} is methyl and the other is F, Cl, or Br. In some embodiments, one of R^{3a} and R^{3b} is C₁-C₆ alkyl and the other is C₁-C₆ haloalkyl containing 1-13 halogen atoms. In some embodiments, one of R^{3a} and R^{3b} is C₁-C₃ alkyl and the other is C₁-C₃ haloalkyl containing 1-7 halogen atoms. In some embodiments, one of R^{3a} and R^{3b} is methyl, ethyl, n-propyl, or isopropyl, and the other is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, one of R^{3a} and R^{3b} is methyl and the other is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, one of R^{3a} and R^{3b} is methyl and the other is —CF₃. In some embodiments, one of R^{3a} and R^{3b} is halo and the other is C₁-C₆ haloalkyl containing 1-13 halogen atoms. In some embodiments, one of R^{3a} and R^{3b} is halo and the other is C₁-C₃ haloalkyl containing 1-7 halogen atoms. In some embodiments, one of R^{3a} and R^{3b} is F, Cl, or Br, and the other is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, one of R^{3a} and R^{3b} is F or Cl, and the other is —CF₃.

In some embodiments,

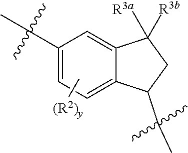

is:

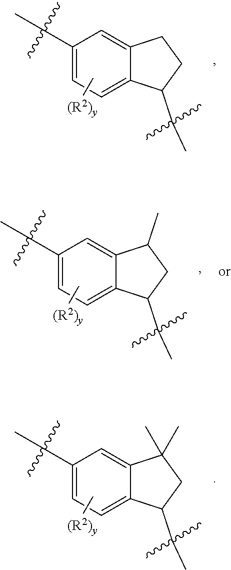

In some embodiments,

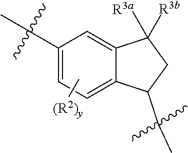

is:

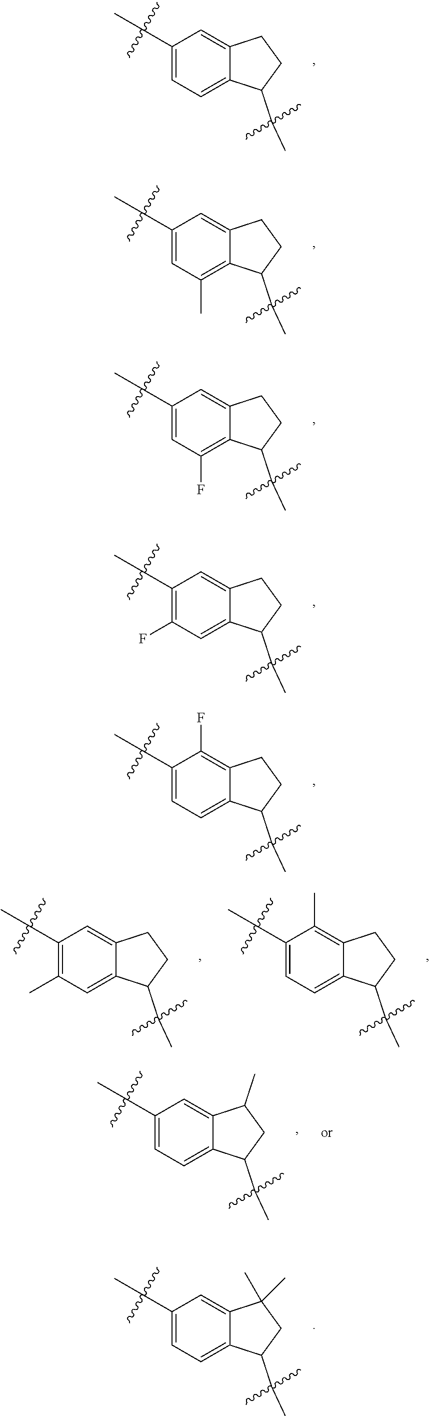

In some embodiments, n is 1, 2, or 3. In some embodiments, n is 1. In some embodiments, n is 2. In some embodiments, n is 3.

In some embodiments, each R⁴ is independently —CO₂H, halo, C₁-C₆ haloalkyl, or C₁-C₆ alkyl, provided that at least one R⁴ group is —CO₂H or contains a —CO₂H moiety.

In some embodiments, each R⁴ is independently —CO₂H, halo, C₁-C₃ haloalkyl, or C₁-C₃ alkyl, provided that at least one R⁴ group is —CO₂H or contains a —CO₂H moiety. In some embodiments, R⁴ is —CO₂H. In some embodiments, R⁴ is halo, provided that at least one other R⁴ group is —CO₂H or contains a —CO₂H moiety. In some embodiments, R⁴ is fluoro, chloro, or bromo, provided that at least one other R⁴ group is —CO₂H or contains a —CO₂H moiety. In some embodiments, R⁴ is C₁-C₆ haloalkyl, provided that at least one other R⁴ group is —CO₂H or contains a —CO₂H moiety. In some embodiments, R⁴ is C₁-C₃ haloalkyl, provided that at least one other R⁴ group is —CO₂H or contains a —CO₂H moiety. In some embodiments, R⁴ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃, provided that at least one other R⁴ group is —CO₂H or contains a —CO₂H moiety. In some embodiments, R⁴ is C₁-C₆ alkyl, provided that at least one other R⁴ group is —CO₂H or contains a —CO₂H moiety. In some embodiments, R⁴ is C₁-C₃ alkyl. In some embodiments, R⁴ is methyl, ethyl, n-propyl, or isopropyl, provided that at least one other R⁴ group is —CO₂H or contains a —CO₂H moiety. In some embodiments, R⁴ is methyl, provided that at least one other R⁴ group is —CO₂H or contains a —CO₂H moiety. In some embodiments, each R⁴ is independently —CO₂H or methyl, provided that at least one R⁴ group is —CO₂H or contains a —CO₂H moiety.

In some embodiments two R⁴ groups are taken together with the carbon atoms to which they are attached to form a fused, bridged, or spiro C₃-C₅ cycloalkyl optionally substituted by —CO₂H, provided that at least one R⁴ group is —CO₂H or contains a —CO₂H moiety. In some embodiments, two R⁴ groups are taken together with the carbon atoms to which they are attached to form a fused C₃-C₅ cycloalkyl optionally substituted by —CO₂H, provided that at least one R⁴ group is —CO₂H or contains a —CO₂H moiety. In some embodiments two R⁴ groups are taken together with the carbon atoms to which they are attached to form a bridged C₃-C₅ cycloalkyl optionally substituted by —CO₂H, provided that at least one R⁴ group is —CO₂H or contains a —CO₂H moiety. In some embodiments two R⁴ groups are taken together with the carbon atoms to which they are attached to form a spiro C₃-C₅ cycloalkyl optionally substituted by —CO₂H, provided that at least one R⁴ group is —CO₂H or contains a —CO₂H moiety. In some embodiments two R⁴ groups are taken together with the carbon atoms to which they are attached to form a fused, bridged, or spiro C₃-C₅ cycloalkyl that is not substituted, provided that at least one other R⁴ group is —CO₂H or contains a —CO₂H moiety. In some embodiments two R⁴ groups are taken together with the carbon atoms to which they are attached to form a fused, bridged, or spiro C₃-C₅ cycloalkyl that is substituted by —CO₂H.

In some embodiments, two R⁴ groups are taken together with the carbon atoms to which they are attached to form a fused cyclopropyl or a spiro cyclobutyl, each of which is optionally substituted by —CO₂H, provided that at least one R⁴ group is —CO₂H or contains a —CO₂H moiety. In some embodiments, two R⁴ groups are taken together with the carbon atoms to which they are attached to form a fused cyclopropyl optionally substituted by —CO₂H, provided that at least one R⁴ group is —CO₂H or contains a —CO₂H moiety. In some embodiments, two R⁴ groups are taken together with the carbon atoms to which they are attached to form a spiro cyclobutyl optionally substituted by —CO₂H, provided that at least one R⁴ group is —CO₂H or contains a —CO₂H moiety.

In some embodiments, z is 1-5. In some embodiments, z is 1-3. In some embodiments, z is 1. In some embodiments, z is 2. In some embodiments, z is 3. In some embodiments, z is 4. In some embodiments, z is 5.

In some embodiments,

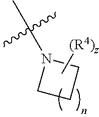

is:

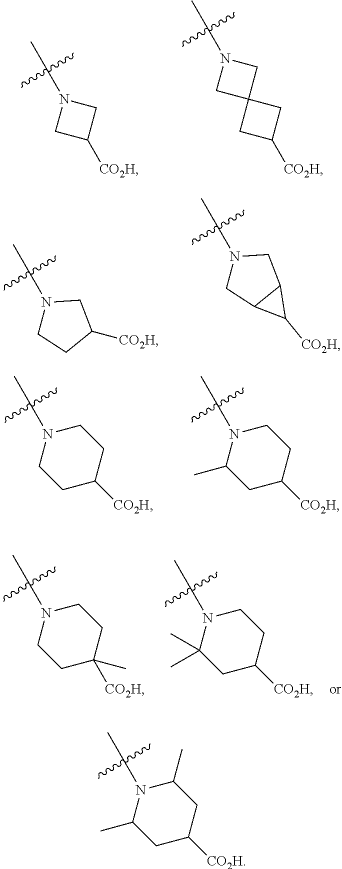

In some embodiments, the compound is Formula (I-a):

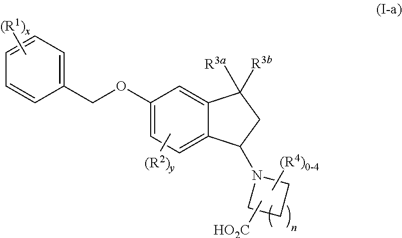

wherein R¹, R², R^{3a}, R^{3b}, x, y, and n are as described for Formula (I), and each R⁴ is independently —CO₂H, halo, C₁-C₆ haloalkyl, or C₁-C₆ alkyl.

In some embodiments, the compound is Formula (I-b):

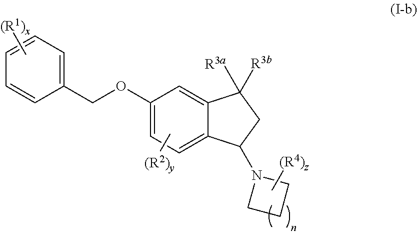

wherein R¹, R², R^{3a}, R^{3b}, x, y, z, and n are as described for Formula (I), and two R⁴ groups are taken together with the carbon atoms to which they are attached to form a fused, bridged, or spiro C₃-C₅ cycloalkyl optionally substituted by —CO₂H, wherein the 4- to 6-membered heterocyclic ring substituted by (R⁴)_{z} is optionally further substituted by up to 3 R⁴ groups, each of which is independently —CO₂H, halo, C₁-C₆ haloalkyl, or C₁-C₆ alkyl, provided that at least one R⁴ group is —CO₂H or contains a —CO₂H moiety.

In some embodiments, the compound of Formula (I) is a compound of Formula (IA), (IB), or (IC):

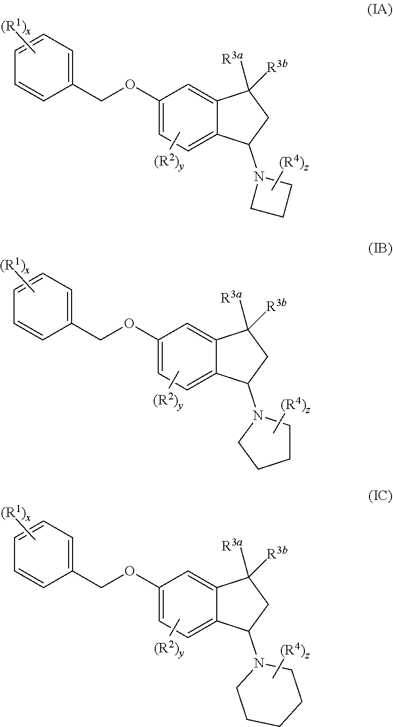

wherein R¹, R², R^{3a}, R^{3b}, R⁴, x, y, and z are as described for Formula (I).

In some embodiments, the compound of Formula (I) is a compound of Formula (Ia), (Ib), or (Ic):

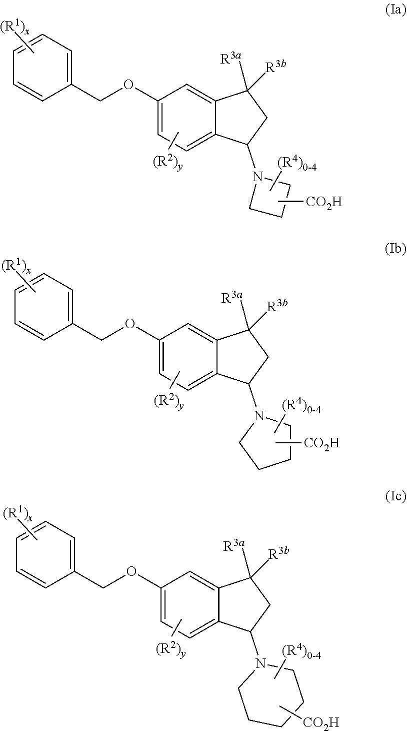

wherein R¹, R², R^{3a}, R^{3b}, R⁴, x, and y are as described for Formula (I).

In some embodiments, the compound of Formula (I) is a compound of Formula (I-1), (I-2), or (I-3):

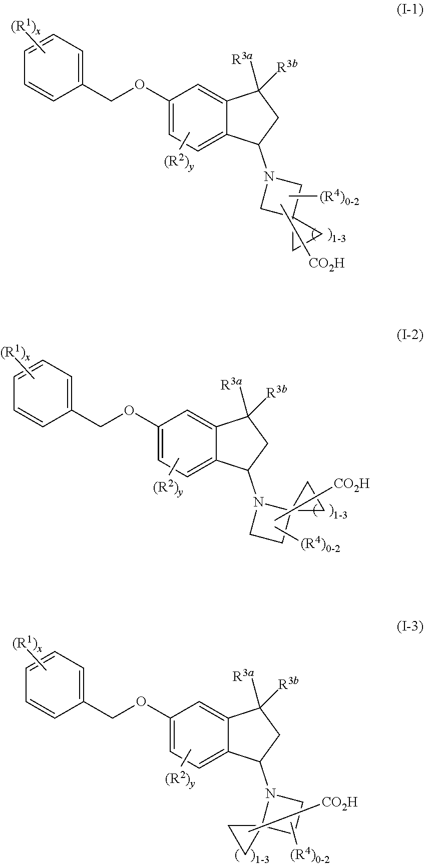

wherein R¹, R², R^{3a}, R^{3b}, x, and y are as described for Formula (I), and each R⁴ is —CO₂H, halo, C₁-C₆ haloalkyl, or C₁-C₆ alkyl. It is understood that when —CO₂H is drawn through both ring systems (i.e., the spiro bicyclic rings), then either of the two rings of the spiro bicyclic ring system can be substituted by —CO₂H. In some embodiments, the heterocyclic ring of the spiro bicyclic ring system is substituted by —CO₂H. In some embodiments, the cycloalkyl ring of the spiro bicyclic ring system is substituted by —CO₂H.

In some embodiments, the compound of Formula (I) is a compound of Formula (I-A), (I-B), or (I-C):

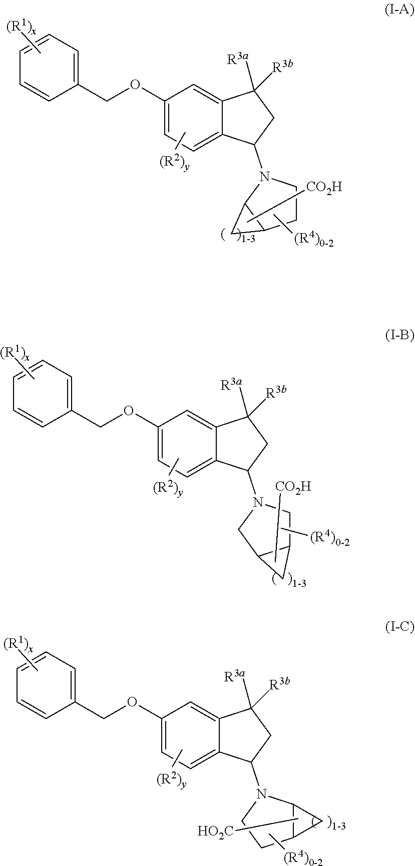

wherein R¹, R², R^{3a}, R^{3b}, x, and y are as described for Formula (I), and each R⁴ is —CO₂H, halo, C₁-C₆ haloalkyl, or C₁-C₆ alkyl. It is understood that when —CO₂H is drawn through both ring systems (i.e., the fused bicyclic rings), then either of the two rings of the fused bicyclic ring system can be substituted by —CO₂H. In some embodiments, the heterocyclic ring of the fused bicyclic ring system is substituted by —CO₂H. In some embodiments, the cycloalkyl ring of the fused bicyclic ring system is substituted by —CO₂H.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of R², R^{3a}, R^{3b}, R⁴, x, y, z, and n, the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (I-a), (I-b), (IA), (IB), (IC), (Ia), (Ib), (Ic), (I-1), (I-2), (I-3), (I-A), (I-B), and (I-C), are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound No. | Structure | Name |
|---|---|---|
| 1 | 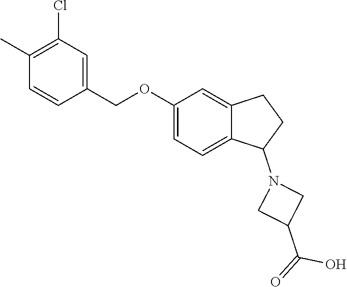 [SMILES: Cc1ccc(COc2ccc3c(c2)CCC3N2CC(C(=O)O)C2)cc1Cl] | 1-(5-((3-chloro-4- methylbenzyl)oxy)-2,3- dihydro-1H-inden-1- yl)azetidine-3-carboxylic acid |
| 2 | 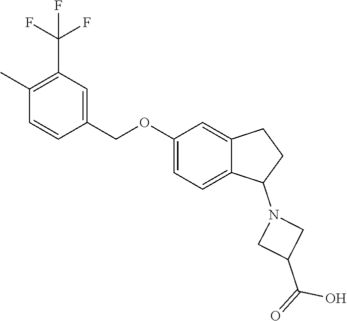 [SMILES: Cc1ccc(COc2ccc3c(c2)CCC3N2CC(C(=O)O)C2)cc1C(F)(F)F] | 1-(5-((4-methyl-3- (trifluoromethyl)benzyl)oxy)- 2,3-dihydro-1H-inden-1- yl)azetidine-3-carboxylic acid |
| 3 | 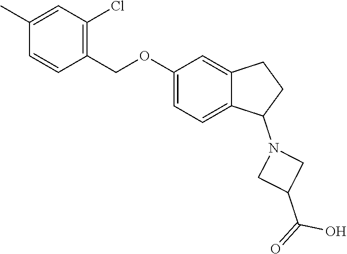 [SMILES: Cc1ccc(COc2ccc3c(c2)CCC3N2CC(C(=O)O)C2)c(Cl)c1] | 1-(5-((2-chloro-4- methylbenzyl)oxy)-2,3- dihydro-1H-inden-1- yl)azetidine-3-carboxylic acid |
| 4 | 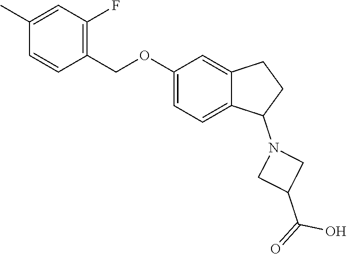 [SMILES: Cc1ccc(COc2ccc3c(c2)CCC3N2CC(C(=O)O)C2)c(F)c1] | 1-(5-((2-fluoro-4- methylbenzyl)oxy)-2,3- dihydro-1H-inden-1- yl)azetidine-3-carboxylic acid |
| 5 | 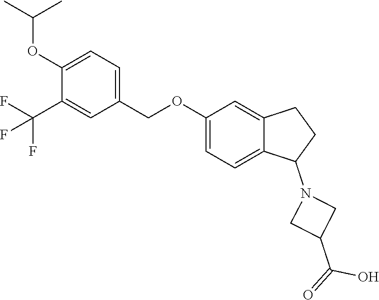 [SMILES: CC(C)Oc1ccc(COc2ccc3c(c2)CCC3N2CC(C(=O)O)C2)cc1C(F)(F)F] | 1-(5-((4-isopropoxy-3- (trifluoromethyl)benzyl)oxy)- 2,3-dihydro-1H-inden-1- yl)azetidine-3-carboxylic acid |
| 6 | 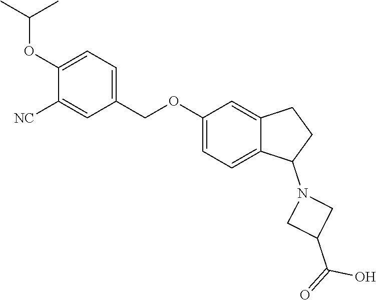 [SMILES: CC(C)Oc1ccc(COc2ccc3c(c2)CCC3N2CC(C(=O)O)C2)cc1C#N] | 1-(5-((3-cyano-4- isopropoxybenzyl)oxy)-2,3- dihydro-1H-inden-1- yl)azetidine-3-carboxylic acid |
| 7 | 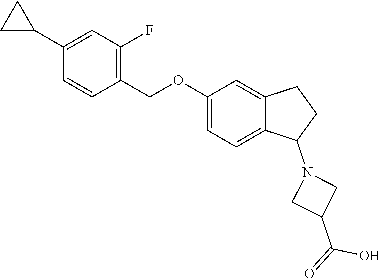 [SMILES: O=C(O)C1CN(C2CCc3cc(OCc4ccc(C5CC5)cc4F)ccc32)C1] | 1-(5-((4-cyclopropyl-2- fluorobenzyl)oxy)-2,3- dihydro-1H-inden-1- yl)azetidine-3-carboxylic acid |
| 8 | 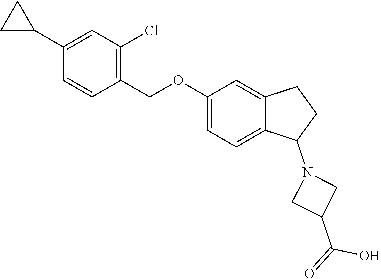 [SMILES: O=C(O)C1CN(C2CCc3cc(OCc4ccc(C5CC5)cc4Cl)ccc32)C1] | 1-(5-((2-chloro-4- cyclopropylbenzyl)oxy)-2,3- dihydro-1H-inden-1- yl)azetidine-3-carboxylic acid |
| 9 | 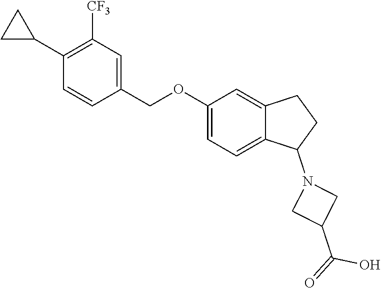 [SMILES: O=C(O)C1CN(C2CCc3cc(OCc4ccc(C5CC5)c(C(F)(F)F)c4)ccc32)C1] | 1-(5-((4-cyclopropyl-3- (trifluoromethyl)benzyl)oxy)- 2,3-dihydro-1H-inden-1- yl)azetidine-3-carboxylic acid |
| 10 | 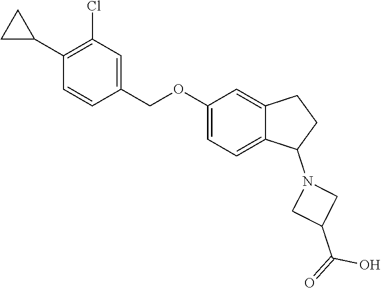 [SMILES: O=C(O)C1CN(C2CCc3cc(OCc4ccc(C5CC5)c(Cl)c4)ccc32)C1] | 1-(5-((3-chloro-4- cyclopropylbenzyl)oxy)-2,3- dihydro-1H-inden-1- yl)azetidine-3-carboxylic acid |
| 11 | 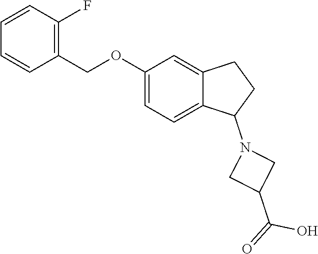 [SMILES: O=C(O)C1CN(C2CCc3cc(OCc4ccccc4F)ccc32)C1] | 1-(5-((2-fluorobenzyl)oxy)- 2,3-dihydro-1H-inden-1- yl)azetidine-3-carboxylic acid |
| 12 | 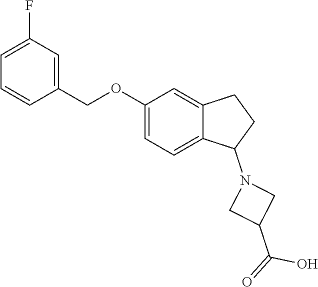 [SMILES: O=C(O)C1CN(C2CCc3cc(OCc4cccc(F)c4)ccc32)C1] | 1-(5-((3-fluorobenzyl)oxy)- 2,3-dihydro-1H-inden-1- yl)azetidine-3-carboxylic acid |
| 13 | 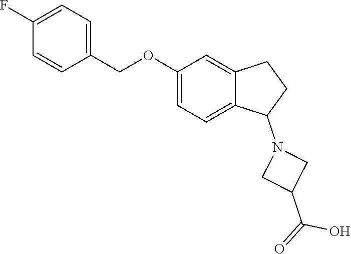 [SMILES: O=C(O)C1CN(C2CCc3cc(OCc4ccc(F)cc4)ccc32)C1] | 1-(5-((4-fluorobenzyl)oxy)- 2,3-dihydro-1H-inden-1- yl)azetidine-3-carboxylic acid |
| 14 | 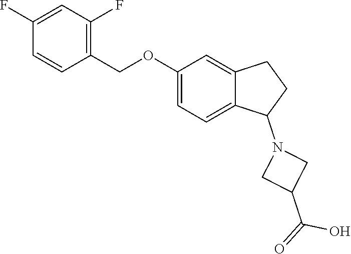 [SMILES: O=C(O)C1CN(C2CCc3cc(OCc4ccc(F)cc4F)ccc32)C1] | 1-(5-((2,4- difluorobenzyl)oxy)-2,3- dihydro-1H-inden-1- yl)azetidine-3-carboxylic acid |
| 15 | 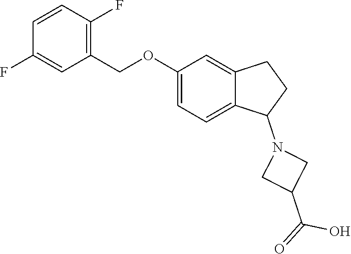 [SMILES: O=C(O)C1CN(C2CCc3cc(OCc4cc(F)ccc4F)ccc32)C1] | 1-(5-((2,5- difluorobenzyl)oxy)-2,3- dihydro-1H-inden-1- yl)azetidine-3-carboxylic acid |
| 16 | 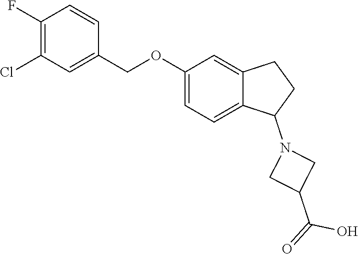 [SMILES: O=C(O)C1CN(C2CCc3cc(OCc4ccc(F)c(Cl)c4)ccc32)C1] | 1-(5-((3-chloro-4- fluorobenzyl)oxy)-2,3- dihydro-1H-inden-1- yl)azetidine-3-carboxylic acid |
| 17 | 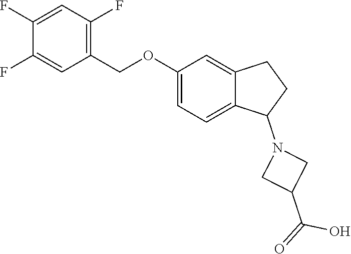 [SMILES: O=C(O)C1CN(C2CCc3cc(OCc4cc(F)c(F)cc4F)ccc32)C1] | 1-(5-((2,4,5- trifluorobenzyl)oxy)-2,3- dihydro-1H-inden-1- yl)azetidine-3-carboxylic acid |
| 18 | 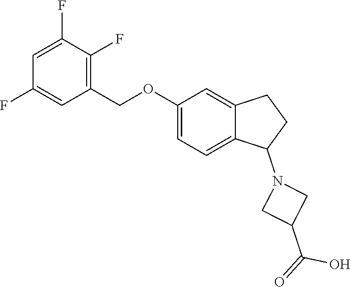 [SMILES: O=C(O)C1CN(C2CCc3cc(OCc4cc(F)cc(F)c4F)ccc32)C1] | 1-(5-((2,3,5- trifluorobenzyl)oxy)-2,3- dihydro-1H-inden-1- yl)azetidine-3-carboxylic acid |
| 19 | 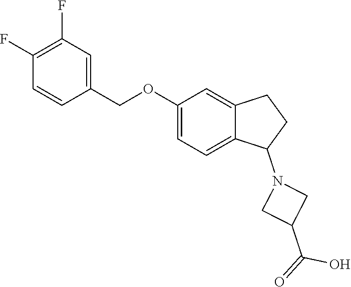 [SMILES: O=C(O)C1CN(C2CCc3cc(OCc4ccc(F)c(F)c4)ccc32)C1] | 1-(5-((3,4- difluorobenzyl)oxy)-2,3- dihydro-1H-inden-1- yl)azetidine-3-carboxylic acid |
| 20 | 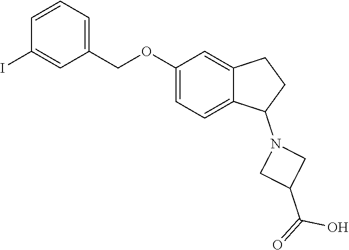 [SMILES: O=C(O)C1CN(C2CCc3cc(OCc4cccc(I)c4)ccc32)C1] | 1-(5-((3-iodobenzyl)oxy)-2,3- dihydro-1H-inden-1- yl)azetidine-3-carboxylic acid |
| 21 | 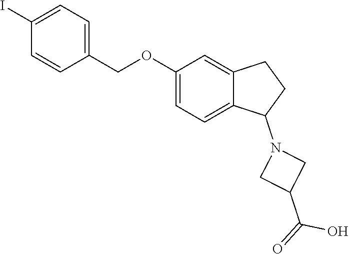 [SMILES: O=C(O)C1CN(C2CCc3cc(OCc4ccc(I)cc4)ccc32)C1] | 1-(5-((4-iodobenzyl)oxy)-2,3- dihydro-1H-inden-1- yl)azetidine-3-carboxylic acid |
| 22 | 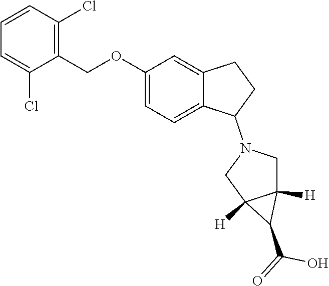 [SMILES: O=C(O)[C@H]1[C@@H]2CN(C3CCc4cc(OCc5c(Cl)cccc5Cl)ccc43)C[C@@H]21] | (1R,5S,6r)-3-(5-((2,6- dichlorobenzyl)oxy)-2,3- dihydro-1H-inden-1-yl)-3- azabicyclo[3.1.0]hexane-6- carboxylic acid |
| 23 | 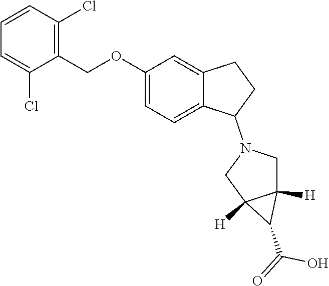 [SMILES: O=C(O)[C@@H]1[C@@H]2CN(C3CCc4cc(OCc5c(Cl)cccc5Cl)ccc43)C[C@@H]21] | (1R,5S,6s)-3-(5-((2,6- dichlorobenzyl)oxy)-2,3- dihydro-1H-inden-1-yl)-3- azabicyclo[3.1.0]hexane-6- carboxylic acid |
| 24a | 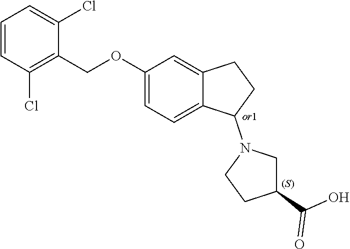 | (3S)-1-(5-((2,6- dichlorobenzyl)oxy)-2,3- dihydro-1H-inden-1- yl)pyrrolidine-3-carboxylic acid |
| 24b | 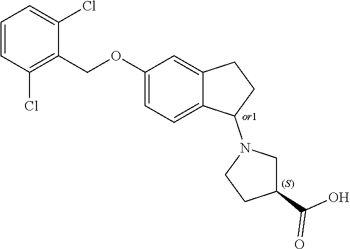 | (3S)-1-(5-((2,6- dichlorobenzyl)oxy)-2,3- dihydro-1H-inden-1- yl)pyrrolidine-3-carboxylic acid |
| 25a | 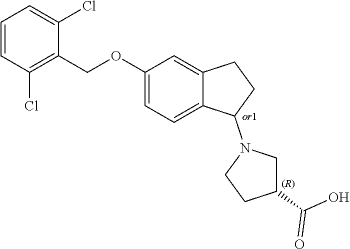 | (3R)-1-(5-((2,6- dichlorobenzyl)oxy)-2,3- dihydro-1H-inden-1- yl)pyrrolidine-3-carboxylic acid |
| 25b | 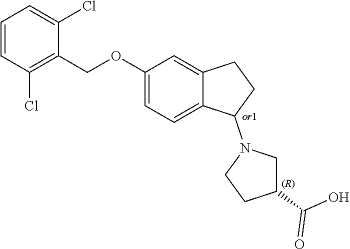 | (3R)-1-(5-((2,6- dichlorobenzyl)oxy)-2,3- dihydro-1H-inden-1- yl)pyrrolidine-3-carboxylic acid |
| 26a | 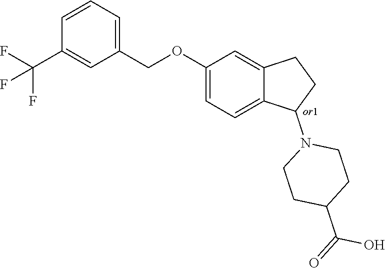 [SMILES: O=C(O)C1CCN(C2CCc3cc(OCc4cccc(C(F)(F)F)c4)ccc32)CC1] | 1-(5-((3- (trifluoromethyl)benzyl)oxy)- 2,3-dihydro-1H-inden-1- yl)piperidine-4-carboxylic acid |
| 26b | 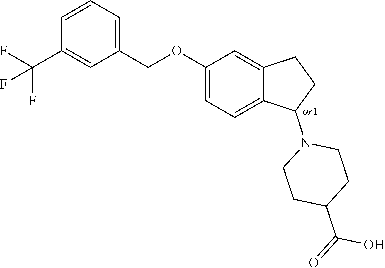 [SMILES: O=C(O)C1CCN(C2CCc3cc(OCc4cccc(C(F)(F)F)c4)ccc32)CC1] | 1-(5-((3- (trifluoromethyl)benzyl)oxy)- 2,3-dihydro-1H-inden-1- yl)piperidine-4-carboxylic acid |
| 27 | 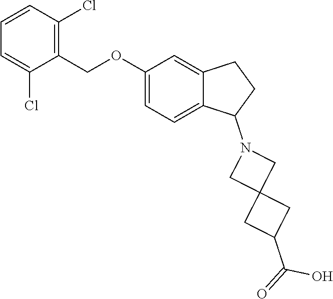 [SMILES: O=C(O)C1CC2(C1)CN(C1CCc3cc(OCc4c(Cl)cccc4Cl)ccc31)C2] | 2-(5-((2,6- dichlorobenzyl)oxy)-2,3- dihydro-1H-inden-1-yl)-2- azaspiro[3.3]heptane-6- carboxylic acid |
| 28 | 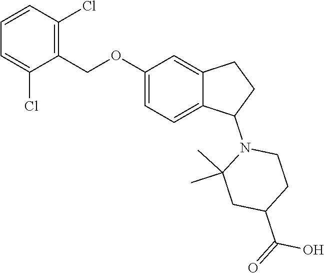 [SMILES: CC1(C)CC(C(=O)O)CCN1C1CCc2cc(OCc3c(Cl)cccc3Cl)ccc21] | 1-(5-((2,6- dichlorobenzyl)oxy)-2,3- dihydro-1H-inden-1-yl)-2,2- dimethylpiperidine-4- carboxylic acid |
| 29 | 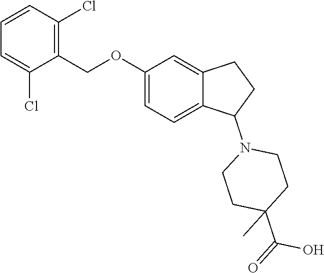 [SMILES: CC1(C(=O)O)CCN(C2CCc3cc(OCc4c(Cl)cccc4Cl)ccc32)CC1] | 1-(5-((2,6- dichlorobenzyl)oxy)-2,3- dihydro-1H-inden-1-yl)-4- methylpiperidine-4-carboxylic acid |
| 30 | 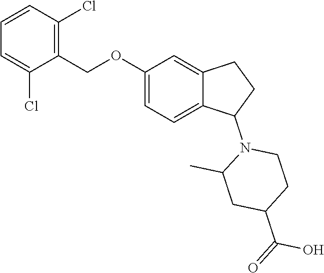 [SMILES: CC1CC(C(=O)O)CCN1C1CCc2cc(OCc3c(Cl)cccc3Cl)ccc21] | 1-(5-((2,6- dichlorobenzyl)oxy)-2,3- dihydro-1H-inden-1-yl)-2- methylpiperidine-4-carboxylic acid |
| 31 | 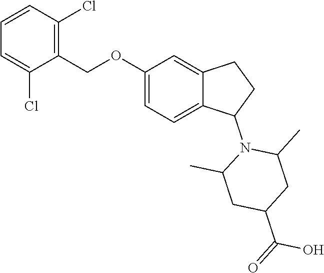 [SMILES: CC1CC(C(=O)O)CC(C)N1C1CCc2cc(OCc3c(Cl)cccc3Cl)ccc21] | 1-(5-((2,6- dichlorobenzyl)oxy)-2,3- dihydro-1H-inden-1-yl)-2,6- dimethylpiperidine-4- carboxylic acid |
| 32 | 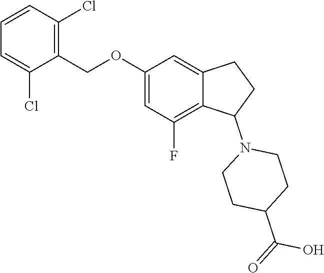 [SMILES: O=C(O)C1CCN(C2CCc3cc(OCc4c(Cl)cccc4Cl)cc(F)c32)CC1] | 1-(5-((2,6- dichlorobenzyl)oxy)-7-fluoro- 2,3-dihydro-1H-inden-1- yl)piperidine-4-carboxylic acid |
| 33 | 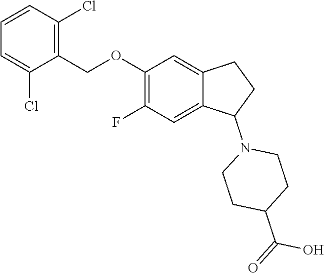 [SMILES: O=C(O)C1CCN(C2CCc3cc(OCc4c(Cl)cccc4Cl)c(F)cc32)CC1] | 1-(5-((2,6- dichlorobenzyl)oxy)-6-fluoro- 2,3-dihydro-1H-inden-1- yl)piperidine-4-carboxylic acid |
| 34 | 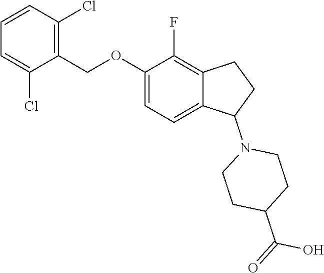 [SMILES: O=C(O)C1CCN(C2CCc3c2ccc(OCc2c(Cl)cccc2Cl)c3F)CC1] | 1-(5-((2,6- dichlorobenzyl)oxy)-4-fluoro- 2,3-dihydro-1H-inden-1- yl)piperidine-4-carboxylic acid |
| 35 | 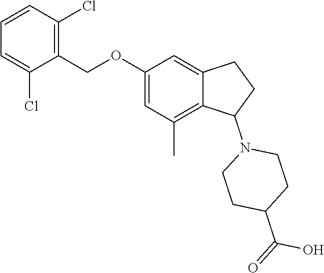 [SMILES: Cc1cc(OCc2c(Cl)cccc2Cl)cc2c1C(N1CCC(C(=O)O)CC1)CC2] | 1-(5-((2,6- dichlorobenzyl)oxy)-7-methyl- 2,3-dihydro-1H-inden-1- yl)piperidine-4-carboxylic acid |
| 36 | 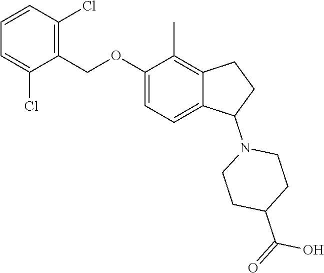 [SMILES: Cc1c(OCc2c(Cl)cccc2Cl)ccc2c1CCC2N1CCC(C(=O)O)CC1] | 1-(5-((2,6- dichlorobenzyl)oxy)-4-methyl- 2,3-dihydro-1H-inden-1- yl)piperidine-4-carboxylic acid |
| 37 | 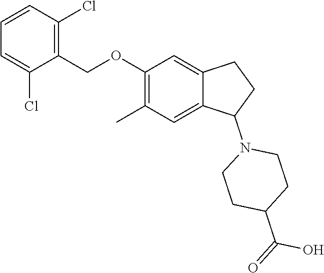 [SMILES: Cc1cc2c(cc1OCc1c(Cl)cccc1Cl)CCC2N1CCC(C(=O)O)CC1] | 1-(5-((2,6- dichlorobenzyl)oxy)-6-methyl- 2,3-dihydro-1H-inden-1- yl)piperidine-4-carboxylic acid |
| 38 | 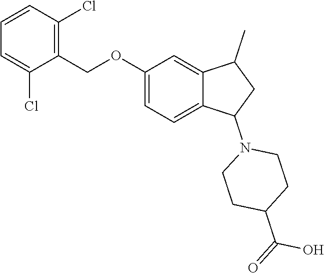 [SMILES: CC1CC(N2CCC(C(=O)O)CC2)c2ccc(OCc3c(Cl)cccc3Cl)cc21] | 1-(5-((2,6- dichlorobenzyl)oxy)-3-methyl- 2,3-dihydro-1H-inden-1- yl)piperidine-4-carboxylic acid |
| 39 | 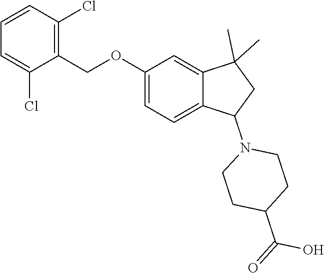 [SMILES: CC1(C)CC(N2CCC(C(=O)O)CC2)c2ccc(OCc3c(Cl)cccc3Cl)cc21] | 1-(5-((2,6- dichlorobenzyl)oxy)-3-methyl- 2,3-dihydro-1H-inden-1- yl)piperidine-4-carboxylic acid |

“or1” indicates that the absolute stereochemistry was not determined.
or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Scheme 1, as well as in the examples set forth herein. It should be noted that one skilled in the art would know how to modify the procedures set forth in the illustrative schemes and examples to arrive at the desired products.

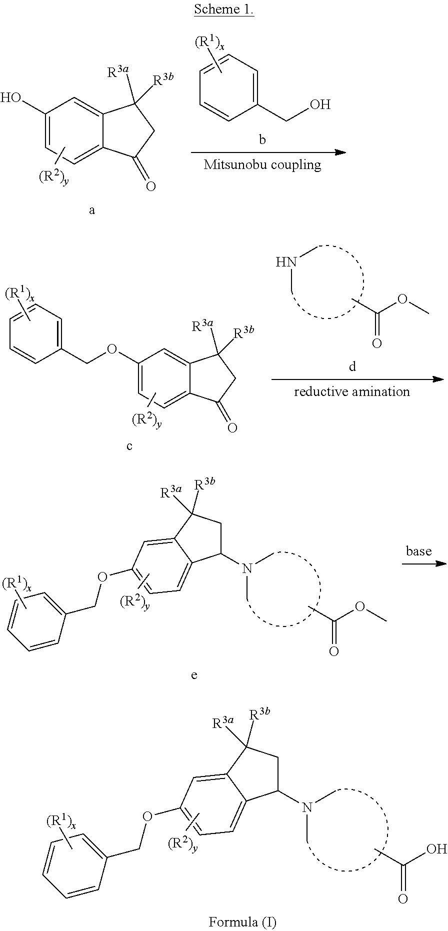

wherein R¹, R², R^{3a}, R^{3b}, x, and y are as described for Formula (I), and

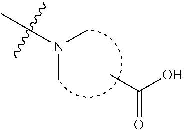

represents a 4-6 membered heterocyclic ring optionally substituted by 1-4 R⁴ groups, wherein each R⁴ is as described for Formula (I), and wherein the 4-6 membered heterocyclic ring or the fused, bridged, or spiro C₃-C₅ cycloalkyl formed by two R⁴ groups being taken together is substituted by —CO₂H.

As outlined in Scheme 1, compounds of Formula (I) can be synthesized from an aryl alcohol a via Mitsunobu coupling with benzyl alcohol b to form intermediate c, which is subsequently reacted with amino acid ester d by reductive amination to form intermediate e. Subsequent hydrolysis of intermediate e provides the compound of Formula (I).

It is understood that compounds of Formula (I) can also be synthesized by other techniques well known to a person skilled in the art. The starting material and intermediates shown in Scheme 1 can undergo derivatizations using techniques well known to a person skilled in the art.

### Methods of Use

Embodiments of the present disclosure provide a method for modulating sphingosine 1-phosphate receptor 5 (S1P5) in a subject in need thereof, the method comprising administering to the subject an effective amount of a compound of Formula (I). Modulation (e.g., inhibition or activation) of S1P5 can be assessed and demonstrated by a wide variety of ways known in the art. Kits and commercially available assays can be utilized for determining whether and to what degree S1P5 has been modulated (e.g., inhibited or activated).

In one aspect, provided herein is a method of modulating S1P5 comprising contacting S1P5 with an effective amount of a compound of Formula (I) or any embodiment or variation thereof. In some embodiments, the compound of Formula (I) inhibits S1P5. In other embodiments, the compound of Formula (I) activates S1P5. In some embodiments, the compound of Formula (I) is an agonist of S1P5. In some embodiments, the compound of Formula (I) is an antagonist of S1P5.

In some embodiments, a compound of Formula (I) modulates the activity of S1P5 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of S1P5 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating a neurological disease in a subject in need thereof, comprising administering to the subject an effective amount of a compound of Formula (I). In some embodiments, provided herein is a method for preventing a neurological disease in a subject in need thereof, comprising administering to the subject an effective amount of a compound of Formula (I). Non-limiting examples of a neurological disease include Alzheimer's disease, multiple sclerosis (MS), amyotrophic lateral schlerosis (ALS), Bell's Palsy, ataxia, cerebral aneurysm, epilepsy, seizures, acute spinal cord injury, Guillain-Barre syndrome, meningitis, Niemann Pick disease, and Parkinson's disease. In some embodiments, the neurological disease is Alzheimer's disease or multiple sclerosis. In some embodiments, the neurological disease is Alzheimer's disease. In some embodiments, the neurological disease is multiple sclerosis.

In some embodiments, administering a compound of Formula (I) to a subject that is predisposed to a neurological disease prevents the subject from developing any symptoms of the neurological disease. In some embodiments, administering a compound of Formula (I) to a subject that is does not yet display symptoms of a neurological disease prevents the subject from developing any symptoms of the neurological disease. In some embodiments, administering a compound of Formula (I) to a subject in need thereof diminishes the extent of the neurological disease in the subject. In some embodiments, administering a compound of Formula (I) to a subject in need thereof stabilizes the neurological disease (prevents or delays the worsening of the neurological disease). In some embodiments, administering a compound of Formula (I) to a subject in need thereof delays the occurrence or recurrence of the neurological disease. In some embodiments, administering a compound of Formula (I) to a subject in need thereof slows the progression of the neurological disease. In some embodiments, administering a compound of Formula (I) to a subject in need thereof provides a partial remission of the neurological disease. In some embodiments, administering a compound of Formula (I) to a subject in need thereof provides a total remission of the neurological disease. In some embodiments, administering a compound of Formula (I) to a subject in need thereof decreases the dose of one or more other medications required to treat the neurological disease. In some embodiments, administering a compound of Formula (I) to a subject in need thereof enhances the effect of another medication used to treat the neurological disease. In some embodiments, administering a compound of Formula (I) to a subject in need thereof delays the progression of the neurological disease. In some embodiments, administering a compound of Formula (I) to a subject in need thereof increases the quality of life of the subject having a neurological disease. In some embodiments, administering a compound of Formula (I) to a subject in need thereof prolongs survival of a subject having a neurological disease.

In one aspect, provided herein is method of preventing a subject that is predisposed to a neurological disease from developing any symptoms of the neurological disease, the method comprising administering a compound of Formula (I) to the subject. In some embodiments, provided herein is a method of preventing a subject that does not yet display symptoms of a neurological disease from developing any symptoms of the neurological disease, the method comprising administering a compound of Formula (I) to the subject.

In some aspects, provided herein is a method of diminishing the extent of a neurological disease in a subject, the method comprising administering a compound of Formula (I) to the subject. In some embodiments, provided herein is a method of stabilizing a neurological disease in a subject, the method comprising administering a compound of Formula (I) to the subject. In some embodiments, the method prevents the worsening of the neurological disease. In some embodiments, the method delays the worsening of the neurological disease.

In another aspect, provided herein is a method of delaying the occurrence or recurrence of a neurological disease in a subject, the method comprising administering a compound of Formula (I) to the subject.

In some embodiments, provided herein is a method of slowing the progression of a neurological disease in a subject, the method comprising administering a compound of Formula (I) to the subject. In some embodiments, the method provides a partial remission of the neurological disease. In some embodiments, the method provides a total remission of the neurological disease.

In further aspects, provided herein is a method of decreasing the dose of one or more other medications required to treat a neurological disease in a subject, the method comprising administering a compound of Formula (I) to the subject. In some embodiments, provided herein is a method of enhancing the effect of another medication used to treat a neurological disease in a subject, the method comprising administering a compound of Formula (I) to the subject.

Also provided here is a method of delaying the progression of a neurological disease in a subject, the method comprising administering a compound of Formula (I) to the subject. In some embodiments, the method increases the quality of life of the subject having a neurological disease. In some embodiments, the method prolongs survival of the subject having a neurological disease.

In another aspect, provided herein is a method for treating neurological symptoms caused by a disease in a subject in need thereof, comprising administering to the subject an effective amount of a compound of Formula (I). In some embodiments, provided herein is a method for preventing neurological symptoms caused by a disease in a subject in need thereof, comprising administering to the subject an effective amount of a compound of Formula (I). In some embodiments, administering a compound of Formula (I) to a subject that is predisposed to a disease which causes neurological symptoms prevents the subject from developing any neurological symptoms. In some embodiments, administering a compound of Formula (I) to a subject that is does not yet display neurological symptoms of a disease which causes neurological symptoms prevents the subject from developing any neurological symptoms. In some embodiments, administering a compound of Formula (I) to a subject in need thereof diminishes the extent of the neurological symptoms caused by the disease in the subject. In some embodiments, administering a compound of Formula (I) to a subject in need thereof stabilizes the neurological symptoms of the disease (prevents or delays the worsening of the neurological symptoms). In some embodiments, administering a compound of Formula (I) to a subject in need thereof delays the occurrence or recurrence of the neurological symptoms caused by the disease. In some embodiments, administering a compound of Formula (I) to a subject in need thereof slows the progression of the neurological symptoms caused by the disease. In some embodiments, administering a compound of Formula (I) to a subject in need thereof provides a partial remission of the disease which causes neurological symptoms. In some embodiments, administering a compound of Formula (I) to a subject in need thereof provides a total remission of the disease which causes neurological symptoms. In some embodiments, administering a compound of Formula (I) to a subject in need thereof decreases the dose of one or more other medications required to treat the disease which causes neurological symptoms. In some embodiments, administering a compound of Formula (I) to a subject in need thereof enhances the effect of another medication used to treat the neurological symptoms of the disease. In some embodiments, administering a compound of Formula (I) to a subject in need thereof delays the progression of the disease which causes neurological symptoms. In some embodiments, administering a compound of Formula (I) to a subject in need thereof increases the quality of life of the subject having a disease which causes neurological symptoms. In some embodiments, administering a compound of Formula (I) to a subject in need thereof prolongs survival of a subject having a disease which causes neurological symptoms. In some embodiments, the disease is Niemann-Pick disease.

In some embodiments, compounds of Formula (I) are useful for treating a disorder selected from Alzheimer's disease, arthritis, rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinemia), dilated cardiomyopathy, infertility, female infertility, ovarian failure, premature ovarian failure, fibrotic lung disease, chronic wound healing, cryptogenic fibrosing alveolitis, post-inflammatory interstitial lung disease, fibrosis, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polytnyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and ThI Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CMIL), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignamt Lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple myeloma, multiple systems degenerations (Mencel Dejerine-Thomas Shi-Drager and Machado-Joseph), myasthenia gravis, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, Pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute pain, age-associated memory impairment (AAMI), anxiety attention deficit disorder, attention deficit disorder in general, attention deficit hyperactivity disorder (ADHD), bipolar disorder, cancer pain, central neuropathic pain syndromes, central post-stroke pain, chemotherapy-induced neuropathy, cognitive deficits and dysfunction in psychiatric disorders, cognitive deficits associated with aging and neurodegeneration, cognitive deficits associated with diabetes, cognitive deficits of schizophrenia, complex regional pain syndrome, declines in cognitive function in Alzheimer's and associated dementias, deficits in attention, dementia, dementia associated with Down's syndrome, dementia associated with Lewy bodies, depression in Cushing's syndrome, diminished CNS function associated with traumatic brain injury, diseases with deficits of memory, dizziness, drug abuse, epilepsy, HIV sensory neuropathy, Huntingdon's disease, hyperalgesia including neuropathic pain, inflammation and inflammatory disorders, inflammatory hyperalgesia, inflammatory pain, insulin resistance syndrome, jet lag, lack of circulation, learning, major depressive disorder, medullary thyroid carcinoma, Meniere's disease, metabolic syndrome, mild cognitive impairment, mood alteration, motion sickness, multiple sclerosis pain, narcolepsy, need for new blood vessel growth associated with vascularization of skin grafts and lack of circulation, need for new blood vessel growth associated with wound healing, neuropathic pain, neuropathy, neuropathy secondary to tumor infiltration, noninflammatory pain, obesity, obsessive compulsive disorder, painful diabetic neuropathy, panic disorder, Parkinson disease pain, pathological sleepiness, phantom limb pain, Pick's Disease, polycystic ovary syndrome, post traumatic stress disorder, post-herpetic neuralgia, post-mastectomy pain, post-surgical pain, psychotic depression, schizoaffective disorder, seizures, senile dementia, sepsis syndrome, sleep disorders, smoking cessation, spinal cord injury pain, steroid-induced acute psychosis, sub-categories of neuropathic pain including peripheral neuropathic pain syndromes, substance abuse including alcohol abuse, Syndrome X, Tourette's syndrome, treatment resistant depression, trigeminal neuralgia, type II diabetes, vertigo, and vestibular disorders.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I) administered will depend on such factors as the solubility of the active component, the formulation used and the route of administration.

In some embodiments, a compound of Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

In a particular embodiment, provided herein are unit dosage formulations comprising about 0.1 mg or 100 mg of a compound of Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (I).

A compound of Formula (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

As used in certain of the chemical structures provided in the following Examples, designation of a particular atom with “or1” indicates that the absolute stereochemistry of the indicated atom was not determined.

The following abbreviations may be relevant for the application.

### Abbreviations

| | |
|---|---|
| 2-Me—THF | 2-methyltetrahydrofuran |
| ACN/MeCN | acetonitrile |
| AcOH | acetic acid |
| aq | aqueous |
| DCM | dichloromethane |
| DIAD | diisopropyl azodicarboxylate |
| DMF | dimethylformamide |
| DMSO | dimethyl sulfoxide |
| EA | ethyl acetate |
| equiv | equivalents |
| ESI | electrospray ionization |
| EtOAc | ethyl acetate |
| EtOH | ethanol |
| h | hour(s) |
| Hex | hexanes |
| HPLC | high performance liquid chromatography |
| LCMS | liquid chromatography mass spectrometry |
| MeOH | methanol |
| min | min(s) |
| Pd(dppf)Cl₂ | [1,1′-bis(diphenylphosphino)ferrocene]dichloropalladium(II) |
| Pd₂(dba)₃ | tris(dibenzylideneacetone)dipalladium(0) |
| PE | petroleum ether |
| Ph | phenyl |
| PPh₃ | triphenylphosphine |
| RockPhos | [(2-di-tert-butylphosphino-3-methoxy-6-methyl-2′,4′,6′- |
| Pd G3 | triisopropyl-1,1′-biphenyl)-2-(2- |
| | aminobiphenyl)]palladium(II) methanesulfonate |
| RT | retention time |
| sat. | saturated |
| t- | 2-(di-tert-butylphosphino)-2′,4′,6′-triisopropyl-3,6- |
| BuBrettphos | dimethoxy-1,1′-biphenyl |
| TFA | trifluoroacetic acid |
| THF | tetrahydrofuran |
| Ti(OiPr)₄ | titanium(IV) isopropoxide |
| TLC | thin layer chromatography |
| TMSCHN₂ | diazomethyl(trimethyl)silane |

### Synthetic Examples

### Example S1. Synthesis of 1-(5-((3-chloro-4-methylbenzyl)oxy)-2,3-dihydro-1H-inden-1-yl)-azetidine-3-carboxylic acid (1)

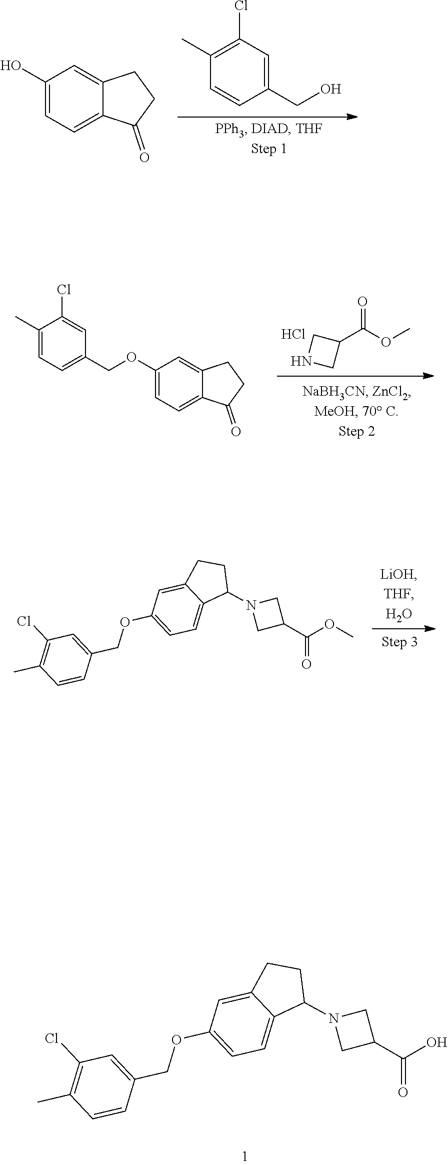

### Synthesis of 5-((3-chloro-4-methylbenzyl)oxy)-2,3-dihydro-1H-inden-1-one

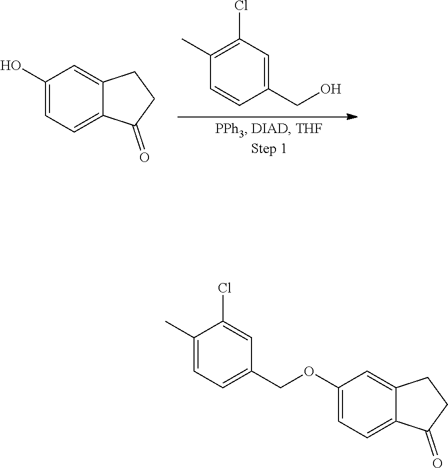

To a solution of 5-hydroxyindan-1-one (1.2 g, 8.11 mmol, 1.00 equiv) in THF (15.0 mL) was added (3-chloro-4-methyl-phenyl) methanol (1.5 g, 9.62 mmol, 1.20 equiv) and PPh₃ (3 mg, 12.2 mmol, 1.50 equiv). The resulting solution was stirred for 10 min at room temperature which was followed by the addition of DIAD (0.8 mL, 12.2 mmol, 1.50 equiv). The resulting mixture was flushed for 5 min with nitrogen and was stirred overnight. LCMS showed the reaction was complete. The mixture was purified by flash chromatography on silica gel (EA:PE=1:6) to afford 5-((3-chloro-4-methylbenzyl)oxy)-2,3-dihydro-1H-inden-1-one (2.0 g, 86%) as a yellow solid. LCMS (ESI, m/z): 287 [M+H]⁺.

### Synthesis of methyl 1-(5-((3-chloro-4-methylbenzyl)oxy)-2,3-dihydro-1H-inden-1-yl)azetidine-3-carboxylate

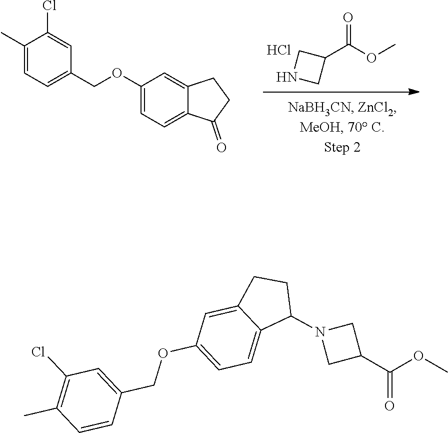

To a solution of NaBH₃CN (3.1 g, 54.3 mmol, 4.10 equiv) in methanol (15.0 mL) was added 2 M ZnCl₂ in THF (14.0 mL, 27.1 mmol, 2.00 equiv) and the mixture was stirred at room temperature for 10 min. 5-((3-Chloro-4-methylbenzyl)oxy)-2,3-dihydro-1H-inden-1-one (3.9 g, 19.9 mmol, 1.50 equiv) and methyl azetidine-3-carboxylate hydrochloride (2.8 g, 13.3 mmol, 1.00 equiv) were added. The resulting mixture was stirred overnight at 70° C. LCMS showed the reaction was complete. The mixture was purified by flash chromatography on silica gel (EA) to afford methyl 1-(5-((3-chloro-4-methylbenzyl)-oxy)-2,3-dihydro-1H-inden-1-yl)azetidine-3-carboxylate (3.4 g, 8.83 mmol, 66%) as a light-yellow oil. LCMS (ESI, m/z): 386 [M+H]⁺.

### Synthesis of 1-(5-((3-chloro-4-methylbenzyl)oxy)-2,3-dihydro-1H-inden-1-yl)-azetidine-3-carboxylic acid

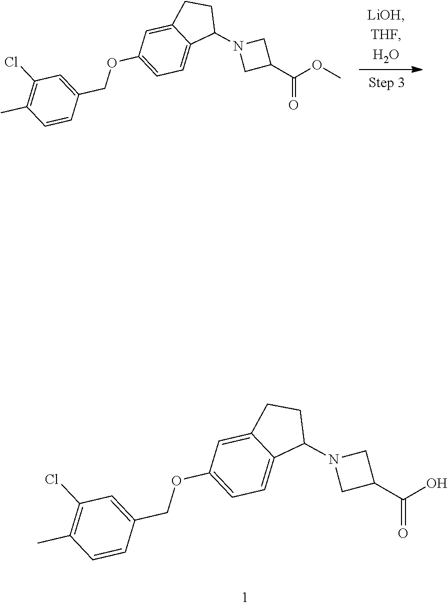

To a solution of methyl 1-(5-((3-chloro-4-methylbenzyl)oxy)-2,3-dihydro-1H-inden-1-yl)azetidine-3-carboxylate (600 mg, 1.55 mmol, 1.00 equiv) in THF (10.0 mL) and water (1.0 mL) was added LiOH H₂O (132 mg, 3.14 mmol, 2.00 equiv). The resulting mixture was stirred overnight. LCMS showed the reaction was complete. The crude product was purified by Prep-HPLC (Column: Sunfire prep C18 column, 30*150 mm, 5 m; Mobile Phase A: Water (0.05% HCl), Mobile Phase B: ACN; Flow rate: 60 mL/min; Gradient: 10% B to 39% B in 7 min; 254/210 nm; RT: 7.32 min) to give 11-(5-((3-chloro-4-methylbenzyl)oxy)-2,3-dihydro-1H-inden-1-yl)azetidine-3-carboxylic acid (106 mg, 18%) as an off-white solid.

LCMS (ESI, m/z): 372 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 95% B in 2.00 min, hold at 95% for 0.70 min, 95% B to 5% B in 0.05 min; 220 nm; RT: 1.584 min.

¹H NMR (400 MHz, Methanol-d₄) δ 7.45 (d, J=8.4 Hz, 1H), 7.40 (s, 1H), 7.34-7.23 (m, 2H), 7.02 (d, J=2.4 Hz, 1H), 6.96 (dd, J=8.5, 2.4 Hz, 1H), 5.10 (s, 2H), 4.50-4.41 (m, 2H), 4.37-4.28 (m, 2H), 3.71-3.62 (m, 1H), 3.16 (dt, J=16.5, 8.2 Hz, 1H), 2.97 (ddd, J=16.8, 9.3, 2.8 Hz, 1H), 2.52 (dq, J=16.6, 8.4 Hz, 1H), 2.37 (s, 3H), 2.20 (dt, J=14.1, 5.4 Hz, 1H).

### Example S2. Synthesis of 1-(5-((4-methyl-3-(trifluoromethyl)benzyl)oxy)-2,3-dihydro-1H-inden-1-yl)azetidine-3-carboxylic acid (2)

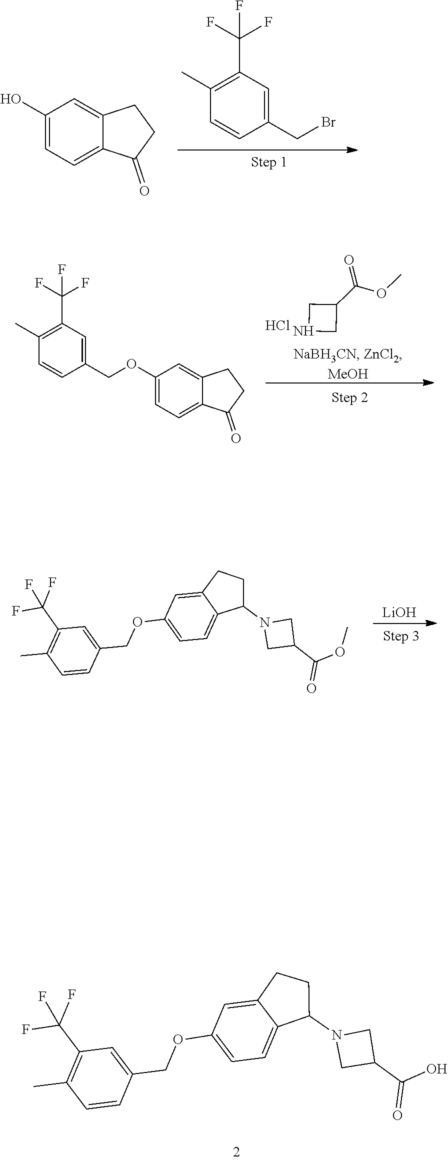

### Synthesis of 5-((4-methyl-3-(trifluoromethyl)benzyl)oxy)-2,3-dihydro-1H-inden-1-one

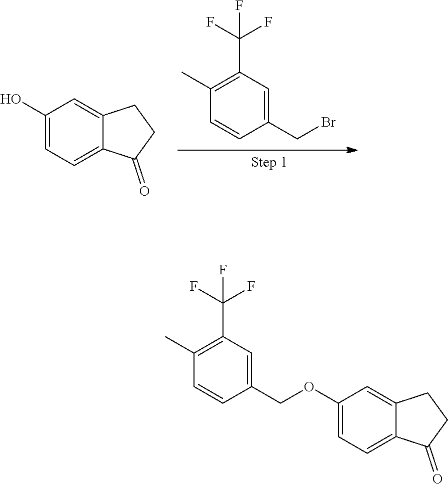 [SMILES: O=C1CCc2cc(O)ccc21 ; Cc1ccc(CBr)cc1C(F)(F)F ; Cc1ccc(COc2ccc3c(c2)CCC3=O)cc1C(F)(F)F]

To a solution of 5-hydroxyindan-1-one (350 mg, 2.36 mmol, 1.00 equiv) and 4-(bromomethyl)-1-methyl-2-(trifluoromethyl)benzene (657 mg, 2.60 mmol, 1.1 equiv) in MeCN (10.0 mL) was added K₂CO₃ (978 mg, 7.09 mmol, 3.00 equiv). The flask was evacuated and flushed with nitrogen. The mixture was stirred overnight at 50° C. under nitrogen atmosphere. LCMS showed the reaction was complete. The resulting solution was diluted with ethyl acetate and filtered. The filtrate was concentrated under reduced pressure. The residue was purified by flash chromatography with ethyl acetate/petroleum ether (1:6) to afford 5-((4-methyl-3-(trifluoromethyl)benzyl)oxy)-2,3-dihydro-1H-inden-1-one (420 mg, 55%) as a yellow solid. LCMS (ESI, m/z): 321 [M+H]⁺.

### Synthesis of methyl 1-(5-((4-methyl-3-(trifluoromethyl)benzyl)oxy)-2,3-dihydro-1H-inden-1-yl)azetidine-3-carboxylate

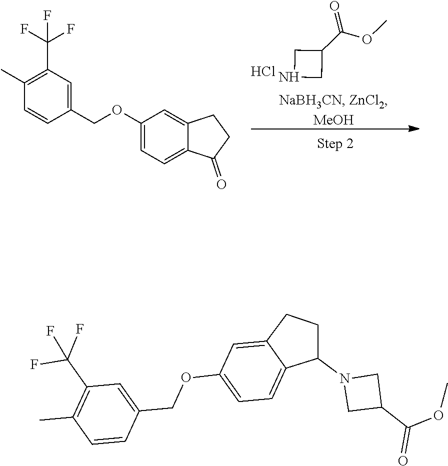

To a solution of NaBH₃CN (330 mg, 5.24 mmol, 4.00 equiv) in methanol (10.0 mL) was added 2 M ZnCl₂ in THF (1.4 mL, 2.62 mmol, 2.00 equiv) and then stirred for 10 min. The resulting mixture was added methyl azetidine-3-carboxylate hydrochloride (298 mg, 1.97 mmol, 1.50 equiv) and 5-((4-methyl-3-(trifluoromethyl)benzyl)oxy)-2,3-dihydro-1H-inden-1-one (420 mg, 1.31 mmol, 1.00 equiv). The flask was evacuated and flushed with nitrogen. The mixture was stirred overnight at 70° C. under nitrogen atmosphere. LCMS showed the reaction was complete. The resulting mixture was concentrated under reduced pressure. The crude product was purified by flash chromatography on silica gel with dichloromethane/methanol (25:1) to afford methyl 1-(5-((4-methyl-3-(trifluoromethyl)benzyl)oxy)-2,3-dihydro-1H-inden-1-yl)azetidine-3-carboxylate (439 mg, 80%) as a yellow solid. LCMS (ESI, m/z): 420 [M+H]⁺.

### Synthesis of 1-(5-((4-methyl-3-(trifluoromethyl)benzyl)oxy)-2,3-dihydro-1H-inden-1-yl)azetidine-3-carboxylic acid

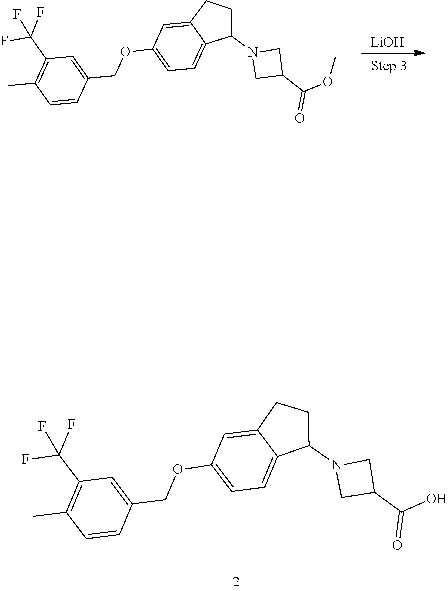 [SMILES: COC(=O)C1CN(C2CCc3cc(OCc4ccc(C)c(C(F)(F)F)c4)ccc32)C1 ; "LiOH" ; Cc1ccc(COc2ccc3c(c2)CCC3N2CC(C(=O)O)C2)cc1C(F)(F)F]

To a solution of methyl 1-(5-((4-methyl-3-(trifluoromethyl)benzyl)oxy)-2,3-dihydro-1H-inden-1-yl)azetidine-3-carboxylate (350 mg, 0.830 mmol, 1.00 equiv) in THE (10.0 mL) and water (1.0 mL) was added LiOH H₂O (70 mg, 1.67 mmol, 2.00 equiv). The mixture was stirred overnight at room temperature. LCMS showed the reaction was complete. The reaction was diluted with 10 mL water and the pH adjusted to 2˜3 with 4 M hydrochloric acid, then extracted with EA. The resulting mixture was concentrated under reduced pressure and the residue was purified by Prep-HPLC (Column: Sunfire prep C18 column, 30*150 mm, 5 m; Mobile Phase A: Water (0.05% HCl), Mobile Phase B: ACN; Flow rate: 60 mL/min; Gradient: 10% B to 39% B in 7 min; 254/210 nm; RT: 7.00 min) to afford 1-(5-((4-methyl-3-(trifluoromethyl)benzyl)-oxy)-2,3-dihydro-1H-inden-1-yl)azetidine-3-carboxylic acid (165 mg, 99%) as an off-white solid.

LCMS (ESI, m/z): 406 [M+H]+. Analytic Conditions: column: Poroshell HPH-C18, 3.0*50 mm, 2.7 m; mobile phase A::Water/0.05% TFA, mobile phase B: Acetonitrile/0.05% TFA, flow rate: 1.20 mL/min; gradient: 5% B to 100% B in 2.00 min, hold at 100% B for 0.70 min, 100% B to 5% B in 0.05 min; 210 nm; RT: 1.629 min.

¹H NMR (400 MHz, Methanol-d₄) δ 7.68 (s, 1H), 7.55 (d, J=7.6 Hz, 1H), 7.46 (d, J=8.4 Hz, 1H), 7.37 (d, J=7.9 Hz, 1H), 7.02 (d, J=2.4 Hz, 1H), 6.95 (dd, J=8.5, 2.5 Hz, 1H), 5.15 (s, 2H), 4.84 (s, 1H), 4.70-4.15 (m, 4H), 3.64 (s, 1H), 3.13 (dt, J=16.6, 8.2 Hz, 1H), 2.95 (ddd, J=16.9, 9.4, 2.9 Hz, 1H), 2.57-2.43 (m, 4H), 2.15 (m, 1H).

### Example S3. Synthesis of 1-(5-((2-chloro-4-methylbenzyl)oxy)-2,3-dihydro-1H-inden-1-yl)-azetidine-3-carboxylic acid (3)

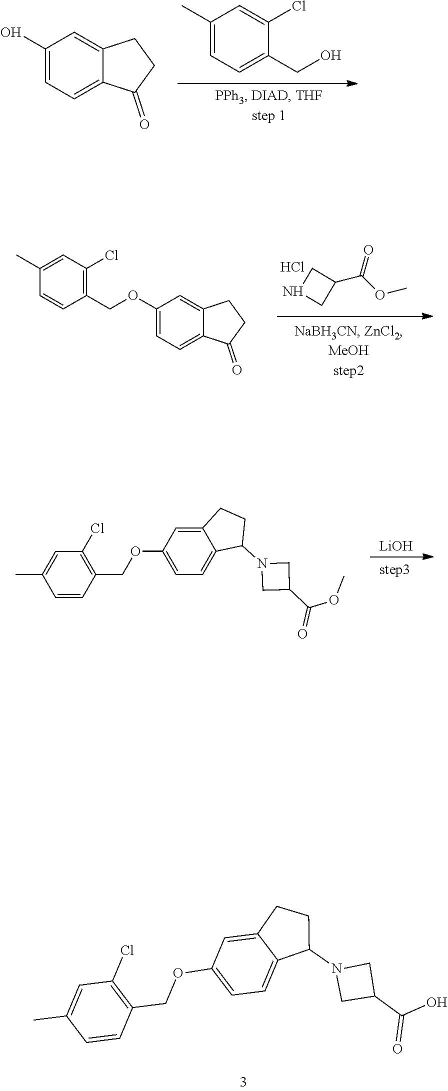

### Synthesis of 5-((2-chloro-4-methylbenzyl)oxy)-2,3-dihydro-1H-inden-1-one

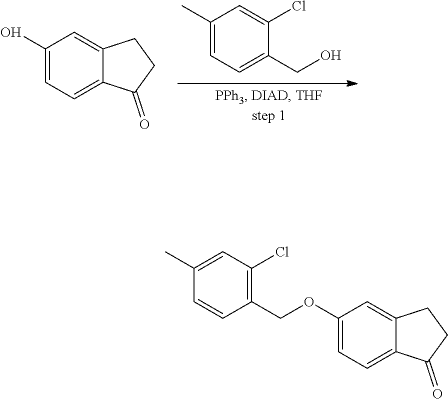

To a solution of (2-chloro-4-methyl-phenyl)methanol (500 mg, 3.19 mmol, 1.00 equiv) in THF (30 mL) was added 5-hydroxyindan-1-one (520 mg, 3.51 mmol, 1.10 equiv), PPh₃ (1256 mg, 4.79 mmol, 1.50 equiv). Then DIAD (0.9 mL, 4.79 mmol, 1.50 equiv) was added dropwise at 0° C. The mixture was stirred at room temperature overnight. The mixture was concentrated and purified by flash chromatography (PE:EA=5:1) to afford 5-((2-chloro-4-methylbenzyl)oxy)-2,3-dihydro-1H-inden-1-one (800 mg, 87%). LCMS (ESI, m/z): 287 [M+H]⁺.

### Synthesis of methyl 1-(5-((2-chloro-4-methylbenzyl)oxy)-2,3-dihydro-1H-inden-1-yl)azetidine-3-carboxylate

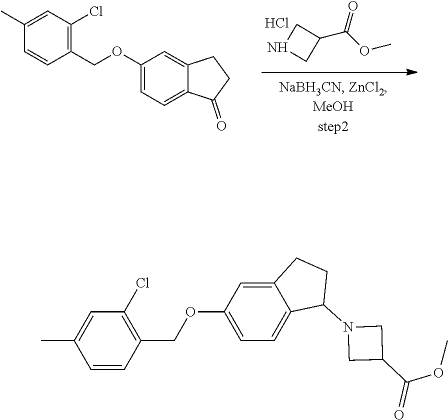

To a solution of NaBH₃CN (439 mg, 6.97 mmol, 4.00 equiv) in methanol (20 mL) was added ZnCl₂ (1.7 mL, 3.49 mmol, 2.00 equiv). Then 5-((2-chloro-4-methyl-benzyl)oxy)-2,3-dihydro-1H-inden-1-one (500 mg, 1.74 mmol, 1.00 equiv) and methyl azetidine-3-carboxylate hydrochloride (528 mg, 3.49 mmol, 2.00 equiv) were added. The mixture was stirred at 70° C. overnight. LCMS showed the desired product. The mixture was diluted with water (5 mL), extracted with EA (2×5 mL). The organic layers were combined and dried with Na₂SO₄. The solid was filtered out and the filtrate under reduced pressure to afford methyl 1-(5-((2-chloro-4-methylbenzyl)-oxy)-2,3-dihydro-1H-inden-1-yl)azetidine-3-carboxylate (300 mg, 44%). LCMS (ESI, m/z): 386 [M+H]⁺.

### Synthesis of 1-(5-((2-chloro-4-methylbenzyl)oxy)-2,3-dihydro-1H-inden-1-yl)azetidine-3-carboxylic acid

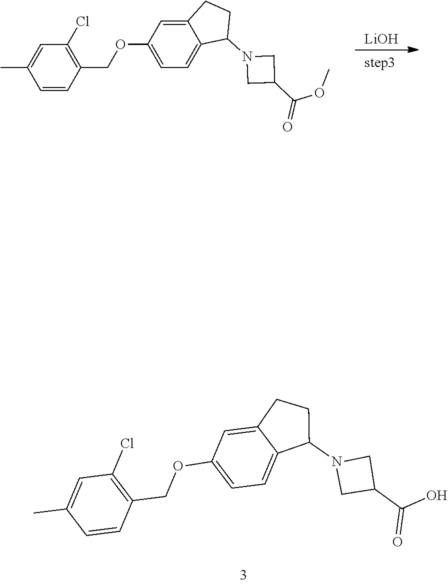

To a solution of methyl 1-(5-((2-chloro-4-methylbenzyl)oxy)-2,3-dihydro-1H-inden-1-yl)azetidine-3-carboxylate (300 mg, 0.780 mmol, 1.00 equiv) in THE (5 mL) and water (0.5 mL) was added LiOH H₂O (65 mg, 1.56 mmol, 2.00 equiv). The mixture was stirred overnight at room temperature. LCMS showed target product formation. The reaction was diluted with 10 mL water and adjusted to pH 6 with 4 M HCl. The solution was concentrated under reduced pressure and the residue was purified by Prep-HPLC (Column: YMC-Actus Triart C18 ExRS. 30*250, 5 m; Mobile Phase A: water (10 mmol/L NH₄HCO₃+0.1% NH₃·H₂O), Mobile Phase B: ACN; Flow rate: 60 mL/min; Gradient: 25% B to 50% B in 7 min; 254/210 nm; RT: 5.65 min) to afford 11-(5-((2-chloro-4-methylbenzyl)oxy)-2,3-dihydro-1H-inden-1-yl)azetidine-3-carboxylic acid (111 mg, 38%) as an off-white solid.

LCMS (ESI, m/z): 372 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 95% B in 2.00 min, hold at 95% B for 0.70 min, 95% B to 5% B in 0.05 min; 220 nm; RT: 1.603 min.

¹H NMR (400 MHz, Methanol-d₄) δ 7.47 (d, J=8.4 Hz, 1H), 7.39 (d, J=7.8 Hz, 1H), 7.27 (s, 1H), 7.13 (d, J=7.9 Hz, 1H), 7.01 (s, 1H), 6.94 (d, J=8.5 Hz, 1H), 5.14 (s, 2H), 4.87 (s, 1H), 4.70-4.20 (m, 4H), 4.32 (s, 2H), 3.65 (s, 1H), 3.14 (dt, J=16.6, 8.1 Hz, 1H), 3.01-2.90 (m, 1H), 2.51 (dq, J=16.3, 8.5 Hz, 1H), 2.33 (s, 3H), 2.23-2.13 (m, 1H).

### Example S4. Synthesis of 1-(5-((2-fluoro-4-methylbenzyl)oxy)-2,3-dihydro-1H-inden-1-yl)-azetidine-3-carboxylic acid (4)

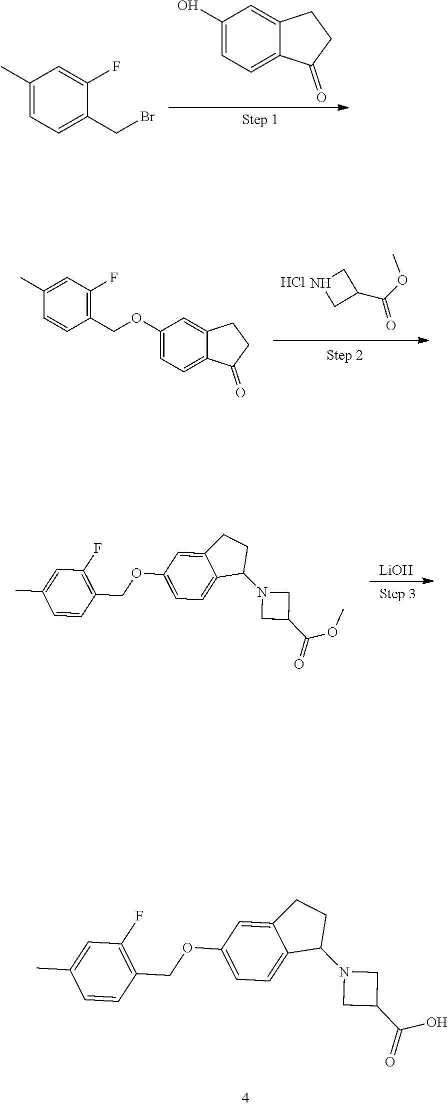

### Synthesis of 5-((2-fluoro-4-methylbenzyl)oxy)-2,3-dihydro-1H-inden-1-one

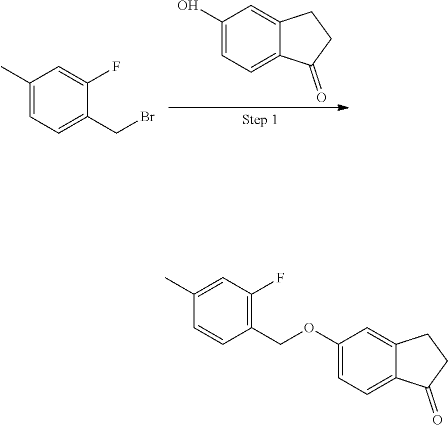

To a solution of 5-hydroxyindan-1-one (270 mg, 1.82 mmol, 1.00 equiv) and 1-(bromomethyl)-2-fluoro-4-methyl-benzene (407 mg, 2.00 mmol, 1.10 equiv) in MeCN (10.0 mL) was added K₂CO₃ (754 mg, 5.47 mmol, 3.00 equiv). The flask was evacuated and flushed with nitrogen. The mixture was stirred for 2 h at 80° C. under nitrogen atmosphere. LCMS showed the reaction was complete. The resulting solution was diluted with EA and filtered. The filtrate was concentrated under reduced pressure. The residue was purified by flash chromatography on silica gel with ethyl acetate/petroleum ether (1:1) to afford 5-((2-fluoro-4-methylbenzyl)oxy)-2,3-dihydro-1H-inden-1-one (393 mg, 80%). LCMS (ESI, m/z): 271 [M+H]⁺.

### Synthesis of methyl 1-(5-((2-fluoro-4-methylbenzyl)oxy)-2,3-dihydro-1H-inden-1-yl)azetidine-3-carboxylate

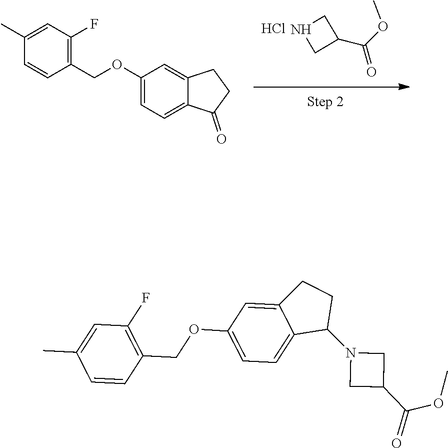

To a solution of NaBH₃CN (279 mg, 4.44 mmol, 4.00 equiv) in methanol (10.0 mL) was added 2 M ZnCl₂ in THF (1.2 mL, 2.22 mmol, 2.00 equiv) and stirred at RT for 10 min. To the resulting mixture was added methyl azetidine-3-carboxylate hydrochloride (252 mg, 1.66 mmol, 1.50 equiv) and 5-((2-fluoro-4-methylbenzyl)oxy)-2,3-dihydro-1H-inden-1-one (300 mg, 1.11 mmol, 1.00 equiv). The mixture was stirred overnight at 70° C. under nitrogen atmosphere. LCMS showed the reaction was complete. The resulting mixture was concentrated and purified by flash chromatography on silica gel with dichloromethane/methanol (25:1) to afford methyl 1-(5-((2-fluoro-4-methyl-benzyl)oxy)-2,3-dihydro-1H-inden-1-yl)azetidine-3-carboxylate (328 mg, 80%) as a yellow solid. LCMS (ESI, m/z): 370 [M+H]⁺.

### Synthesis of 1-(5-((2-fluoro-4-methylbenzyl)oxy)-2,3-dihydro-1H-inden-1-yl)-azetidine-3-carboxylic acid

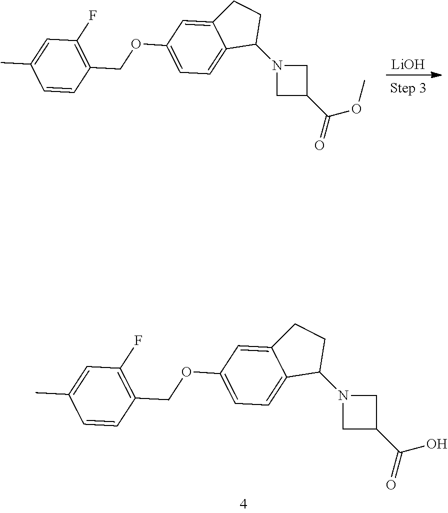

To a solution of methyl 1-(5-((2-fluoro-4-methylbenzyl)oxy)-2,3-dihydro-1H-inden-1-yl)azetidine-3-carboxylate (300 mg, 0.810 mmol, 1.00 equiv) in THE (10.0 mL) and water (1.0 mL) was added LiOH H₂O (68 mg, 1.62 mmol, 2.00 equiv). The mixture was stirred overnight at room temperature. LCMS showed the reaction was complete. The reaction was diluted with 10 mL water and the pH was adjusted to 2 with 4 M HCl. The resulting solution was concentrated under reduced pressure and purified by Prep-HPLC (Column: Sunfire prep C18 column, 30*150 mm, 5 m; Mobile Phase A: Water (0.05% HCl), Mobile Phase B: ACN; Flow rate: 60 mL/min; Gradient: 10% B to 39% B in 7 min; 254/210 nm; RT: 7.42 min) to afford 1-(5-((2-fluoro-4-methylbenzyl)oxy)-2,3-dihydro-1H-inden-1-yl)azetidine-3-carboxylic acid (138 mg, 95%) as an off-white solid.

LCMS (ESI, m/z): 356 [M+H]⁺. Analytic Conditions: column: Shim-pack XR-ODS, 3.0*50 mm, 2.2 m; mobile phase A: water (Water/0.05% TFA), mobile phase B: Acetonitrile/0.05% TFA, flow rate: 1.20 mL/min; gradient: 15% B to 100% B in 2.00 min, hold at 100% B for 0.70 min, 100% B to 5% B in 0.05 min; 210 nm; RT: 1.524 min.

¹H NMR (400 MHz, Methanol-d₄) δ 7.46 (d, J=8.4 Hz, 1H), 7.32 (t, J=7.8 Hz, 1H), 7.02-6.89 (m, 4H), 5.11 (s, 2H), 4.83 (s, 1H), 4.50-4.00 (br, 4H), 3.63 (s, 1H), 3.12 (dt, J=16.5, 8.2 Hz, 1H), 2.94 (ddd, J=16.8, 9.3, 2.8 Hz, 1H), 2.50 (dq, J=16.8, 8.5 Hz, 1H), 2.34 (s, 3H), 2.20 (m, 1H).

### Example S5. Synthesis of 1-(5-((4-isopropoxy-3-(trifluoromethyl)benzyl)oxy)-2,3-dihydro-1H-inden-1-yl)azetidine-3-carboxylic acid (5)

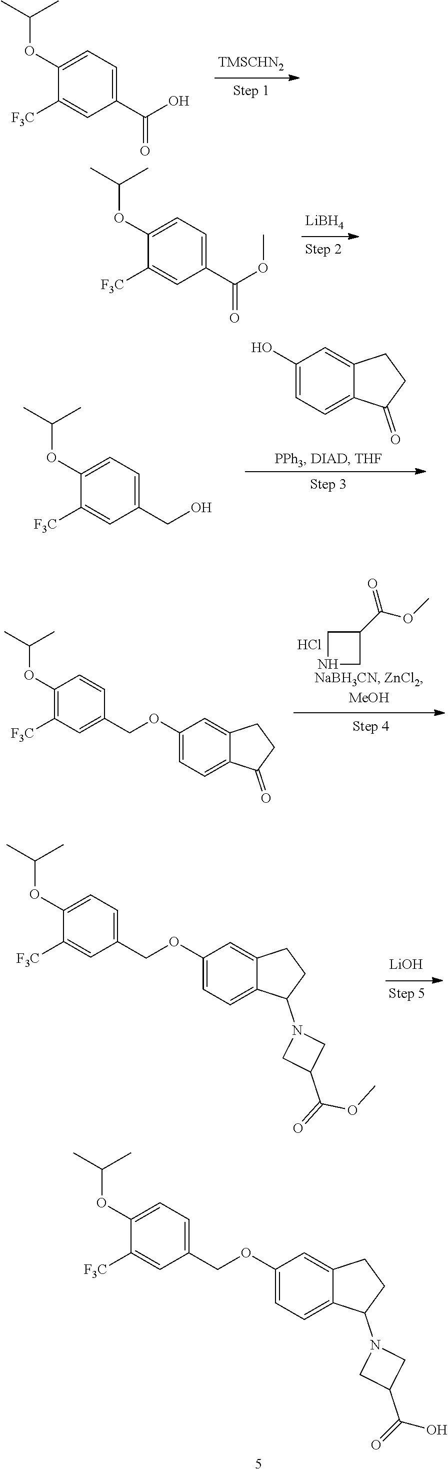

### Synthesis of methyl 4-isopropoxy-3-(trifluoromethyl) benzoate

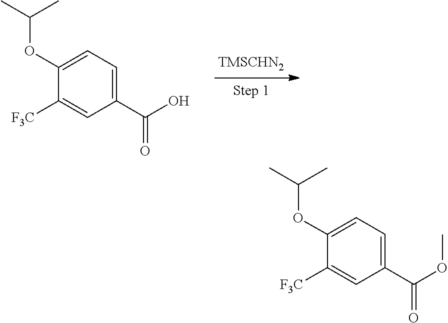 [SMILES: CC(C)Oc1ccc(C(=O)O)cc1C(F)(F)F ; COC(=O)c1ccc(OC(C)C)c(C(F)(F)F)c1]

Into a 200 ml round bottom flask was placed a solution of 4-isopropoxy-3-(trifluoromethyl) benzoic acid (600 mg, 2.42 mmol, 1.00 equiv) in DCM (8.0 mL) and methanol (2.0 mL). This was followed by the addition of diazomethyl(trimethyl)silane (1.8 mL, 3.63 mmol, 1.50 equiv) dropwise over a period of 3 min at 0° C. under a nitrogen atmosphere. The pale yellow solution was allowed to come to room temperature over a period of 15 min. The reaction was stirred for 1.5 h at this temperature. The excess diazomethyl(trimethyl)silane was quenched by the slow addition of acetic acid (0.5 ml) at room temperature until the yellow color was discharged. The reaction mixture was concentrated under vacuum and diluted with EA 20.0 mL. The organic phase was washed with and saturated NaHCO₃ solution 10 mL and brine 10 mL, dried over sodium sulfate and concentrated to give methyl 4-isopropoxy-3-(trifluoromethyl) benzoate (620 mg, 97%) (crude) as a yellow oil. LCMS (ESI, m/z): 263 [M+H]⁺.

### Synthesis of (4-isopropoxy-3-(trifluoromethyl)phenyl)methanol

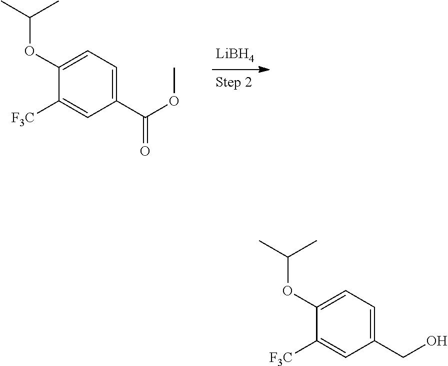 [SMILES: COC(=O)c1ccc(OC(C)C)c(C(F)(F)F)c1 ; "LiBH4" ; CC(C)Oc1ccc(CO)cc1C(F)(F)F]

To a solution of methyl 4-isopropoxy-3-(trifluoromethyl)benzoate (693 mg, 2.64 mmol, 1.00 equiv) in THE (5.0 mL) and ethanol (0.5 mL) was added LiBH₄ (0.2 mL, 7.93 mmol, 3.00 equiv). The reaction was stirred for 1 h at room temperature. LCMS showed the reaction was complete. The mixture was diluted with 10 mL water and extracted with EA (1×10 mL). The organic layer was washed with 3 mL HCl (0.5 mol/L). The organic phase was dried over anhydrous Na₂SO₄. The solid was filtered out and the filtrate was concentrated under reduced pressure to give (4-iso-propoxy-3-(trifluoromethyl)phenyl)methanol (553 mg, 89%) as a yellow oil. LCMS (ESI, m/z): 235 [M+H]⁺.

### Synthesis of 5-((4-isopropoxy-3-(trifluoromethyl)benzyl)oxy)-2,3-dihydro-1H-inden-1-one

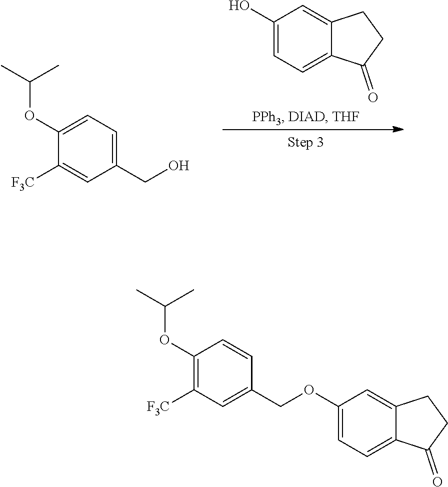 [SMILES: CC(C)Oc1ccc(CO)cc1C(F)(F)F ; O=C1CCc2cc(O)ccc21 ; CC(C)Oc1ccc(COc2ccc3c(c2)CCC3=O)cc1C(F)(F)F]

Into a 50 ml 3-necked round bottom flask was placed a solution of (4-isopropoxy-3-(trifluoromethyl)phenyl)methanol (610 mg, 2.60 mmol, 1.00 equiv) and 5-hydroxy-indan-1-one (501 mg, 3.39 mmol, 1.30 equiv) in THF (10.0 mL). Under nitrogen atmosphere PPh₃ (1024 mg, 3.91 mmol, 1.50 equiv) in THF was added. The reaction was stirred for 20 min. This was followed by the addition of DIAD (789 mg, 3.91 mmol, 1.50 equiv) in THF, which was added dropwise with stirring at 0° C. in 15 min. The reaction was allowed to warm up to room temperature and stirred for 2 h. LCMS showed the reaction was complete. The reaction was quenched with 50 mL water and extracted with 1×50 mL EA. The organic phase was dried over anhydrous Na₂SO₄. The solid was filtered out and the filtrate was concentrated under vacuum. The residue was purified by flash chromatography on silica gel (EA:PE=1:6) to give 5-((4-isopropoxy-3-(trifluoromethyl)benzyl)oxy)-2,3-dihydro-1H-inden-1-one (690 mg, 72%) as a yellow oil. LCMS (ESI, m/z): 365 [M+H]⁺.

### Synthesis of methyl 1-(5-((4-isopropoxy-3-(trifluoromethyl)benzyl)oxy)-2,3-dihydro-1H-inden-1-yl)azetidine-3-carboxylate

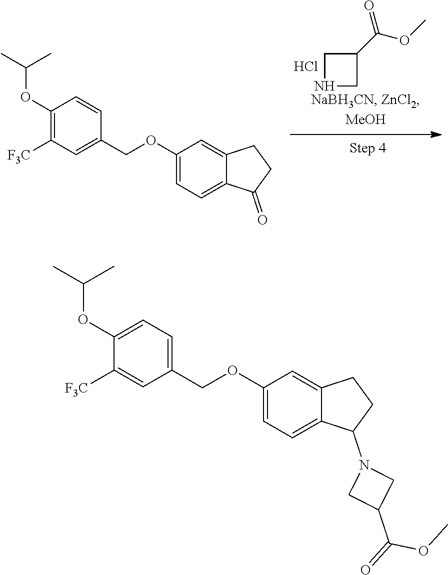

To a solution of the NaBH₃CN (476 mg, 7.57 mmol, 3.00 equiv) in MeOH (10.0 mL) was added ZnCl₂ (2.0 mL, 3.79 mmol, 2.00 equiv). The resulting mixture was stirred for 15 min at room temperature. This was followed by the addition of 5-((4-isopropoxy-3-(trifluoromethyl)benzyl)oxy)-2,3-dihydro-1H-inden-1-one (690 mg, 1.89 mmol, 1.00 equiv) and methyl azetidine-3-carboxylate (327 mg, 2.84 mmol, 1.50 equiv). The reaction mixture was stirred at 60° C. for 12 h. LCMS showed the reaction was complete. The resulting solution was quenched with 30 mL water and extracted with EA 1×30 mL. The organic phase was dried over anhydrous Na₂SO₄ and the solid was filtered out. The filtrate was concentrated under reduced pressure to give methyl 1-(5-((4-isopropoxy-3-(trifluoromethyl)benzyl)oxy)-2,3-dihydro-1H-inden-1-yl)azetidine-3-carboxylate (1.0 g, 118%) (crude) as a yellow oil. LCMS (ESI, m/z): 464 [M+H]⁺.

### Synthesis of 1-(5-((4-isopropoxy-3-(trifluoromethyl)benzyl)oxy)-2,3-dihydro-1H-inden-1-yl)azetidine-3-carboxylic acid

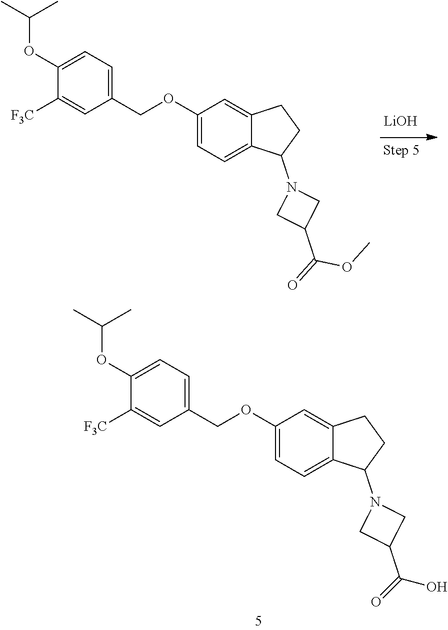

To a solution of the methyl 1-(5-((4-isopropoxy-3-(trifluoromethyl)-benzyl)oxy)-2,3-dihydro-1H-inden-1-yl)azetidine-3-carboxylate (300 mg, 0.650 mmol, 1.00 equiv) in THE (10.0 mL) and water (1.0 mL) was added LiOH·H₂O (81 mg, 1.94 mmol, 3.00 equiv). The reaction was stirred for 2 h at room temperature. LCMS showed the reaction was complete. The mixture was concentrated under vacuum and diluted with water. The pH value of the solution was adjusted to 5-6 with 4 M HCl. The residue was purified by Prep-HPLC (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 22% B to 52% B in 7 min; 254/210 nm; RT: 7.77 min) to give 1-(5-((4-isopropoxy-3-(trifluoromethyl)benzyl)oxy)-2,3-dihydro-1H-inden-1-yl)azetidine-3-carboxylic acid (110 mg, 37%) as a white solid.

LCMS (ESI, m/z): 450 [M+H]⁺. Analytic Conditions: column: Titank C18, 3.0*50 mm, 3.0 m; mobile phase A: water (5 mM NH₄HCO₃), mobile phase B: acetonitrile, flow rate: 1.50 mL/min; gradient: 10% B to 95% B in 1.80 min, 60% B to 95% B in 0.45 min, hold at 95% for 0.80 min, 95% B to 10% B in 0.15 min; 224 nm; RT: 1.443 min.

¹H NMR (400 MHz, Methanol-d₄) δ 7.61 (d, J=9.5 Hz, 2H), 7.44 (d, J=8.4 Hz, 1H), 7.19 (d, J=8.5 Hz, 1H), 7.00 (d, J=2.4 Hz, 1H), 6.94 (dd, J=8.6, 2.4 Hz, 1H), 5.08 (s, 2H), 4.81-4.72 (m, 1H), 4.30 (s, 1H), 4.15 (s, 2H), 3.30 (s, 1H), 3.10 (m, 2H), 2.90 (m, 1H), 2.45 (m, 1H), 2.15 (m, 1H), 1.35 (s, 6H).

### Example S6. Synthesis of 1-(5-((3-cyano-4-isopropoxybenzyl)oxy)-2,3-dihydro-1H-inden-1-yl)-azetidine-3-carboxylic acid (6)

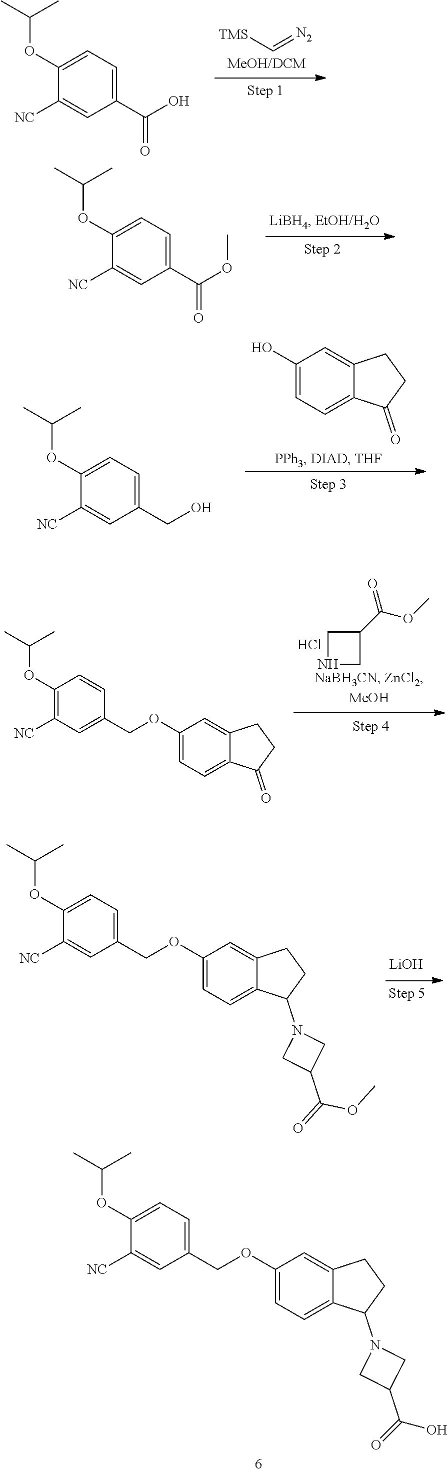

### Synthesis of methyl 3-cyano-4-isopropoxy-benzoate

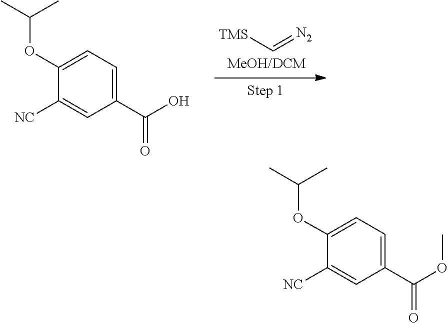

To a solution of 3-cyano-4-isopropoxy-benzoic acid (600 mg, 2.92 mmol, 1.00 equiv) in MeOH (2.0 mL) and DCM (8.0 mL) was added diazomethyl(trimethyl)silane (1.9 mL, 3.80 mmol, 1.30 equiv) dropwise at 0° C. The reaction was stirred for 2 h at room temperature. LCMS showed the reaction was complete. AcOH (1.5 mL) was added into the reaction mixture slowly until no more bubbles appeared. The resulting solution was concentrated under vacuum and then extracted with EA (3×50 mL). The organic layer was washed with a solution of NaHCO₃ and brine. The combined organic phase was dried with anhydrous Na₂SO₄. The solid was filtered out and the filtrate was concentrated under vacuum to give methyl 3-cyano-4-isopropoxy-benzoate (610 mg, 95%) as a yellow oil. LCMS (ESI, m/z): 220 [M+H]⁺.

### Synthesis of 5-(hydroxymethyl)-2-isopropoxy-benzonitrile

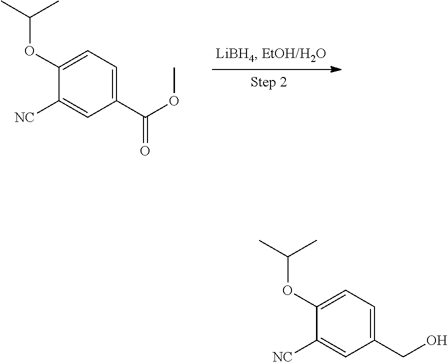 [SMILES: COC(=O)c1ccc(OC(C)C)c(C#N)c1 ; "LiBH4, EtOH/H2O" ; CC(C)Oc1ccc(CO)cc1C#N]

To a solution of methyl 3-cyano-4-isopropoxy-benzoate (570 mg, 2.60 mmol, 1.00 equiv) in THE (20.0 mL) and EtOH (2.0 mL) was added LiBH₄ (170 mg, 7.80 mmol, 3.00 equiv). The reaction was stirred for 16 h at room temperature. TLC showed the reaction was complete. The reaction mixture was quenched with ice-water (40 mL) and extracted with EA (3×50 mL). The combined organic phase was dried over Na₂SO₄. The solid was filtered out and the filtrate was concentrated under reduced pressure to give 5-(hydroxymethyl)-2-isopropoxy-benzonitrile (480 mg, 97%) as a yellow oil. LCMS (ESI, m/z): 192 [M+H]⁺.

### Synthesis of 2-isopropoxy-5-(((1-oxo-2,3-dihydro-1H-inden-5-yl)oxy)methyl)-benzonitrile

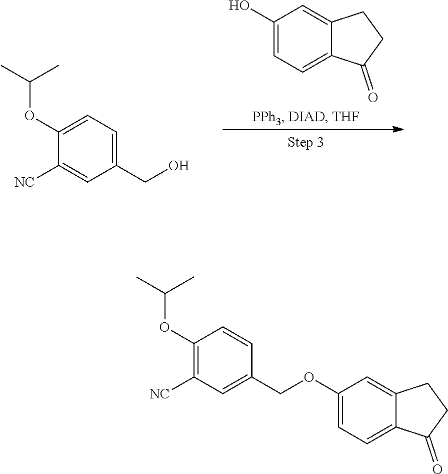 [SMILES: CC(C)Oc1ccc(CO)cc1C#N ; O=C1CCc2cc(O)ccc21 ; CC(C)Oc1ccc(COc2ccc3c(c2)CCC3=O)cc1C#N]

To a solution of 5-(hydroxymethyl)-2-isopropoxy-benzonitrile (430 mg, 2.25 mmol, 1.00 equiv) in THE (30.0 mL) was added 5-hydroxyindan-1-one (400 mg, 2.70 mmol, 1.20 equiv). To the above mixture was added PPh₃ (885 mg, 3.37 mmol, 1.50 equiv) at 0° C. and the reaction was stirred for 0.5 h at room temperature. This was followed by dropwise addition of DIAD (0.66 mL, 3.37 mmol, 1.50 equiv) at 0° C. The reaction was stirred for 2 h at room temperature. TLC showed the reaction was complete. The mixture was quenched with water (50 mL), extracted with EA (3×50 mL). The organic layer was washed with brine, concentrated and purified by flash chromatography on silica gel (PE/EA=3/1) to afford 2-isopropoxy-5-(((1-oxo-2,3-dihydro-1H-inden-5-yl)oxy)methyl)benzonitrile (440 mg, 61%) as a yellow solid. LCMS (ESI, m/z): 322 [M+H]⁺.

### Synthesis of methyl 1-(5-((3-cyano-4-isopropoxybenzyl)oxy)-2,3-dihydro-1H-inden-1-yl)azetidine-3-carboxylate

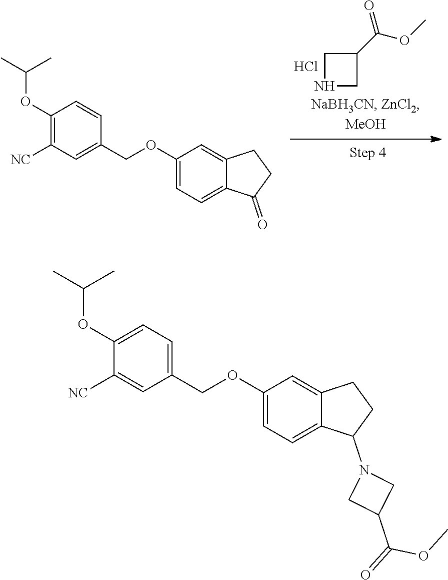

To a solution of NaBH₃CN (297 mg, 4.73 mmol, 4.00 equiv) in MeOH (10.0 mL) was added ZnCl₂ (2 M in THF) (1.2 mL, 2.36 mmol, 2.00 equiv). The reaction was stirred for 15 min at room temperature. 2-Isopropoxy-5-(((1-oxo-2,3-dihydro-1H-inden-5-yl)oxy)methyl)benzonitrile (380 mg, 1.18 mmol, 1.00 equiv) and methyl azetidine-3-carboxylate hydrochloride (233 mg, 1.54 mmol, 2.00 equiv) were added. The reaction was stirred for 16 h at 60° C. TLC showed the reaction was complete. The mixture was quenched with water (40.0 mL), extracted with EA (3×50 mL). The organic layer was washed with brine, dried over anhydrous Na₂SO₄, filtered and concentrated to give methyl 1-(5-((3-cyano-4-isopropoxybenzyl)oxy)-2,3-dihydro-1H-inden-1-yl)azetidine-3-carboxylate (450 mg, 91%) as a yellow oil. LCMS (ESI, m/z): 421 [M+H]⁺.

### Synthesis of 1-(5-((3-cyano-4-isopropoxybenzyl)oxy)-2,3-dihydro-1H-inden-1-yl)azetidine-3-carboxylic acid

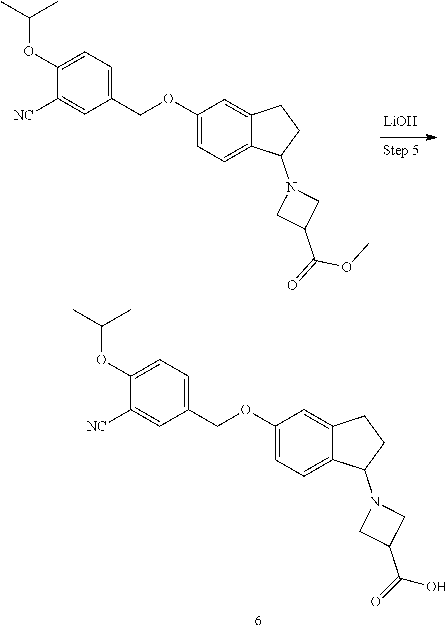

To a solution of the compound methyl 1-(5-((3-cyano-4-isopropoxybenzyl)oxy)-2,3-dihydro-1H-inden-1-yl)azetidine-3-carboxylate (200 mg, 0.480 mmol, 1.00 equiv) in THE (5.0 mL) and water (1.0 mL) was added LiOH H₂O (80 mg, 1.90 mmol, 4.00 equiv). The reaction was stirred for 1 h at room temperature. LCMS showed the reaction was completed. The reaction was diluted with 10 mL water. The pH value of the solution was adjusted to 6 with AcOH and then the solution was concentrated under vacuum. The residue was purified by Prep-HPLC (Column: XBridge Prep OBD C18 Column, 30x 150 mm 5 m; Mobile Phase A: Water (10 mmol/L NH₄HCO₃+0.1% NH₃·H₂O), Mobile Phase B: ACN; Flow rate: 60 mL/min; Gradient: 17% B to 47% B in 7 min; 254/210 nm; RT: 6.48 min) to give 1-(5-((3-cyano-4-isopropoxybenzyl)oxy)-2,3-dihydro-1H-inden-1-yl)azetidine-3-carboxylic acid (101 mg, 52%) as a white solid.

LCMS (ESI, m/z): 407 [M+H]⁺. Analytic Conditions: Column: Shim-pack XR-ODS Column 3.0*50 mm, 2.2 m; Mobile Phase A: Water/0.05% TFA, Mobile Phase B: Acetonitrile/0.05% TFA; Flow rate: 1.20 mL/min; Gradient: 5% B to 100% B in 2.0 min, hold at 95% B for 0.70 min, 100% B to 5% B in 0.05 min; 220 nm; RT: 1.520 min.

¹H NMR (400 MHz, Methanol-d4) δ 7.66 (dq, J=4.5, 2.3 Hz, 2H), 7.45 (d, J=8.4 Hz, 1H), 7.19 (d, J=9.4 Hz, 1H), 7.00 (d, J=2.3 Hz, 1H), 6.90 (dd, J=8.5, 2.5 Hz, 1H), 5.10 (s, 2H), 4.83-4.73 (m, 2H), 4.34-4.24 (m, 2H), 4.16 (t, J=8.7 Hz, 2H), 3.41-3.28 (m, 1H), 3.12 (dt, J=16.4, 8.2 Hz, 1H), 2.96 (ddd, J=16.8, 9.2, 3.0 Hz, 1H), 2.47 (dq, J=15.0, 8.3 Hz, 1H), 2.16 (ddt, J=14.2, 8.1, 2.8 Hz, 1H), 1.39 (d, J=6.1 Hz, 6H).

### Example S7. Synthesis of 1-(5-((4-cyclopropyl-2-fluorobenzyl)oxy)-2,3-dihydro-1H-inden-1-yl)-azetidine-3-carboxylic acid (7)

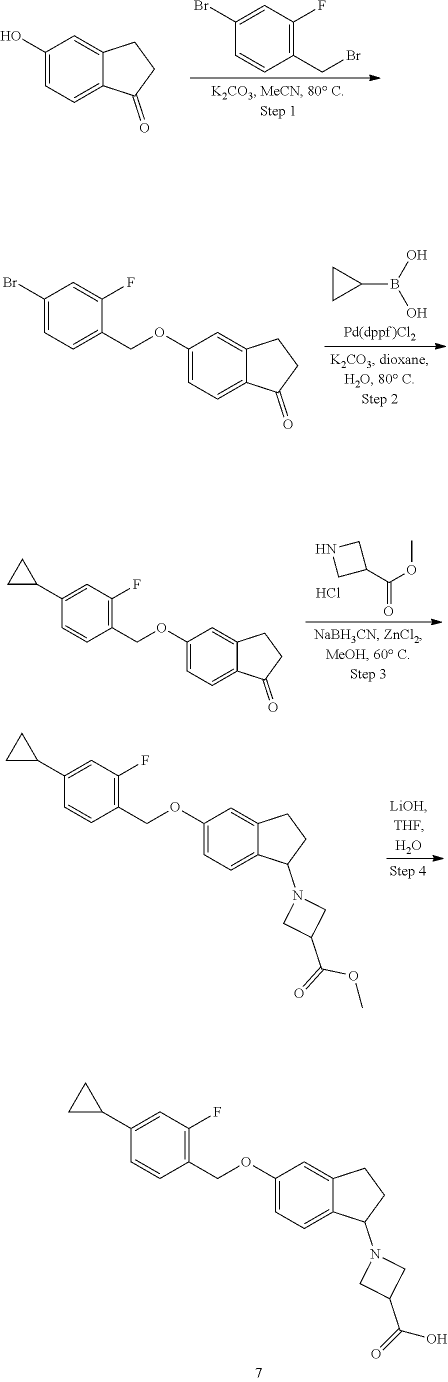

### Synthesis of 5-((4-bromo-2-fluorobenzyl)oxy)-2,3-dihydro-1H-inden-1-one

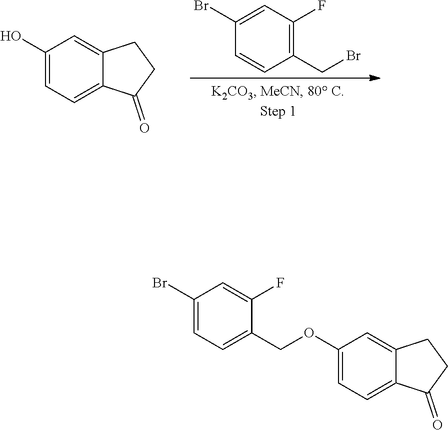 [SMILES: O=C1CCc2cc(O)ccc21 ; Fc1cc(Br)ccc1CBr ; O=C1CCc2cc(OCc3ccc(Br)cc3F)ccc21]

To a solution of 5-hydroxyindan-1-one (600 mg, 4.05 mmol, 1.00 equiv) in MeCN (20.0 mL) was added 4-bromo-1-(bromomethyl)-2-fluoro-benzene (1301 mg, 4.86 mmol, 1.20 equiv) and K₂CO₃ (1676 mg, 12.1 mmol, 3.00 equiv). The resulting mixture was flushed with nitrogen and was stirred for 2 h at 80° C. LCMS showed the reaction was complete. The solid filtered out and the filtrate was concentrated under reduced pressure. The residue was purified by flash chromatography to afford 5-((4-bromo-2-fluorobenzyl)oxy)-2,3-dihydro-1H-inden-1-one (1.3 g, 96%) as a brown solid. LCMS (ESI, m/z): 335 [M+H]⁺.

### Synthesis of 5-((4-cyclopropyl-2-fluorobenzyl)oxy)-2,3-dihydro-1H-inden-1-one

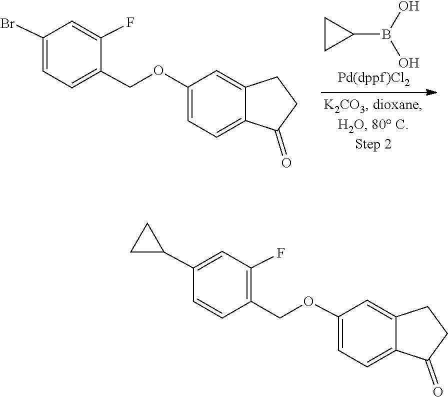 [SMILES: O=C1CCc2cc(OCc3ccc(Br)cc3F)ccc21 ; OB(O)C1CC1 ; O=C1CCc2cc(OCc3ccc(C4CC4)cc3F)ccc21]

To a solution of 5-((4-bromo-2-fluorobenzyl)oxy)-2,3-dihydro-1H-inden-1-one (350 mg, 1.04 mmol, 1.00 equiv) in 1,4-dioxane (10 mL) and water (1 mL) was added cyclopropylboronic acid (179 mg, 2.09 mmol, 2.00 equiv), Pd(dppf)Cl₂ (170 mg, 0.210 mmol, 0.20 equiv)) and K₂CO₃ (432 mg, 3.13 mmol, 3.00 equiv). The resulting mixture was flushed with nitrogen and was stirred for 2 h at 80° C. LCMS showed the reaction was complete. The mixture was purified by Prep-TLC (PE:EA=1:1) to afford 5-((4-cyclopropyl-2-fluorobenzyl)oxy)-2,3-dihydro-1H-inden-1-one (178 mg, 59%) as an off-white solid. LCMS (ESI, m/z): 297 [M+H]⁺.

### Synthesis of methyl 1-(5-((4-cyclopropyl-2-fluorobenzyl)oxy)-2,3-dihydro-1H-inden-1-yl)azetidine-3-carboxylate

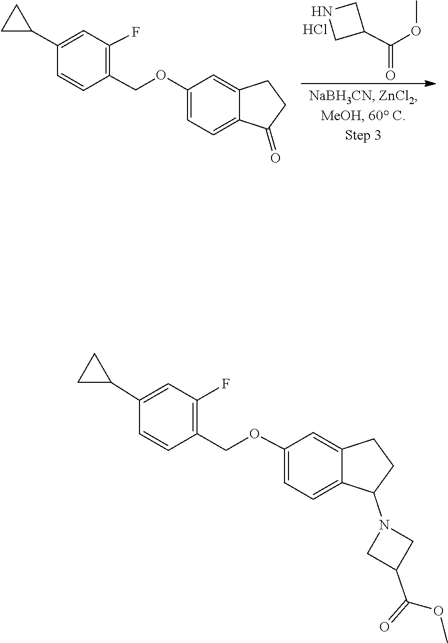

To a solution of NaBH₃CN (127 mg, 2.02 mmol, 4.00 equiv) in methanol (5.0 mL) was added 2 M ZnCl₂ in THF (0.1 mL, 1.01 mmol, 2.00 equiv). The resulting mixture was stirred for 10 min and then 5-((4-cyclopropyl-2-fluorobenzyl)oxy)-2,3-dihydro-1H-inden-1-one (150 mg, 0.510 mmol, 1.00 equiv) and methyl azetidine-3-carboxylate hydrochloride (115 mg, 0.760 mmol, 1.50 equiv) were added. The resulting mixture was flushed with nitrogen and was stirred at 60° C. overnight. LCMS showed the reaction was complete. The mixture was purified by Prep-TLC (MeOH:DCM=1:17) to afford methyl 1-(5-((4-cyclopropyl-2-fluorobenzyl)oxy)-2,3-dihydro-1H-inden-1-yl)azetidine-3-carboxylate (144 mg, 71%) as a light-yellow oil. LCMS (ESI, m/z): 396 [M+H]⁺.

### Synthesis of 1-(5-((4-cyclopropyl-2-fluorobenzyl)oxy)-2,3-dihydro-1H-inden-1-yl)azetidine-3-carboxylic acid

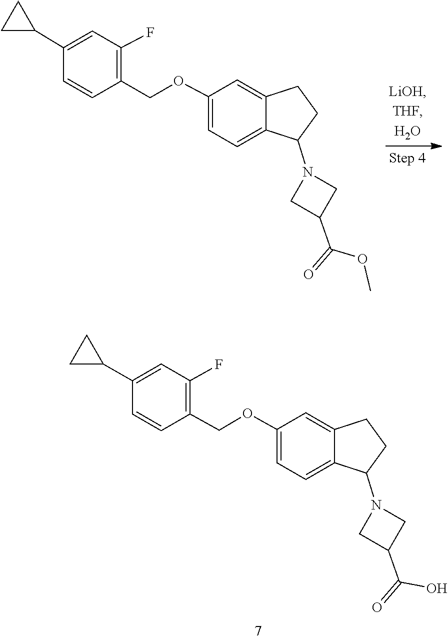 [SMILES: COC(=O)C1CN(C2CCc3cc(OCc4ccc(C5CC5)cc4F)ccc32)C1 ; O=C(O)C1CN(C2CCc3cc(OCc4ccc(C5CC5)cc4F)ccc32)C1]

To a solution of methyl 1-(5-((4-cyclopropyl-2-fluorobenzyl)oxy)-2,3-dihydro-1H-inden-1-yl)azetidine-3-carboxylate (140 mg, 0.350 mmol, 1.00 equiv) in THF (5.0 mL) and water (1.0 mL) was added LiOH H₂O (59 mg, 1.42 mmol, 4.00 equiv). The resulting mixture was stirred overnight. LCMS showed the reaction was complete. The reaction was diluted with 10 mL water. THF was removed under reduced pressure and the water phase was adjusted to pH 2 with 4 M HCl. The mixture was concentrated under reduced pressure. The residue was purified by Prep-HPLC ((Column: Sunfire prep C18 column, 30*150 mm, 5 m; Mobile Phase A: Water (0.05% HCl), Mobile Phase B: ACN; Flow rate: 60 mL/min; Gradient: 10% B to 39% B in 7 min; 254/210 nm; RT: 7.12 min)) to afford 1-(5-((4-cyclopropyl-2-fluorobenzyl)oxy)-2,3-dihydro-1H-inden-1-yl)-azetidine-3-carboxylic acid (100 mg, 74%) as an off-white solid.

LCMS (ESI, m/z): 382 [M+H]⁺. Analytic Conditions: column: Shim-pack XR-ODS, 50 mm, 3.0 mm, 2.2 m; mobile phase A: Water (0.05% TFA), mobile phase B: Acetonitrile (0.05% TFA), flow rate: 1.20 mL/min; gradient: 20% B to 60% B in 2.50 min, 60% B to 95% B in 0.50 min, hold at 95% B for 0.60 min, 95% B to 20% B in 0.10 min; 210 nm; RT: 2.142 min.

¹H NMR (400 MHz, Methanol-d₄) δ 7.48-7.46 (d, J=8.5 Hz, 1H), 7.36-7.32 (t, J=7.9 Hz, 1H), 7.02 (d, J=2.4 Hz, 1H), 6.96-6.90 (ddd, J=12.1, 8.2, 2.1 Hz, 2H), 6.85-6.81 (dd, J=11.5, 1.8 Hz, 1H), 5.09 (s, 2H), 4.84-4.20 (m, 4H), 3.65 (s, 1H), 3.18-3.10 (dt, J=16.5, 8.2 Hz, 1H), 3.00-2.93 (ddd, J=16.8, 9.3, 2.8 Hz, 1H), 2.56-2.46 (dq, J=16.7, 8.4 Hz, 1H), 2.21-2.15 (m, 1H), 1.96-1.90 (m, 1H), 1.01-0.98 (m, 2H), 0.72-0.68 (dt, J=6.6, 4.6 Hz, 2H).

### Example S8. Synthesis of 1-(5-((2-chloro-4-cyclopropylbenzyl)oxy)-2,3-dihydro-1H-inden-1-yl)-azetidine-3-carboxylic acid (8)

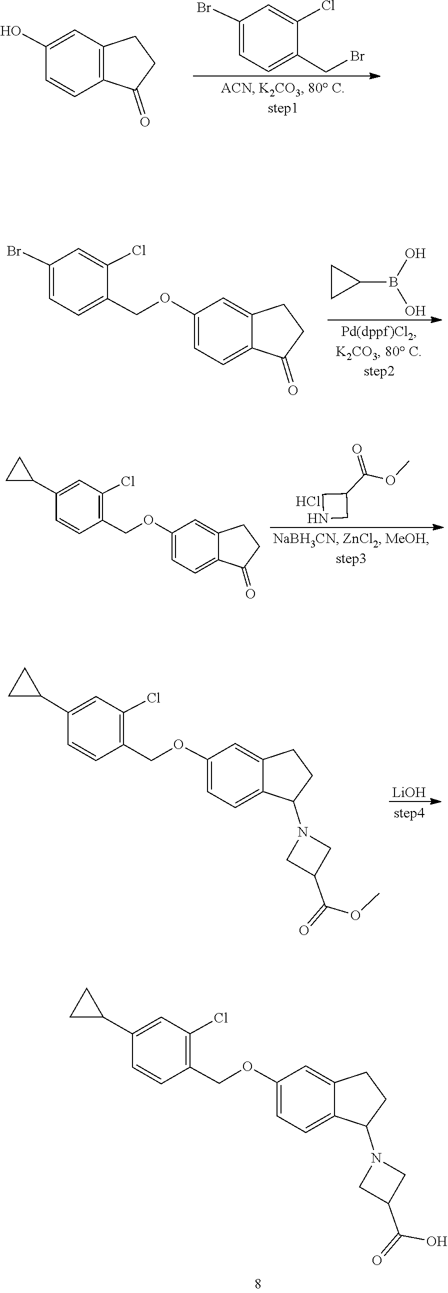

### Synthesis of 5-((4-bromo-2-chlorobenzyl)oxy)-2,3-dihydro-1H-inden-1-one

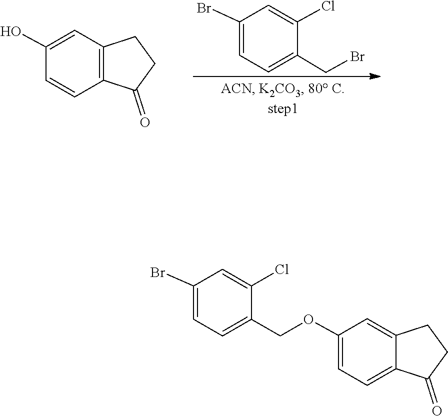 [SMILES: O=C1CCc2cc(O)ccc21 ; Clc1cc(Br)ccc1CBr ; O=C1CCc2cc(OCc3ccc(Br)cc3Cl)ccc21]

To a solution of 5-hydroxyindan-1-one (500 mg, 3.37 mmol, 1.00 equiv) in MeCN (10.0 mL) was added 4-bromo-1-(bromomethyl)-2-chloro-benzene (1151 mg, 4.05 mmol, 1.20 equiv) and K₂CO₃ (1398 mg, 10.1 mmol, 3.00 equiv). The resulting mixture was stirred at 80° C. overnight. The solid was filtered out and the filtrate was concentrated and purified by flash chromatography on silica gel (PE:EA=1:1) to give product 5-((4-bromo-2-chlorobenzyl)oxy)-2,3-dihydro-1H-inden-1-one (1.0 g, 84%) as a yellow solid. LCMS (ESI, m/z): 353 [M+H]⁺.

### Synthesis of 5-((2-chloro-4-cyclopropylbenzyl)oxy)-2,3-dihydro-1H-inden-1-one

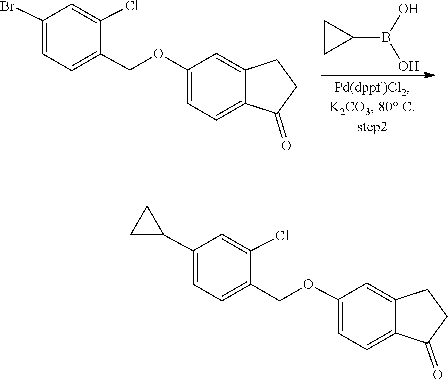

To a solution of 5-((4-bromo-2-chlorobenzyl)oxy)-2,3-dihydro-1H-inden-1-one (600 mg, 1.71 mmol, 1.00 equiv) in 1,4-dioxane (20 mL) and water (2.0 mL) was added Pd(dppf)Cl₂ (278 mg, 0.340 mmol, 2.00 equiv), cyclopropylboronic acid (293 mg, 3.41 mmol, 2.00 equiv) and K₂CO₃ (707 mg, 5.12 mmol, 3.00 equiv). The mixture was stirred at 80° C. overnight. LCMS showed the reaction was complete. The resulting solution was diluted with 20 mL EA and dried over anhydrous Na₂SO₄. The solid was filtered out and the filtrate was concentrated under reduced pressure. The residue was purified by column chromatography (PE:EA=3:1) to give product 5-((2-chloro-4-cyclopropylbenzyl)oxy)-2,3-dihydro-1H-inden-1-one (380 mg, 71%). LCMS (ESI, m/z): 313 [M+H]⁺.

### Synthesis of methyl 1-(5-((2-chloro-4-cyclopropylbenzyl)oxy)-2,3-dihydro-1H-inden-1-yl)azetidine-3-carboxylate

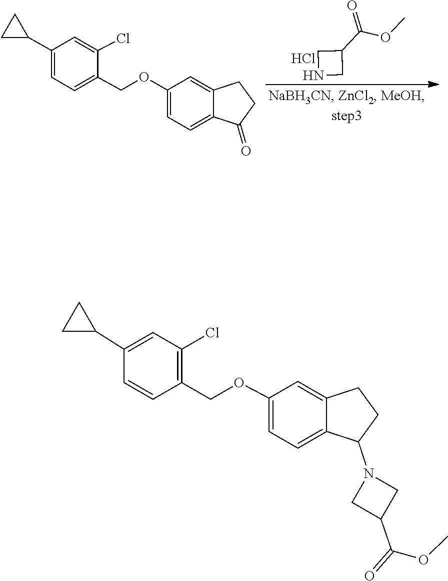

To a solution of NaBH₃CN (306 mg, 4.86 mmol, 4.00 equiv) in methanol (20.0 mL) was added ZnCl₂ (1.2 mL, 2.43 mmol, 2.00 equiv). Then 5-((2-chloro-4-cyclo-propylbenzyl)oxy)-2,3-dihydro-1H-inden-1-one (380 mg, 1.21 mmol, 1.00 equiv) and methyl azetidine-3-carboxylate; hydrochloride (368 mg, 2.43 mmol, 2.00 equiv) were added. The mixture was stirred at 60° C. for overnight. The mixture was diluted with 20 mL water and extracted with EA (3×20 mL). The organic layers were combined and dried with anhydrous Na₂SO₄. The solid was filtered out and the filtrate concentrated under reduced pressure. The residue was purified by flash chromatography on silica gel (MeOH:DCM=1:20) to afford methyl 1-(5-((2-chloro-4-cyclopropylbenzyl)oxy)-2,3-dihydro-1H-inden-1-yl)azetidine-3-carboxylate (400 mg, 80%). LCMS (ESI, m/z): 412 [M+H]⁺.

### Synthesis of 1-(5-((2-chloro-4-cyclopropylbenzyl)oxy)-2,3-dihydro-1H-inden-1-yl)azetidine-3-carboxylic acid

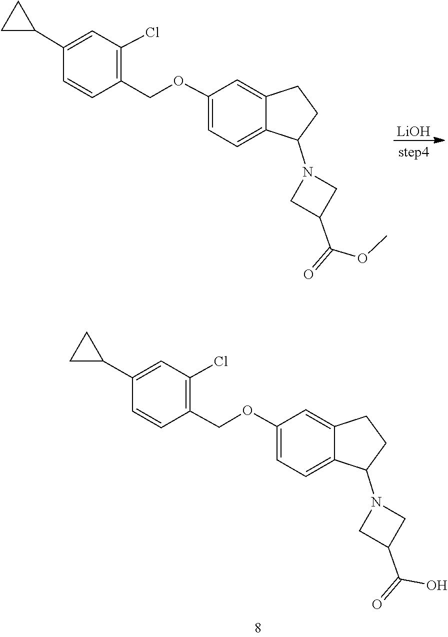 [SMILES: COC(=O)C1CN(C2CCc3cc(OCc4ccc(C5CC5)cc4Cl)ccc32)C1 ; "LiOH" ; O=C(O)C1CN(C2CCc3cc(OCc4ccc(C5CC5)cc4Cl)ccc32)C1]

To a solution of methyl 1-(5-((2-chloro-4-cyclopropylbenzyl)oxy)-2,3-dihydro-1H-inden-1-yl)azetidine-3-carboxylate (400 mg, 0.97 mmol, 1.00 equiv) in THE (5.0 mL) and water (0.5 mL) was added LiOH H₂O (81.57 mg, 1.94 mmol, 2.00 equiv). The mixture was stirred overnight at room temperature. The reaction was diluted with 10 mL water and THF was removed under vacuum and adjusted to pH 6 with 2 M HCl. The solvent was concentrated under reduced pressure and the residue was purified by Prep-HPLC (Column: YMC-Actus Triart C18 ExRS. 30*250, 5 m; Mobile Phase A: water (10 mmol/L NH₄HCO₃+0.1% NH₃·H₂O), Mobile Phase B: ACN; Flow rate: 60 mL/min; Gradient: 25% B to 50% B in 7 min; 254/210 nm; RT: 5.70 min) to afford 1-(5-((2-chloro-4-cyclopropylbenzyl)oxy)-2,3-dihydro-1H-inden-1-yl)azetidine-3-carboxylic acid (115 mg, 30%) as an off-white solid.

LCMS (ESI, m/z): 398 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 95% B in 2.00 min, hold at 95% for 0.70 min, 95% B to 5% B in 0.05 min; 220 nm; RT: 1.678 min.

¹H NMR (400 MHz, Methanol-d₄) δ 7.47 (d, J=8.5 Hz, 1H), 7.38 (d, J=8.0 Hz, 1H), 7.14 (s, 1H), 7.01 (d, J=7.6 Hz, 2H), 6.94 (d, J=8.5 Hz, 1H), 5.13 (s, 2H), 4.87 (s, 1H), 4.70-4.20 (m, 4H), 3.65 (s, 1H), 3.18-3.08 (m, 1H), 2.95 (dd, J=16.9, 9.3 Hz, 1H), 2.49 (d, J=15.2 Hz, 1H), 2.18 (s, 1H), 1.94-1.87 (m, 1H), 1.00 (dd, J=8.5, 2.6 Hz, 2H), 0.69 (d, J=5.3 Hz, 2H).

### Example S9. Synthesis of 1-(5-((4-cyclopropyl-3-(trifluoromethyl)benzyl)oxy)-2,3-dihydro-1H-inden-1-yl)azetidine-3-carboxylic acid (9)

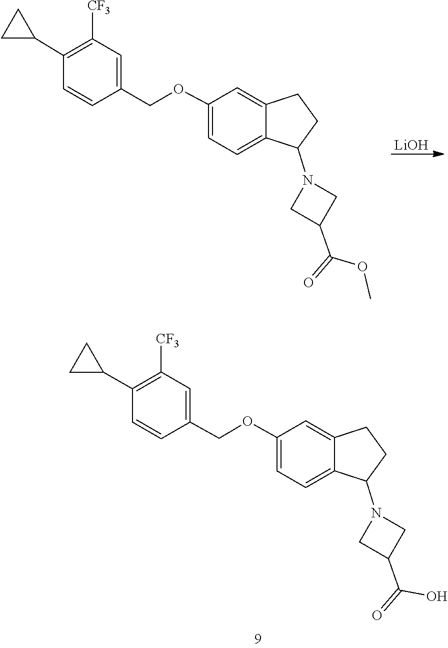 [SMILES: COC(=O)C1CN(C2CCc3cc(OCc4ccc(C5CC5)c(C(F)(F)F)c4)ccc32)C1 ; "LiOH" ; O=C(O)C1CN(C2CCc3cc(OCc4ccc(C5CC5)c(C(F)(F)F)c4)ccc32)C1]

A solution of methyl 1-(5-((4-cyclopropyl-3-(trifluoromethyl)benzyl)oxy)-2,3-dihydro-1H-inden-1-yl)azetidine-3-carboxylate (480 mg, 1.08 mmol, 1.00 equiv) and LiOH H₂O (91 mg, 2.16 mmol, 2.00 equiv) in water (1.0 mL) and THE (10.0 mL) was stirred at RT overnight. LCMS showed the reaction was complete. The pH value of the solution was adjusted to 3˜5 with 2 M HCl. The mixture was purified by Prep-HPLC (Column: XSelect CSH Prep C18 OBD Column, 19*250 mm, 5 m; Mobile Phase A: Water (0.05% HCl), Mobile Phase B: ACN; Flow rate: 25 mL/min; Gradient: 50% B to 80% B in 7 min, 80% B; Wave Length: 254/210 nm; RT: 6.78) to give 1-(5-((4-cyclo-propyl-3-(trifluoromethyl)benzyl)oxy)-2,3-dihydro-1H-inden-1-yl)azetidine-3-carboxylic acid (136.8 mg, 29%) as an off-white solid.

LCMS (ESI, m/z): 432 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 100% B in 2.00 min, hold at 100% for 0.70 min, 100% B to 5% B in 0.05 min; 210 nm; RT: 1.704 min.

¹H NMR (400 MHz, Methanol-d₄) δ 7.65 (d, J=1.9 Hz, 1H), 7.56-7.49 (m, 1H), 7.44 (d, J=8.4 Hz, 1H), 7.10 (d, J=7.8 Hz, 1H), 6.99 (d, J=2.9 Hz, 1H), 6.92 (dd, J=8.4, 2.6 Hz, 1H), 5.11 (s, 2H), 4.89-4.83 (s, 1H), 4.57 (t, J=10.3 Hz, 1H), 4.40-4.20 (m, 3H), 3.72-3.52 (m, 1H), 3.11 (dt, J=17.1, 8.5 Hz, 1H), 2.98-2.87 (m, 1H), 2.46 (dq, J=17.4, 8.7 Hz, 1H), 2.21-2.11 (m, 2H), 1.06-0.95 (m, 2H), 0.80-0.72 (m, 2H).

### Example S10. Synthesis of 1-(5-((3-chloro-4-cyclopropylbenzyl)oxy)-2,3-dihydro-1H-inden-1-yl)azetidine-3-carboxylic acid (10)

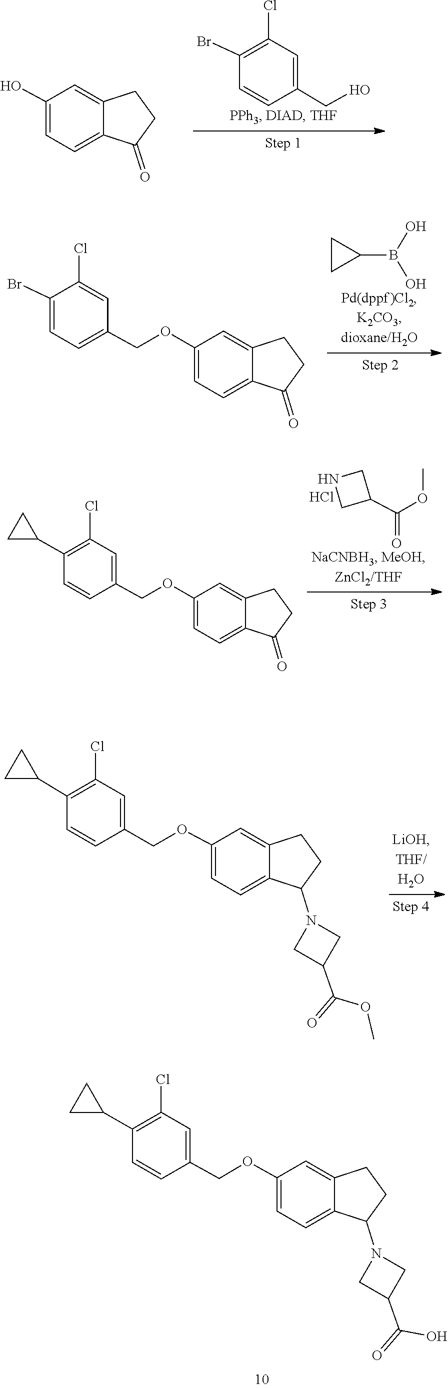

### Synthesis of 5-((4-bromo-3-chlorobenzyl)oxy)-2,3-dihydro-1H-inden-1-one

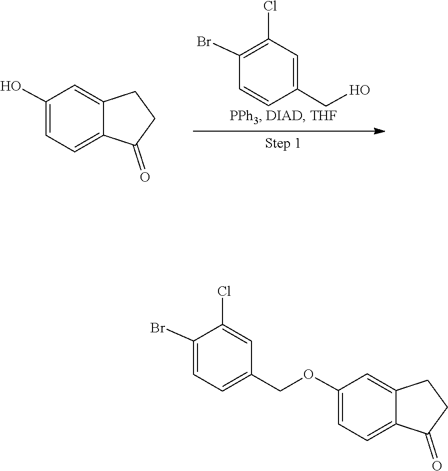 [SMILES: O=C1CCc2cc(O)ccc21 ; OCc1ccc(Br)c(Cl)c1 ; O=C1CCc2cc(OCc3ccc(Br)c(Cl)c3)ccc21]

To a stirred solution of 5-hydroxyindan-1-one (1.0 g, 6.75 mmol, 1.00 equiv), (4-bromo-3-chloro-phenyl)methanol (1.5 g, 6.75 mmol, 1.00 equiv), PPh₃ (2.7 g, 10.12 mmol, 1.50 equiv) in THF (50.0 mL) was added DIAD (2.0 mL, 10.1 mmol, 1.50 equiv) at 0° C. under N₂ atmosphere. The resulting mixture was stirred overnight at room temperature. LCMS showed the reaction was complete. The resulting mixture was concentrated under vacuum. The residue was purified by flash chromatography on silica gel (PE/EA=4:1) to afford 5-((4-bromo-3-chlorobenzyl)oxy)-2,3-dihydro-1H-inden-1-one (1.2 g, 51%) as a light yellow solid. LCMS (ESI, m/z): 351 [M+H]⁺.

### Synthesis of 5-((3-chloro-4-cyclopropylbenzyl)oxy)-2,3-dihydro-1H-inden-1-one

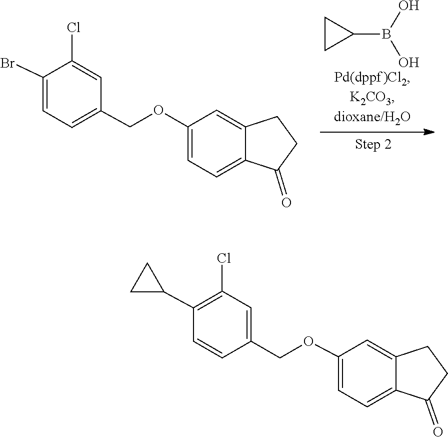 [SMILES: O=C1CCc2cc(OCc3ccc(Br)c(Cl)c3)ccc21 ; OB(O)C1CC1 ; O=C1CCc2cc(OCc3ccc(C4CC4)c(Cl)c3)ccc21]

To a stirred solution of 5-((4-bromo-3-chlorobenzyl)oxy)-2,3-dihydro-1H-inden-1-one (1.0 g, 2.84 mmol, 1.00 equiv), cyclopropylboronic acid (0.49 g, 5.69 mmol, 2.00 equiv), K₂CO₃ (1.2 g, 8.53 mmol, 3.00 equiv) in 1,4-dioxane (50.0 mL) and water (5.0 mL) was added Pd(dppf)Cl₂ (0.5 g, 0.570 mmol, 0.20 equiv) under N₂ atmosphere. The reaction mixture was stirred overnight at 80° C. LCMS showed the reaction was complete. The resulting mixture was filtered and the filtrate was concentrated under reduced pressure. The residue was purified by flash chromatography on silica gel (PE:EA=5:1) to afford 5-((3-chloro-4-cyclopropylbenzyl)oxy)-2,3-dihydro-1H-inden-1-one (475 mg, 50%) as a light yellow solid. LCMS (ESI, m/z): 313 [M+H]⁺.

### Synthesis of methyl 1-(5-((3-chloro-4-cyclopropylbenzyl)oxy)-2,3-dihydro-1H-inden-1-yl)azetidine-3-carboxylate

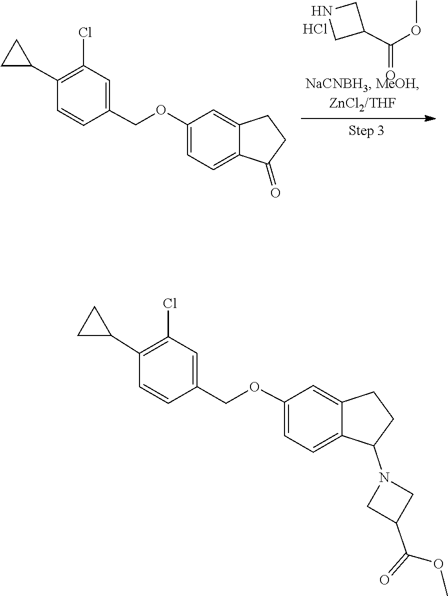

Into a 100 mL round-bottom flask were placed a solution of NaBH₃CN (337 mg, 5.37 mmol, 4.00 equiv) and ZnCl₂/THF (1.5 mL, 2.69 mmol, 2.00 equiv) in methanol (30.0 mL). To the above solution was added 5-((3-chloro-4-cyclopropylbenzyl)oxy)-2,3-dihydro-1H-inden-1-one (420 mg, 1.34 mmol, 1.00 equiv) and methyl azetidine-3-carboxylate hydrochloride (305 mg, 2.01 mmol, 1.50 equiv). The resulting mixture was stirred at 70° C. overnight under a nitrogen atmosphere. The reaction was quenched with NH₄Cl (sat. aq, 80 mL) and extracted with EA (3×80 mL). The combined organic phase was dried over Na₂SO₄. The solid was filtered out and the filtrate was concentrated under reduced pressure and then purified by TLC chromatography (DCM: CH₂Cl₂=30:1) to afford methyl 1-(5-((3-chloro-4-cyclopropylbenzyl)oxy)-2,3-dihydro-1H-inden-1-yl)azetidine-3-carboxylate (340 mg, 58%) as a light yellow oil. LCMS (ESI, m/z): 412 [M+H]⁺.

### Synthesis of 1-(5-((3-chloro-4-cyclopropylbenzyl)oxy)-2,3-dihydro-1H-inden-1-yl)azetidine-3-carboxylic acid

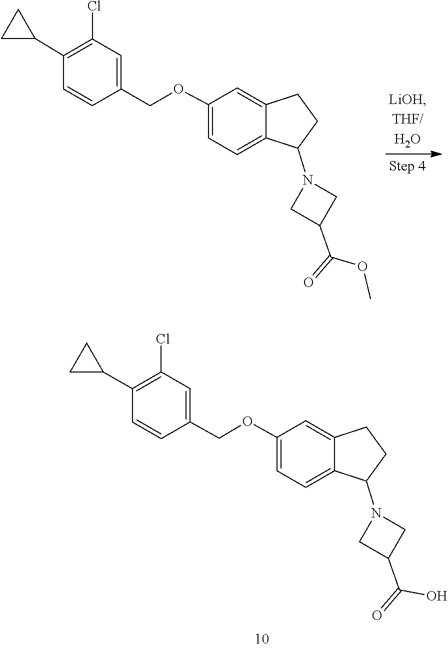 [SMILES: COC(=O)C1CN(C2CCc3cc(OCc4ccc(C5CC5)c(Cl)c4)ccc32)C1 ; O=C(O)C1CN(C2CCc3cc(OCc4ccc(C5CC5)c(Cl)c4)ccc32)C1]

To a stirred solution of methyl 1-(5-((3-chloro-4-cyclopropylbenzyl)oxy)-2,3-dihydro-1H-inden-1-yl)azetidine-3-carboxylate (310 mg, 0.750 mmol, 1.00 equiv) in THE (20 mL) and water (2 mL) was added LiOH H₂O (63 mg, 1.51 mmol, 2.00 equiv). The resulting mixture was stirred overnight at room temperature. The reaction was diluted with 20 mL water and THF removed under vacuum. The aqueous layer was adjusted to pH 2 with 4 M HCl and concentrated under reduced pressure. The residue was purified by Prep-HPLC (Column: Sunfire prep C18 column, 30*150, 5 m; Mobile Phase A: Water (0.05% HCl), Mobile Phase B: ACN; Flow rate: 60 mL/min; Gradient: 15% B to 38% B in 7 min; 254/210 nm; RT: 10.8 min) to afford 1-(5-((3-chloro-4-cyclopropyl-benzyl)oxy)-2,3-dihydro-1H-inden-1-yl)azetidine-3-carboxylic acid (174.3 mg, 58.092%) as an off-white solid.

LCMS (ESI, m/z): 398 [M+H]⁺. Analytic Conditions: Column: Shim-pack XR-ODS Column 3*50 mm, 2.2 m; Mobile Phase A: Water/0.05% TFA, Mobile Phase B: Acetonitrile/0.05% TFA; Flow rate: 1.20 mL/min; Gradient: 5% B to 100% B in 2.0 min, hold at 100% for 0.70 min, 100% B to 5% B in 0.05 min; 210 nm; RT: 1.686 min.

¹H NMR (400 MHz, DMSO-d₆) δ 7.5-7.46 (d, J=8.0 Hz, 1H), 7.31 (dd, J=8.1, 1.8 Hz, 1H), 7.04 (m, 2H), 6.89 (dd, J=8.4, 2.4 Hz, 1H), 5.08 (s, 2H), 4.82-4.76 (m, 1H), 4.29 (s, 2H), 4.10 (t, J=9.2 Hz, 2H), 3.56 (p, J=8.7 Hz, 2H), 3.25-3.12 (m, 2H), 2.80 (ddd, J=16.5, 9.1, 2.8 Hz, 1H), 2.33 (dq, J=16.5, 8.5 Hz, 1H), 2.12 (tdd, J=11.1, 8.4, 5.3 Hz, 2H), 1.07-0.94 (m, 2H), 0.74-0.66 (m, 2H).

### Example S11. Synthesis of 1-(5-((2-fluorobenzyl)oxy)-2,3-dihydro-1H-inden-1-yl)azetidine-3-carboxylic acid (11)

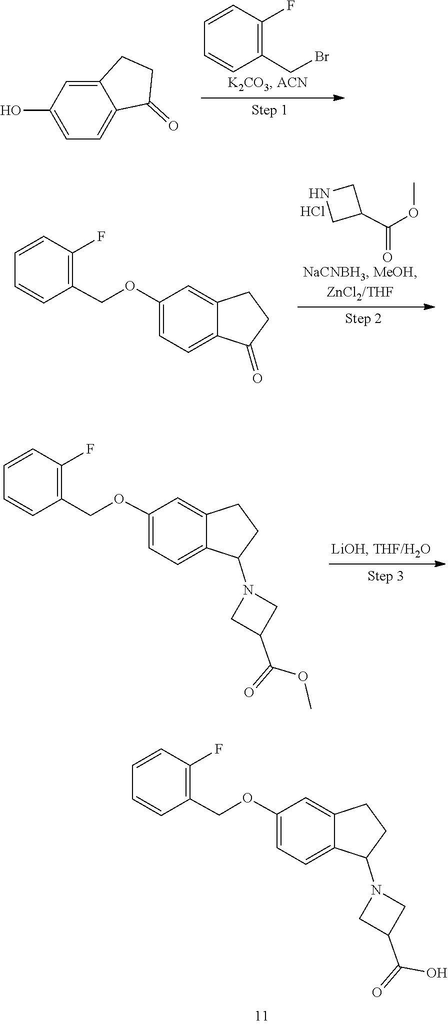

### Synthesis of 5-((2-fluorobenzyl)oxy)-2,3-dihydro-1H-inden-1-one

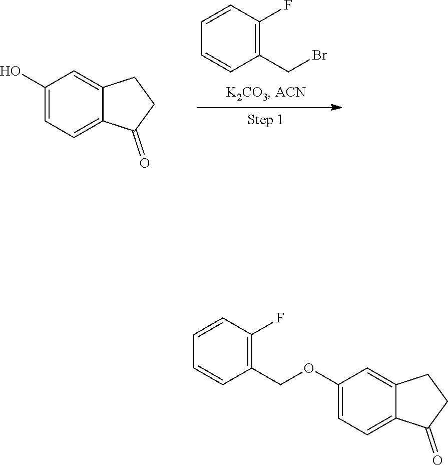 [SMILES: O=C1CCc2cc(O)ccc21 ; Fc1ccccc1CBr ; O=C1CCc2cc(OCc3ccccc3F)ccc21]

In a 100 mL round-bottom flask was placed a solution of 5-hydroxyindan-1-one (1.0 g, 6.75 mmol), 1-(bromomethyl)-2-fluoro-benzene (1.53 g, 8.1 mmol) and K₂CO₃ (2.79 g, 20.25 mmol) in MeCN (40.0 mL). The reaction mixture was stirred overnight at 50° C. under a nitrogen atmosphere. The solid was filtered out and the filtrate was concentrated under reduced pressure. The residue was purified by flash chromatography on silica gel (EA:PE=1:6) to afford 5-((2-fluorobenzyl)oxy)-2,3-dihydro-1H-inden-1-one (1.6 g, 85%) as a yellow solid. LCMS (ESI, m/z): 257 [M+H]⁺.

### Synthesis of methyl 1-(5-((2-fluorobenzyl)oxy)-2,3-dihydro-1H-inden-1-yl)azetidine-3-carboxylate

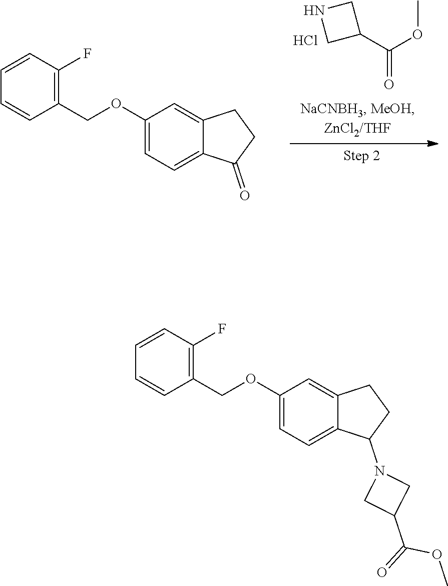

In a 100 mL round-bottom flask was placed a solution of NaBH₃CN (981 mg, 15.6 mmol, 4.00 equiv), ZnCl₂/THF (4.0 mL, 7.80 mmol, 2.00 equiv) in methanol (30.0 mL). The mixture was stirred at room temperature for 10 mins. This was followed by the addition of 5-((2-fluorobenzyl)oxy)-2,3-dihydro-1H-inden-1-one (1.0 g, 3.90 mmol, 1.00 equiv) and methyl azetidine-3-carboxylate hydrochloride (0.9 g, 5.85 mmol, 1.50 equiv). The resulting solution was stirred at 70° C. overnight under a nitrogen atmosphere. The mixture was concentrated and purified by flash chromatography on silica gel with dichloromethane/methanol (25:1) to afford methyl 1-(5-((2-fluorobenzyl)oxy)-2,3-dihydro-1H-inden-1-yl)azetidine-3-carboxylate (1.0 g, 72%) as a yellow oil. LCMS (ESI, m/z): 356 [M+H]⁺.

### Synthesis of 1-(5-((2-fluorobenzyl)oxy)-2,3-dihydro-1H-inden-1-yl)azetidine-3-carboxylic acid

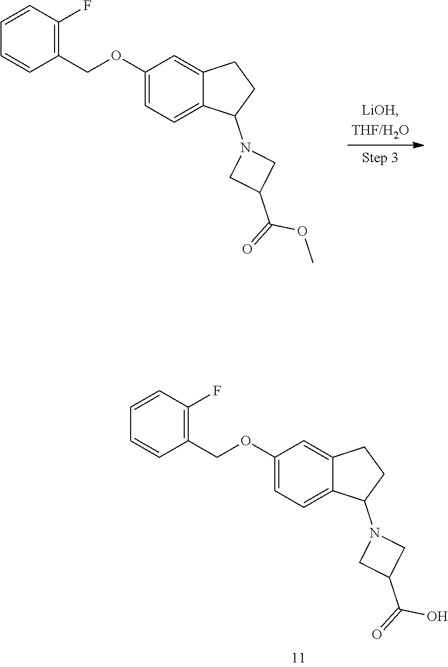 [SMILES: COC(=O)C1CN(C2CCc3cc(OCc4ccccc4F)ccc32)C1 ; O=C(O)C1CN(C2CCc3cc(OCc4ccccc4F)ccc32)C1]

To a stirred solution of methyl 1-(5-((2-fluorobenzyl)oxy)-2,3-dihydro-1H-inden-1-yl)azetidine-3-carboxylate (300 mg, 0.840 mmol, 1.00 equiv) in THE (20.0 mL) and water (2.0 mL) was added LiOH H₂O (71 mg, 1.69 mmol, 2.00 equiv). The resulting mixture was stirred overnight. The reaction mixture was diluted with 20 mL water. THE was removed under reduced pressure and the pH adjusted to 6 with 2 M HCl. The solid was collected by filtration and recrystallized from water (3 mL) to afford 1-(5-((2-fluorobenzyl)oxy)-2,3-dihydro-1H-inden-1-yl)azetidine-3-carboxylic acid (105 mg, 36%) as an off-white solid.

LCMS (ESI, m/z): 342 [M+H]⁺. Analytic Conditions: Column: Shim-pack XR-ODS Column 3*50 mm, 2.2 m; Mobile Phase A: Water/0.05% TFA, Mobile Phase B: Acetonitrile/0.05% TFA; Flow rate: 1.20 mL/min; Gradient: 5% B to 100% B in 2.00 min, hold at 100% for 0.70 min, 100% B to 5% B in 0.05 min 210 nm; Rt: 1.433 min.

¹H NMR (400 MHz, Methanol-d₄) δ 7.56-7.44 (m, 2H), 7.38 (tdd, J=7.5, 5.3, 1.8 Hz, 1H), 7.20-7.15 (m, 1H), 7.03 (d, J=2.4 Hz, 1H), 6.97 (dd, J=8.5, 2.4 Hz, 1H), 5.18 (s, 2H), 4.80 (dd, J=7.7, 2.2 Hz, 1H), 4.38-4.27 (m, 2H), 4.19 (dd, J=10.1, 8.0 Hz, 2H), 3.43-3.34 (m, 1H), 3.14 (dt, J=16.6, 8.3 Hz, 1H), 2.96 (ddd, J=16.7, 9.2, 2.9 Hz, 1H), 2.45 (ddt, J=16.0, 9.3, 8.0 Hz, 1H), 2.18 (ddt, J=14.7, 8.0, 2.7 Hz, 1H).

### Example S12. Synthesis of 1-(5-((3-fluorobenzyl)oxy)-2,3-dihydro-1H-inden-1-yl)azetidine-3-carboxylic acid (12)

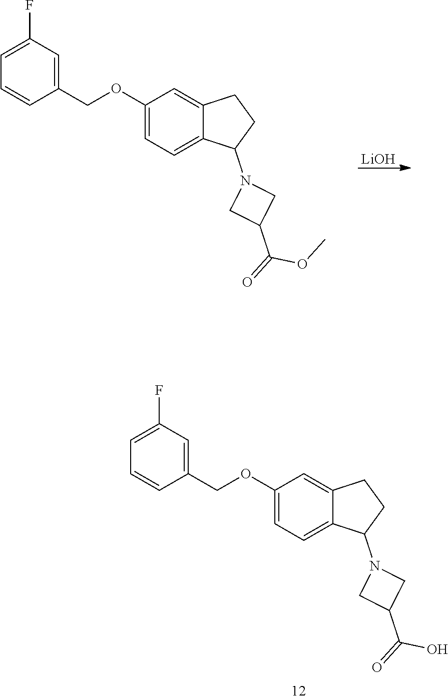 [SMILES: COC(=O)C1CN(C2CCc3cc(OCc4cccc(F)c4)ccc32)C1 ; "LiOH" ; O=C(O)C1CN(C2CCc3cc(OCc4cccc(F)c4)ccc32)C1]

To a stirred solution of methyl 1-(5-((3-fluorobenzyl)oxy)-2,3-dihydro-1H-inden-1-yl)azetidine-3-carboxylate (750 mg, 2.11 mmol, 1.00 equiv) in THE (10.0 mL) and water (1.0 mL) was added LiOH H₂O (133 mg, 3.17 mmol, 1.50 equiv). The resulting solution was stirred at room temperature for 2 h. The reaction was diluted with 10 mL water and the pH adjusted to 2 with 4 M HCl. The solvent was removed under reduced pressure. The residue was purified by Prep-HPLC (Column: YMC-Actus Triart C18 ExRS. 30*250, 5 m; Mobile Phase A: water (0.05 HCl), Mobile Phase B: ACN; Flow rate: 60 mL/min; Gradient: 25% B to 50% B in 7 min; 254/210 nm; RT: 5.90 min) to afford 1-(5-((3-fluorobenzyl)oxy)-2,3-dihydro-1H-inden-1-yl)azetidine-3-carboxylic acid (175 mg, 24%) as an off-white solid.

LCMS (ESI, m/z): 342 [M+H]⁺. Analytic Conditions: column: Shim-pack XR-ODS, 3.0*50 mm, 2.2 m; mobile phase A: water (0.05% TFA), mobile phase B: acetonitrile (0.05% TFA), flow rate: 1.20 mL/min; gradient: 5% B to 55% B in 2.80 min, 55% B to 95% B in 0.40 min, hold at 95% B for 0.50 min, 95% B to 10% B in 0.10 min; 210 nm; RT: 2.440 min.

¹H NMR (400 MHz, Methanol-d4) δ 7.47 (d, J=8.4 Hz, 1H), 7.37 (td, J=8.0, 5.8 Hz, 1H), 7.22 (d, J=7.7 Hz, 1H), 7.19 (dd, J=9.5, 2.6 Hz, 1H), 7.10-7.01 (m, 2H), 6.97 (dd, J=8.5, 2.5 Hz, 1H), 5.13 (s, 2H), 4.70-(br, 4H), 3.67 (s, 1H), 3.15 (dt, J=16.6, 8.2 Hz, 1H), 2.95 (m, 1H), 2.52 (dq, J=16.3, 8.4 Hz, 1H), 2.15 (m, 1H).

### Example S13. Synthesis of 1-(5-((4-fluorobenzyl)oxy)-2,3-dihydro-1H-inden-1-yl)azetidine-3-carboxylic acid (13)

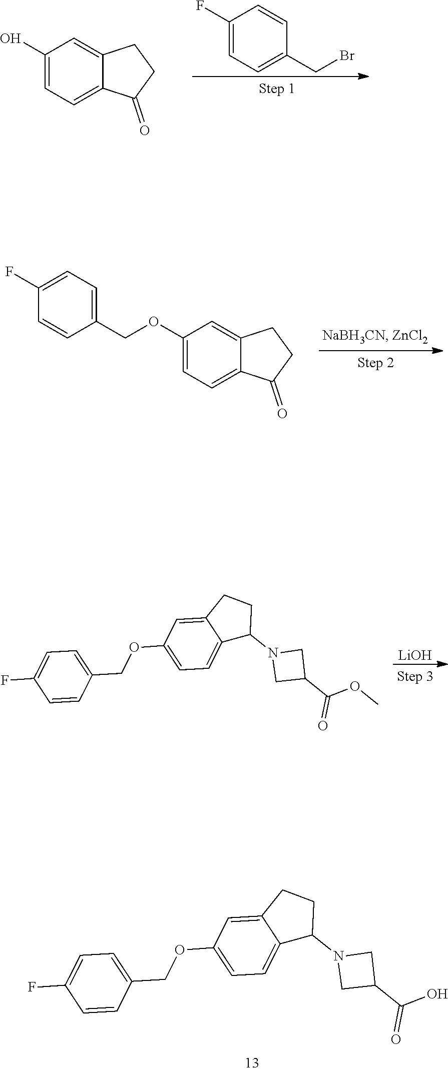

### Synthesis of 5-((4-fluorobenzyl)oxy)-2,3-dihydro-1H-inden-1-one

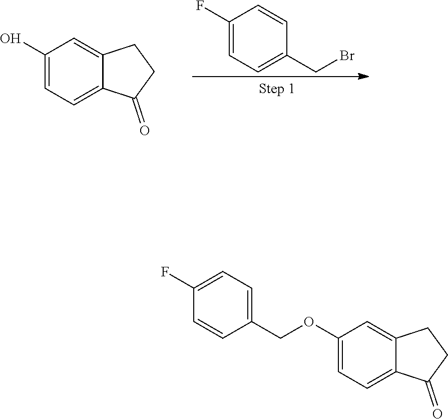 [SMILES: O=C1CCc2cc(O)ccc21 ; Fc1ccc(CBr)cc1 ; O=C1CCc2cc(OCc3ccc(F)cc3)ccc21]

To a solution of 5-hydroxyindan-1-one (1.0 g, 6.75 mmol, 1.00 equiv) and 1-(bromomethyl)-4-fluoro-benzene (1.4 g, 7.42 mmol, 1.10 equiv) in MeCN (20.0 mL) was added K₂CO₃ (2.8 g, 20.3 mmol, 3.00 equiv). The flask was evacuated and flushed five times with nitrogen. The mixture was stirred overnight at 50° C. under nitrogen atmosphere. LCMS showed the reaction was complete. The resulting solution was diluted with 20 mL EA. The solid was filtered out and the filtrate was concentrated under reduced pressure. The residue was purified by flash chromatography on silica gel (EA:PE=1:3) to afford 5-((4-fluorobenzyl)oxy)-2,3-dihydro-1H-inden-1-one (1.5 g, 86.72%) as a yellow solid. LCMS (ESI, m/z): 256 [M+H]⁺.

### Synthesis of methyl 1-(5-((4-fluorobenzyl)oxy)-2,3-dihydro-1H-inden-1-yl)azetidine-3-carboxylate

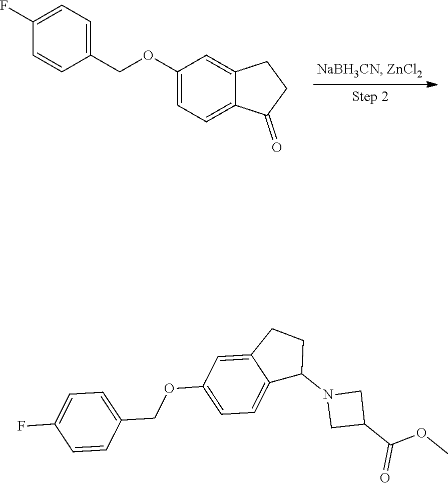 [SMILES: O=C1CCc2cc(OCc3ccc(F)cc3)ccc21 ; COC(=O)C1CN(C2CCc3cc(OCc4ccc(F)cc4)ccc32)C1]

To a solution of NaBH₃CN (492 mg, 7.80 mmol, 4.00 equiv) in methanol (10.0 mL) was added 2 M ZnCl₂ in THF (2.0 mL, 3.90 mmol, 2.00 equiv) and the mixture was stirred for 10 min, followed by the addition of methyl azetidine-3-carboxylate hydrochloride (443.64 mg, 2.93 mmol, 1.50 equiv) and 5-((4-fluoro-benzyl)oxy)-2,3-dihydro-1H-inden-1-one (500 mg, 1.95 mmol, 1.00 equiv). The reaction was stirred overnight at 70° C. LCMS showed the reaction was complete. The resulting mixture was concentrated and purified by flash chromatography on silica gel with dichloromethane/methanol (25:1) to afford methyl 1-(5-((4-fluorobenzyl)oxy)-2,3-dihydro-1H-inden-1-yl)-azetidine-3-carboxylate (600 mg, 87%) as a yellow solid. LCMS (ESI, m/z): 356 [M+H]⁺.

### Synthesis of 1-(5-((4-fluorobenzyl)oxy)-2,3-dihydro-1H-inden-1-yl)azetidine-3-carboxylic acid

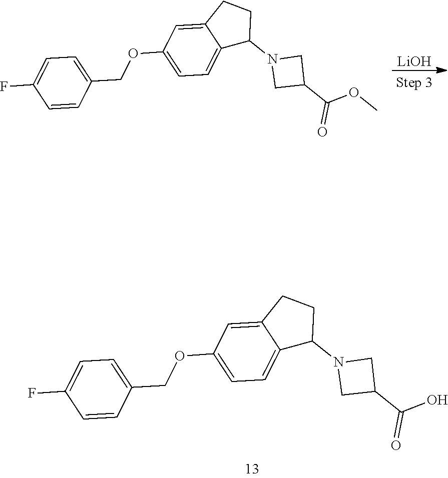 [SMILES: COC(=O)C1CN(C2CCc3cc(OCc4ccc(F)cc4)ccc32)C1 ; "LiOH" ; O=C(O)C1CN(C2CCc3cc(OCc4ccc(F)cc4)ccc32)C1]

To a solution of methyl 1-(5-((4-fluorobenzyl)oxy)-2,3-dihydro-1H-inden-1-yl)azetidine-3-carboxylate (600 mg, 1.69 mmol, 1.00 equiv) in THF (10.0 mL) and water (1.0 mL) was added LiOH H₂O (141.81 mg, 3.38 mmol, 2.00 equiv). The mixture was stirred overnight at room temperature. The reaction was diluted with 10 mL water and the pH was adjusted to 5-6 with 1 M HCl. The solid was collected by filtration to give 1-(5-((4-fluorobenzyl)oxy)-2,3-dihydro-1H-inden-1-yl)azetidine-3-carboxylic acid (164.3 mg, 28%) as an off-white solid.

LCMS (ESI, m/z): 342 [M+H]⁺. Analytic Conditions: column: Shim-pack XR-ODS, 3.0*50 mm, 2.2 m; mobile phase A: water (0.05% TFA), mobile phase B: acetonitrile (0.05% TFA), flow rate: 1.20 mL/min; gradient: 5% B to 100% B in 2.00 min, hold at 100% B for 0.70 min, 100% B to 5% B in 0.05 min; 210 nm; RT: 1.458 min.

¹H NMR (400 MHz, Methanol-d4) δ 7.50-7.41 (m, 3H), 7.16-7.06 (m, 2H), 7.01 (d, J=2.3 Hz, 1H), 6.95 (dd, J=8.4, 2.4 Hz, 1H), 5.07 (s, 2H), 4.75 (dd, J=7.7, 2.2 Hz, 1H), 4.37-4.26 (m, 2H), 4.18-4.13 (dd, J=10.2, 7.7 Hz, 2H), 3.35 (m, 1H), 3.15-3.07 (dt, J=16.5, 8.2 Hz, 1H), 2.95-2.88 (ddd, J=16.7, 9.3, 2.9 Hz, 1H), 2.49-2.43 (m, 1H), 2.18-2.13 (ddt, J=14.3, 8.1, 2.7 Hz, 1H).

### Example S14. Synthesis of 1-(5-((2,4-difluorobenzyl)oxy)-2,3-dihydro-1H-inden-1-yl)azetidine-3-carboxylic acid (14)

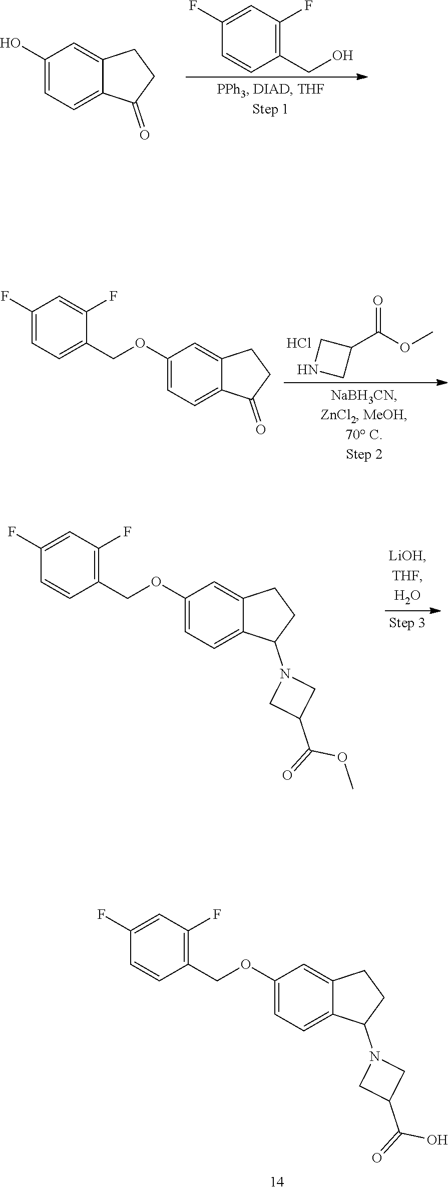

### Synthesis of 5-((2,4-difluorobenzyl)oxy)-2,3-dihydro-1H-inden-1-one

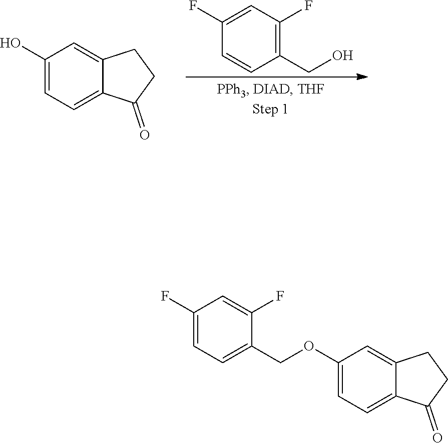 [SMILES: O=C1CCc2cc(O)ccc21 ; OCc1ccc(F)cc1F ; O=C1CCc2cc(OCc3ccc(F)cc3F)ccc21]

To a solution of 5-hydroxyindan-1-one (1000 mg, 6.75 mmol, 1.00 equiv) in THF (30.0 mL) was added (2,4-difluorophenyl)methanol (1167 mg, 8.10 mmol, 1.20 equiv), PPh₃ (2665 mg, 10.2 mmol, 1.50 equiv) and DIAD (2047 mg, 10.1 mmol, 1.50 equiv). The resulting mixture was flushed with nitrogen and stirred overnight at room temperature. The reaction was concentrated under reduced pressure and the residue was purified by flash chromatography on silica gel (EA:PE=1:6) to afford 5-((2,4-difluoro-benzyl)oxy)-2,3-dihydro-1H-inden-1-one (1.47 g, 80%) as a yellow solid. LCMS (ESI, m/z): 275 [M+H]⁺.

### Synthesis of methyl 1-(5-((2,4-difluorobenzyl)oxy)-2,3-dihydro-1H-inden-1-yl)-azetidine-3-carboxylate

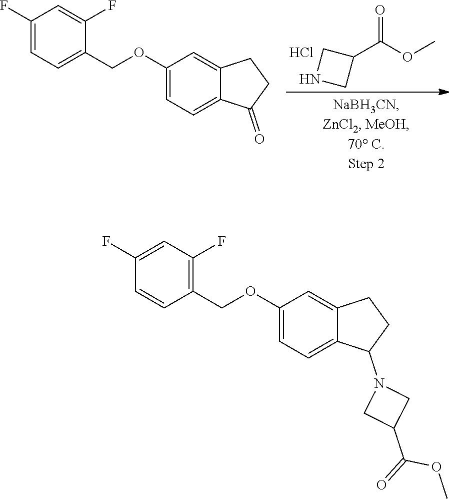

To a solution of NaBH₃CN (559 mg, 8.87 mmol, 4.00 equiv) in MeOH (20.0 mL) was added 2 M ZnCl₂ in THF (2.1 mL, 4.43 mmol, 2.00 equiv). The resulting solution was stirred at room temperature for 10 min, followed by the addition of 5-((2,4-difluorobenzyl)oxy)-2,3-dihydro-1H-inden-1-one (600 mg, 2.19 mmol, 1.00 equiv) and methyl azetidine-3-carboxylate hydrochloride (497 mg, 3.28 mmol, 1.50 equiv). The reaction was flushed with nitrogen and stirred overnight at 70° C. The reaction was quenched with 20 mL saturated NH₄C₁ solution and extracted with EA (3×30 mL). The organic phase was combined and dried over Na₂SO₄. The solid was filtered out and the filtrate was concentrated under reduced pressure. The residue was purified by TLC chromatography on silica gel (100% EA) to afford methyl 1-(5-((2,4-difluorobenzyl)oxy)-2,3-dihydro-1H-inden-1-yl)azetidine-3-carboxylate (192 mg, 23%) as a yellow solid. LCMS (ESI, m/z): 374 [M+H]⁺.

### Synthesis of 1-(5-((2,4-difluorobenzyl)oxy)-2,3-dihydro-1H-inden-1-yl)azetidine-3-carboxylic acid

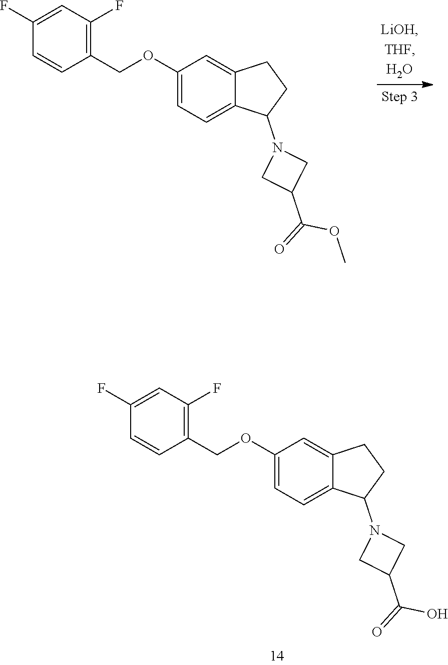 [SMILES: COC(=O)C1CN(C2CCc3cc(OCc4ccc(F)cc4F)ccc32)C1 ; O=C(O)C1CN(C2CCc3cc(OCc4ccc(F)cc4F)ccc32)C1]

To a solution of methyl 1-(5-((2,4-difluorobenzyl)oxy)-2,3-dihydro-1H-inden-1-yl)azetidine-3-carboxylate (192 mg, 0.510 mmol, 1.00 equiv) in THF (10.0 mL) and water (1.0 mL) was added LiOH H₂O (43 mg, 1.03 mmol, 2.00 equiv). The resulting mixture was stirred overnight at room temperature. The reaction was diluted with 10 mL water. THF was removed under reduced pressure and the water phase was adjusted to pH 3 with 4 M HCl. The solution was concentrated under reduced pressure and the residue was purified by Prep-HPLC (Column: Sunfire prep C18 column, 30*150, 5 m; Mobile Phase A: Water (0.05% HCl), Mobile Phase B: ACN; Flow rate: 60 mL/min; Gradient: 15% B to 38% B in 7 min; 254/210 nm; RT: 9.8 min) to afford 1-(5-((2,4-difluorobenzyl)oxy)-2,3-dihydro-1H-inden-1-yl)azetidine-3-carboxylic acid (78 mg, 41%) as an off-white solid.

LCMS (ESI, m/z): 360 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 60% B in 3.00 min, 60% B to 95% B in 0.30 min, hold at 95% for 0.40 min, 95% B to 5% B in 0.10 min; 220 nm; RT: 2.355 min.

¹H NMR (400 MHz, DMSO-d₆) δ 13.14 (s, 1H), 10.61 (s, 1H), 7.61 (td, J=8.5, 6.5 Hz, 1H), 7.50 (d, J=8.4 Hz, 1H), 7.31 (td, J=9.9, 2.6 Hz, 1H), 7.18-7.09 (m, 1H), 7.04 (d, J=2.3 Hz, 1H), 6.92 (dd, J=8.3, 2.4 Hz, 1H), 5.13 (s, 2H), 4.82 (s, 1H), 4.70-4.10 (m, 4H), 3.55 (d, J=9.6 Hz, 1H), 3.14-3.03 (m, 1H), 2.84 (dd, J=16.7, 9.1 Hz, 1H), 2.41-2.33 (m, 1H), 2.13-2.02 (m, 1H).

### Example S15. Synthesis of 1-(5-((2,5-difluorobenzyl)oxy)-2,3-dihydro-1H-inden-1-yl)azetidine-3-carboxylic acid (15)

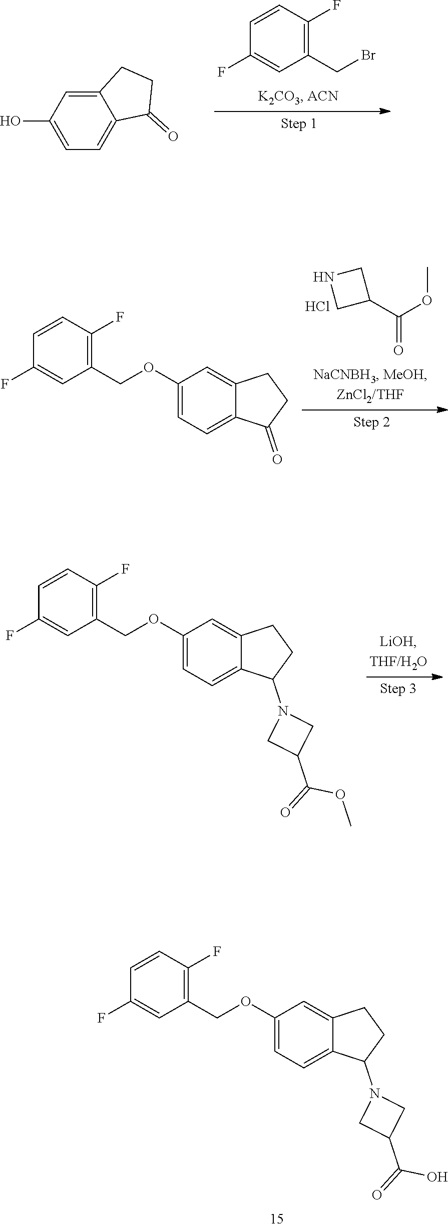

### Synthesis of 5-((2,5-difluorobenzyl)oxy)-2,3-dihydro-1H-inden-1-one

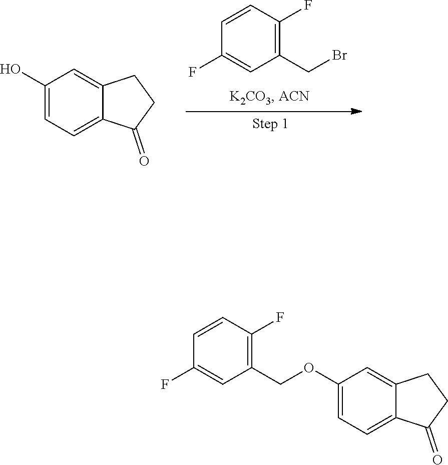 [SMILES: O=C1CCc2cc(O)ccc21 ; Fc1ccc(F)c(CBr)c1 ; O=C1CCc2cc(OCc3cc(F)ccc3F)ccc21]

In a 100 mL round-bottom flask was placed a solution of 5-hydroxyindan-1-one (700.0 mg, 4.72 mmol), 2-(bromomethyl)-1,4-difluoro-benzene (1.2 g, 5.67 mmol), K₂CO₃ (2.0 g, 14.2 mmol) and MeCN (30.0 mL). The reaction mixture was stirred for 2 h at 80° C. under nitrogen. The solid was filtered out and the filtrate was concentrated under reduced pressure. The residue was recrystallized from water (5 mL) and acetone (5 mL) to afford 5-((2,5-difluorobenzyl)oxy)-2,3-dihydro-1H-inden-1-one (1.26 g, 95.878%) as a yellow solid. LCMS (ESI, m/z): 275 [M+H]⁺.

### Synthesis of methyl 1-(5-((2,5-difluorobenzyl)oxy)-2,3-dihydro-1H-inden-1-yl)azetidine-3-carboxylate

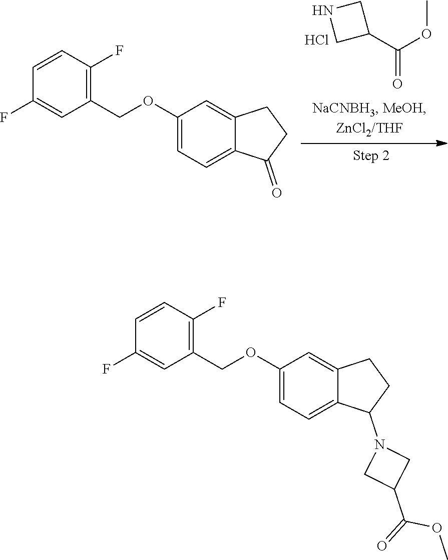

In a 100 mL round-bottom flask was placed a solution of NaBH₃CN (1.1 g, 17.5 mmol) and ZnCl₂/THF (1.19 g, 8.75 mmol) in methanol (50 mL). The resulting mixture was stirred at room temperature for 10 min, followed by the addition of 5-((2,5-difluoro-benzyl)oxy)-2,3-dihydro-1H-inden-1-one (1.2 g, 4.38 mmol) and methyl azetidine-3-carboxylate hydrochloride (0.99 g, 6.56 mmol). The resulting mixture was stirred at 70° C. overnight under nitrogen. The reaction was quenched with NH₄Cl (sat. aq, 80 mL) and extracted with EA (3×80 mL). The organic phase was combined and dried over Na₂SO₄. The solid was filtered out and the filtrate was concentrated under reduced pressure. The residue was purified by TLC on silica gel (DCM:CH₂Cl₂=30:1) to afford methyl 1-(5-((2,5-difluorobenzyl)oxy)-2,3-dihydro-1H-inden-1-yl)azetidine-3-carboxylate (1.4 g, 84%) as a yellow oil. LCMS (ESI, m/z): 374 [M+H]⁺.

### Synthesis of 1-(5-((2,5-difluorobenzyl)oxy)-2,3-dihydro-1H-inden-1-yl)azetidine-3-carboxylic acid

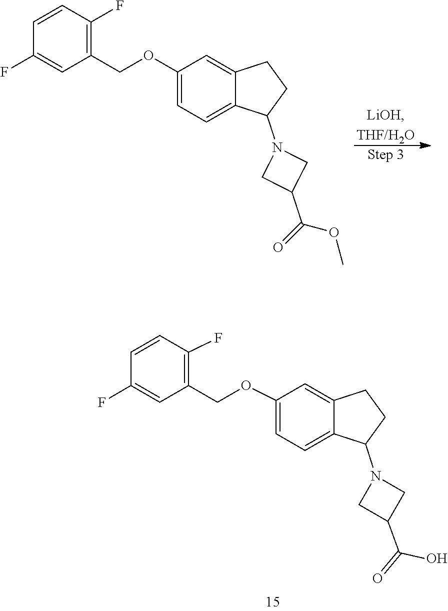 [SMILES: COC(=O)C1CN(C2CCc3cc(OCc4cc(F)ccc4F)ccc32)C1 ; O=C(O)C1CN(C2CCc3cc(OCc4cc(F)ccc4F)ccc32)C1]

To a stirred solution of methyl 1-(5-((2,5-difluorobenzyl)oxy)-2,3-dihydro-1H-inden-1-yl)azetidine-3-carboxylate (500.0 mg, 1.34 mmol) in THF (40 mL) and water (4 mL) was added LiOH H₂O (56.19 mg, 1.34 mmol) in portions at 25° C. The resulting mixture was stirred overnight. LCMS showed the reaction was complete. The reaction was diluted with 10 mL water and the pH adjusted to 3 with 1 M HCl. The solid was collected by filtration and recrystallized from 5 mL water to afford 1-(5-((2,5-difluoro-benzyl)oxy)-2,3-dihydro-1H-inden-1-yl)azetidine-3-carboxylic acid (114 mg, 23%) as an off-white solid.

LCMS (ESI, m/z): 360 [M+H]⁺. Analytic Conditions: column: EVO C18, 3.0*50 mm, 2.6 m; mobile phase A: Water/5 mM NH₄HCO₃, mobile phase B: Acetonitrile, flow rate: 1.20 mL/min; gradient: 10% B to 45% B in 1.74 min, 45% B to 95% B in 0.55 min, hold at 95% B for 0.40 min, 95% B to 10% B in 0.10 min; 254 nm; RT: 1.497 min.

¹H NMR (400 MHz, Methanol-d₄) δ 7.49 (d, J=8.4 Hz, 1H), 7.28 (m, 1H), 7.19-7.05 (m, 3H), 6.99 (dd, J=8.4, 2.5 Hz, 1H), 5.18 (s, 2H), 4.87 (dd, J=7.7, 2.2 Hz, 1H), 4.50-4.40 (m, 2H), 4.36-4.26 (m, 2H), 3.67-3.54 (m, 1H), 3.17 (dt, J=16.6, 8.2 Hz, 1H), 2.98 (ddd, J=16.9, 9.3, 2.9 Hz, 1H), 2.52 (dq, J=16.7, 8.5, 7.7 Hz, 1H), 2.20 (ddt, J=14.9, 8.2, 2.7 Hz, 1H).

### Example S16. Synthesis of 1-(5-((3-chloro-4-fluorobenzyl)oxy)-2,3-dihydro-1H-inden-1-yl)-azetidine-3-carboxylic acid (16)

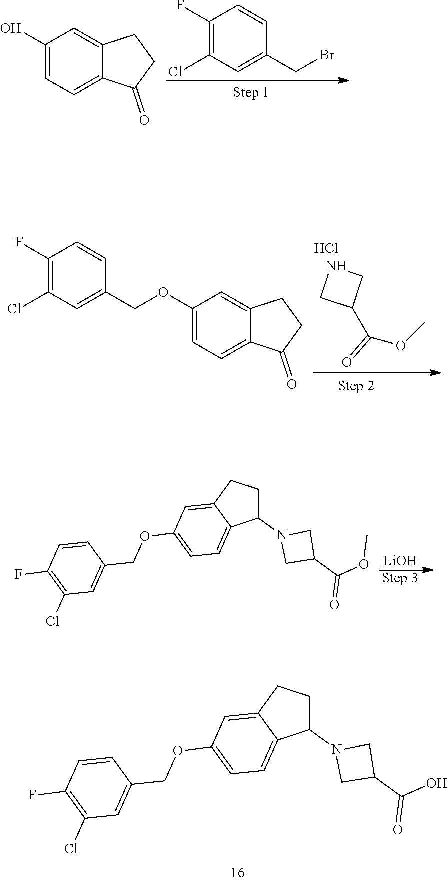

### Synthesis of 5-((3-chloro-4-fluorobenzyl)oxy)-2,3-dihydro-1H-inden-1-one

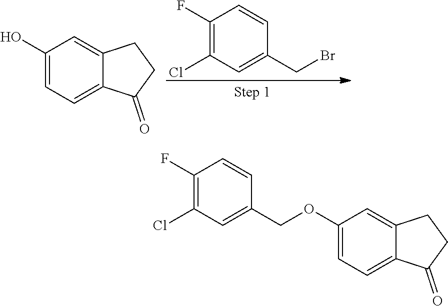

To a solution of 5-hydroxyindan-1-one (600 mg, 4.05 mmol, 1.00 equiv) and 4-(bromomethyl)-2-chloro-1-fluoro-benzene (995 mg, 4.45 mmol, 1.10 equiv) in MeCN (12.0 mL) was added K₂CO₃ (1679 mg, 12.2 mmol, 3.00 equiv). The mixture was stirred overnight at 50° C. LCMS showed the reaction was complete. The solid was filtered out and the filtrate was concentrated under reduce pressure. The residue was purified by flash chromatography on silica gel with petroleum ether/ethyl acetate (6:1) to give 5-((3-chloro-4-fluorobenzyl)oxy)-2,3-dihydro-1H-inden-1-one (1.06 g, 90%) as a yellow solid. LCMS (ESI, m/z): 291 [M+H]⁺.

### Synthesis of methyl 1-(5-((3-chloro-4-fluorobenzyl)oxy)-2,3-dihydro-1H-inden-1-yl)azetidine-3-carboxylate

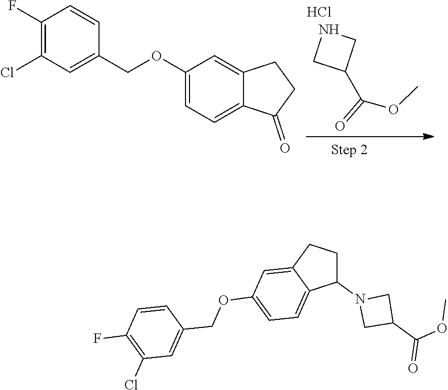

To a solution of NaBH₃CN (433 mg, 6.88 mmol, 4.00 equiv) in methanol (10.0 mL) was added 2 M ZnCl₂ in THF (1.8 mL, 3.44 mmol, 2.00 equiv). The resulting solution was stirred at room temperature for 5 min which was followed by the addition of 5-((3-chloro-4-fluorobenzyl)oxy)-2,3-dihydro-1H-inden-1-one (500 mg, 1.72 mmol, 1.00 equiv) and methyl azetidine-3-carboxylate; hydrochloride (391 mg, 2.58 mmol, 1.50 equiv). The resulting mixture was stirred at 70° C. overnight under nitrogen. LCMS showed the reaction was complete. The reaction was quenched with 20 mL saturated NH₄C₁ solution and extracted with 3×30 mL EA. The organic phases were combined and dried over Na₂SO₄ followed by filtration. The filtrate was concentrated under reduced pressure and purified by TLC with dichloromethane/methanol (20:1) to give methyl 1-(5-((3-chloro-4-fluorobenzyl)oxy)-2,3-dihydro-1H-inden-1-yl)azetidine-3-carboxylate (250 mg, 37%) as a colorless oil. LCMS (ESI, m/z): 390 [M+H]⁺.

### Synthesis of 1-(5-((3-chloro-4-fluorobenzyl)oxy)-2,3-dihydro-1H-inden-1-yl)azetidine-3-carboxylic acid

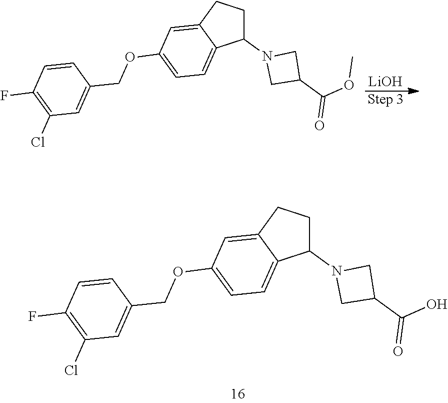 [SMILES: COC(=O)C1CN(C2CCc3cc(OCc4ccc(F)c(Cl)c4)ccc32)C1 ; O=C(O)C1CN(C2CCc3cc(OCc4ccc(F)c(Cl)c4)ccc32)C1]

To a solution of methyl 1-(5-((3-chloro-4-fluorobenzyl)oxy)-2,3-dihydro-1H-inden-1-yl)azetidine-3-carboxylate (250 mg, 0.640 mmol, 1.00 equiv) in THE (5 mL) and water (0.5 mL) was added LiOH H₂O (54 mg, 1.28 mmol, 2.00 equiv). The mixture was stirred overnight at room temperature. The reaction was diluted with 10 mL water and adjusted to pH 3-4 with 2 M HCl. The solvent was removed under reduced pressure and the residue was purified by Prep-HPLC (Column: XSelect CSH Prep C18 OBD Column, 19*250 mm, 5 m; Mobile Phase A: Water (0.05% HCl), Mobile Phase B: ACN; Flow rate: 25 mL/min; Gradient: 50% B to 80% B in 7 min, 80% B; Wave Length: 254/210 nm; RT: 6.78) to give 1-(5-((3-chloro-4-fluorobenzyl)-oxy)-2,3-dihydro-1H-inden-1-yl)azetidine-3-carboxylic acid (134 mg, 99%) as an off-white solid.

LCMS (ESI, m/z): 376 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 60% B in 3.00 min, 60% B to 95% B in 0.30 min, hold at 95% B for 0.40 min, 95% B to 5% B in 0.10 min; 220 nm; RT: 2.547 min.

¹H NMR (400 MHz, Methanol-d4) δ 7.55 (dd, J=7.1, 2.2 Hz, 1H), 7.47 (d, J=8.5 Hz, 1H), 7.40 (ddd, J=7.0, 4.6, 2.1 Hz, 1H), 7.24 (t, J=8.9 Hz, 1H), 7.02 (d, J=2.4 Hz, 1H), 6.95 (dd, J=8.4, 2.5 Hz, 1H), 5.10 (s, 2H), 4.70-4.21 (m, 4H), 3.65 (s, 1H), 3.14 (dt, J=16.5, 8.2 Hz, 1H), 2.96 (ddd, J=16.8, 9.2, 2.8 Hz, 1H), 2.57-2.49 (m, 1H), 2.18 (s, 1H).

### Example S17. Synthesis of 1-(5-((2,4,5-trifluorobenzyl)oxy)-2,3-dihydro-1H-inden-1-yl)azetidine-3-carboxylic acid (17)

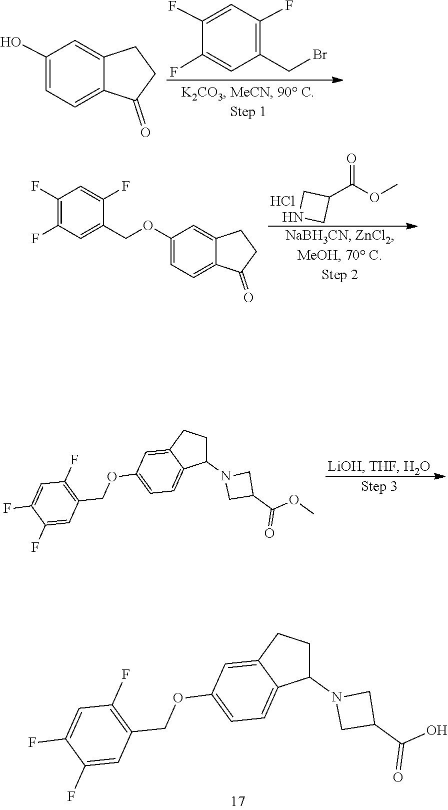

### Synthesis of 5-((2,4,5-trifluorobenzyl)oxy)-2,3-dihydro-1H-inden-1-one

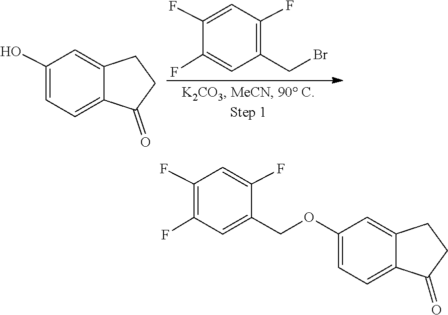

To a solution of 5-hydroxyindan-1-one (1000 mg, 6.75 mmol, 1.00 equiv) in MeCN (30.0 mL) was added 1-(bromomethyl)-2,4,5-trifluoro-benzene (1800 mg, 8.00 mmol, 1.20 equiv) and K₂CO₃ (2778 mg, 13.1 mmol, 3.00 equiv). The resulting mixture was flushed with nitrogen and stirred for 3 h at 90° C. LCMS showed the reaction was complete. The solid was filtered out and the filtrate was concentrated under reduced pressure. The residue was purified by flash chromatography on silica gel (EA:PE=1:6) to afford 5-((2,4,5-trifluorobenzyl)oxy)-2,3-dihydro-1H-inden-1-one (2.0 g, 93%) as a brown solid. LCMS (ESI, m/z): 293 [M+H]⁺.

### Synthesis of methyl 1-(5-((2,4,5-trifluorobenzyl)oxy)-2,3-dihydro-1H-inden-1-yl)azetidine-3-carboxylate

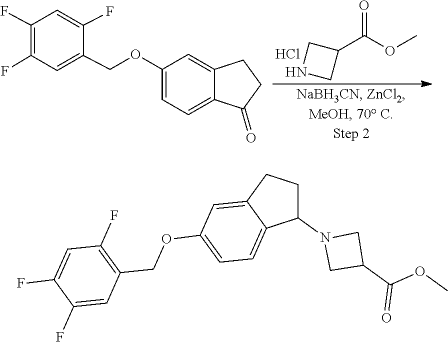

To a solution of NaBH₃CN (689 mg, 11.0 mmol, 4.00 equiv) in methanol (15.0 mL) was added 2 M ZnCl₂ in THF (2.7 mL, 1.82 mmol, 2.00 equiv) and the mixture was stirred for 10 min, followed by the addition of 5-((2,4,5-trifluorobenzyl)oxy)-2,3-dihydro-1H-inden-1-one (800 mg, 2.74 mmol, 1.00 equiv) and methyl azetidine-3-carboxylate hydrochloride (622 mg, 4.11 mmol, 1.50 equiv). The resulting mixture was flushed with nitrogen and stirred overnight at 70° C. LCMS showed the reaction was complete. The mixture was purified by Prep-TLC (PE:EA=1:1) to afford methyl 1-(5-((2,4,5-trifluoro-benzyl)oxy)-2,3-dihydro-1H-inden-1-yl)azetidine-3-carboxylate (782 mg, 73%) as a yellow solid. LCMS (ESI, m/z): 392 [M+H]⁺.

### Synthesis of 1-(5-((2,4,5-trifluorobenzyl)oxy)-2,3-dihydro-1H-inden-1-yl)azetidine-3-carboxylic acid

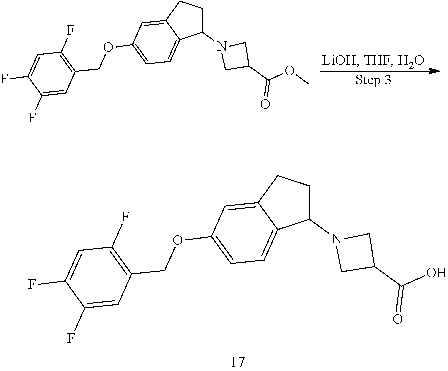 [SMILES: COC(=O)C1CN(C2CCc3cc(OCc4cc(F)c(F)cc4F)ccc32)C1 ; O=C(O)C1CN(C2CCc3cc(OCc4cc(F)c(F)cc4F)ccc32)C1]

To a solution of methyl 1-(5-((2,4,5-trifluorobenzyl)oxy)-2,3-dihydro-1H-inden-1-yl)azetidine-3-carboxylate (782 mg, 2.00 mmol, 1.00 equiv) in THF (15.0 mL) and water (1.5 mL) was added LiOH H₂O (167 mg, 4.00 mmol, 4.00 equiv), the mixture was stirred overnight at room temperature. The reaction was diluted with 15 mL water and the pH adjusted to 3 with 4 M HCl. The solvent was removed under reduced pressure. The crude product was purified by Prep-HPLC (Column: YMC-Actus Triart C18 ExRS. 30*250, 5 m; Mobile Phase A: water (0.05% HCl), Mobile Phase B: ACN; Flow rate: 60 mL/min; Gradient: 25% B to 50% B in 7 min; 254/210 nm; RT: 6.00 min) to afford 1-(5-((2,4,5-trifluorobenzyl)oxy)-2,3-dihydro-1H-inden-1-yl)azetidine-3-carboxylic acid (139.2 mg, 18%) as an off-white solid.

LCMS (ESI, m/z): 378 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 30% B to 65% B in 3.00 min, 65% B to 90% B in 0.20 min, hold at 95% B for 0.10 min, 95% B to 5% B in 0.20 min; 210 nm; RT: 1.195 min.

¹H NMR (400 MHz, CD30D-d₄) δ 7.51-7.41 (m, 2H), 7.27-7.18 (m, 1H), 7.06-7.01 (d, 1H), 6.95 (d, 2H), 5.12 (s, 1H), 4.86 (s, 1H), 4.70-4.27 (m, 4H), 3.65 (s, 1H), 3.17 (m, 1H), 2.97 (m, 1H), 2.51 (m, 1H), 2.18 (m, 1H).

### Example S18. Synthesis of 1-(5-((2,3,5-trifluorobenzyl)oxy)-2,3-dihydro-1H-inden-1-yl)azetidine-3-carboxylic acid (18)

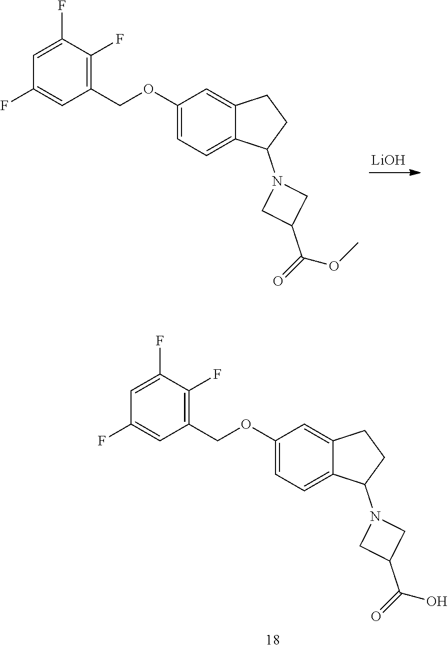 [SMILES: COC(=O)C1CN(C2CCc3cc(OCc4cc(F)cc(F)c4F)ccc32)C1 ; "LiOH" ; O=C(O)C1CN(C2CCc3cc(OCc4cc(F)cc(F)c4F)ccc32)C1]

To a stirred solution of methyl 1-(5-((2,3,5-trifluorobenzyl)oxy)-2,3-dihydro-1H-inden-1-yl)azetidine-3-carboxylate (150 mg, 0.380 mmol, 1.00 equiv) in THF (5.0 mL) was added LiOH H₂O (33 mg, 0.770 mmol, 2.00 equiv) in THF (5.0 mL) and water (0.5 mL). The reaction was stirred at RT overnight. LCMS showed the reaction was complete. The pH value of the solution was adjusted to 4-5 with 2 M HCl. The solvent was removed under reduced pressure. The residue was purified by Prep-HPLC (Column: XBridge Shield RP18 OBD Column, 30*150 mm, 5 m; Mobile Phase A: Water (10 mmol/L NH₄HCO₃), Mobile Phase B: ACN; Flow rate: 60 mL/min; Gradient: 12% B to 40% B in 7 min, 40% B; 254/210 nm; RT: 6.97) to give 1-(5-((2,3,5-trifluorobenzyl)oxy)-2,3-dihydro-1H-inden-1-yl)azetidine-3-carboxylic acid (57 mg, 38%) as an off-white solid.

LCMS (ESI, m/z): 378 [M+H]⁺. Analytic Conditions: column: EVO C18, 50 mm, 3.0 mm, 2.6 m; mobile phase A: Water (5 mM NH₄HCO₃), mobile phase B: Acetonitrile, flow rate: 1.20 mL/min; gradient: 10% B to 95% B in 2.00 min, hold at 95% B for 0.60 min, 95% B to 10% B in 0.15 min; 210 nm; RT: 1.040 min.

¹H NMR (400 MHz, DMSO-d₆) δ 7.47 (dddd, J=11.2, 9.0, 6.2, 3.2 Hz, 1H), 7.5 (m, 1H), 7.13 (d, J=8.2 Hz, 1H), 6.85 (d, J=2.4 Hz, 1H), 6.75 (dd, J=8.2, 2.5 Hz, 1H), 5.10 (s, 2H), 3.65 (dd, J=6.8, 2.8 Hz, 1H), 3.25 (d, J=7.4 Hz, 3H), 3.20-3.00 (m, 2H), 2.80 (m, 1H), 2.65 (ddd, J=16.3, 8.8, 3.7 Hz, 1H), 1.95 (dq, J=15.2, 8.1 Hz, 1H), 1.75 (ddt, J=12.2, 7.6, 3.5 Hz, 1H).

### Example S19. Synthesis of 1-(5-((3,4-difluorobenzyl)oxy)-2,3-dihydro-1H-inden-1-yl)azetidine-3-carboxylic acid (19)

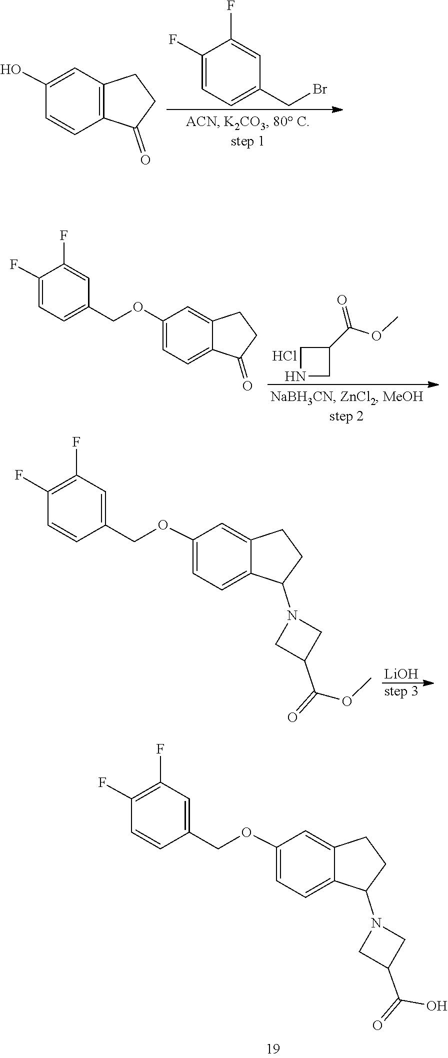

### Synthesis of 5-((3,4-difluorobenzyl)oxy)-2,3-dihydro-1H-inden-1-one

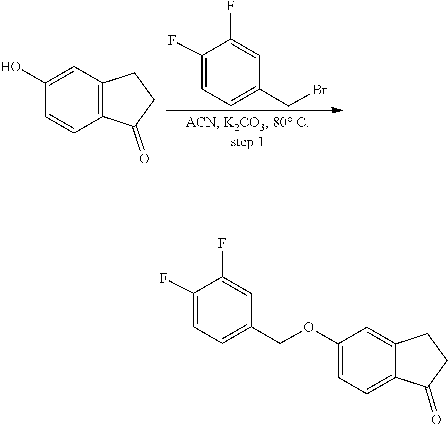 [SMILES: O=C1CCc2cc(O)ccc21 ; Fc1ccc(CBr)cc1F ; O=C1CCc2cc(OCc3ccc(F)c(F)c3)ccc21]

To a solution of 4-(bromomethyl)-1,2-difluoro-benzene (500 mg, 2.42 mmol, 1.00 equiv) and K₂CO₃ (1.0 g, 7.26 mmol, 3.00 equiv) in MeCN (10.0 mL) was added 5-hydroxyindan-1-one (429 mg, 2.90 mmol, 2.00 equiv). The mixture was stirred at 80° C. overnight. LCMS showed desired product was formed. The solid was filtered out and the filtrate was concentrated under reduced pressure. The residue was purified by Prep-TLC (EA:PE=1:3) to afford 5-((3,4-difluorobenzyl)oxy)-2,3-dihydro-1H-inden-1-one (610 mg, 92%). LCMS (ESI, m/z): 275 [M+H]⁺.

### Synthesis of methyl 1-(5-((3,4-difluorobenzyl)oxy)-2,3-dihydro-1H-inden-1-yl)azetidine-3-carboxylate

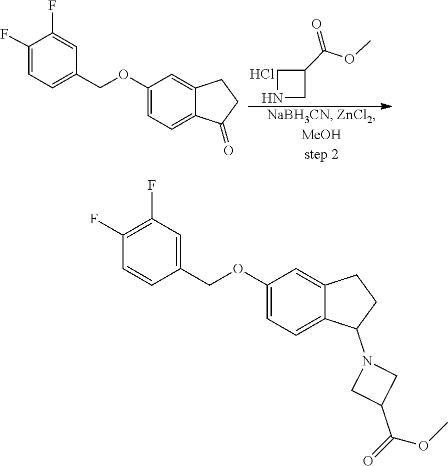

To a solution of NaBH₃CN (560 mg, 8.90 mmol, 4.00 equiv) in methanol (20.0 mL) was added 2 M ZnCl₂ (2.2 mL, 4.45 mmol, 2.00 equiv). Then 5-((3,4-difluoro-benzyl)oxy)-2,3-dihydro-1H-inden-1-one (610 mg, 2.22 mmol, 1.00 equiv) and methyl azetidine-3-carboxylate hydrochloride (674 mg, 4.45 mmol, 2.00 equiv) were added. The mixture was stirred at 70° C. overnight. LCMS showed the desire product was formed. The mixture was diluted with water 10 mL and extracted with EA (3×20 mL). The organic layers were combined, washed with brine (3×10 mL), concentrated and purified by flash chromatography on silica gel (MeOH:DCM=1:3) to afford methyl 1-(5-((3,4-difluorobenzyl)oxy)-2,3-dihydro-1H-inden-1-yl)azetidine-3-carboxylate (420 mg, 50%) as a yellow oil. LCMS (ESI, m/z): 374 [M+H]⁺.

### Synthesis of 1-(5-((3,4-difluorobenzyl)oxy)-2,3-dihydro-1H-inden-1-yl)azetidine-3-carboxylic acid

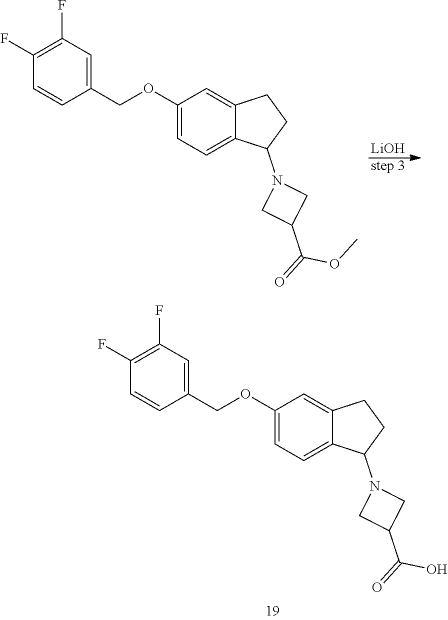 [SMILES: COC(=O)C1CN(C2CCc3cc(OCc4ccc(F)c(F)c4)ccc32)C1 ; O=C(O)C1CN(C2CCc3cc(OCc4ccc(F)c(F)c4)ccc32)C1]

To a solution of methyl 1-(5-((3,4-difluorobenzyl)oxy)-2,3-dihydro-1H-inden-1-yl)azetidine-3-carboxylate (420 mg, 1.12 mmol, 1.00 equiv) in THF (5.0 mL) and water (0.5 mL) was added LiOH H₂O (94 mg, 2.25 mmol, 2.00 equiv). The mixture was stirred at room temperature overnight. The reaction was diluted with water (10 mL) and the pH adjusted to 4-5 with 2 M HCl. The solvent was removed under reduced pressure. The residue was purified by Prep-HPLC (Column: XSelect CSH Prep C18 OBD Column,, 19*250 mm, 5 m; Mobile Phase A: Water (0.05% HCl), Mobile Phase B: ACN; Flow rate: 25 mL/min; Gradient: 25% B to 49% B in 7 min; 210/254 nm; RT: 6.45 min) to afford 11-(5-((3,4-difluorobenzyl)oxy)-2,3-dihydro-1H-inden-1-yl)-azetidine-3-carboxylic acid (176 mg, 43%) as an off-white solid.

LCMS (ESI, m/z): 360 [M+H]⁺. Analytic Conditions: column: Shim-pack XR-ODS, 3.0*50 mm, 2.2 m; mobile phase A: Water/0.05% TFA, mobile phase B: Acetonitrile/0.05% TFA, flow rate: 1.20 mL/min; gradient: 5% B to 95% B in 2.00 min, hold at 95% B for 0.70 min, 95% B to 5% B in 0.25 min; 210 nm; RT: 1.494 min.

¹H NMR (400 MHz, Methanol-d4) δ 7.46 (d, J=8.5 Hz, 1H), 7.40-7.27 (m, 1H), 7.30-7.21 (m, 1H), 7.24 (s, 1H), 7.01 (d, J=2.4 Hz, 1H), 6.95 (dd, J=8.5, 2.5 Hz, 1H), 5.1-5.01 (s, 2H), 4.87 (s, 1H), 4.70-4.20 (m, 4H), 3.65 (s, 1H), 3.13 (dt, J=16.5, 8.2 Hz, 1H), 3.01-2.90 (m, 1H), 2.50 (m, J=16.7, 8.6 Hz, 1H), 2.18 (s, 1H).

### Example S20. Synthesis of 1-(5-((3-iodobenzyl)oxy)-2,3-dihydro-1H-inden-1-yl)azetidine-3-carboxylic acid (20)

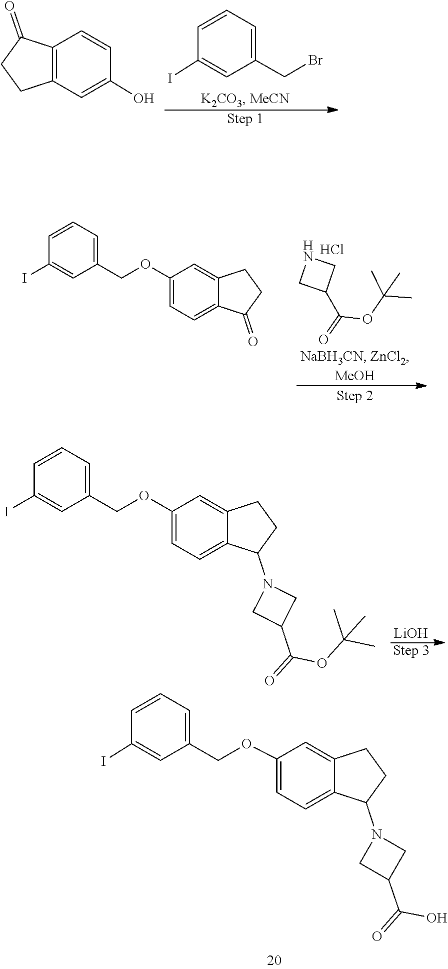

### Synthesis of 5-((3-iodobenzyl)oxy)-2,3-dihydro-1H-inden-1-one

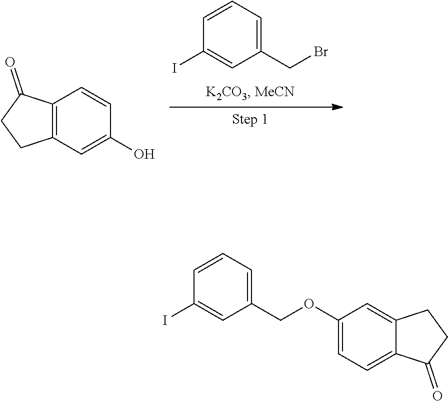 [SMILES: O=C1CCc2cc(O)ccc21 ; BrCc1cccc(I)c1 ; O=C1CCc2cc(OCc3cccc(I)c3)ccc21]

To a solution of the 5-hydroxyindan-1-one (1.3 g, 9.25 mmol, 1.00 equiv) in MeCN (10.0 mL) was added 1-(bromomethyl)-3-iodo-benzene (3.0 g, 10.1 mmol, 1.00 equiv) and K₂CO₃ (3.8 g, 27.7 mmol, 3.00 equiv). The reaction was stirred for 1 h at 80° C. LCMS showed the reaction was complete. The solid was filtered out and the filtrate was concentrated under reduced pressure. The residue was purified by flash chromatography on silica gel (EA:PE=1:6) to give the desired product 5-((3-iodo-benzyl)oxy)-2,3-dihydro-1H-inden-1-one (3.1 g, 92%) as a yellow solid. LCMS (ESI, m/z): 365 [M+H].

### Synthesis of tert-butyl 1-(5-((3-iodobenzyl)oxy)-2,3-dihydro-1H-inden-1-yl)-azetidine-3-carboxylate

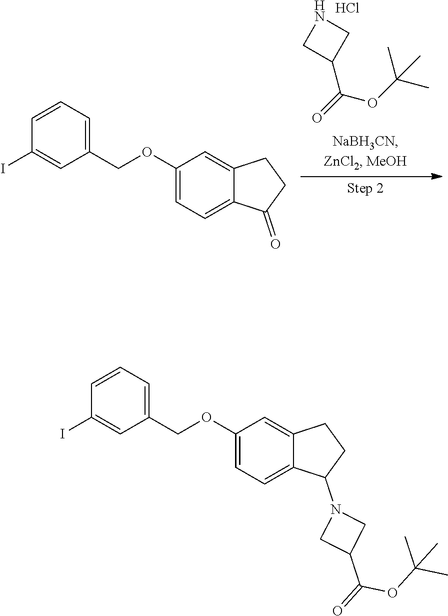 [SMILES: Cl ; O=C1CCc2cc(OCc3cccc(I)c3)ccc21 ; CC(C)(C)OC(=O)C1CNC1 ; CC(C)(C)OC(=O)C1CN(C2CCc3cc(OCc4cccc(I)c4)ccc32)C1]

To a solution of NaBH₃CN (0.7 g, 12.4 mmol, 3.00 equiv) in methanol (5.0 mL) was added ZnCl₂ (4.1 mL, 8.24 mmol, 2.00 equiv) (2 M in THF). The reaction was stirred for 15 min at room temperature. Then 5-((3-iodobenzyl)oxy)-2,3-dihydro-1H-inden-1-one (1.5 g, 4.12 mmol, 1.00 equiv) and tert-butyl azetidin-1-ium-3-carboxylate hydrogen chloride (1.2 g, 6.180 mmol, 1.50 equiv) were added separately. The reaction mixture was then stirred at 60° C. for 12 h. LCMS showed the reaction was complete. The reaction mixture was quenched with water (20 mL) and extracted with EA (3×30 mL). The organic layers were combined and washed with brine (30.0 mL), dried over anhydrous Na₂SO₄, and filtered. The filtrate was concentrated under reduced pressure. The residue was purified by flash chromatography on silica gel (MeOH:DCM=25:1) to afford tert-butyl 1-(5-((3-iodobenzyl)oxy)-2,3-dihydro-1H-inden-1-yl)azetidine-3-carboxylate (1.0 g, 48%) as a yellow oil. LCMS (ESI, m/z): 506 [M+H]⁺.

### Synthesis of 1-(5-((3-iodobenzyl)oxy)-2,3-dihydro-1H-inden-1-yl)azetidine-3-carboxylic acid

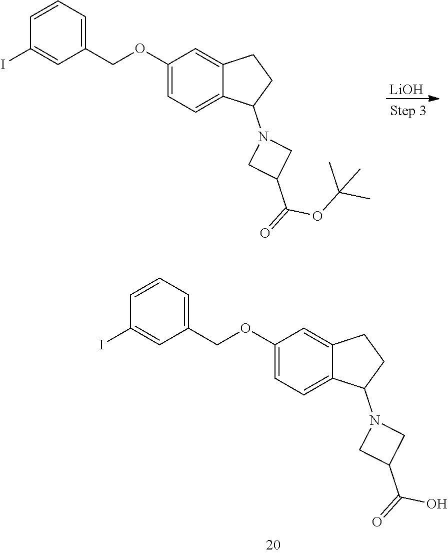 [SMILES: CC(C)(C)OC(=O)C1CN(C2CCc3cc(OCc4cccc(I)c4)ccc32)C1 ; "LiOH" ; O=C(O)C1CN(C2CCc3cc(OCc4cccc(I)c4)ccc32)C1]

To a solution of the compound tert-butyl 1-(5-((3-iodobenzyl)oxy)-2,3-dihydro-1H-inden-1-yl)azetidine-3-carboxylate (200 mg, 0.400 mmol, 1.00 equiv) in THE (3.0 mL) and water (0.6 mL) was added LiOH H₂O (24 mg, 0.590 mmol, 2.00 equiv). The reaction was stirred for 12 h at room temperature. The reaction was diluted with 10 mL water. THE was removed under reduced pressure. 4 M HCl was added to the reaction mixture to adjust the pH to 6. The solvent was removed under reduced pressure. The residue was purified by Perp-HPLC (Column: XBridge Shield RP18 OBD Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 15% B to 44% B in 7 min; Wave Length: 254/210 nm; RT: 6.60 min) to give 1-(5-((3-iodobenzyl)oxy)-2,3-dihydro-1H-inden-1-yl)azetidine-3-carboxylic acid (46 mg, 25%) as a white solid.

LCMS (ESI, m/z): 450 [M+H]⁺. Analytic Conditions: column: EVO C18, 3.0*50 mm, 2.6 m; mobile phase A: Water/5mMNH4HCO₃, mobile phase B: acetonitrile, flow rate: 1.20 mL/min; gradient: 10% B to 95% B in 2.00 min, hold at 95% for 0.60 min, 95% B to 10% B in 0.15 min; 254 nm; RT: 1.141 min.

¹H NMR (400 MHz, Methanol-d₄) δ 7.79 (d, J=1.8 Hz, 1H), 7.66 (d, J=7.7 Hz, 1H), 7.43 (dd, J=8.3, 4.5 Hz, 2H), 7.14 (t, J=7.8 Hz, 1H), 6.99-6.93 (m, 1H), 5.08 (s, 2H), 4.74 (d, J=7.5 Hz, 1H), 4.26 (d, J=7.4 Hz, 2H), 4.13 (t, J=8.7 Hz, 2H), 3.34 (d, J=8.6 Hz, 1H), 3.10 (dt, J=16.5, 8.0 Hz, 1H), 2.93 (ddd, J=16.8, 9.3, 3.0 Hz, 1H), 2.46 (dq, J=16.5, 8.4 Hz, 1H), 2.13 (dd, J=14.5, 7.9 Hz, 1H).

### Example S21. Synthesis of 1-(5-((4-iodobenzyl)oxy)-2,3-dihydro-1H-inden-1-yl)azetidine-3-carboxylic acid (21)

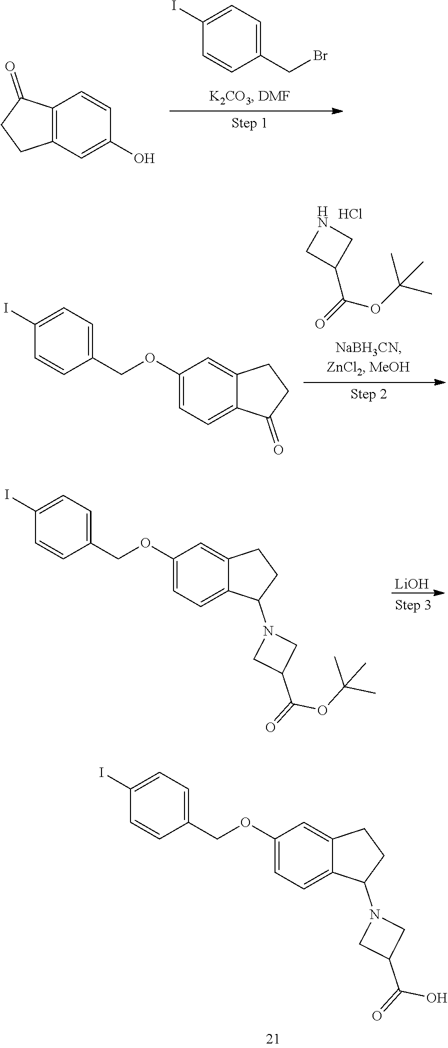

### Synthesis of 5-((4-iodobenzyl)oxy)-2,3-dihydro-1H-inden-1-one

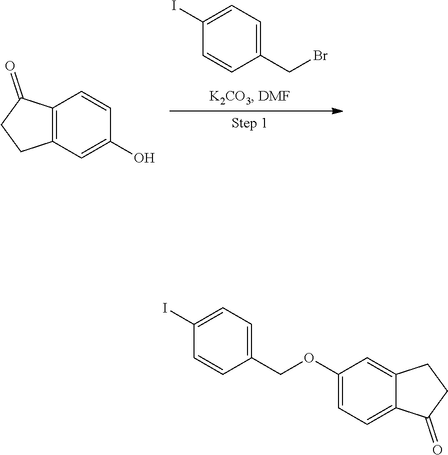 [SMILES: O=C1CCc2cc(O)ccc21 ; BrCc1ccc(I)cc1 ; O=C1CCc2cc(OCc3ccc(I)cc3)ccc21]

To a solution of 5-hydroxyindan-1-one (2.5 g, 16.8 mmol, 1.00 equiv) in DMF (20.0 mL) were added 1-(bromomethyl)-4-iodo-benzene (5.5 g, 18.560 mmol, 1.10 equiv) and K₂CO₃ (7.0 g, 50.6 mmol, 3.00 equiv). The reaction was stirred for at 60° C. 12 h. LCMS showed the reaction was complete. The solid was filtered out and the filtrate was concentrated under reduced pressure. The residue was purified by flash chromatography on silica gel (EA:PE=1:6) to afford the 5-((4-iodobenzyl)oxy)-2,3-dihydro-1H-inden-1-one (5.6 g, 91%). LCMS (ESI, m/z): 365 [M+H]⁺.

### Synthesis of tert-butyl 1-(5-((4-iodobenzyl)oxy)-2,3-dihydro-1H-inden-1-yl)-azetidine-3-carboxylate

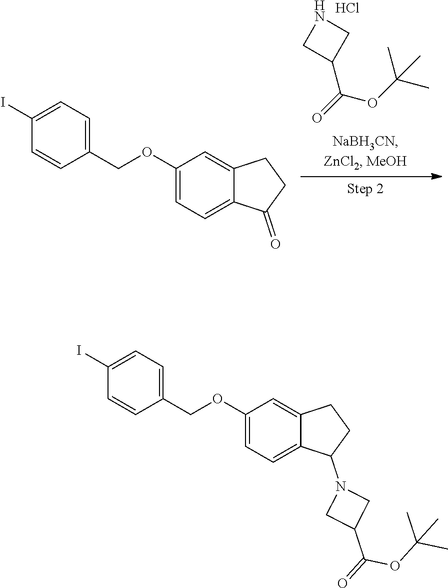 [SMILES: Cl ; O=C1CCc2cc(OCc3ccc(I)cc3)ccc21 ; CC(C)(C)OC(=O)C1CNC1 ; CC(C)(C)OC(=O)C1CN(C2CCc3cc(OCc4ccc(I)cc4)ccc32)C1]

To a solution of NaBH₃CN (776 mg, 12.360 mmol, 3.00 equiv) in methanol (8.0 mL) was added ZnCl₂ (4.0 mL, 8.24 mmol, 2.00 equiv) (2 M in THF). The reaction was stirred for 15 min at room temperature. Then 5-((4-iodobenzyl)oxy)-2,3-dihydro-1H-inden-1-one (1.5 g, 4.120 mmol, 1.00 equiv) and tert-butyl azetidine-3-carboxylate hydrochloride (1.2 g, 6.18 mmol, 1.50 equiv) were added separately. The reaction mixture was heated for 12 h at 60° C. LCMS showed the reaction was complete. The reaction was poured into 50 mL H₂O and extracted with EA (3×50 mL). The combined organic layer was washed with brine 1×50 mL) and dried over anhydrous Na₂SO₄. The solid was filtered out and the filtrate was concentrated under reduced pressure. The residue was purified by column chromatography on silica gel (MeOH:DCM=25:1) to give tert-butyl 1-(5-((4-iodobenzyl)oxy)-2,3-dihydro-1H-inden-1-yl)azetidine-3-carboxylate (1.5 g, 72%) LCMS (ESI, m/z): 506 [M+H]⁺.

### Synthesis of 1-(5-((4-iodobenzyl)oxy)-2,3-dihydro-1H-inden-1-yl)azetidine-3-carboxylic acid

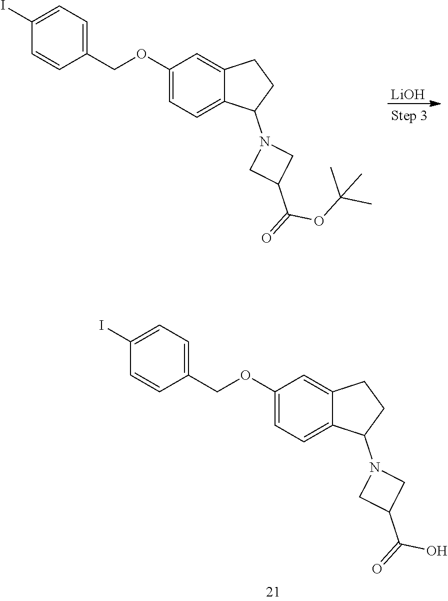 [SMILES: CC(C)(C)OC(=O)C1CN(C2CCc3cc(OCc4ccc(I)cc4)ccc32)C1 ; "LiOH" ; O=C(O)C1CN(C2CCc3cc(OCc4ccc(I)cc4)ccc32)C1]

To a solution of tert-butyl 1-(5-((4-iodobenzyl)oxy)-2,3-dihydro-1H-inden-1-yl)-azetidine-3-carboxylate (100 mg, 0.200 mmol, 1.00 equiv) in THF (3.0 mL) and water (0.5 mL) was added LiOH—H₂O (17 mg, 0.400 mmol, 2.00 equiv) at 25° C. The mixture was stirred at 25° C. for 24 h. The reaction was diluted with 10 mL water. THF was removed under reduced pressure. The pH was adjusted to 6 with 4 M hydrochloric acid. The mixture was concentrated and purified by Prep-HPLC (Column: X Bridge Prep C18 OBD Column, 19×150 mm 5 m; Mobile Phase A: Water (10 mmol/L NH₄HCO₃), Mobile Phase B: ACN; Flow rate: 25 mL/min; Gradient: 26 B % to 40 B % in 7 min; 210/254 nm; RT: 6.83 min) to give 1-(5-((4-iodobenzyl)oxy)-2,3-dihydro-1H-inden-1-yl)azetidine-3-carboxylic acid (26.9 mg) as an off-white solid.

LCMS (ESI, m/z): 450 [M+H]⁺. Analytic Conditions: Column: Shim-pack XR-ODS 3.0*50 mm, 2.2 m; Mobile Phase A: Water/0.05% TFA, Mobile Phase B: Acetonitrile/0.05% TFA; Flow rate: 1.20 mL/min; gradient: 5% B to 95% B in 1.99 min, hold at 95% B for 0.70 min, 95% B to 5% B in 0.05 min; 220 nm; Rt: 1.640 min.

¹H NMR (400 MHz, Methanol-d₄) δ 7.72-7.69 (m, 2H), 7.42 (d, J=8.4 Hz, 1H), 7.23 (d, J=8.3 Hz, 2H), 6.99 (s, 1H), 6.94 (dd, J=8.6, 2.5 Hz, 1H), 5.07 (s, 2H), 4.70 (d, J=7.4 Hz, 1H), 4.27 (t, J=9.3 Hz, 2H), 4.13 (m, J=7.5 Hz, 2H), 3.34 (s, 1H), 3.08 (m, 1H), 2.91 (m, 1H), 2.44 (dq, J=16.3, 8.3 Hz, 1H), 2.15 (dd, J=14.6, 8.1 Hz, 1H).

### Example S22. Synthesis of (1R,5S,6r)-3-(5-((2,6-dichlorobenzyl)oxy)-2,3-dihydro-1H-inden-1-yl)-3-azabicyclo[3.1.0]hexane-6-carboxylic acid (22)

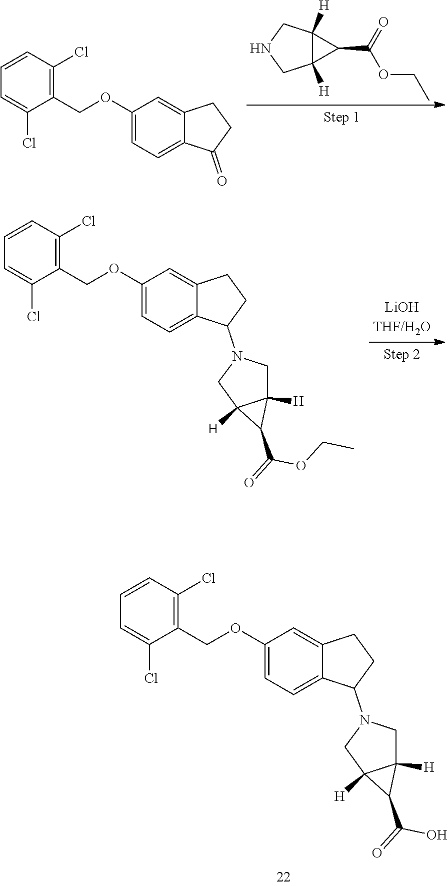 [SMILES: O=C1CCc2cc(OCc3c(Cl)cccc3Cl)ccc21 ; CCOC(=O)[C@H]1[C@@H]2CNC[C@@H]21 ; CCOC(=O)[C@H]1[C@@H]2CN(C3CCc4cc(OCc5c(Cl)cccc5Cl)ccc43)C[C@@H]21 ; O=C(O)[C@H]1[C@@H]2CN(C3CCc4cc(OCc5c(Cl)cccc5Cl)ccc43)C[C@@H]21]

### Synthesis of ethyl (1R,5S,6r)-3-(5-((2,6-dichlorobenzyl)oxy)-2,3-dihydro-1H-inden-1-yl)-3-azabicyclo[3.1.0]hexane-6-carboxylate

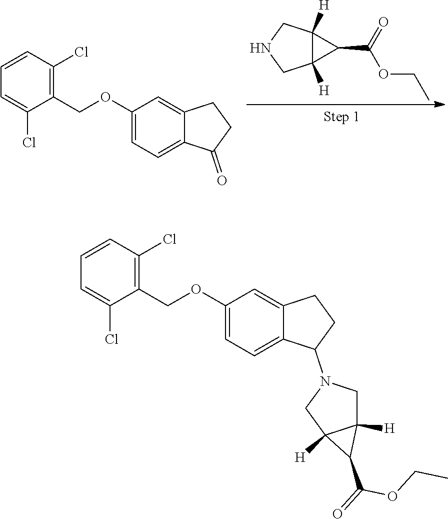 [SMILES: O=C1CCc2cc(OCc3c(Cl)cccc3Cl)ccc21 ; CCOC(=O)[C@H]1[C@@H]2CNC[C@@H]21 ; CCOC(=O)[C@H]1[C@@H]2CN(C3CCc4cc(OCc5c(Cl)cccc5Cl)ccc43)C[C@@H]21]

In to a 25 mL round-bottom flask was placed a solution of NaBH₃CN (98 mg, 1.56 mmol, 4.00 equiv) and ZnCl_{2/2}-Me-THF (2.0 M) (0.39 mL, 0.780 mmol, 2.00 equiv) in methanol (2.0 mL). The resulting solution was stirred at room temperature for 10 min which was followed by the addition of 5-((2,6-dichlorobenzyl)oxy)-2,3-dihydro-1H-inden-1-one (120 mg, 0.390 mmol, 1.00 equiv) and ethyl (1R,5S,6r)-3-azabicyclo[3.1.0]hexane-6-carboxylate (91 mg, 0.590 mmol, 1.50 equiv). The reaction solution was stirred at 60° C. for 16 h under nitrogen. LCMS showed the reaction was complete. The reaction was quenched with 10 mL saturated NH₄C₁ solution and extracted with EA (3×10 mL). Organic phase was combined and dried over Na₂SO₄. The solid was filtered out and the filtrate was concentrated under reduced pressure. The crude product was purified by flash chromatography on silica gel with ethyl acetate/petroleum ether (1:5) to give ethyl (1R,5S,6r)-3-(5-((2,6-dichlorobenzyl)oxy)-2,3-dihydro-1H-inden-1-yl)-3-azabicyclo[3.1.0]hexane-6-carboxylate (75 mg, 43%) as a colorless semi-solid. LCMS (ESI, m/z): 446 [M+H]⁺.

### Synthesis of (1R,5S,6r)-3-(5-((2,6-dichlorobenzyl)oxy)-2,3-dihydro-1H-inden-1-yl)-3-azabicyclo[3.1.0]hexane-6-carboxylic acid

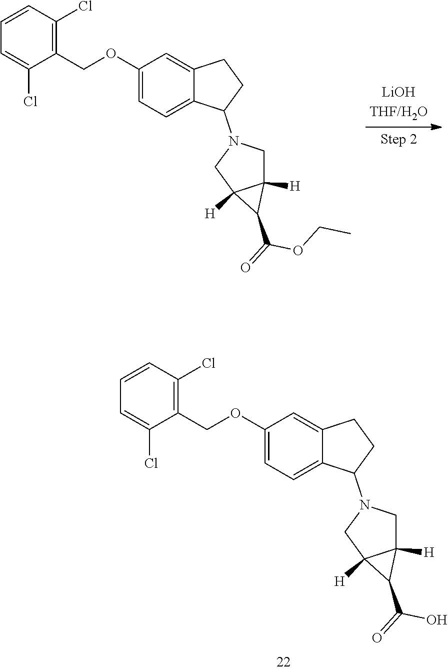 [SMILES: CCOC(=O)[C@H]1[C@@H]2CN(C3CCc4cc(OCc5c(Cl)cccc5Cl)ccc43)C[C@@H]21 ; O=C(O)[C@H]1[C@@H]2CN(C3CCc4cc(OCc5c(Cl)cccc5Cl)ccc43)C[C@@H]21]

Into a 25 mL round-bottom flask was placed a solution of ethyl (1R,5S,6r)-3-(5-((2,6-dichlorobenzyl)oxy)-2,3-dihydro-1H-inden-1-yl)-3-azabicyclo[3.1.0]hexane-6-carboxylate (50 mg, 0.110 mmol, 1.00 equiv), LiOH H₂O (47 mg, 1.120 mmol, 10.00 equiv) in a mixed solvent of THF (1.0 mL) and water (1.0 mL). The resulting mixture was stirred at 80° C. for 5 days. LCMS showed the reaction was complete. The resulting mixture was filtered and purified directly by Prep-HPLC (Column: Xselect CSH OBD Column, 30*150 mm, 5 m; Mobile Phase A: Water (10 mmol/L NH₄HCO₃), Mobile Phase B: ACN; Flow rate: 25 mL/min; Gradient: 25% B to 45% B in 7 min; 254/210 nm; Rt: 6.63 min) to give (1R,5S,6r)-3-(5-((2,6-dichlorobenzyl)-oxy)-2,3-dihydro-1H-inden-1-yl)-3-azabicyclo[3.1.0]hexane-6-carboxylic acid (4.2 mg, 9%) as a white solid.

LCMS (ESI, m/z): 418 [M+H]⁺. Analytic Conditions: Column: Poroshell HPH-C18 Column 3.0*50 mm, 2.7 m; Mobile Phase A: Water/5 mM NH₄HCO₃, Mobile Phase B: Acetonitrile; Flow rate: 1.20 mL/min; Gradient: 10% B to 95% B in 2 min; hold at 95% B for 0.60 min, 95% B to 10% B in 0.20 min; 220 nm; Rt: 1.363 min.

¹H NMR (400 MHz, DMSO-d₆) δ 7.58 (d, J=8.0 Hz, 2H), 7.49 (dd, J=9.0, 7.2 Hz, 1H), 7.32 (d, J=8.4 Hz, 1H), 7.04 (s, 1H), 6.93 (d, J=8.2 Hz, 1H), 5.23 (s, 2H), 4.45 (s, 1H), 3.07 (t, J=7.5 Hz, 5H), 2.85 (s, 1H), 2.21 (d, J=7.1 Hz, 2H), 1.95 (d, J=8.1 Hz, 2H), 1.59 (t, J=8.1 Hz, 1H).

### Example S23. Synthesis of (1R,5S,6s)-3-(5-((2,6-dichlorobenzyl)oxy)-2,3-dihydro-1H-inden-1-yl)-3-azabicyclo[3.1.0]hexane-6-carboxylic acid (23)

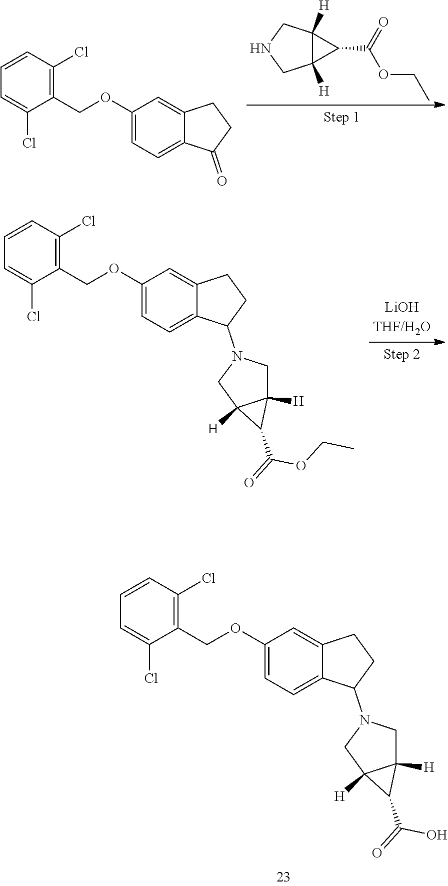 [SMILES: O=C1CCc2cc(OCc3c(Cl)cccc3Cl)ccc21 ; CCOC(=O)[C@@H]1[C@@H]2CNC[C@@H]21 ; CCOC(=O)[C@@H]1[C@@H]2CN(C3CCc4cc(OCc5c(Cl)cccc5Cl)ccc43)C[C@@H]21 ; O=C(O)[C@@H]1[C@@H]2CN(C3CCc4cc(OCc5c(Cl)cccc5Cl)ccc43)C[C@@H]21]

### Synthesis of ethyl (1R,5S,6s)-3-(5-((2,6-dichlorobenzyl)oxy)-2,3-dihydro-1H-inden-1-yl)-3-azabicyclo[3.1.0]hexane-6-carboxylate

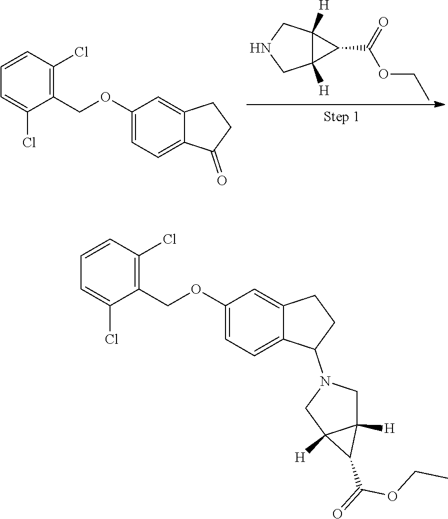 [SMILES: O=C1CCc2cc(OCc3c(Cl)cccc3Cl)ccc21 ; CCOC(=O)[C@@H]1[C@@H]2CNC[C@@H]21 ; CCOC(=O)[C@@H]1[C@@H]2CN(C3CCc4cc(OCc5c(Cl)cccc5Cl)ccc43)C[C@@H]21]

In to a round-bottom flask purged and maintained with an inert atmosphere of nitrogen was placed a solution of NaBH₃CN (98 mg, 1.560 mmol, 4.00 equiv) in methanol (2.0 mL). ZnCl₂/2-Me-THF (2.0 M) (0.39 mL, 0.7800 mmol, 2.00 equiv) was added. The resulting solution was stirred at room temperature for 5-10 min. 5-((2,6-dichlorobenzyl)oxy)-2,3-dihydro-1H-inden-1-one (120 mg, 0.390 mmol, 1.00 equiv) and ethyl (1R,5S,6s)-3-azabicyclo[3.1.0]hexane-6-carboxylate (91 mg, 0.590 mmol, 1.50 equiv) were added. The mixture was stirred at 60° C. for 16 h under nitrogen. LCMS showed the reaction was complete. The reaction mixture was purified by flash chromatography on silica gel with ethyl acetate/petroleum ether (1:5) to give ethyl (1R,5S,6s)-3-(5-((2,6-dichlorobenzyl)oxy)-2,3-dihydro-1H-inden-1-yl)-3-azabicyclo-[3.1.0]hexane-6-carboxylate. LCMS (ESI, m/z): 446 [M+H]⁺.

### Synthesis of (1R,5S,6s)-3-(5-((2,6-dichlorobenzyl)oxy)-2,3-dihydro-1H-inden-1-yl)-3-azabicyclo[3.1.0]hexane-6-carboxylic acid

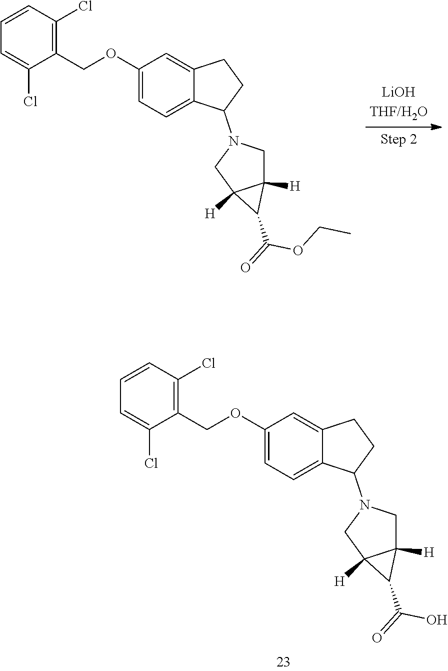 [SMILES: CCOC(=O)[C@@H]1[C@@H]2CN(C3CCc4cc(OCc5c(Cl)cccc5Cl)ccc43)C[C@@H]21 ; O=C(O)[C@@H]1[C@@H]2CN(C3CCc4cc(OCc5c(Cl)cccc5Cl)ccc43)C[C@@H]21]

Into a 25 mL round-bottom flask was placed a solution of ethyl (1R,5S,6s)-3-(5-((2,6-dichlorobenzyl)oxy)-2,3-dihydro-1H-inden-1-yl)-3-azabicyclo[3.1.0]hexane-6-carboxylate (100 mg, 0.220 mmol, 1.00 equiv), LiOH H₂O (56 mg, 1.340 mmol, 6.00 equiv), THE (1.0 mL) and water (1.0 mL). The resulting mixture was stirred at 80° C. for 14 h. LCMS showed the reaction was complete. The resulting mixture was filtered and purified directly by Prep-HPLC (Column: XBridge Prep C18 OBD Column, 19×150 mm 5 m; Mobile Phase A: Water (10 mmol/L NH₄HCO₃), Mobile Phase B: ACN; Flow rate: 25 mL/min; Gradient: 28% B to 42% B in 7 min; 254/210 nm; Rt: 6.22 min) to give (1R,5S,6s)-3-(5-((2,6-dichloro-benzyl)oxy)-2,3-dihydro-1H-inden-1-yl)-3-azabicyclo[3.1.0]hexane-6-carboxylic acid (55.4 mg, 58%) as a white solid.

LCMS (ESI, m/z): 418 [M+H]⁺. Analytic Conditions: Column: Poroshell HPH-C18 Column 3.0*50 mm, 2.7 m; Mobile Phase A: Water/5 mM NH₄HCO₃, Mobile Phase B: Acetonitrile; Flow rate: 1.20 mL/min; Gradient: 10% B to 95% B in 2 min, hold at 95% B for 0.60 min, 95% B to 10% B in 0.20 min; 220 nm; RT: 1.296 min.

¹H NMR (400 MHz, DMSO-d₆) δ 7.61-7.55 (m, 2H), 7.48 (dd, J=8.9, 7.2 Hz, 1H), 7.16 (d, J=8.2 Hz, 1H), 6.94 (d, J=2.4 Hz, 1H), 6.84 (dd, J=8.3, 2.6 Hz, 1H), 5.20 (s, 2H), 4.20-4.13 (m, 1H), 2.89 (dd, J=17.7, 8.3 Hz, 2H), 2.76 (dd, J=18.3, 8.4 Hz, 2H), 2.61-2.52 (m, 2H), 2.08-1.93 (m, 2H), 1.87-1.78 (m, 3H).

### Example S24. Synthesis of (3S)-1-(5-((2,6-dichlorobenzyl)oxy)-2,3-dihydro-1H-inden-1-yl)pyrrolidine-3-carboxylic acid (24a & 24b)

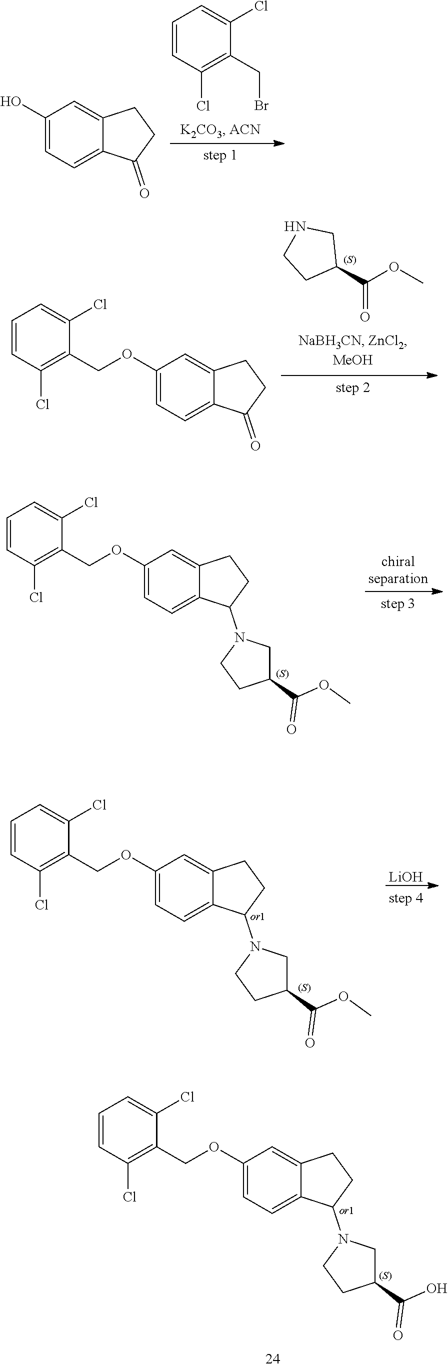

### Synthesis of 5-((2,6-dichlorobenzyl)oxy)-2,3-dihydro-1H-inden-1-one

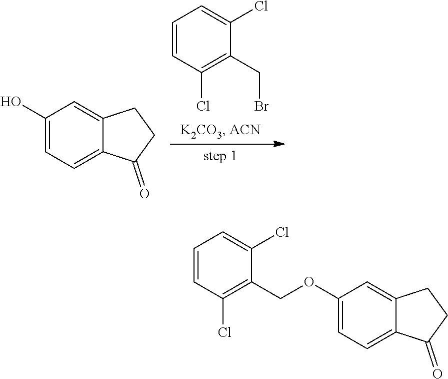 [SMILES: O=C1CCc2cc(O)ccc21 ; Clc1cccc(Cl)c1CBr ; O=C1CCc2cc(OCc3c(Cl)cccc3Cl)ccc21]

To a solution of 5-hydroxyindan-1-one (2.0 g, 13.5 mmol, 1.00 equiv) in MeCN (10 mL) were added 2-(bromomethyl)-1,3-dichloro-benzene (0.5 mL, 13.5 mmol, 1.10 equiv) and K₂CO₃ (5.6 g, 40.5 mmol, 3.00 equiv). The reaction was stirred for 12 h at 60° C. LCMS showed the reaction was complete. The mixture was filtered through celite and the filtrate was concentrated under vacuum. The residue was purified by flash chromatography on C18 silica (eluted with water/MeCN, 20/80) to give 5-((2,6-dichlorobenzyl)oxy)-2,3-dihydro-1H-inden-1-one (3.6 g, 87%) as a yellow solid. LCMS (ESI, m/z): 307 [M+H]⁺.

### Synthesis of methyl (3S)-1-(5-((2,6-dichlorobenzyl)oxy)-2,3-dihydro-1H-inden-1-yl)-pyrrolidine-3-carboxylate

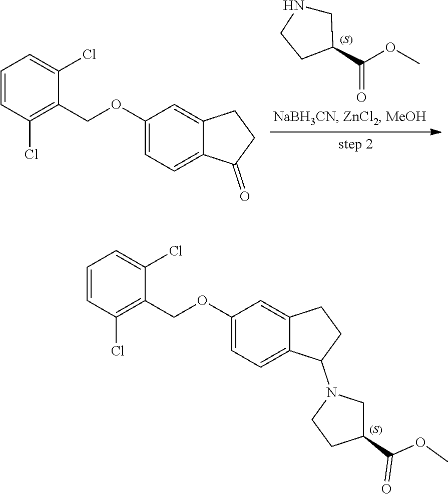

To a solution of NaBH₃CN (184 mg, 2.93 mmol, 3.00 equiv) in methanol (10 mL) was added ZnCl₂ (2 M in THF, 1.0 mL, 1.95 mmol, 2.00 equiv). The mixture was stirred for 15 min at room temperature. Then 5-((2,6-dichlorobenzyl)oxy)-2,3-dihydro-1H-inden-1-one (300 mg, 0.98 mmol, 1.00 equiv) and methyl (3S)-pyrrolidine-3-carboxylate (378 mg, 2.93 mmol, 3.00 equiv) were added. The resulting mixture was stirred overnight at 60° C. LCMS showed the reaction was complete. The mixture was filtered through celite and the filtrate was concentrated under vacuum. The residue was purified by flash chromatography on C18 silica (eluted with water/MeCN, 35/65) to give methyl (3S)-1-(5-((2,6-dichlorobenzyl)oxy)-2,3-dihydro-1H-inden-1-yl)pyrrolidine-3-carboxylate (200 mg, 49%) as a yellow oil. LCMS (ESI, m/z): 420 [M+H]⁺.

### Chiral Separation of methyl (3S)-1-(5-((2,6-dichlorobenzyl)oxy)-2,3-dihydro-1H-inden-1-yl)pyrrolidine-3-carboxylate

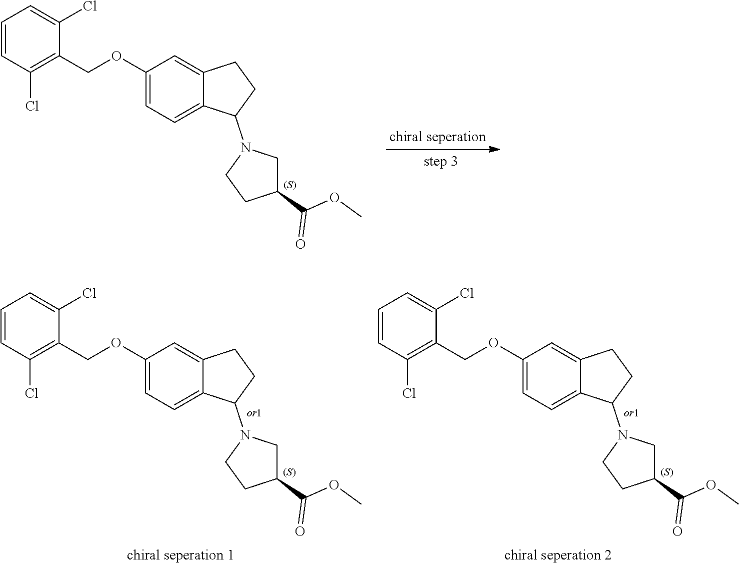

The racemic methyl (3S)-1-(5-((2,6-dichlorobenzyl)oxy)-2,3-dihydro-1H-inden-1-yl)-pyrrolidine-3-carboxylate (200 mg) was resolved by Prep-Chiral-HPLC (column: CHIRALPAK IG, 2*25 cm, 5 m; mobile phase A: Hex (8 mM NH₃·MeOH)-HPLC, mobile phase B: EtOH-HPLC; Flow rate: 20 mL/min; Gradient: 10% B to 10% B in 16 min; 220/254 nm; RT1: 9.4 min; RT2: 12.8 min) to give 60 mg of both enantiomers. LCMS (ESI, m/z): 420 [M+H]⁺.

### Synthesis of (3S)-1-(5-((2,6-dichlorobenzyl)oxy)-2,3-dihydro-1H-inden-1-yl)pyrrolidine-3-carboxylic acid (24a)

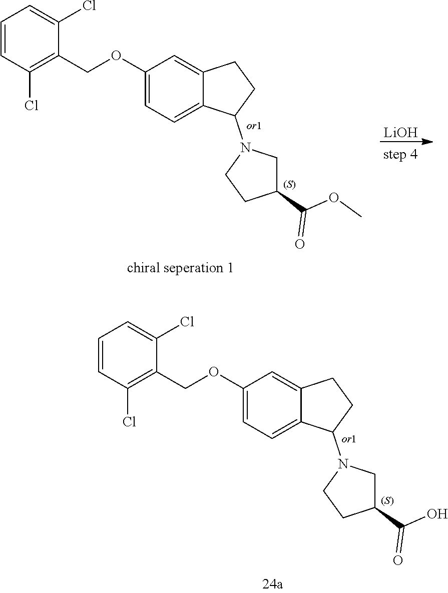

To a solution of methyl (3S)-1-(5-((2,6-dichlorobenzyl)oxy)-2,3-dihydro-1H-inden-1-yl)pyrrolidine-3-carboxylate (chiral separation 1, 60 mg, 0.14 mmol, 1.00 equiv) in THE (3 mL) and water (1 mL) was added LiOH H₂O (18 mg, 0.43 mmol, 3.00 equiv). The resulting mixture was stirred for 6 h at room temperature. LCMS showed the reaction was complete. The reaction mixture was acidified to pH 4-5 using 1 M HCl and then was concentrated. The residue was purified by Prep-HPLC (Column: XBridge Prep C18 OBD Column, 19*150 mm, 5 m; Mobile Phase A: water (10 mM NH₄HCO₃), Mobile Phase B: ACN; Flow rate: 25 mL/min; Gradient: 25% B to 43% B in 7 min; Wavelength: 210/254 nm; RT: 6.32 min) to give (3S)-1-(5-((2,6-dichlorobenzyl)oxy)-2,3-dihydro-1H-inden-1-yl)pyrrolidine-3-carboxylic acid (39.8 mg, 67%) as an off-white solid.

LCMS (ESI, m/z): 406 [M+H]⁺. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 60% B in 2.80 min, 60% B to 95% B in 0.40 min, hold at 95% for 0.50 min, 95% B to 5% B in 0.10 min; 210 nm; RT: 2.616 min.

¹H NMR (300 MHz, CD30D-d₄) δ 7.53 (d, J=8.7 Hz, 1H), 7.47-7.44 (m, 2H), 7.39-7.34 (m, 1H), 7.07-7.05 (m, 1H), 7.00-6.96 (m, 1H), 5.31 (s, 2H), 4.81-4.85 (m, 1H), 3.61-3.55 (m, 1H), 3.42-3.33 (m, 3H), 3.24-3.16 (m, 1H), 3.10-2.93 (m, 2H), 2.61-2.39 (m, 2H), 2.34-2.15 (m, 2H).

### Synthesis of (3S)-1-(5-((2,6-dichlorobenzyl)oxy)-2,3-dihydro-1H-inden-1-yl)pyrrolidine-3-carboxylic acid (24b)

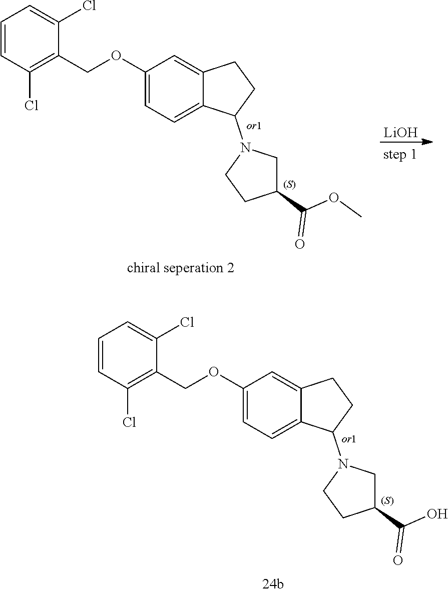

To a solution of methyl (3S)-1-(5-((2,6-dichlorobenzyl)oxy)-2,3-dihydro-1H-inden-1-yl)pyrrolidine-3-carboxylate (chiral separation 2, 60 mg, 0.14 mmol, 1.00 equiv) in THE (3 mL) and water (1 mL) was added LiOH H₂O (18 mg, 0.43 mmol, 3.00 equiv). The resulting mixture was stirred for 6 h at room temperature. LCMS showed the reaction was complete. The reaction mixture was acidified to pH 4-5 using 1 M HCl and then was concentrated. The residue was purified by Prep-HPLC (Column: XBridge Prep C18 OBD Column, 19*150 mm, 5 m; Mobile Phase A: water (10 mM NH₄HCO₃), Mobile Phase B: ACN; Flow rate: 25 mL/min; Gradient: 25% B to 42% B in 7 min; Wavelength: 210/254 nm; RT: 6.32 min) to give (3S)-1-(5-((2,6-dichlorobenzyl)oxy)-2,3-dihydro-1H-inden-1-yl)pyrrolidine-3-carboxylic acid (24.7 mg, 42%) as an off-white solid.

LCMS (ESI, m/z): 406 [M+H]⁺. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 60% B in 2.80 min, 60% B to 95% B in 0.40 min, hold at 95% for 0.50 min, 95% B to 5% B in 0.10 min; 210 nm; RT: 2.621 min.

¹H NMR (300 MHz, DMSO-d₆) δ 7.60-7.57 (m, 2H), 7.51-7.46 (m, 1H), 7.27-7.24 (m, 1H), 6.98-6.95 (m, 1H), 6.89-6.84 (m, 1H), 5.22 (s, 2H), 4.14-4.07 (m, 1H), 2.96-2.85 (m, 3H), 2.76-2.73 (m, 2H), 2.66-2.63 (m, 2H), 2.13-2.07 (m, 2H), 1.97-1.90 (m, 2H).

### Example S25. Synthesis of (3R)-1-(5-((2,6-dichlorobenzyl)oxy)-2,3-dihydro-1H-inden-1-yl)pyrrolidine-3-carboxylic acid (25a & 25b)

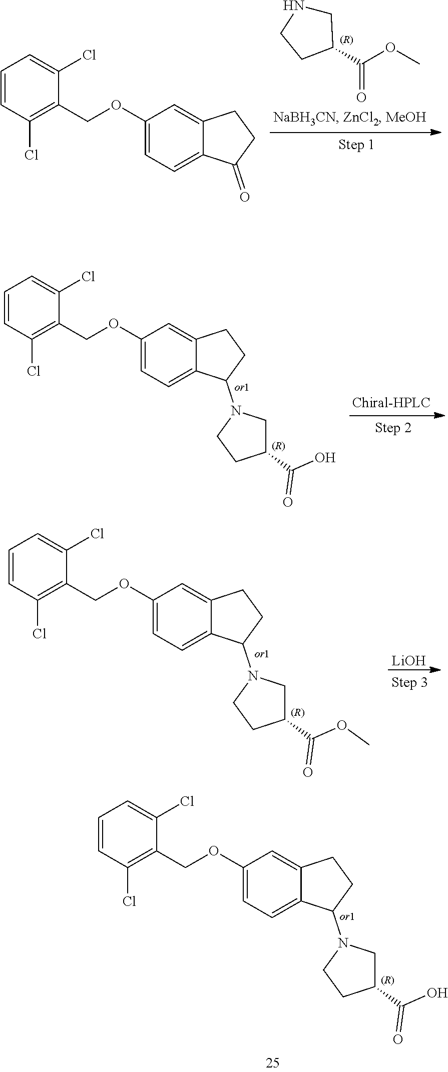

### Synthesis of methyl (3R)-1-(5-((2,6-dichlorobenzyl)oxy)-2,3-dihydro-1H-inden-1-yl)-pyrrolidine-3-carboxylate

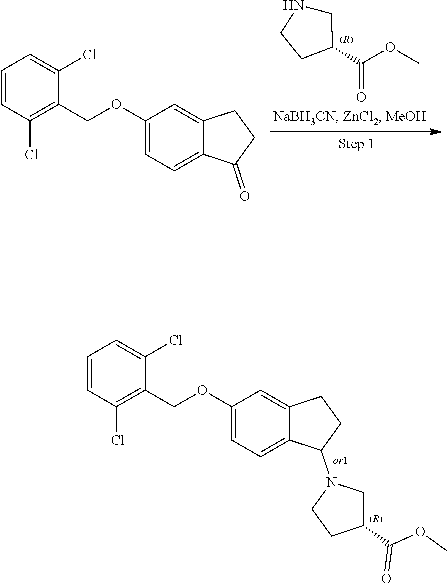

To a solution of NaBH₃CN (184 mg, 2.93 mmol, 3.00 equiv) in methanol (10 mL) was added ZnCl₂ (2 M in THF, 1.0 mL, 1.95 mmol, 2.00 equiv). The mixture was stirred for 15 min at room temperature. Then 5-((2,6-dichlorobenzyl)oxy)-2,3-dihydro-1H-inden-1-one (300 mg, 0.98 mmol, 1.00 equiv) and methyl (3R)-pyrrolidine-3-carboxylate (378 mg, 2.93 mmol, 3.00 equiv) were added. The resulting mixture was stirred overnight at 60° C. LCMS showed the reaction was complete. The mixture was filtered through celite and the filtrate was concentrated under vacuum. The residue was purified by flash chromatography on C18 silica (eluted with water/MeCN, 35/65) to give methyl (3R)-1-(5-((2,6-dichlorobenzyl)oxy)-2,3-dihydro-1H-inden-1-yl)pyrrolidine-3-carboxylate (200 mg, 49%) as a yellow oil. LCMS (ESI, m/z): 420 [M+H]⁺.

### Chiral Separation of methyl (3R)-1-(5-((2,6-dichlorobenzyl)oxy)-2,3-dihydro-1H-inden-1-yl)pyrrolidine-3-carboxylate

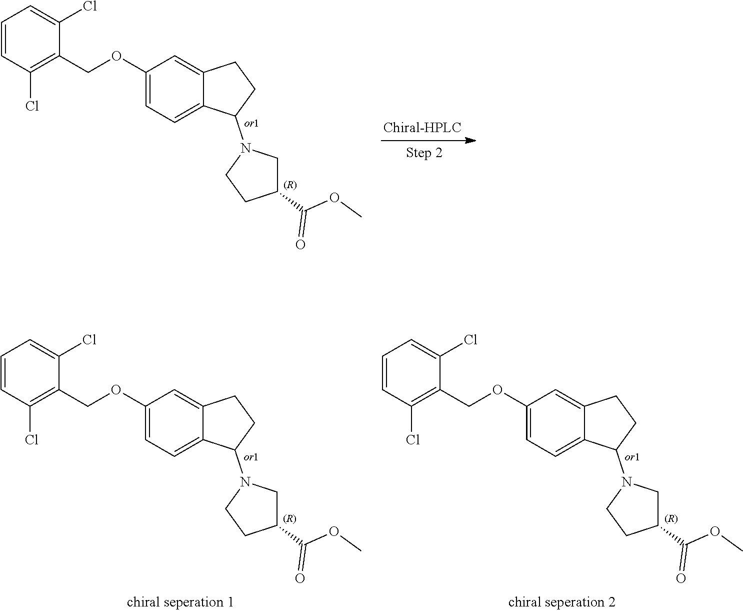

The racemate (200 mg) was resolved by Prep-Chiral-HPLC (column: CHIRALPAK IG, 2*25 cm, 5 m; mobile phase A: Hex (8 mM NH₃·MeOH)-HPLC, mobile phase B: EtOH-HPLC; Flow rate: 20 mL/min; Gradient: 10% B to 10% B in 10 min; 220/254 nm; RT1: 7.17 min; RT2: 8.523 min) to give 70 mg of both enantiomers. LCMS (ESI, m/z): 420 [M+H]⁺.

### Synthesis of (3R)-1-(5-((2,6-dichlorobenzyl)oxy)-2,3-dihydro-1H-inden-1-yl)pyrrolidine-3-carboxylic acid (25a)

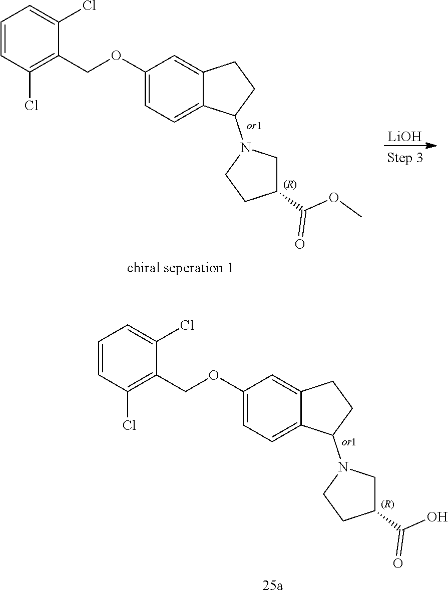

To a stirred solution of methyl (3R)-1-(5-((2,6-dichlorobenzyl)oxy)-2,3-dihydro-1H-inden-1-yl)pyrrolidine-3-carboxylate (chiral separation 1, 70 mg, 0.17 mmol, 1.00 equiv) in THE (2.0 mL) and water (0.2 mL) was added LiOH H₂O (21 mg, 0.50 mmol, 3.00 equiv). The resulting mixture was stirred at room temperature for 2 h. LCMS showed the reaction was complete. The reaction mixture was acidified to pH 4-5 using 1 M HCl and then was concentrated. The residue was purified by Prep-HPLC (Column: XBridge Shield RP18 OBD Column, 30*150 mm, 5 m; Mobile Phase A: water (10 mM NH₄HCO₃+0.10% NH₃·H₂O), Mobile Phase B: ACN; Flow rate: 60 mL/min; Gradient: 22% B to 40% B in 7 min; Wavelength: 254 nm; RT: 6.85 min) to give (3R)-1-(5-((2,6-dichlorobenzyl)oxy)-2,3-dihydro-1H-inden-1-yl)pyrrolidine-3-carboxylic acid (14.7 mg, 21%) as a white solid.

LCMS (ESI, m/z): 406 [M+H]⁺. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 95% B in 2.00 min, hold at 95% for 0.70 min, 95% B to 5% B in 0.05 min; 210 nm; RT: 1.586 min.

¹H NMR (300 MHz, DMSO-d₆) δ 7.58-7.55 (m, 2H), 7.49-7.44 (m, 1H), 7.21-7.19 (m, 1H), 6.96-6.92 (m, 1H), 6.85-6.82 (m, 1H), 5.19 (s, 2H), 4.07-4.03 (m, 1H), 2.98-2.82 (m, 2H), 2.78-2.63 (m, 3H), 2.58-2.53 (m, 2H), 2.08-2.01 (m, 2H), 1.93-1.88 (m, 2H).

### Synthesis of (3R)-1-(5-((2,6-dichlorobenzyl)oxy)-2,3-dihydro-1H-inden-1-yl)pyrrolidine-3-carboxylic acid (25b)

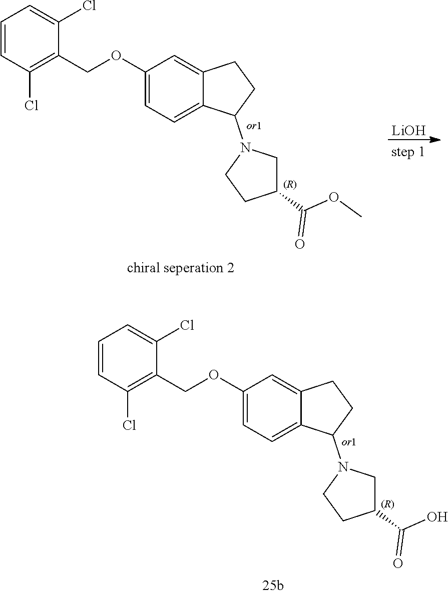

To a stirred solution of methyl (3R)-1-(5-((2,6-dichlorobenzyl)oxy)-2,3-dihydro-1H-inden-1-yl)pyrrolidine-3-carboxylate (chiral separation 2, 70 mg, 0.17 mmol, 1.00 equiv) in THE (2.0 mL) and water (0.2 mL) was added LiOH H₂O (21 mg, 0.50 mmol, 3.00 equiv). The resulting mixture was stirred at room temperature for 2 h. LCMS showed the reaction was complete. The reaction mixture was acidified to pH 4-5 using 1 M HCl and then was concentrated. The residue was purified by Prep-HPLC (Column: XBridge Shield RP18 OBD Column, 30*150 mm, 5 m; Mobile Phase A: water (10 mM NH₄HCO₃+0.1% NH₃·H₂O), Mobile Phase B: ACN; Flow rate: 60 mL/min; Gradient: 20% B to 43% B in 7 min; Wavelength: 254 nm; RT: 6.67 min) to give (3R)-1-(5-((2,6-dichlorobenzyl)oxy)-2,3-dihydro-1H-inden-1-yl)pyrrolidine-3-carboxylic acid (26.8 mg, 39%) as a white solid.

LCMS (ESI, m/z): 406 [M+H]⁺. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 95% B in 2.00 min, hold at 95% for 0.70 min, 95% B to 5% B in 0.05 min; 210 nm; RT: 1.580 min.

¹H NMR (300 MHz, DMSO-d₆) δ 7.58-7.55 (m, 2H), 7.49-7.44 (m, 1H), 7.22 (d, J=8.1 Hz, 1H), 6.95 (d, J=2.4 Hz, 1H), 6.84 (dd, J=8.1, 2.4 Hz, 1H), 5.19 (s, 2H), 4.05 (t, J=2.4 Hz, 1H), 2.99-2.83 (m, 2H), 2.80-2.66 (m, 3H), 2.62-2.55 (m, 2H), 2.10-2.03 (m, 2H), 1.95-1.88 (m, 2H).

### Example S26. Synthesis of 1-(5-((3-(trifluoromethyl)benzyl)oxy)-2,3-dihydro-1H-inden-1-yl)piperidine-4-carboxylic acid (26a & 26b)

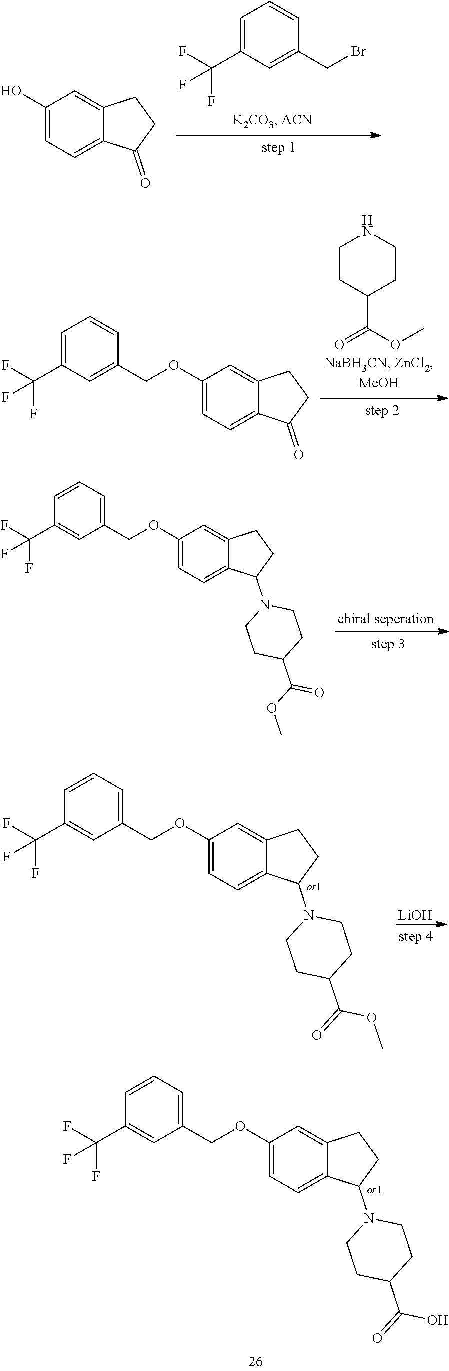

### Synthesis of 5-((3-(trifluoromethyl)benzyl)oxy)-2,3-dihydro-1H-inden-1-one

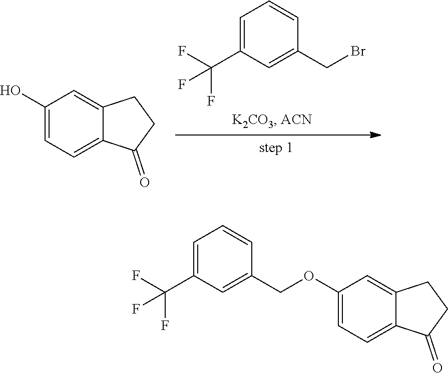 [SMILES: O=C1CCc2cc(O)ccc21 ; FC(F)(F)c1cccc(CBr)c1 ; O=C1CCc2cc(OCc3cccc(C(F)(F)F)c3)ccc21]

To a solution of 5-hydroxyindan-1-one (2.0 g, 13.5 mmol, 1.00 equiv) in MeCN (10.0 mL) were added 1-(bromomethyl)-3-(trifluoromethyl) benzene (3.2 g, 13.5 mmol, 1.10 equiv) and K₂CO₃ (5.5 g, 40.5 mmol, 3.00 equiv). The resulting mixture was stirred overnight at 60° C. LCMS showed the reaction was complete. The reaction mixture was filtered and the filtrate was concentrated under reduced pressure. The residue was purified by flash chromatography on C18 silica (eluted with water/MeCN, 20/80) to give 5-((3-(trifluoromethyl)-benzyl)oxy)-2,3-dihydro-1H-inden-1-one (3.2 g, 77%) as a yellow solid. LCMS (ESI, m/z): 307 [M+H]⁺.

### Synthesis of methyl 1-(5-((3-(trifluoromethyl)benzyl)oxy)-2,3-dihydro-1H-inden-1-yl)piperidine-4-carboxylate

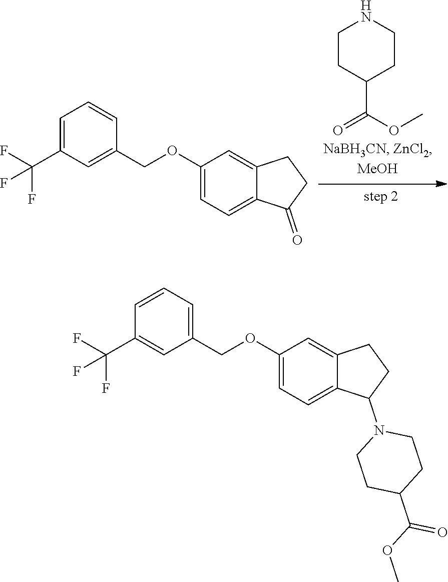

A solution of ZnCl₂ (2 M in THF, 1.3 mL, 2.61 mmol, 2.00 equiv) and NaBH₃CN (328 mg, 5.22 mmol, 4.00 equiv) in methanol (5.0 mL) was stirred for 0.5 h at room temperature. Then 5-((3-(trifluoromethyl)benzyl)oxy)-2,3-dihydro-1H-inden-1-one (400 mg, 1.31 mmol, 1.00 equiv) and methyl piperidine-4-carboxylate (280 mg, 1.96 mmol, 1.50 equiv) were added. The resulting mixture was stirred overnight at 60° C. LCMS showed the reaction was complete. The mixture was filtered through celite and the filtrate was concentrated under vacuum. The residue was purified by flash chromatography on C18 silica (eluted with water/MeCN, 35/65) to give methyl 1-(5-((3-(trifluoromethyl)benzyl)oxy)-2,3-dihydro-1H-inden-1-yl)piperidine-4-carboxylate (200 mg, 35%) as a yellow solid. LCMS (ESI, m/z): 434 [M+H]⁺.

### Chiral separation of methyl 1-(5-((3-(trifluoromethyl)benzyl)oxy)-2,3-dihydro-1H-inden-1-yl)piperidine-4-carboxylate

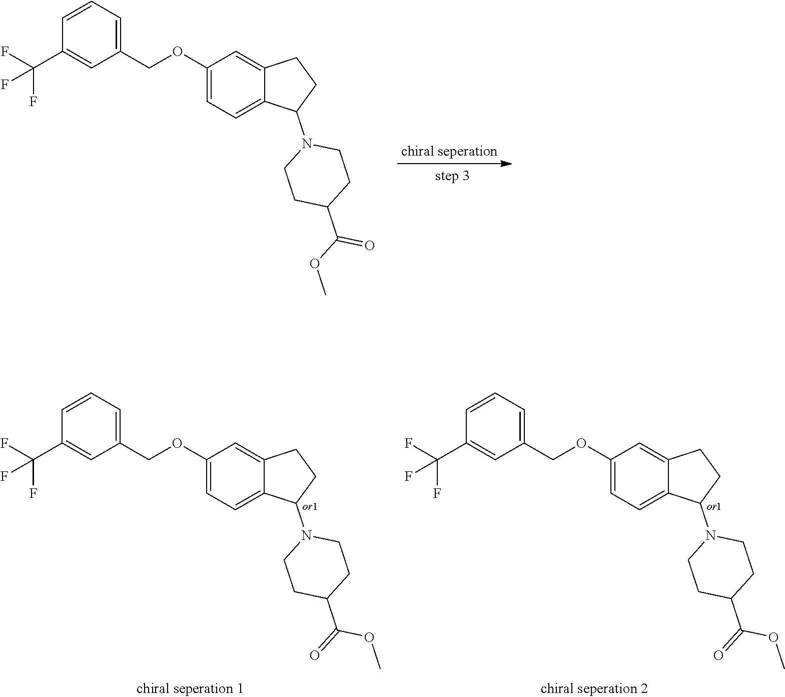 [SMILES: COC(=O)C1CCN(C2CCc3cc(OCc4cccc(C(F)(F)F)c4)ccc32)CC1 ; COC(=O)C1CCN(C2CCc3cc(OCc4cccc(C(F)(F)F)c4)ccc32)CC1 ; COC(=O)C1CCN(C2CCc3cc(OCc4cccc(C(F)(F)F)c4)ccc32)CC1]

The racemic methyl 1-(5-((3-(trifluoromethyl)benzyl)oxy)-2,3-dihydro-1H-inden-1-yl)piperidine-4-carboxylate (200 mg) was resolved by Prep-chiral-HPLC (CHIRALPAK IE, 2*25 cm, 5 m; Mobile Phase A: Hex (8 mM NH₃·MeOH)—-HPLC, Mobile Phase B: EtOH-HPLC; Flow rate: 20 mL/min; Gradient: 5% B to 5% B in 15 min; 220/254 nm; RT1: 10.785 min; RT2: 11.612 min) to afford 63 mg of both enantiomers. LCMS (ESI, m/z): 434 [M+H]⁺.

### Synthesis of 1-(5-((3-(trifluoromethyl)benzyl)oxy)-2,3-dihydro-1H-inden-1-yl)piperidine-4-carboxylic acid (26a)

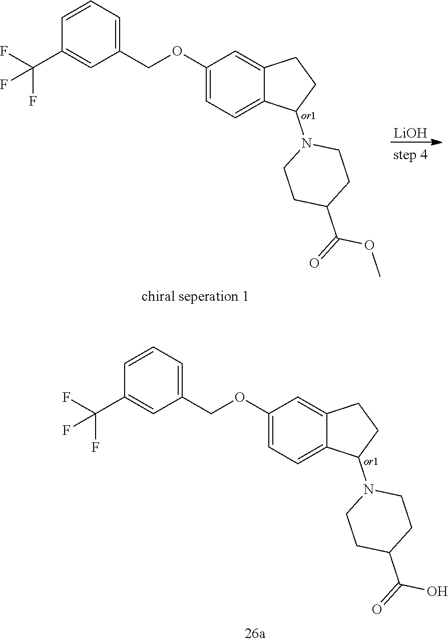

To a solution of methyl 1-(5-((3-(trifluoromethyl)benzyl)oxy)-2,3-dihydro-1H-inden-1-yl)piperidine-4-carboxylate (chiral separation 1, 63 mg, 0.15 mmol, 1.00 equiv) in THE (3.0 mL) and water (0.6 mL) was added LiOH H₂O (18 mg, 0.44 mmol, 3.00 equiv). The resulting mixture was stirred for 6 h at room temperature. LCMS showed the reaction was complete. The reaction mixture was acidified to pH 4-5 using 1 M HCl and then was concentrated. The residue was purified by Prep-HPLC (Column: Xselect CSH C18 OBD Column 30*150 mm, 5 m; Mobile Phase A: water (10 mM NH₄HCO₃+0.10% NH₃·H₂O), Mobile Phase B: ACN; Flow rate: 60 mL/min; Gradient: 22% B to 52% B in 7 min; Wavelength: 254/210 nm; RT: 6.42 min) to give 1-(5-((3-(trifluoromethyl)benzyl)oxy)-2,3-dihydro-1H-inden-1-yl)piperidine-4-carboxylic acid (35.8 mg, 58.3%) as a white solid.

LCMS (ESI, m/z): 420 [M+H]⁺. Analytic Conditions: column: Titank C18, 3.0*50 mm, 3.0 m; mobile phase A: water (5 mM NH₄HCO₃), mobile phase B: acetonitrile; flow rate: 1.50 mL/min; gradient: 10% B to 95% B in 1.40 min, hold at 95% for 0.80 min, 95% B to 10% B in 0.03 min; 220 nm; RT: 1.164 min.

¹H NMR (300 MHz, DMSO-d₆) δ 11.73 (br, 1H), 7.80-7.75 (m, 2H), 7.72-7.62 (m, 2H), 7.17 (d, J=8.1 Hz, 1H), 6.90-6.83 (m, 2H), 5.18 (s, 2H), 4.24-4.18 (m, 1H), 2.86-2.66 (m, 3H), 2.46-2.43 (m, 1H), 2.26-2.08 (m, 3H), 2.00-1.96 (m, 2H), 1.82-1.72 (m, 2H), 1.62-1.42 (m, 2H).

### Synthesis of 1-(5-((3-(trifluoromethyl)benzyl)oxy)-2,3-dihydro-1H-inden-1-yl)piperidine-4-carboxylic acid (26b)

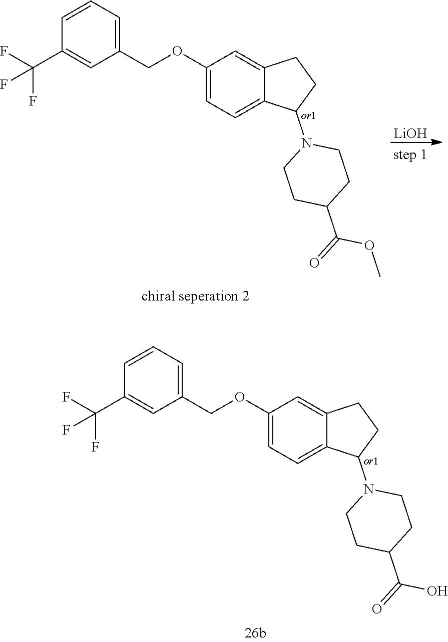

To a solution of methyl 1-(5-((3-(trifluoromethyl)benzyl)oxy)-2,3-dihydro-1H-inden-1-yl)piperidine-4-carboxylate (chiral separation 2, 63 mg, 0.15 mmol, 1.00 equiv) in THE (3.0 mL) and water (0.6 mL) was added LiOH H₂O (18 mg, 0.44 mmol, 3.00 equiv). The resulting mixture was stirred for 6 h at room temperature. LCMS showed the reaction was complete. The reaction mixture was acidified to pH 4-5 using 1 M HCl and then was concentrated. The residue was purified by Prep-HPLC (Column: Xselect CSH C18 OBD Column 30*150 mm, 5 m; Mobile Phase A: water (10 mM NH₄HCO₃+0.1% NH₃·H₂O), Mobile Phase B: ACN; Flow rate: 60 mL/min; Gradient: 22% B to 52% B in 7 min; Wavelength: 254/210 nm; RT: 6.77 min) to give 1-(5-((3-(trifluoromethyl)benzyl)oxy)-2,3-dihydro-1H-inden-1-yl)piperidine-4-carboxylic acid (36.3 mg, 59.0%) as a white solid.

LCMS (ESI, m/z): 420 [M+H]⁺. Analytic Conditions: column: Titank C18, 3.0*50 mm, 3.0 m; mobile phase A: water (5 mM NH₄HCO₃), mobile phase B: acetonitrile; flow rate: 1.50 mL/min; gradient: 10% B to 95% B in 1.40 min, hold at 95% for 0.80 min, 95% B to 10% B in 0.03 min; 220 nm; RT: 1.167 min.

¹H NMR (300 MHz, DMSO-d₆) δ 7.80-7.75 (m, 2H), 7.72-7.62 (m, 2H), 7.17 (d, J=8.4 Hz, 1H), 6.90-6.84 (m, 2H), 5.18 (s, 2H), 4.24-4.18 (m, 1H), 2.83-2.65 (m, 3H), 2.45-2.41 (m, 1H), 2.27-2.12 (m, 3H), 2.01-1.96 (m, 2H), 1.82-1.72 (m, 2H), 1.58-1.42 (m, 2H).

### Example S27. Synthesis of 2-(5-((2,6-dichlorobenzyl)oxy)-2,3-dihydro-1H-inden-1-yl)-2-azaspiro[3.3]-heptane-6-carboxylic acid (27)

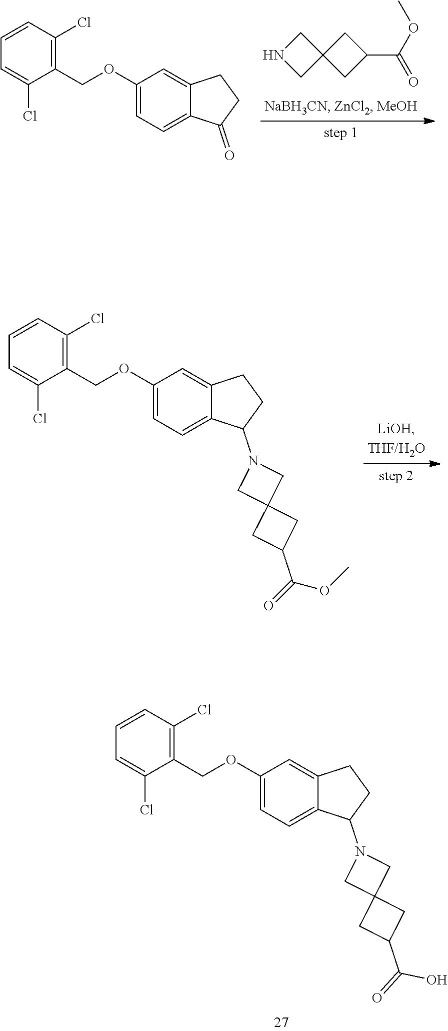 [SMILES: O=C1CCc2cc(OCc3c(Cl)cccc3Cl)ccc21 ; COC(=O)C1CC2(CNC2)C1 ; COC(=O)C1CC2(C1)CN(C1CCc3cc(OCc4c(Cl)cccc4Cl)ccc31)C2 ; O=C(O)C1CC2(C1)CN(C1CCc3cc(OCc4c(Cl)cccc4Cl)ccc31)C2]

### Synthesis of methyl 2-(5-((2,6-dichlorobenzyl)oxy)-2,3-dihydro-1H-inden-1-yl)-2-azaspiro-[3.3]heptane-6-carboxylate

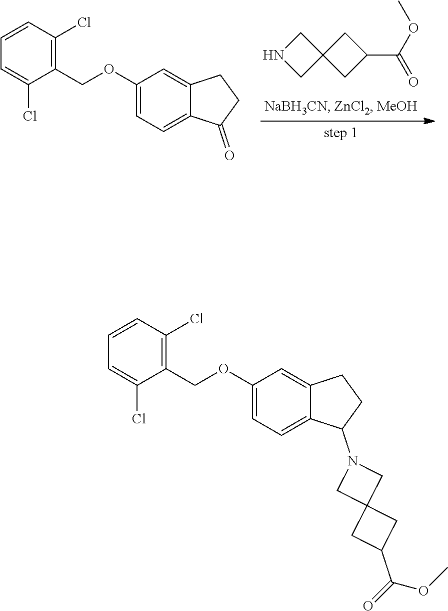 [SMILES: O=C1CCc2cc(OCc3c(Cl)cccc3Cl)ccc21 ; COC(=O)C1CC2(CNC2)C1 ; COC(=O)C1CC2(C1)CN(C1CCc3cc(OCc4c(Cl)cccc4Cl)ccc31)C2]

A mixture of 5-((2,6-dichlorobenzyl)oxy)-2,3-dihydro-1H-inden-1-one (300 mg, 0.980 mmol, 1.00 equiv), methyl 2-azaspiro[3.3]heptane-6-carboxylate (152 mg, 0.980 mmol, 1.00 equiv), Zinc chloride (1.9 M in 2-Me-THF, 1.5 mL, 2.00 equiv) and NaBH₃CN (250 mg, 3.920 mmol, 4.00 equiv) in methanol (8.0 mL) was stirred at 60° C. for 12 h. LCMS showed the reaction was complete. The reaction mixture was quenched with water (10 mL) and extracted with DCM (3×20 mL). The combined organic phase was dried over Na₂SO₄, filtered and concentrated under reduced pressure. The crude product was purified by flash chromatography on silica gel (DCM: MeOH=50:1) to give the desired product methyl 2-(5-((2,6-dichlorobenzyl)oxy)-2,3-dihydro-1H-inden-1-yl)-2-azaspiro[3.3]heptane-6-carboxylate (100 mg, 23%) as a light yellow oil. LCMS (ESI, m/z): 446 [M+H]⁺.

### Synthesis of 2-(5-((2,6-dichlorobenzyl)oxy)-2,3-dihydro-1H-inden-1-yl)-2-azaspiro[3.3]-heptane-6-carboxylic acid

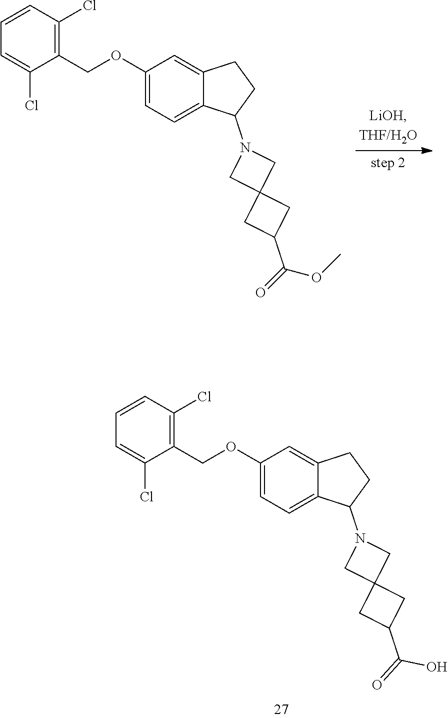 [SMILES: COC(=O)C1CC2(C1)CN(C1CCc3cc(OCc4c(Cl)cccc4Cl)ccc31)C2 ; O=C(O)C1CC2(C1)CN(C1CCc3cc(OCc4c(Cl)cccc4Cl)ccc31)C2]

A mixture of methyl 2-(5-((2,6-dichlorobenzyl)oxy)-2,3-dihydro-1H-inden-1-yl)-2-azaspiro-[3.3]heptane-6-carboxylate (100 mg, 0.220 mmol, 1.00 equiv) and LiOH H₂O (47 mg, 1.110 mmol, 5.00 equiv) in THE (4.0 mL) and water (4.0 mL) was stirred at rt for 1 h. LCMS showed the reaction was complete. The reaction mixture was acidified to pH 4-5 using 1 M HCl and then was concentrated. The residue was purified by Prep-HPLC (Column: XBridge Shield RP18 OBD Column, 5 m, 19*150 mm; Mobile Phase A: water (10 mM NH₄HCO₃+0.1% NH₃·H₂O), Mobile Phase B: ACN; Flow rate: 60 mL/min; Gradient: 15% B to 35% B in 10 min, hold at 35% for 3 min; 210/254 nm; RT: 11.6 min) to give 2-(5-((2,6-dichlorobenzyl)oxy)-2,3-dihydro-1H-inden-1-yl)-2-azaspiro[3.3]heptane-6-carboxylic acid (38.7 mg, 38%) as a white solid.

LCMS (ESI, m/z): 432 [M+H]+. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 210 nm; RT: 1.594 min.

¹H NMR (400 MHz, CD30D-d₄) δ 7.49-7.43 (m, 3H), 7.38 (dd, J=8.8, 7.2 Hz, 1H), 7.06 (d, J=2.4 Hz, 1H), 6.97 (dd, J=8.4, 2.4 Hz, 1H), 5.32 (s, 2H), 4.62-4.58 (m, 1H), 4.16-4.09 (m, 2H), 4.03-3.97 (m, 2H), 3.19-3.11 (m, 1H), 2.99-2.94 (m, 1H), 2.89-2.82 (m, 1H), 2.45-2.30 (m, 5H), 2.14-2.07 (m, 1H).

### Example S28. Synthesis of 1-(5-((2,6-dichlorobenzyl)oxy)-2,3-dihydro-1H-inden-1-yl)-2,2-dimethyl-piperidine-4-carboxylic acid (28)

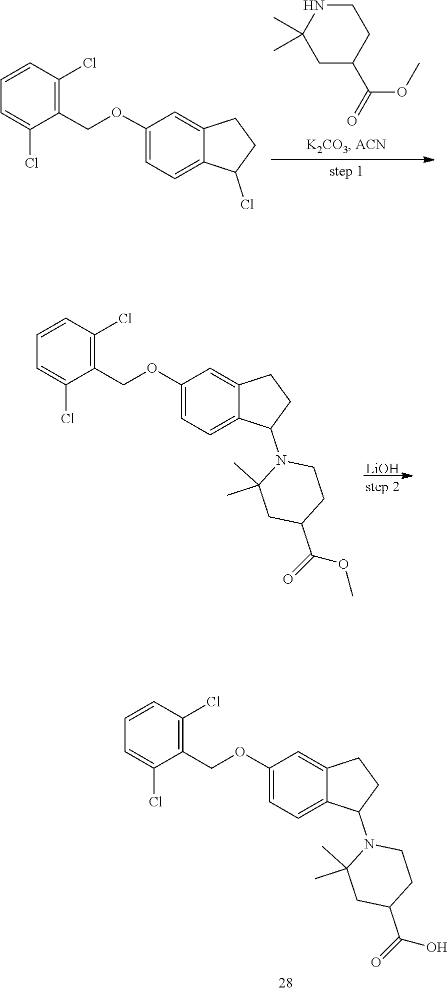

### Synthesis of methyl 1-(5-((2,6-dichlorobenzyl)oxy)-2,3-dihydro-1H-inden-1-yl)-2,2-dimethylpiperidine-4-carboxylate

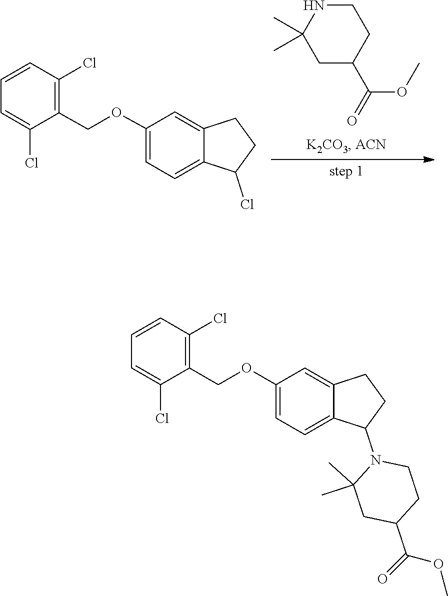

To a stirred solution of 1-chloro-5-((2,6-dichlorobenzyl)oxy)-2,3-dihydro-1H-indene (300 mg, 0.920 mmol, 1.00 equiv) and methyl 2,2-dimethylpiperidine-4-carboxylate (156 mg, 0.920 mmol, 1.00 equiv) in MeCN (10.0 mL) were added Cs₂CO₃ (895 mg, 2.750 mmol, 3.00 equiv). The mixture was stirred at 90° C. for 16 h. LCMS showed the reaction was complete. The reaction mixture was poured into water (10 mL), and extracted with EtOAc (2*10 mL). The combined organic layers were washed with brine, dried over anhydrous Na₂SO₄, and concentrated under reduced pressure. The residue was purified by flash chromatography on silica gel (PE/EtOAc, 3/1) to give methyl 1-(5-((2,6-dichloro-benzyl)oxy)-2,3-dihydro-1H-inden-1-yl)-2,2-dimethylpiperidine-4-carboxylate (70 mg, 16% yield) as an off-white solid. LCMS (ESI, m/z): 462 [M+H]⁺.

### Synthesis of 1-(5-((2,6-dichlorobenzyl)oxy)-2,3-dihydro-1H-inden-1-yl)-2,2-dimethyl-piperidine-4-carboxylic acid

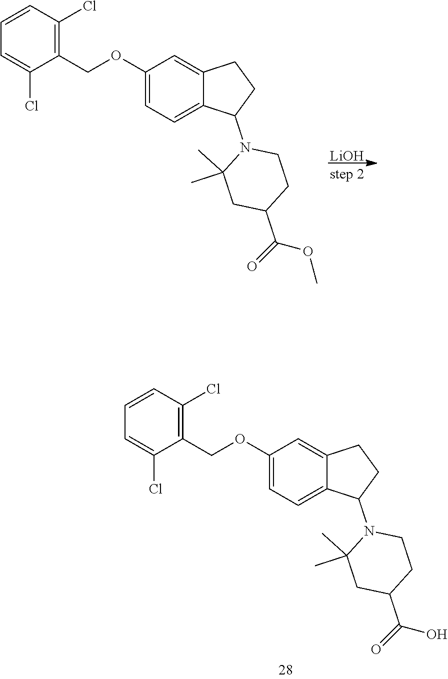

To a stirred solution of methyl 1-(5-((2,6-dichlorobenzyl)oxy)-2,3-dihydro-1H-inden-1-yl)-2,2-dimethylpiperidine-4-carboxylate (55 mg, 0.120 mmol, 1.00 equiv) in THE (1.0 mL) and water (0.1 mL) was added LiOH·H₂O (15 mg, 0.360 mmol, 3.00 equiv). The mixture was stirred at room temperature for 16 h. LCMS showed that the reaction was complete. The reaction was acidified to pH 4-5 by adding 1 N HCl. Then the mixture was concentrated under reduced pressure. The residue was purified by Prep-IPLC (Column: YMC-Actus Triart C18, 30*250 mm, 5 m; Mobile Phase A: water (10 mM NH₄HCO₃+0.1% NH₃·H₂O), Mobile Phase B: ACN; Flow rate: 60 mL/min; Gradient: 21% B to 51% B in 7 min; 254/210 nm; RT: 6.42 min) to give 1-(5-((2,6-dichlorobenzyl)oxy)-2,3-dihydro-1H-inden-1-yl)-2,2-dimethyl-piperidine-4-carboxylic acid (15.2 mg, 27% yield) as an off-white solid.

LCMS (ESI, m/z): 448 [M+H]⁺. Analytic Conditions: EVO C18, 3.0*50 mm, 2.6 m; Mobile Phase A: water (5 mM NH₄HCO₃), Mobile Phase B: ACN; Flow rate: 1.20 mL/min; Gradient: 10% B to 95% B in 2.0 min, hold at 95% B for 0.60 min, 95% B to 10% B in 0.15 min; 210 nm; RT: 1.261 min.

¹H NMR (300 MHz, CD30D-d₄) δ 7.47-7.42 (m, 3H), 7.39-7.33 (m, 1H), 7.07-6.97 (m, 2H), 5.31 (d, J=2.7 Hz, 2H), 5.19-5.15 (m, 1H), 3.21-2.85 (m, 3H), 2.67-2.36 (m, 4H), 2.06-1.89 (m, 3H), 1.62-1.44 (m, 7H).

### Example S29. Synthesis of 1-(5-((2,6-dichlorobenzyl)oxy)-2,3-dihydro-1H-inden-1-yl)-4-methylpiperidine-4-carboxylic acid (29)

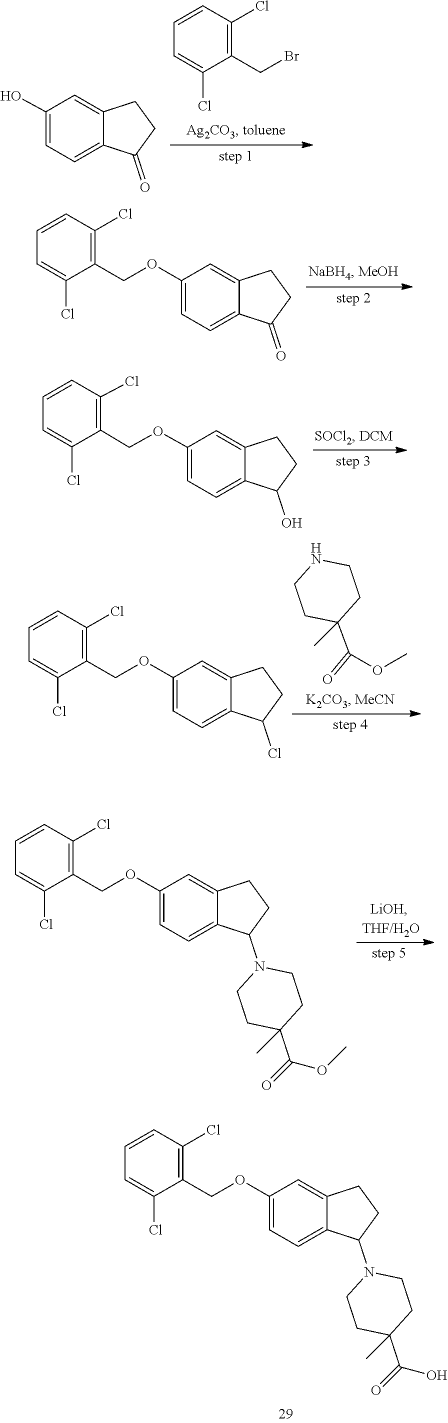

### Synthesis of 5-((2,6-dichlorobenzyl)oxy)-2,3-dihydro-1H-inden-1-one

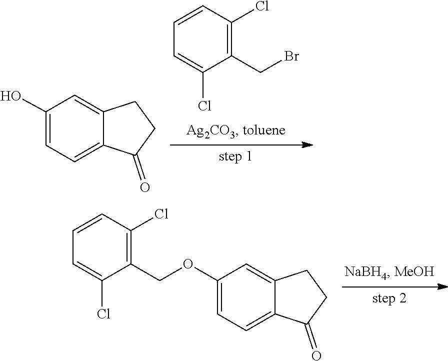

A mixture of 5-hydroxyindan-1-one (2.00 g, 13.5 mmol, 1.00 equiv), 2-(bromomethyl)-1,3-dichloro-benzene (6.48 g, 27.0 mmol, 2.00 equiv) and Ag₂CO₃ (7.45 g, 27.0 mmol, 2.00 equiv) in toluene (40 mL) was stirred at 80° C. overnight. LCMS showed that the reaction was complete. The reaction mixture was concentrated under reduced pressure. The crude was diluted with water (100 mL) and extracted with DCM (3*100 mL). The combined organic layers were washed with brine, dried over Na₂SO₄, and concentrated under reduced pressure. The residue was purified by flash chromatography on silica gel (PE: EA=1:1) to give 5-((2,6-dichlorobenzyl)oxy)-2,3-dihydro-1H-inden-1-one (2.80 g, 67.5%) as a light yellow solid. LCMS (ESI, m/z): 307 [M+H]⁺.

### Synthesis of 5-((2,6-dichlorobenzyl)oxy)-2,3-dihydro-1H-inden-1-ol

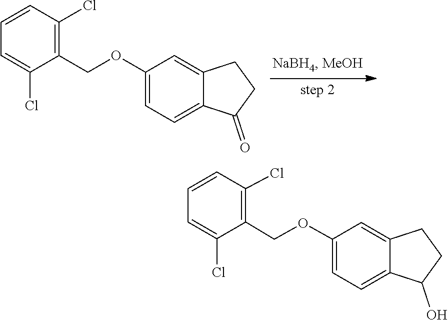

To a stirred solution of 5-((2,6-dichlorobenzyl)oxy)-2,3-dihydro-1H-inden-1-one (1.00 g, 3.26 mmol, 1.00 equiv) in methanol (4 mL) was added sodium borohydride (246 mg, 6.51 mmol, 2.00 equiv) in portions at 0° C. The resulting mixture was stirred at 0° C. for 30 min. TLC showed that the reaction was complete. The reaction was quenched by adding NH₄C₁ aqueous solution at 0° C. Then the mixture was concentrated under reduced pressure. The residue was purified by flash column chromatography on silica gel (DCM/MeOH, 91/9) to give 5-((2,6-dichlorobenzyl)oxy)-2,3-dihydro-1H-inden-1-ol (700 mg, 69.5%) as a light yellow solid. LCMS (ESI, m/z): 291 [M+H]⁺.

### Synthesis of 1-chloro-5-((2,6-dichlorobenzyl)oxy)-2,3-dihydro-1H-indene

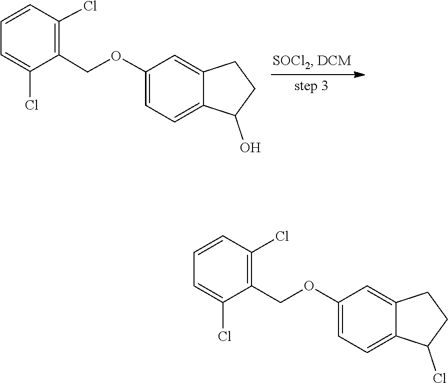 [SMILES: OC1CCc2cc(OCc3c(Cl)cccc3Cl)ccc21 ; Clc1cccc(Cl)c1COc1ccc2c(c1)CCC2Cl]

To a stirred solution of 5-((2,6-dichlorobenzyl)oxy)-2,3-dihydro-1H-inden-1-ol (200 mg, 0.650 mmol, 1.00 equiv) in DCM (3 mL) was added SOCl₂ (0.24 mL, 3.23 mmol, 5.00 equiv) dropwise at 0° C. The resulting mixture was stirred at 0° C. for 20 min. TLC showed that the reaction was complete. The reaction mixture was concentrated and dried under vacuum for 20 min. The obtained crude purple oil 1-chloro-5-((2,6-dichlorobenzyl)oxy)-2,3-dihydro-1H-indene (160 mg, 75.5%) was used directly in next step.

### Synthesis of methyl 1-(5-((2,6-dichlorobenzyl)oxy)-2,3-dihydro-1H-inden-1-yl)-4-methylpiperidine-4-carboxylate

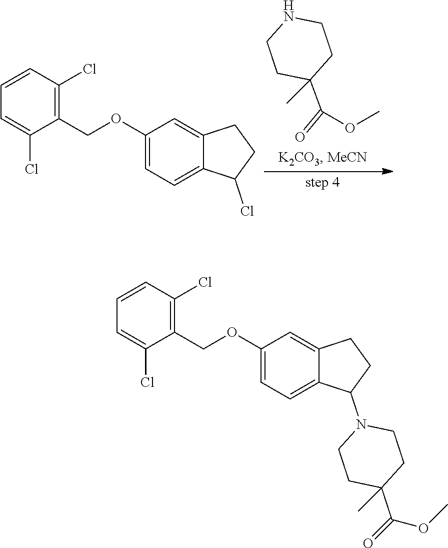 [SMILES: Clc1cccc(Cl)c1COc1ccc2c(c1)CCC2Cl ; COC(=O)C1(C)CCNCC1 ; COC(=O)C1(C)CCN(C2CCc3cc(OCc4c(Cl)cccc4Cl)ccc32)CC1]

A mixture of 1-chloro-5-((2,6-dichlorobenzyl)oxy)-2,3-dihydro-1H-indene (140 mg, 0.430 mmol, 1.00 equiv), methyl 4-methylpiperidine-4-carboxylate (67.2 mg, 0.430 mmol, 1.00 equiv) and K₂CO₃ (118 mg, 0.860 mmol, 2.00 equiv) in MeCN (4 mL) was stirred at 60° C. overnight. The reaction was concentrated under reduced pressure. The residue was purified by flash chromatography on silica gel (eluted with PE/EtOAc, 1/1) to give methyl 1-(5-((2,6-dichlorobenzyl)oxy)-2,3-dihydro-1H-inden-1-yl)-4-methylpiperidine-4-carboxylate (80.0 mg, 41.7%) as a grey solid. LCMS (ESI, m/z): 448 [M+H]⁺.

### Synthesis of 1-(5-((2,6-dichlorobenzyl)oxy)-2,3-dihydro-1H-inden-1-yl)-4-methylpiperidine-4-carboxylic acid

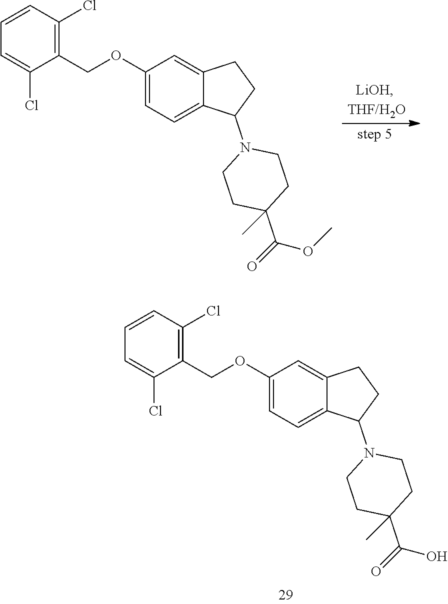 [SMILES: COC(=O)C1(C)CCN(C2CCc3cc(OCc4c(Cl)cccc4Cl)ccc32)CC1 ; CC1(C(=O)O)CCN(C2CCc3cc(OCc4c(Cl)cccc4Cl)ccc32)CC1]

A mixture of methyl 1-(5-((2,6-dichlorobenzyl)oxy)-2,3-dihydro-1H-inden-1-yl)-4-methylpiperidine-4-carboxylate (80.0 mg, 0.180 mmol, 1.00 equiv) and sodium hydroxide (14.3 mg, 0.360 mmol, 2.00 equiv) in THE (1 mL) and water (1 mL) was stirred at 60° C. overnight. The reaction mixture was acidified to pH 4-5 with 1 N HCl aqueous solution. The resulting mixture was concentrated under reduced pressure. The residue was purified by prep-HPLC (Column: XBridge Shield RP18 OBD Column, 30*150 mm, 5 m; Mobile Phase A: water (10 mmoL/L NH₄HCO₃+0.1% NH₃·H₂O), Mobile Phase B: ACN; Flow rate: 60 mL/min; Gradient: 20% B to 50% B in 7 min; 210/254 nm; RT: 6.05 min) to give 1-(5-((2,6-dichlorobenzyl)oxy)-2,3-dihydro-1H-inden-1-yl)-4-methylpiperidine-4-carboxylic acid (25.9 mg, 33.1%) as a white solid.

¹H NMR (400 MHz, DMSO-d₆) δ 12.07 (br, 1H), 7.58 (d, J=1.2 Hz, 1H), 7.56 (s, 1H), 7.50-7.46 (m, 1H), 7.17 (d, J=8.0 Hz, 1H), 6.94 (d, J=2.4 Hz, 1H), 6.86 (dd, J=8.0, 2.4 Hz, 1H), 5.19 (s, 2H), 4.24-4.20 (m, 1H), 2.90-2.82 (m, 1H), 2.78-2.70 (m, 1H), 2.64-2.58 (m, 1H), 2.46-2.39 (m, 1H), 2.28-2.22 (m, 2H), 2.02-1.92 (m, 4H), 1.43-1.30 (m, 2H), 1.11 (s, 3H).

LCMS (ESI, m/z): 434 [M+H]. Analytic Conditions: EVO C18, 3.0*50 mm, 2.6 m; Mobile Phase A: water/5 mM NH₄HCO₃, Mobile Phase B: ACN; Flow rate: 1.20 mL/min; Gradient: 10% B to 95% B in 2.0 min, hold at 95% B for 0.6 min, 95% B to 10% B in 0.15 min; 210 nm; RT: 1.125 min.

### Example S30. Synthesis of 1-(5-((2,6-dichlorobenzyl)oxy)-2,3-dihydro-1H-inden-1-yl)-2-methylpiperidine-4-carboxylic acid (30)

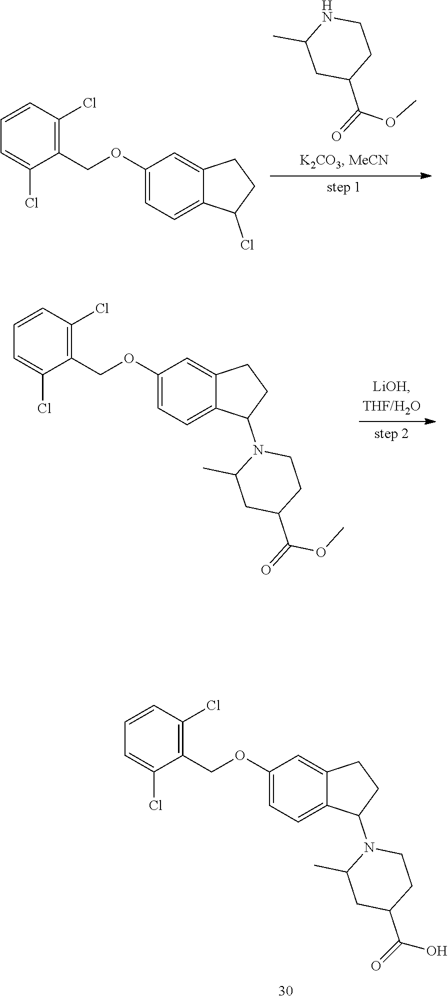

### Synthesis of methyl 1-(5-((2,6-dichlorobenzyl)oxy)-2,3-dihydro-1H-inden-1-yl)-2-methyl-piperidine-4-carboxylate

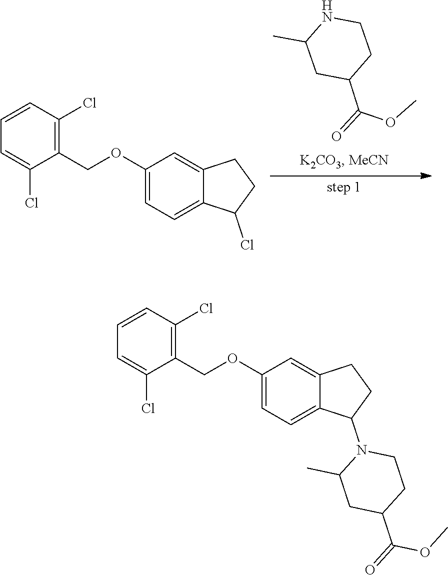

To a stirred solution of 1-chloro-5-((2,6-dichlorobenzyl)oxy)-2,3-dihydro-1H-indene (160 mg, 0.490 mmol, 1.00 equiv) and methyl 2-methylpiperidine-4-carboxylate (76.8 mg, 0.490 mmol, 1.00 equiv) in MeCN (4 mL) was added K₂CO₃ (135 mg, 0.980 mmol, 2.00 equiv). The resulting mixture was stirred at 60° C. overnight. LCMS showed that the reaction was complete. The reaction mixture was concentrated under reduced pressure. The residue was diluted with water (10 mL) and extracted with DCM (2*10 mL). The combined organic layers were washed with brine, dried over anhydrous Na₂SO₄ and concentrated under reduced pressure. The residue was purified by flash chromatography on silica gel (eluted with DCM/MeOH, 97/3) to give methyl 1-(5-((2,6-dichlorobenzyl)oxy)-2,3-dihydro-1H-inden-1-yl)-2-methyl-piperidine-4-carboxylate (160 mg, 73.1% yield) as a grey solid. LCMS (ESI, m/z): 448 [M+H]⁺.

### Synthesis of 1-(5-((2,6-dichlorobenzyl)oxy)-2,3-dihydro-1H-inden-1-yl)-2-methylpiperidine-4-carboxylic acid

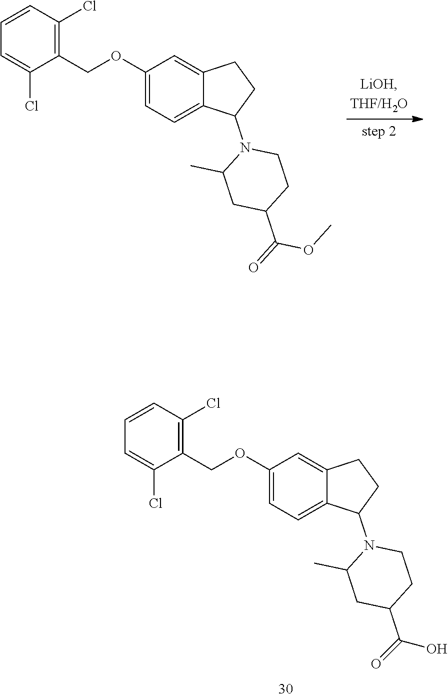 [SMILES: COC(=O)C1CCN(C2CCc3cc(OCc4c(Cl)cccc4Cl)ccc32)C(C)C1 ; CC1CC(C(=O)O)CCN1C1CCc2cc(OCc3c(Cl)cccc3Cl)ccc21]

A mixture of methyl 1-(5-((2,6-dichlorobenzyl)oxy)-2,3-dihydro-1H-inden-1-yl)-2-methyl-piperidine-4-carboxylate (120 mg, 0.270 mmol, 1.00 equiv) and sodium hydroxide (16.1 mg, 0.400 mmol, 1.50 equiv) in THE (1 mL) and water (1 mL) was stirred at 60° C. overnight. The reaction was acidified to pH 4-5 with 1 M HCl solution. The resulting mixture was concentrated under reduced pressure. The residue was purified by prep-HPLC (Column: YMC-Actus Triart C18, 30*250, 5 m; Mobile Phase A: water (10 mmolL/L NH₄HCO₃+0.1% NH₃·H₂O), Mobile Phase B: ACN; Flow rate: 60 mL/min; Gradient: 30% B to 48% B in 7 min; 254/210 nm; RT: 6.63 min) to give 1-(5-((2,6-dichlorobenzyl)oxy)-2,3-dihydro-1H-inden-1-yl)-2-methyl-piperidine-4-carboxylic acid (74.1 mg, 62.8% yield) as a white solid.

¹H NMR (400 MHz, DMSO-d₆) δ 7.58-7.55 (m, 2H), 7.49-7.45 (m, 1H), 7.14 (dd, J=13.6, 8.0 Hz, 1H), 6.93 (dd, J=2.4, 2.0 Hz, 1H), 6.88-6.81 (m, 1H), 5.18 (s, 2H), 4.69-4.65 (m, 1H), 3.00-2.92 (m, 1H), 2.81-2.65 (m, 2H), 2.42-2.39 (m, 1H), 2.34-2.29 (m, 1H), 2.24-2.07 (m, 1H), 2.03-1.95 (m, 1H), 1.93-1.67 (m, 3H), 1.45-1.25 (m, 2H), 1.20 (dd, J=8.8, 6.0 Hz, 3H).

LCMS (ESI, m/z): 434 [M+H]⁺. Analytic Conditions: EVO C18, 3.0*50 mm, 2.6 m; Mobile Phase A: water/5 mM NH₄HCO₃, Mobile Phase B: ACN; Flow rate: 1.20 mL/min; Gradient: 10% B to 50% B in 2.0 min, 50% B to 95% B in 0.25 min, hold at 95% B for 0.45 min, 95% B to 10% B in 0.10 min; 210 nm; RT: 1.789/1.815 min (isomers).

### Example S31. Synthesis of 1-(5-((2,6-dichlorobenzyl)oxy)-2,3-dihydro-1H-inden-1-yl)-2,6-dimethyl-piperidine-4-carboxylic acid (31)

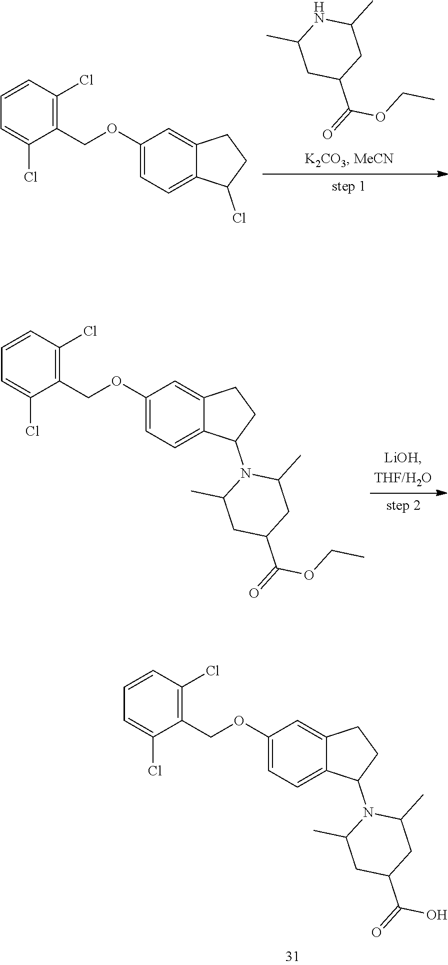 [SMILES: Clc1cccc(Cl)c1COc1ccc2c(c1)CCC2Cl ; CCOC(=O)C1CC(C)NC(C)C1 ; CCOC(=O)C1CC(C)N(C2CCc3cc(OCc4c(Cl)cccc4Cl)ccc32)C(C)C1 ; CC1CC(C(=O)O)CC(C)N1C1CCc2cc(OCc3c(Cl)cccc3Cl)ccc21]

### Synthesis of ethyl 1-(5-((2,6-dichlorobenzyl)oxy)-2,3-dihydro-2H-inden-6-yl)-2,6-dimethylpiperidine-4-carboxylate

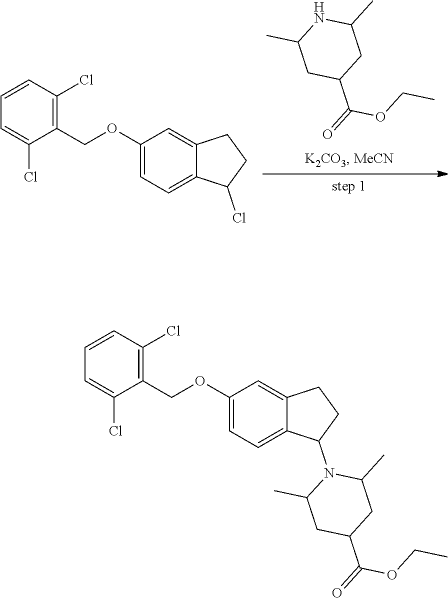 [SMILES: Clc1cccc(Cl)c1COc1ccc2c(c1)CCC2Cl ; CCOC(=O)C1CC(C)NC(C)C1 ; CCOC(=O)C1CC(C)N(C2CCc3cc(OCc4c(Cl)cccc4Cl)ccc32)C(C)C1]

A mixture of 1-chloro-5-((2,6-dichlorobenzyl)oxy)-2,3-dihydro-1H-indene (200 mg, 0.650 mmol, 1.00 equiv), ethyl 2,6-dimethylpiperidine-4-carboxylate (144 mg, 0.780 mmol, 1.2 equiv) and K₂CO₃ (179 mg, 1.29 mmol, 2.00 equiv) in MeCN (4 mL) was stirred at 90° C. for 4 days. The reaction mixture was poured into water (10 mL), and extracted with EtOAc (2*10 mL). The combined organic layer was washed with brine, dried over anhydrous Na₂SO₄, and concentrated under reduced pressure. The residue was purified by flash chromatography on silica gel (PE/EtOAc, 2/1) to give ethyl 1-(5-((2,6-dichlorobenzyl)oxy)-2,3-dihydro-1H-inden-1-yl)-2,6-dimethylpiperidine-4-carboxylate (30 mg, 9.7%) as a light-yellow oil. LCMS (ESI, m/z): 476 [M+H]⁺.

### Synthesis of 1-(5-((2,6-dichlorobenzyl)oxy)-2,3-dihydro-1H-inden-1-yl)-2,6-dimethyl-piperidine-4-carboxylic acid

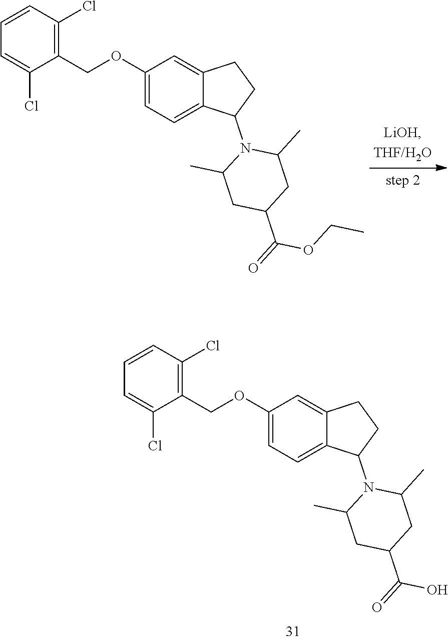 [SMILES: CCOC(=O)C1CC(C)N(C2CCc3cc(OCc4c(Cl)cccc4Cl)ccc32)C(C)C1 ; CC1CC(C(=O)O)CC(C)N1C1CCc2cc(OCc3c(Cl)cccc3Cl)ccc21]

A mixture of ethyl 1-(5-((2,6-dichlorobenzyl)oxy)-2,3-dihydro-1H-inden-1-yl)-2,6-dimethyl-piperidine-4-carboxylate (30 mg, 0.060 mmol, 1.00 equiv) and lithium hydroxide (3.0 mg, 0.120 mmol, 2.00 equiv) in THE (1 mL) and water (1 mL) was stirred at room temperature overnight. LCMS showed that the reaction was complete. The reaction mixture was acidified to pH 4-5 by adding 1 N HCl. Then the mixture was concentrated under reduced pressure. The residue was purified by Prep-HPLC (Column: YMC-Triart Diol Hilic, 20*150 mm 5 m; Mobile Phase A: water (10 mM NH₄HCO₃+0.1% NH₃·H₂O), Mobile Phase B: ACN; Flow rate: 60 mL/min; Gradient: 70% B to 95% B in 7 min; 254/210 nm; RT: 6.32 min) to give 1-(5-((2,6-dichlorobenzyl)oxy)-2,3-dihydro-1H-inden-1-yl)-2,6-dimethylpiperidine-4-carboxylic acid (1.1 mg, 3.6%) a as white solid.

¹H NMR (400 MHz, DMSO-d₆) δ 7.58-7.55 (m, 2H), 7.48-7.44 (m, 1H), 7.17 (dd, J=8.4 Hz, 1H), 6.87 (d, J=2.4 Hz, 1H), 6.81 (dd, J=8.4, 2.4 Hz, 1H), 5.17 (s, 2H), 4.75 (t, J=8.8 Hz, 1H), 2.94-2.88 (m, 1H), 2.80-2.73 (m, 1H), 2.70-2.64 (m, 1H), 2.60-2.55 (m, 1H), 2.34-2.25 (m, 1H), 2.09-2.03 (m, 2H), 1.84-1.81 (m, 1H), 1.75-1.71 (m, 1H), 1.38-1.24 (m, 2H), 1.13 (d, J=6.0 Hz, 3H), 0.65 (d, J=6.0 Hz, 3H).

LCMS (ESI, m/z): fragmental 291 [M+H]⁺. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 220 nm; RT: 1.617 min.

### Example S32. Synthesis of 1-(5-((2,6-dichlorobenzyl)oxy)-7-fluoro-2,3-dihydro-1H-inden-1-yl)piperidine-4-carboxylic acid (32)

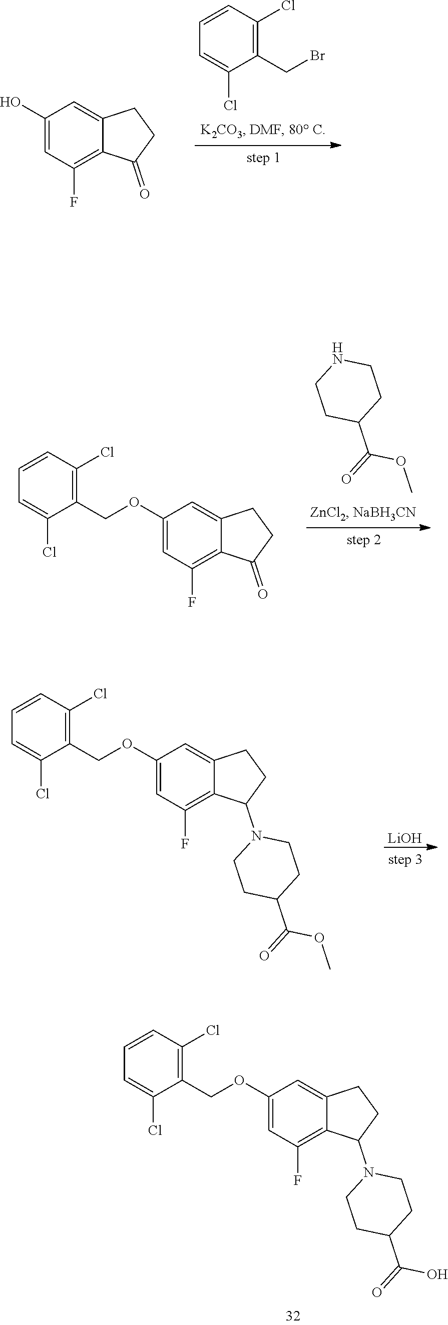

### Synthesis of 5-((2,6-dichlorobenzyl)oxy)-7-fluoro-2,3-dihydro-1H-inden-1-one

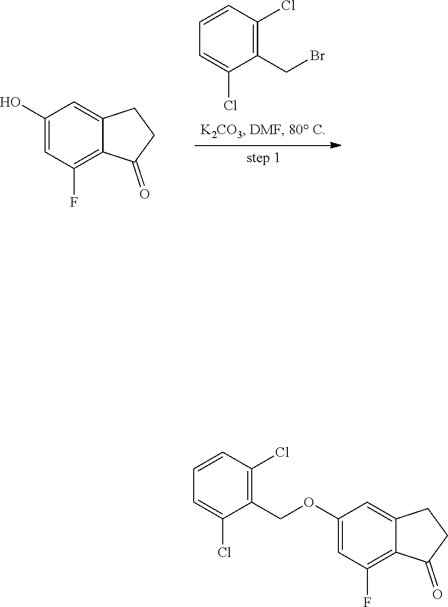 [SMILES: O=C1CCc2cc(O)cc(F)c21 ; Clc1cccc(Cl)c1CBr ; O=C1CCc2cc(OCc3c(Cl)cccc3Cl)cc(F)c21]

A mixture of 7-fluoro-5-hydroxy-indan-1-one (300 mg, 1.810 mmol, 1.00 equiv), 2-(bromomethyl)-1,3-dichloro-benzene (520 mg, 2.170 mmol, 1.50 equiv) and K₂CO₃ (748 mg, 5.420 mmol, 3.00 equiv) in DMF (10 mL) was stirred at 80° C. for 15 h under N₂ atmosphere. TLC showed the reaction was complete. The reaction mixture was concentrated under reduced pressure. The residue was purified by flash chromatography on silica gel (EA/PE=30%) to give 5-((2,6-dichlorobenzyl)oxy)-7-fluoro-2,3-dihydro-1H-inden-1-one (320 mg, 55%) as a white solid. LCMS (ESI, m/z): 325 [M+H]⁺.

### Synthesis of methyl 1-(5-((2,6-dichlorobenzyl)oxy)-7-fluoro-2,3-dihydro-1H-inden-1-yl)piperidine-4-carboxylate

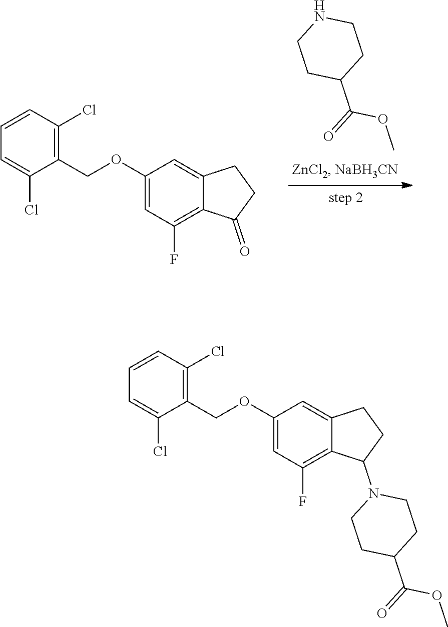

A mixture of 5-((2,6-dichlorobenzyl)oxy)-7-fluoro-2,3-dihydro-1H-inden-1-one (300 mg, 0.920 mmol, 1.00 equiv), methyl piperidine-4-carboxylate (198 mg, 1.380 mmol, 1.50 equiv), NaBH₃CN (236 mg, 3.690 mmol, 4.00 equiv) and ZnCl₂ (1.9 M in THF, 1.0 mL, 1.850 mmol, 2.00 equiv) in methanol (20 mL) was stirred at 60° C. for 15 h. LCMS showed the reaction was complete. The reaction was quenched with 50 mL water and filtered. The filtrate was extracted with ethyl acetate (20 mL*3). The combined organic layers were dried over anhydrous Na₂SO₄ and concentrated under vacuum. The residue was purified by flash chromatography on silica gel (EA/PE=25%) to give methyl 1-(5-((2,6-dichloro-benzyl)oxy)-7-fluoro-2,3-dihydro-1H-inden-1-yl)piperidine-4-carboxylate (200 mg, 48%) as a yellow oil. LCMS (ESI, m/z): 452 [M+H]⁺.

### Synthesis of 1-(5-((2,6-dichlorobenzyl)oxy)-7-fluoro-2,3-dihydro-1H-inden-1-yl)piperidine-4-carboxylic acid

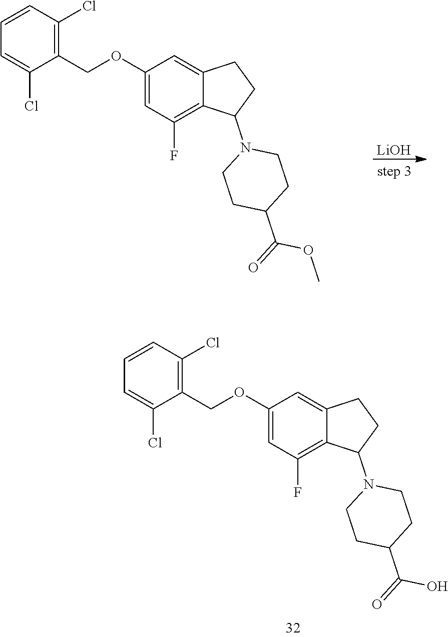 [SMILES: COC(=O)C1CCN(C2CCc3cc(OCc4c(Cl)cccc4Cl)cc(F)c32)CC1 ; "LiOH" ; O=C(O)C1CCN(C2CCc3cc(OCc4c(Cl)cccc4Cl)cc(F)c32)CC1]

A mixture of methyl 1-(5-((2,6-dichlorobenzyl)oxy)-7-fluoro-2,3-dihydro-1H-inden-1-yl)piperidine-4-carboxylate (100 mg, 0.220 mmol, 1.00 equiv) and LiGH (16 mg, 0.660 mmol, 3.00 equiv) in THE (2 mL) and water (1 mL) was stirred at room temperature for 1 h. LCMS showed the reaction was complete. The mixture was acidified to pH 4-5 with 1 M HCl, and concentrated under reduced pressure. The residue was purified by prep-HPLC (Column: YMC-Actus Triart C18, 30*250 mm, 5 m; Mobile Phase A: water (10 mM NH₄HCO₃+0.1% NH₃·H₂O), Mobile Phase B: ACN; Flow rate: 60 mL/min; Gradient: 20% B to 35% B in 10 min; 254/210 nm; RT: 9.67 min) to give 1-(5-((2,6-dichlorobenzyl)oxy)-7-fluoro-2,3-dihydro-1H-inden-1-yl)piperidine-4-carboxylic acid (27.8 mg, 28%) as a white solid.

LCMS (ESI, m/z): 438 [M+H]⁺. Analytic Conditions: column: HALO C18, 3.0*30 mm, 2.0 m; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 210 nm; RT: 0.918 min.

¹H NMR (400 MHz, DMSO-d₆) δ 7.59-7.57 (m, 2H), 7.50-7.46 (m, 1H), 6.81-6.79 (m, 1H), 6.75-6.72 (m, 1H), 5.21 (s, 2H), 4.33-4.30 (m, 1H), 2.97-2.89 (m, 1H), 2.80-2.70 (m, 2H), 2.60-2.57 (m, 1H), 2.20-1.98 (m, 5H), 1.76-1.73 (m, 2H), 1.55-1.35 (m, 2H).

### Example S33. Synthesis of 1-(5-((2,6-dichlorobenzyl)oxy)-6-fluoro-2,3-dihydro-1H-inden-1-yl)piperidine-4-carboxylic acid (33)

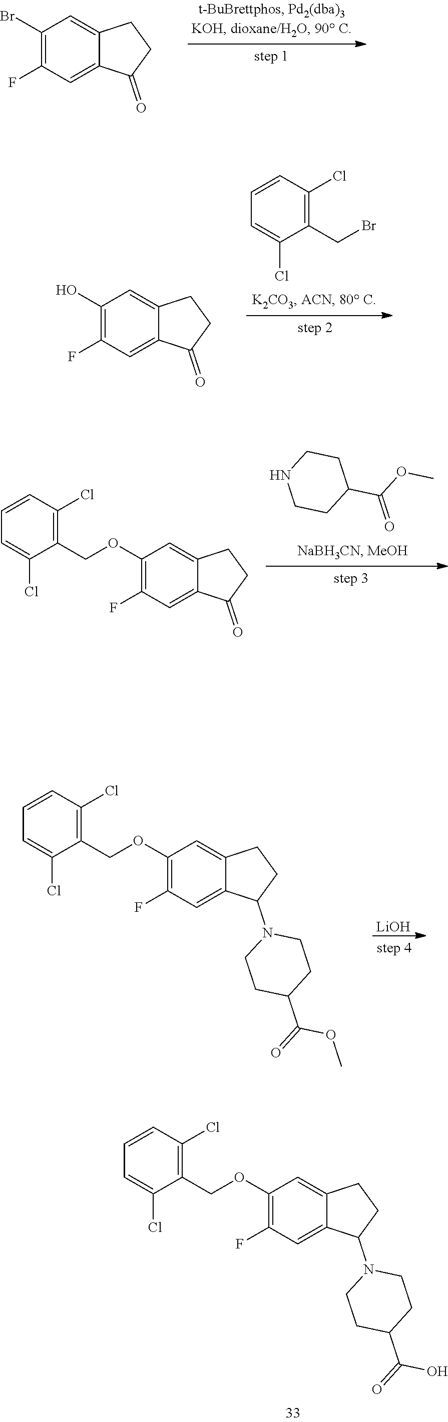

### Synthesis of 6-fluoro-5-hydroxy-2,3-dihydro-1H-inden-1-one

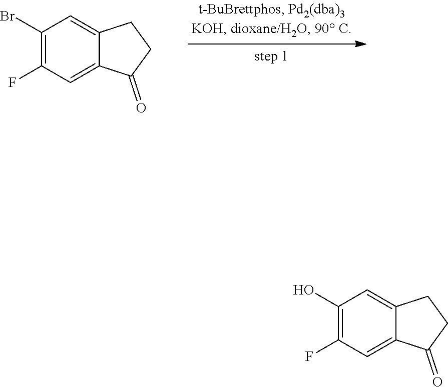 [SMILES: O=C1CCc2cc(Br)c(F)cc21 ; O=C1CCc2cc(O)c(F)cc21]

To a stirred solution of 5-bromo-6-fluoro-indan-1-one (300 mg, 1.310 mmol, 1.00 equiv) and t-BuBrettphos (126 mg, 0.260 mmol, 0.20 equiv) in 1,4-dioxane (5.0 mL) and water (0.5 mL) were added Pd₂(dba)₃CHCl₃ (135 mg, 0.130 mmol, 0.10 equiv) and KOH (220 mg, 3.930 mmol, 3.00 equiv). The mixture was stirred at 90° C. for 16 h. LCMS showed the reaction was complete. The reaction was acidified to pH 4-5 with 1 M HCl. The resulting mixture was concentrated under reduced pressure. The residue was purified by flash chromatography on C18 silica (eluted with water (5 mM NH₄HCO₃/ACN, 80/20) to give 6-fluoro-5-hydroxy-2,3-dihydro-1H-inden-1-one (180 mg, 82% yield) as an off-white solid. LCMS (ESI, m/z): 167 [M+H]⁺.

### Synthesis of P5-((2,6-dichlorobenzyl)oxy)-6-fluoro-2,3-dihydro-1H-inden-1-one

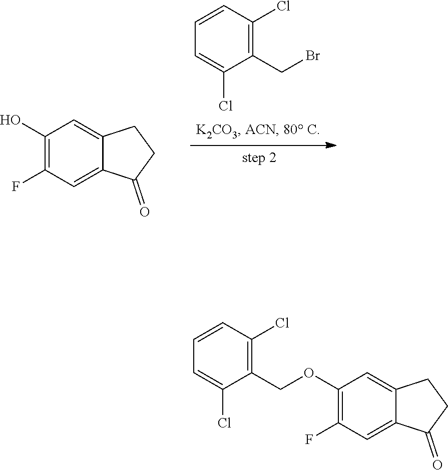 [SMILES: Clc1cccc(Cl)c1CBr ; O=C1CCc2cc(O)c(F)cc21 ; O=C1CCc2cc(OCc3c(Cl)cccc3Cl)c(F)cc21]

To a stirred solution of 6-fluoro-5-hydroxy-2,3-dihydro-1H-inden-1-one (160 mg, 0.960 mmol, 1.00 equiv) and 2-(bromomethyl)-1,3-dichloro-benzene (254 mg, 1.060 mmol, 1.10 equiv) in MeCN (5.0 mL) was added K₂CO₃ (398 mg, 2.890 mmol, 3.00 equiv). The mixture was stirred at 80° C. for 16 h. LCMS showed the reaction was complete. The reaction mixture was concentrated under reduced pressure and the residue was purified by column chromatography (EA/PE=55/45) to give 5-((2,6-dichlorobenzyl)oxy)-6-fluoro-2,3-dihydro-1H-inden-1-one (260 mg, 83% yield) as an off-white solid. LCMS (ESI, m/z): 325 [M+H]⁺.

### Synthesis of methyl 1-(5-((2,6-dichlorobenzyl)oxy)-6-fluoro-2,3-dihydro-1H-inden-1-yl)piperidine-4-carboxylate

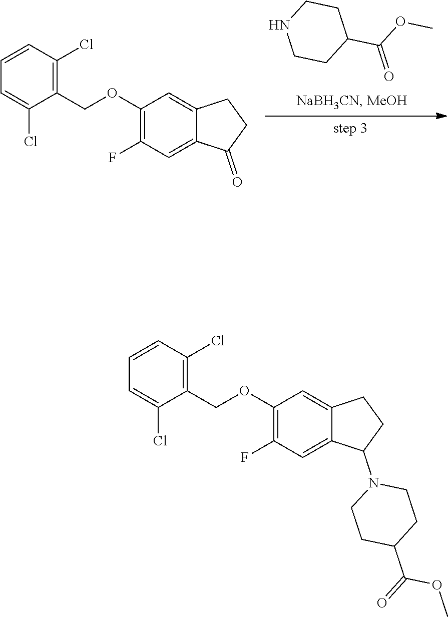

To a stirred solution of 5-((2,6-dichlorobenzyl)oxy)-6-fluoro-2,3-dihydro-1H-inden-1-one (240 mg, 0.740 mmol, 1.00 equiv) and methyl piperidine-4-carboxylate (211 mg, 1.480 mmol, 2.00 equiv) in methanol (10.0 mL) were added NaBH₃CN (141 mg, 2.210 mmol, 3.00 equiv) and ZnCl₂ (2 M in THF, 0.7 mL, 1.480 mmol, 2.00 equiv). The mixture was stirred at 60° C. for 16 h. LCMS showed the reaction was complete. The reaction was quenched by addition of 50 mL of water and filtered. The filtrate was extracted with ethyl acetate (20 mL*3). The combined organic layers were dried over anhydrous Na₂SO₄ and concentrated under vacuum. The residue was purified by flash chromatography on silica gel (EA/PE=25/75) to give methyl 1-(5-((2,6-dichlorobenzyl)oxy)-6-fluoro-2,3-dihydro-1H-inden-1-yl)piperidine-4-carboxylate (150 mg, 44% yield) as an off-white solid. LCMS (ESI, m/z): 452 [M+H]⁺.

### Synthesis of 1-(5-((2,6-dichlorobenzyl)oxy)-6-fluoro-2,3-dihydro-1H-inden-1-yl)piperidine-4-carboxylic acid

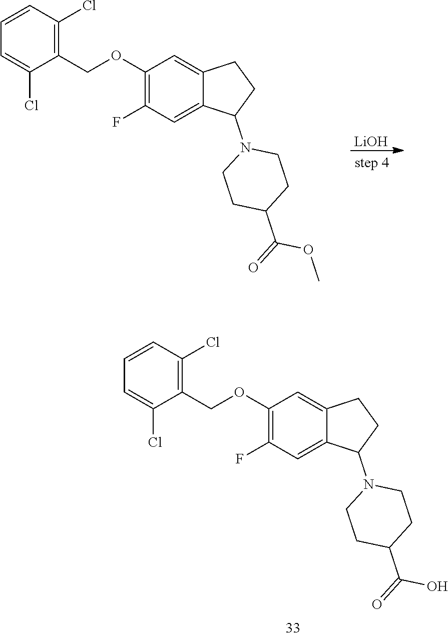 [SMILES: COC(=O)C1CCN(C2CCc3cc(OCc4c(Cl)cccc4Cl)c(F)cc32)CC1 ; "LiOH" ; O=C(O)C1CCN(C2CCc3cc(OCc4c(Cl)cccc4Cl)c(F)cc32)CC1]

To a stirred solution of methyl 1-(5-((2,6-dichlorobenzyl)oxy)-6-fluoro-2,3-dihydro-1H-inden-1-yl)piperidine-4-carboxylate (100 mg, 0.220 mmol, 1.00 equiv) in THF (1.0 mL) and water (0.1 mL) was added LiOH*H₂O (27 mg, 0.660 mmol, 3.00 equiv). The resulting mixture was stirred at room temperature for 16 h. LCMS showed the reaction was complete. The mixture was acidified to pH 4-5 with 1 M HCl, and concentrated under reduced pressure. The residue was purified by prep-HPLC (Column: XBridge C18 OBD Prep Column, 5 μm, 19*250 mm; Mobile Phase A: water (10 mM NH₄HCO₃+0.1% NH₃·H₂O), Mobile Phase B: ACN; Flow rate: 25 mL/min; Gradient: 20% B to 43% B in 7 min; 254/210 nm; RT: 6.16 min) to give 1-(5-((2,6-dichlorobenzyl)oxy)-6-fluoro-2,3-dihydro-1H-inden-1-yl)piperidine-4-carboxylic acid (33 mg, 33%) as an off-white solid.

LCMS (ESI, m/z): 438 [M+H]⁺. Analytic Conditions: column: Poroshell HPH-C18, 3.0*50 mm, 2.7 m; mobile phase A: water (5 mM NH₄HCO₃), mobile phase B: acetonitrile; flow rate: 1.20 mL/min; gradient: 10% B to 95% B in 2.00 min, hold at 95% for 0.60 min, 95% B to 10% B in 0.15 min; 254 nm; RT: 1.145 min.

¹H NMR (300 MHz, DMSO-d₆) δ 7.60-7.57 (m, 2H), 7.52-7.47 (m, 1H), 7.24 (d, J=7.8 Hz, 1H), 7.02 (d, J=11.1 Hz, 1H), 5.27 (s, 2H), 4.25 (t, J=6.9 Hz, 1H), 2.92-2.70 (m, 3H), 2.49-2.44 (m, 1H), 2.28-2.11 (m, 3H), 2.05-1.98 (m, 2H), 1.83-1.74 (m, 2H), 1.65-1.40 (m, 2H).

### Example S34. Synthesis of 1-(5-((2,6-dichlorobenzyl)oxy)-4-fluoro-2,3-dihydro-1H-inden-1-yl)piperidine-4-carboxylic acid (34)

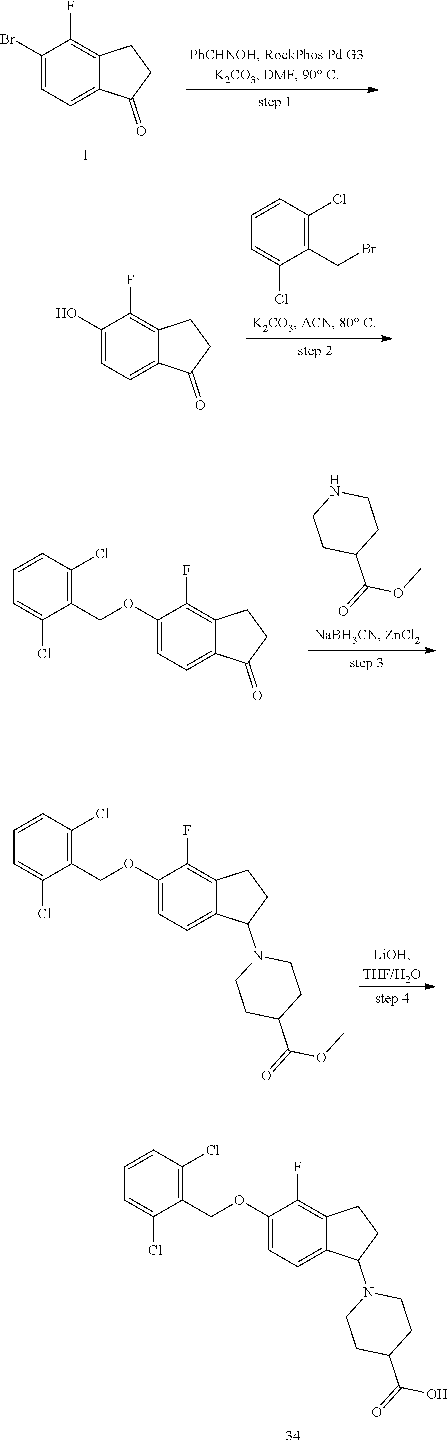

### Synthesis of 4-fluoro-5-hydroxy-2,3-dihydro-1H-inden-1-one

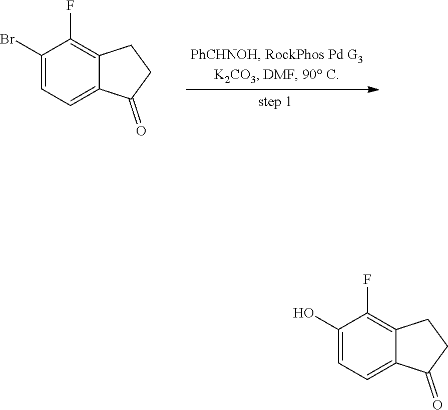 [SMILES: O=C1CCc2c1ccc(Br)c2F ; O=C1CCc2c1ccc(O)c2F]

To a stirred solution of 5-bromo-4-fluoro-indan-1-one (450 mg, 1.96 mmol, 1.00 equiv) and K₂CO₃ (814 mg, 5.88 mmol, 3.00 equiv) in DMF (5 mL) were added PhCHNOH (442 mg, 2.95 mmol, 1.50 equiv) and RockPhos Pd G3 (163 mg, 0.196 mmol, 0.100 equiv). The resulting mixture was stirred at 90° C. overnight. LCMS showed the reaction was complete. The reaction was acidified to pH 4-5 with 1 M HCl. The resulting mixture was concentrated under reduced pressure. The residue was purified by flash chromatography on C18 silica (5 mM NH₄HCO₃/ACN, 70/30) to give 4-fluoro-5-hydroxy-2,3-dihydro-1H-inden-1-one (300 mg, 1.81 mmol, 91.9%) as a yellow oil. LCMS (ESI, m/z): 167 [M+H]⁺.

### Synthesis of 5-((2,6-dichlorobenzyl)oxy)-4-fluoro-2,3-dihydro-1H-inden-1-one

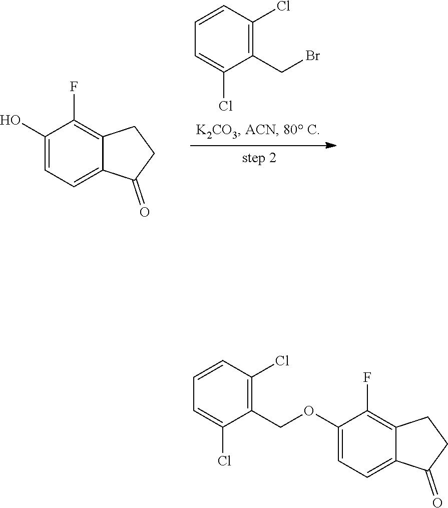 [SMILES: O=C1CCc2c1ccc(O)c2F ; Clc1cccc(Cl)c1CBr ; O=C1CCc2c1ccc(OCc1c(Cl)cccc1Cl)c2F]

To a stirred solution of 4-fluoro-5-hydroxy-2,3-dihydro-1H-inden-1-one (300 mg, 1.81 mmol, 1.00 equiv) and 2-(bromomethyl)-1,3-dichloro-benzene (650 mg, 2.71 mmol, 1.50 equiv) in MeCN (6 mL) was added K₂CO₃ (748 mg, 5.42 mmol, 3.00 equiv). The resulting mixture was stirred at 80° C. overnight. LCMS showed the reaction was complete. The reaction mixture was concentrated under reduced pressure. The crude product was purified by Prep-TLC (PE/EA, 2/1) to afford 5-((2,6-dichlorobenzyl)oxy)-4-fluoro-2,3-dihydro-1H-inden-1-one (300 mg, 0.923 mmol, 51.1%) as a yellow oil. LCMS (ESI, m/z): 325 [M+H]⁺.

### Synthesis of methyl 1-(5-((2,6-dichlorobenzyl)oxy)-4-fluoro-2,3-dihydro-1H-inden-1-yl)piperidine-4-carboxylate

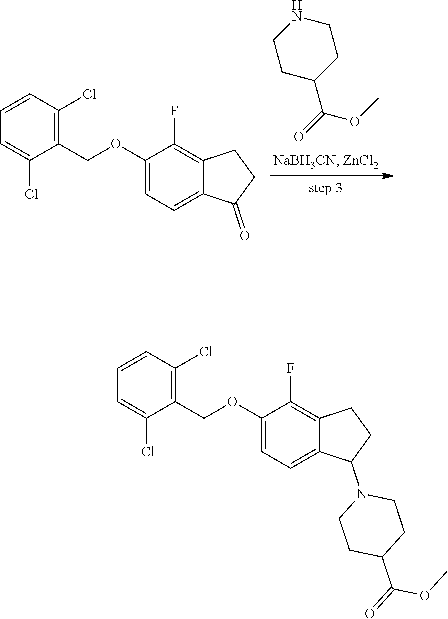

To a stirred solution of ZnCl₂ (1.0 mL, 1.9 M in Me-THF) and NaBH₃CN (236 mg, 3.69 mmol, 4.00 equiv) in methanol (5 mL) were added 5-((2,6-dichlorobenzyl)oxy)-4-fluoro-2,3-dihydro-1H-inden-1-one (300 mg, 0.920 mmol, 1.00 equiv) and methyl piperidine-4-carboxylate (264 mg, 1.85 mmol, 2.00 equiv). The resulting mixture was stirred at 80° C. overnight. LCMS showed the reaction was complete. The reaction was quenched with 50 mL of water and filtered. The filtrate was extracted with ethyl acetate (20 mL*3). The combined organic layers were dried over anhydrous Na₂SO₄ and concentrated under vacuum. The residue was purified by flash chromatography on silica gel (eluted with PE/EA, 1/1) to afford methyl 1-(5-((2,6-dichlorobenzyl)oxy)-4-fluoro-2,3-dihydro-1H-inden-1-yl)piperidine-4-carboxylate (100 mg, 0.221 mmol, 24.0%) as a yellow oil. LCMS (ESI, m/z): 452 [M+H]⁺.

### Synthesis of 1-(5-((2,6-dichlorobenzyl)oxy)-4-fluoro-2,3-dihydro-1H-inden-1-yl)piperidine-4-carboxylic acid

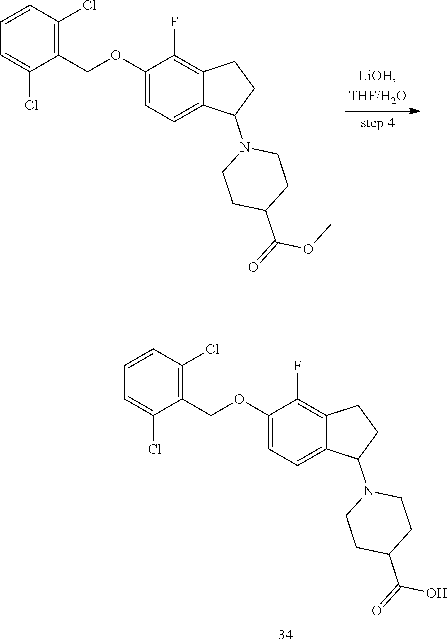 [SMILES: COC(=O)C1CCN(C2CCc3c2ccc(OCc2c(Cl)cccc2Cl)c3F)CC1 ; O=C(O)C1CCN(C2CCc3c2ccc(OCc2c(Cl)cccc2Cl)c3F)CC1]

To a stirred solution of methyl 1-(5-((2,6-dichlorobenzyl)oxy)-4-fluoro-2,3-dihydro-1H-inden-1-yl)piperidine-4-carboxylate (70 mg, 0.150 mmol, 1.00 equiv) in THE (2 mL) and water (2 mL) was added LiOH (11 mg, 0.450 mmol, 3.00 equiv). The resulting mixture was stirred at room temperature for 2 h. LCMS showed the reaction was complete. The mixture was acidified to pH 4-5 with 1 M HCl, and concentrated under reduced pressure. The residue was purified by prep-HPLC (Column: XBridge Prep OBD C18 Column, 30*150 mm, 5 m; Mobile Phase A: water (10 mM NH₄HCO₃), Mobile Phase B: ACN; Flow rate: 60 mL/min; Gradient: 80% B to 95% B in 7 min; 254 nm; RT: 7.5 min) to afford 1-(5-((2,6-dichlorobenzyl)oxy)-4-fluoro-2,3-dihydro-1H-inden-1-yl)piperidine-4-carboxylic acid (53.5 mg, 77.6%) as a white solid.

LCMS (ESI, m/z): 438 [M+H]⁺. Analytic Conditions: EVO C18, 3.0*50 mm, 2.6 m; Mobile Phase A: water (5 mM NH₄HCO₃), Mobile Phase B: ACN; Flow rate: 1.20 mL/min; Gradient: 10% B to 95% B in 2.0 min, hold at 95% B for 0.60 min, 95% B to 10% B in 0.15 min; 210 nm; RT: 1.125 min.

¹H NMR (400 MHz, DMSO-d₆) δ 12.03 (br, 1H), 7.59-7.57 (m, 2H), 7.51-7.47 (m, 1H), 7.20 (t, J=8.0 Hz, 1H), 7.04 (d, J=8.4 Hz, 1H), 5.27 (s, 2H), 4.28 (t, J=7.2 Hz, 1H), 2.92-2.72 (m, 3H), 2.49-2.47 (m, 1H), 2.25-2.15 (m, 3H), 2.07-2.01 (m, 2H), 1.82-1.75 (m, 2H), 1.63-1.57 (m, 1H), 1.54-1.42 (m, 1H).

### Example S35. Synthesis of 1-(5-((2,6-dichlorobenzyl)oxy)-7-methyl-2,3-dihydro-1H-inden-1-yl)piperidine-4-carboxylic acid (35)

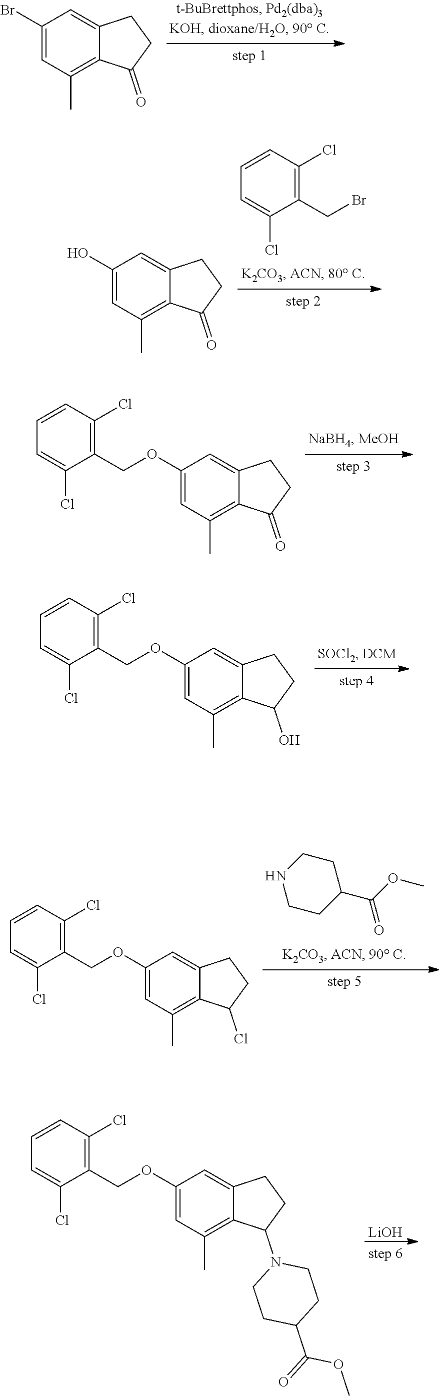

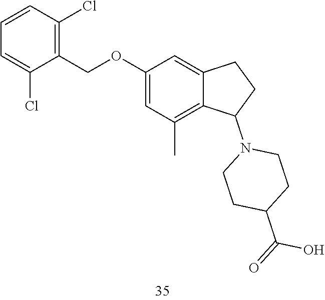 [SMILES: Cc1cc(OCc2c(Cl)cccc2Cl)cc2c1C(N1CCC(C(=O)O)CC1)CC2]

### Synthesis of 5-hydroxy-7-methyl-2,3-dihydro-1H-inden-1-one

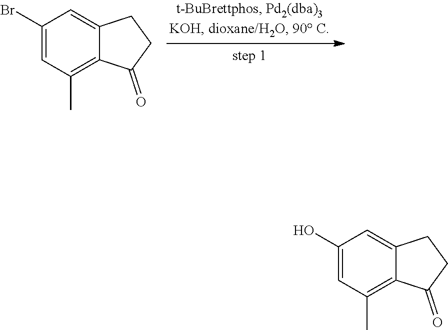

To a stirred solution of 5-bromo-7-methyl-indan-1-one (300 mg, 1.330 mmol, 1.00 equiv) and t-BuBrettPhos (129 mg, 0.270 mmol, 0.20 equiv) in 1,4-dioxane (5.0 mL) and water (0.5 mL) were added Pd₂(dba)₃CHCl₃ (122 mg, 0.130 mmol, 0.10 equiv) and KOH (224 mg, 4.000 mmol, 3.00 equiv). The resulting mixture was stirred at 90° C. for 16 h. LCMS showed the reaction was complete. The resulting solution was extracted with ethyl acetate (3×300 mL). The reaction was acidified to pH 4-5 with 1 M HCl. The resulting mixture was concentrated under reduced pressure. The residue was purified by flash column chromatography on C18 silica (5 mM NH₄HCO₃/ACN, 33/67) to give 5-hydroxy-7-methyl-2,3-dihydro-1H-inden-1-one (180 mg, 83% yield) as an off-white solid. LCMS (ESI, m/z): 163 [M+H]⁺.

### Synthesis of 5-((2,6-dichlorobenzyl)oxy)-7-methyl-2,3-dihydro-1H-inden-1-one

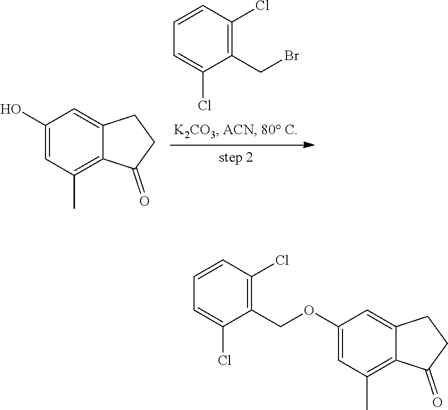 [SMILES: Clc1cccc(Cl)c1CBr ; Cc1cc(O)cc2c1C(=O)CC2 ; Cc1cc(OCc2c(Cl)cccc2Cl)cc2c1C(=O)CC2]

To a stirred solution of 5-hydroxy-7-methyl-2,3-dihydro-1H-inden-1-one (160 mg, 0.990 mmol, 1.00 equiv) and 2-(bromomethyl)-1,3-dichloro-benzene (260 mg, 1.090 mmol, 1.10 equiv) in MeCN (10.0 mL) was added K₂CO₃ (408 mg, 2.960 mmol, 3.00 equiv). The mixture was stirred at 80° C. for 16 h. LCMS showed the reaction was complete. The mixture was concentrated under reduced pressure and the residue was purified by column chromatography (EA/PE, 3/2) to give 5-((2,6-dichlorobenzyl)oxy)-7-methyl-2,3-dihydro-1H-inden-1-one (260 mg, 82% yield) as an off-white solid. LCMS (ESI, m/z): 321 [M+H]⁺.

### Synthesis of 5-((2,6-dichlorobenzyl)oxy)-7-methyl-2,3-dihydro-1H-inden-1-ol

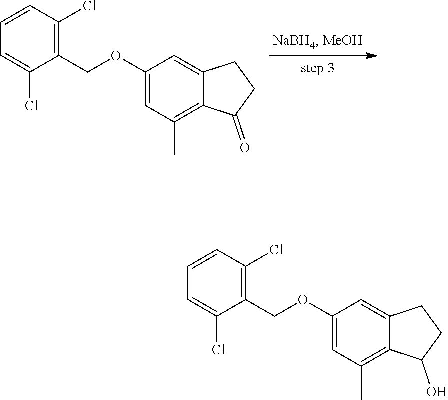 [SMILES: Cc1cc(OCc2c(Cl)cccc2Cl)cc2c1C(=O)CC2 ; Cc1cc(OCc2c(Cl)cccc2Cl)cc2c1C(O)CC2]

To a stirred solution of 5-((2,6-dichlorobenzyl)oxy)-7-methyl-2,3-dihydro-1H-inden-1-one (260 mg, 0.810 mmol, 1.00 equiv) in methanol (10.0 mL) was added NaBH₄ (91 mg, 2.430 mmol, 3.00 equiv). The resulting mixture was stirred at 0° C. for 2 h. LCMS showed the reaction was complete. The resulting solution was quenched with water (20 mL) and extracted with ethyl acetate (10 mL*3). The organic layers were combined, dried and concentrated under vacuum. The residue was purified by flash chromatography on silica gel (EA/PE, 5/1) to give 5-((2,6-dichlorobenzyl)oxy)-7-methyl-2,3-dihydro-1H-inden-1-ol (240 mg, 91%) as an off-white solid. LCMS (ESI, m/z): 323 [M+H]⁺.

### Synthesis of 1-chloro-5-((2,6-dichlorobenzyl)oxy)-7-methyl-2,3-dihydro-1H-indene

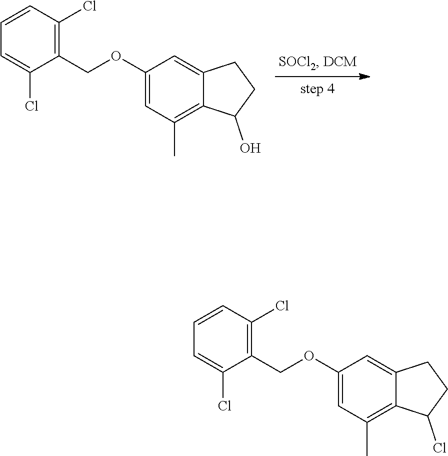 [SMILES: Cc1cc(OCc2c(Cl)cccc2Cl)cc2c1C(O)CC2 ; Cc1cc(OCc2c(Cl)cccc2Cl)cc2c1C(Cl)CC2]

To a stirred solution of 5-((2,6-dichlorobenzyl)oxy)-7-methyl-2,3-dihydro-1H-inden-1-ol (240 mg, 0.740 mmol, 1.00 equiv) in 1,4-dioxane (10.0 mL) were added SOCl₂ (0.2 mL, 2.230 mmol, 3.00 equiv). The mixture was stirred at 0° C. for 1 h. LCMS showed the reaction was complete. The reaction was concentrated under reduced pressure, and the crude 1-chloro-5-((2,6-dichlorobenzyl)oxy)-7-methyl-2,3-dihydro-1H-indene was used in the next step directly without further purification. LCMS (ESI, m/z): 342 [M+H]⁺.

### Synthesis of methyl 1-(5-((2,6-dichlorobenzyl)oxy)-7-methyl-2,3-dihydro-1H-inden-1-yl)piperidine-4-carboxylate

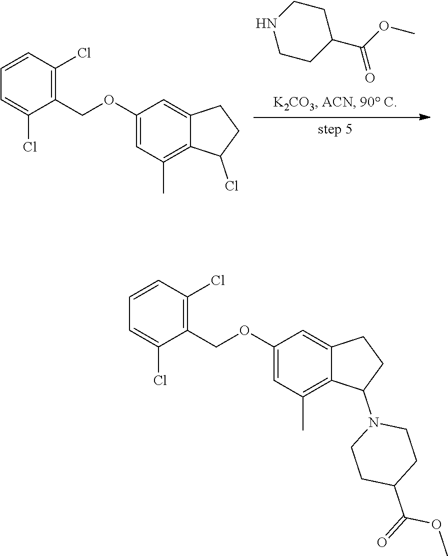

To a stirred solution of 1-chloro-5-((2,6-dichlorobenzyl)oxy)-7-methyl-2,3-dihydro-1H-indene (240 mg, 0.700 mmol, 1.00 equiv) and methyl piperidine-4-carboxylate (201 mg, 1.400 mmol, 2.00 equiv) in MeCN (10.0 mL) was added K₂CO₃ (290 mg, 2.110 mmol, 3.00 equiv). The resulting mixture was stirred at 90° C. for 16 h. LCMS showed the reaction was complete. The reaction mixture was concentrated under reduced pressure and the residue was purified by column chromatography (EA/PE, 3/2) to give methyl 1-(5-((2,6-dichlorobenzyl)oxy)-7-methyl-2,3-dihydro-1H-inden-1-yl)piperidine-4-carboxylate (50 mg, 15%) as an off-white solid. LCMS (ESI, m/z): 448 [M+H]⁺.

### Synthesis of 1-(5-((2,6-dichlorobenzyl)oxy)-7-methyl-2,3-dihydro-1H-inden-1-yl)piperidine-4-carboxylic acid

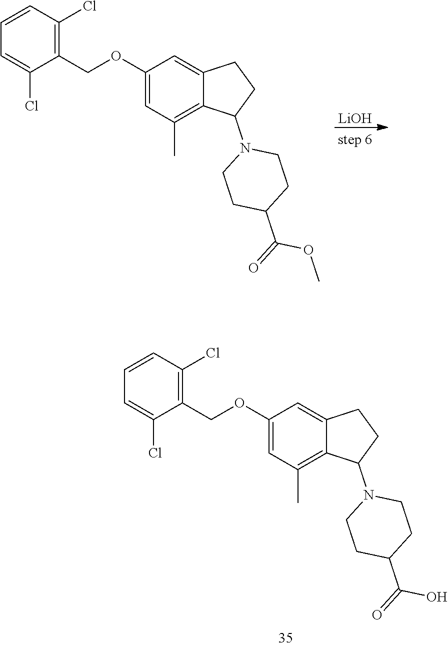 [SMILES: COC(=O)C1CCN(C2CCc3cc(OCc4c(Cl)cccc4Cl)cc(C)c32)CC1 ; "LiOH" ; Cc1cc(OCc2c(Cl)cccc2Cl)cc2c1C(N1CCC(C(=O)O)CC1)CC2]

To a stirred solution of methyl 1-(5-((2,6-dichlorobenzyl)oxy)-7-methyl-2,3-dihydro-1H-inden-1-yl)piperidine-4-carboxylate (50 mg, 0.110 mmol, 1.00 equiv) in THE (1.0 mL) and water (0.5 mL) was added LiOH H₂O (14 mg, 0.330 mmol, 3.00 equiv). The mixture was stirred at room temperature for 16 h. LCMS showed the reaction was complete. The mixture was acidified to pH 4-5 with 1 M HCl, and concentrated under reduced pressure. The residue was purified by prep-HPLC (Column: XBridge Prep C18 OBD Column, 19*150 mm, 5 m; Mobile Phase A: water (10 mM NH₄HCO₃), Mobile Phase B: ACN; Flow rate: 25 mL/min; Gradient: 47% B to 67% B in 7 min; 254/210 nm, RT: 5.78 min) to give 1-(5-((2,6-dichloro-benzyl)oxy)-7-methyl-2,3-dihydro-1H-inden-1-yl)piperidine-4-carboxylic acid (10.5 mg, 20%) as an off-white solid.

LCMS (ESI, m/z): 434 [M+H]⁺. Analytic Conditions: column: Poroshell HPH-C18, 3.0*50 mm, 2.7 m; mobile phase A: water (5 mM NH₄HCO₃), mobile phase B: acetonitrile; flow rate: 1.20 mL/min; gradient: 10% B to 95% B in 2.00 min, hold at 95% for 0.60 min, 95% B to 10% B in 0.15 min; 254 nm; RT: 1.215 min.

¹H NMR (300 MHz, Methanol-d₄) δ 7.49-7.46 (m, 2H), 7.40-7.35 (m, 1H), 6.87-6.79 (m, 2H), 5.30 (s, 2H), 4.70-4.67 (m, 1H), 3.25-3.12 (m, 3H), 2.94-2.80 (m, 3H), 2.54-2.49 (m, 1H), 2.45 (s, 3H), 2.37-2.24 (m, 2H), 2.05-1.87 (m, 4H).

### Example S36. Synthesis of 1-(5-((2,6-dichlorobenzyl)oxy)-4-methyl-2,3-dihydro-1H-inden-1-yl)piperidine-4-carboxylic acid (36)

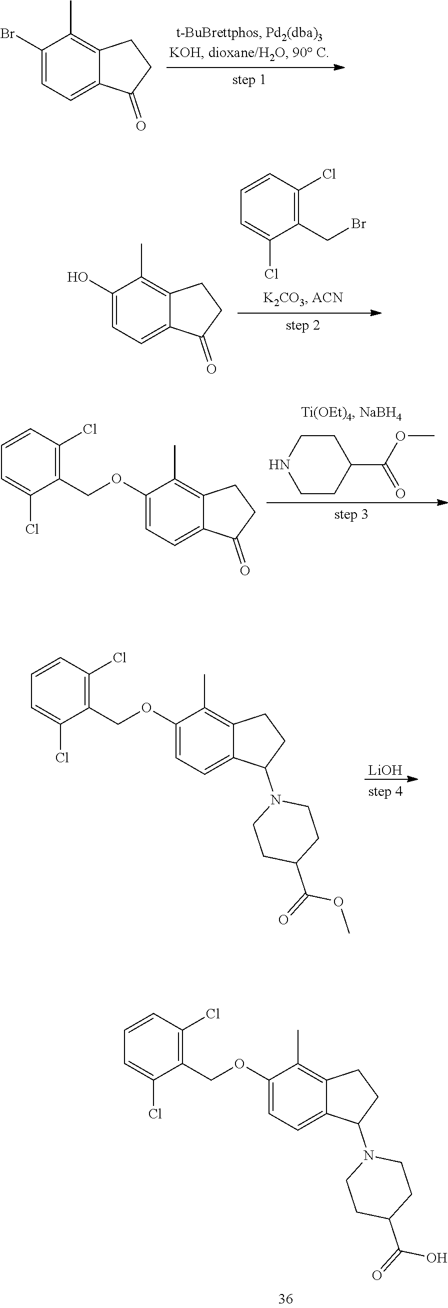

### Synthesis of 5-hydroxy-4-methyl-2,3-dihydro-1H-inden-1-one

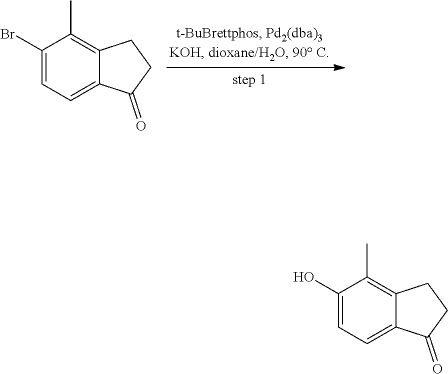

To a stirred solution of 5-bromo-4-methyl-indan-1-one (400 mg, 1.780 mmol, 1.00 equiv) in 1,4-dioxane (10.0 mL) and water (2.0 mL) were added t-BuBrettPhos (172 mg, 0.360 mmol, 0.20 equiv), Pd₂(dba)₃ (162 mg, 0.180 mmol, 0.10 equiv) and KOH (298 mg, 5.330 mmol, 3.00 equiv). The reaction was stirred at 90° C. for 3 h. LCMS showed the reaction was complete. The reaction was acidified to pH 4-5 with 1 M HCl. The resulting mixture was concentrated under reduced pressure. The residue was purified by flash chromatography on C18 silica (5 mM NH₄HCO₃/ACN, 80/20) to give 5-hydroxy-4-methyl-2,3-dihydro-1H-inden-1-one (200 mg, 69%) as an off white solid. LCMS (ESI, m/z): 163 [M+H]⁺.

### Synthesis of 5-((2,6-dichlorobenzyl)oxy)-4-methyl-2,3-dihydro-1H-inden-1-one

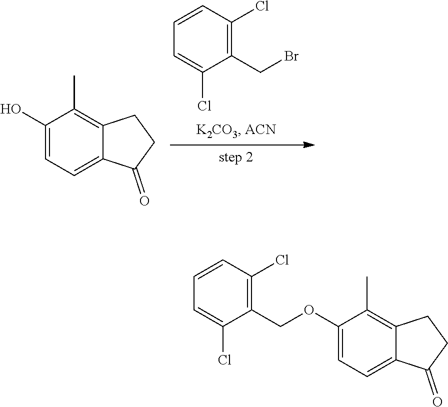 [SMILES: Cc1c(O)ccc2c1CCC2=O ; Clc1cccc(Cl)c1CBr ; Cc1c(OCc2c(Cl)cccc2Cl)ccc2c1CCC2=O]

To a stirred solution of 5-hydroxy-4-methyl-2,3-dihydro-1H-inden-1-one (100 mg, 0.620 mmol, 1.00 equiv) in MeCN (5.0 mL) were added 2-(bromomethyl)-1,3-dichloro-benzene (222 mg, 0.920 mmol, 1.50 equiv) and K₂CO₃ (255 mg, 1.850 mmol, 3.00 equiv). The reaction was stirred at 60° C. for 12 h. LCMS showed the reaction was complete. The reaction mixture was concentrated under reduced pressure and the residue was purified by column chromatography (EA/PE, 3/2) to give 5-((2,6-dichlorobenzyl)oxy)-4-methyl-2,3-dihydro-1H-inden-1-one (150 mg, 75% yield) as an off white solid. LCMS (ESI, m/z): 321 [M+H]⁺.

### Synthesis of methyl 1-(5-((2,6-dichlorobenzyl)oxy)-4-methyl-2,3-dihydro-1H-inden-1-yl)piperidine-4-carboxylate

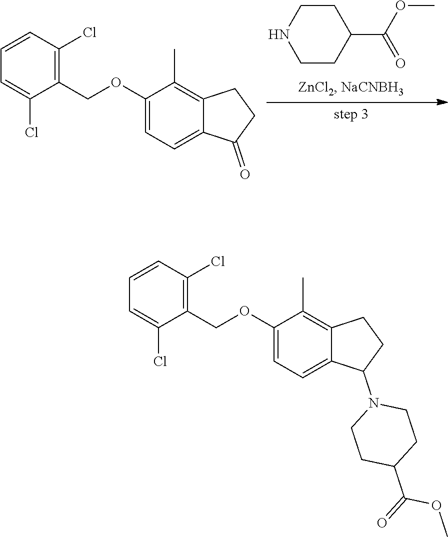

To a stirred solution of 5-((2,6-dichlorobenzyl)oxy)-4-methyl-2,3-dihydro-1H-inden-1-one (300 mg, 0.93 mmol, 1.00 equiv) and methyl piperidine-4-carboxylate (267 mg, 1.86 mmol, 2.00 equiv) in methanol (10.0 mL) were added NaBH₃CN (120 mg, 2.79 mmol, 3.00 equiv) and ZnCl₂ (2 M in THF, 0.9 mL, 1.86 mmol, 2.00 equiv). The mixture was stirred at 60° C. for 16 h. LCMS showed the reaction was complete. The reaction was quenched with 50 mL of water and filtered. The filtrate was extracted with ethyl acetate (20 mL*3). The combined organic layer was dried over anhydrous Na₂SO₄ and concentrated under vacuum. The residue was purified by flash chromatography on silica gel (EA/PE, 3/1) to give methyl 1-(5-((2,6-dichlorobenzyl)oxy)-4-methyl-2,3-dihydro-1H-inden-1-yl)piperidine-4-carboxylate (300 mg, 72%) as an off-white solid. LCMS (ESI, m/z): 448 [M+H]⁺.

### Synthesis of 1-(5-((2,6-dichlorobenzyl)oxy)-4-methyl-2,3-dihydro-1H-inden-1-yl)piperidine-4-carboxylic acid

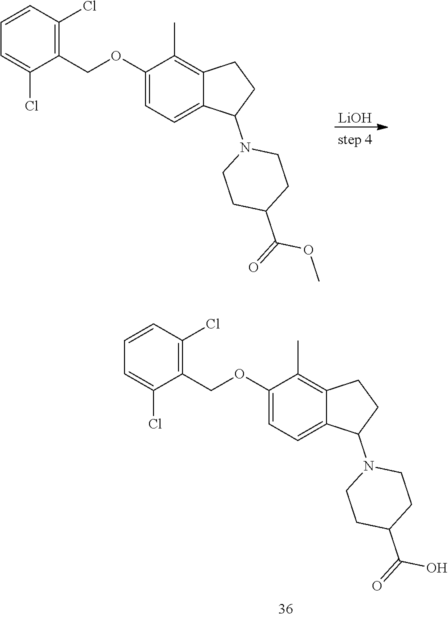 [SMILES: COC(=O)C1CCN(C2CCc3c2ccc(OCc2c(Cl)cccc2Cl)c3C)CC1 ; "LiOH" ; Cc1c(OCc2c(Cl)cccc2Cl)ccc2c1CCC2N1CCC(C(=O)O)CC1]

To a stirred solution of methyl 1-(5-((2,6-dichlorobenzyl)oxy)-4-methyl-2,3-dihydro-1H-inden-1-yl)piperidine-4-carboxylate (300 mg, 0.67 mmol, 1.00 equiv) in THE (3.0 mL) and water (1.0 mL) was added LiOH H₂O (84 mg, 2.01 mmol, 3.00 equiv). The reaction was stirred at room temperature for 3 h. LCMS showed the reaction was complete. The mixture was acidified to pH 4-5 with 1 M HCl, and concentrated under reduced pressure. The residue was purified by prep-HPLC (Column: XBridge Prep C18 OBD Column, 19*150 mm, 5 m; Mobile Phase A: water (10 mM NH₄HCO₃), Mobile Phase B: ACN; Flow rate: 25 mL/min; Gradient: 26% B to 47% B in 7 min; 254/210 nm; RT: 5.93 min) to give 1-(5-((2,6-dichloro-benzyl)oxy)-4-methyl-2,3-dihydro-1H-inden-1-yl)piperidine-4-carboxylic acid (18.5 mg, 6%) as a white solid.

LCMS (ESI, m/z): 434 [M+H]⁺. Analytic Conditions: column: Xbridge Shield RP18, 4.6*50 mm, 3.5 m; mobile phase A: water (5 mM NH₄HCO₃), mobile phase B: acetonitrile; flow rate: 1.50 mL/min; gradient: 10% B to 95% B in 1.75 min, hold at 95% for 0.05 min, 95% B to 10% B in 0.01 min; 210 nm; RT: 1.416 min.

¹H NMR (400 MHz, DMSO-d₆) δ 12.05 (s, 1H), 7.59-7.57 (m, 2H), 7.49-7.45 (m, 1H), 7.09-7.02 (m, 2H), 5.20 (s, 2H), 4.26-4.22 (m, 1H), 2.82-2.74 (m, 2H), 2.68-2.61 (m, 2H), 2.28-2.10 (m, 3H), 2.03-1.95 (m, 5H), 1.83-1.74 (m, 2H), 1.62-1.42 (m, 2H).

### Example S37. Synthesis of 1-(5-((2,6-dichlorobenzyl)oxy)-6-methyl-2,3-dihydro-1H-inden-1-yl)piperidine-4-carboxylic acid (37)

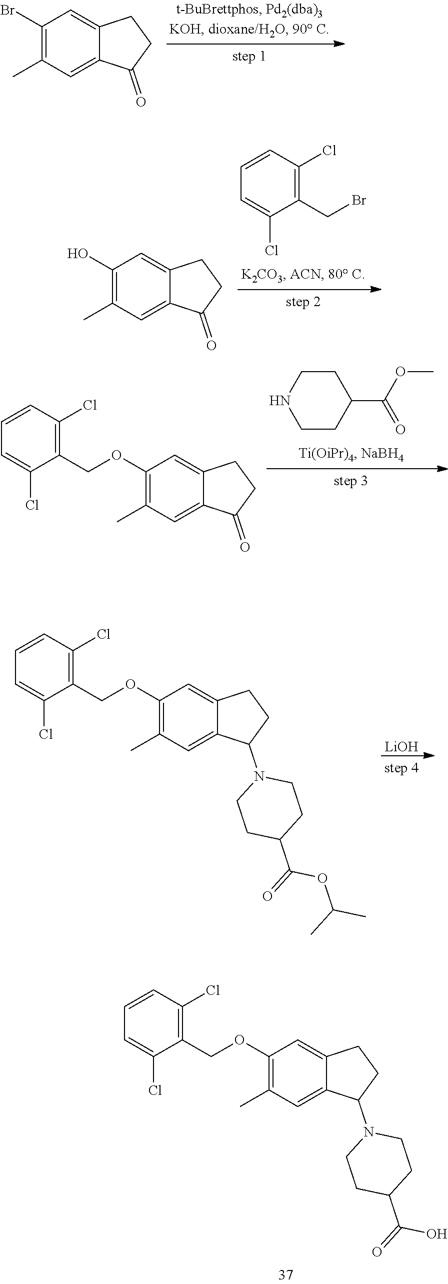

### Synthesis of 5-hydroxy-6-methyl-2,3-dihydro-1H-inden-1-one

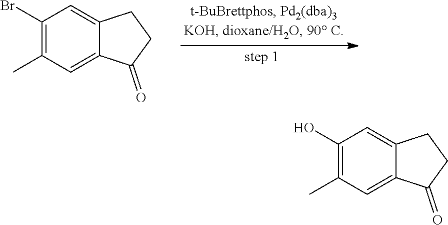

To a solution of 5-bromo-6-methyl-indan-1-one (400 mg, 1.780 mmol, 1.00 equiv) in 1,4-dioxane (2.0 mL) and water (0.4 mL) were added KOH (299 mg, 5.330 mmol, 3.00 equiv). Pd₂(dba)₃ (162 mg, 0.180 mmol, 0.10 equiv) and t-BuBrettPhos (172 mg, 0.360 mmol, 0.20 equiv) under a nitrogen atmosphere. The reaction was stirred for 12 h at 80° C. LCMS showed the reaction was complete. The reaction was acidified to pH 4-5 with 1 M HCl. The resulting mixture was concentrated under reduced pressure. The residue was purified by flash chromatography on C18 silica (5 mM NH₄HCO₃/ACN, 40/60) to give 5-hydroxy-6-methyl-2,3-dihydro-1H-inden-1-one (250 mg, 86%) as a yellow solid. LCMS (ESI, m/z): 163 [M+H]⁺.

### Synthesis of 5-((2,6-dichlorobenzyl)oxy)-6-methyl-2,3-dihydro-1H-inden-1-one

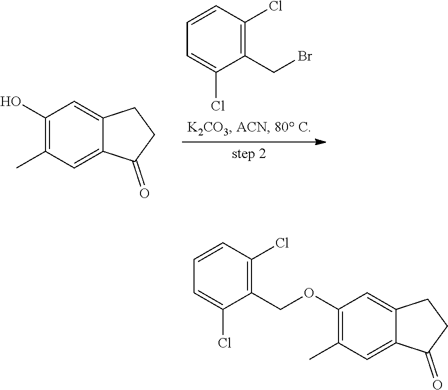 [SMILES: Cc1cc2c(cc1O)CCC2=O ; Clc1cccc(Cl)c1CBr ; Cc1cc2c(cc1OCc1c(Cl)cccc1Cl)CCC2=O]

To a solution of 5-hydroxy-6-methyl-2,3-dihydro-1H-inden-1-one (250 mg, 1.54 mmol, 1.00 equiv) in MeCN (5.0 mL) was added 2-(bromomethyl)-1,3-dichloro-benzene (554 mg, 2.31 mmol, 1.50 equiv) and K₂CO₃ (638 mg, 4.620 mmol, 3.00 equiv). The reaction was stirred for 12 h at 60° C. LCMS showed the reaction was complete. The reaction mixture was concentrated under reduced pressure and the residue was purified by column chromatography (EA/PE, 2/3) to give 5-((2,6-dichlorobenzyl)oxy)-6-methyl-2,3-dihydro-1H-inden-1-one (250 mg, 50%) as a yellow solid. LCMS (ESI, m/z): 321 [M+H]⁺.

### Synthesis of isopropyl 1-(5-((2,6-dichlorobenzyl)oxy)-6-methyl-2,3-dihydro-1H-inden-1-yl)piperidine-4-carboxylate

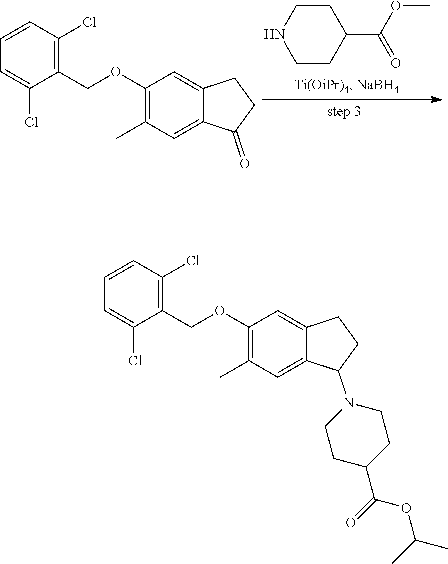

To a solution of 5-((2,6-dichlorobenzyl)oxy)-6-methyl-2,3-dihydro-1H-inden-1-one (250 mg, 0.780 mmol, 1.00 equiv) in THE (3.0 mL) were added methyl piperidine-4-carboxylate (334 mg, 2.330 mmol, 3.00 equiv) and Ti(OiPr)₄ (663 mg, 2.330 mmol, 3.00 equiv). The resulting mixture was stirred for 12 h at 60° C. LCMS indicated the formation of imine intermediate. The reaction was cooled to room temperature and NaBH₄ (88 mg, 2.330 mmol, 3.00 equiv) was added. The resulting mixture was stirred for 0.5 h. LCMS showed the reaction was complete. The reaction was quenched with 10 mL of water and filtered. The filtrate was extracted with ethyl acetate (20 mL*3). The combined organic layers were dried over anhydrous Na₂SO₄ and concentrated under vacuum. The residue was purified by flash chromatography on C18 silica (5 mM NH₄HCO₃)/ACN, 30/70) to give isopropyl 1-(5-((2,6-dichlorobenzyl)oxy)-6-methyl-2,3-dihydro-1H-inden-1-yl)piperidine-4-carboxylate (100 mg, 26%) as a yellow solid. LCMS (ESI, m/z): 476 [M+H]⁺.

### Synthesis of 1-(5-((2,6-dichlorobenzyl)oxy)-6-methyl-2,3-dihydro-1H-inden-1-yl)piperidine-4-carboxylic acid

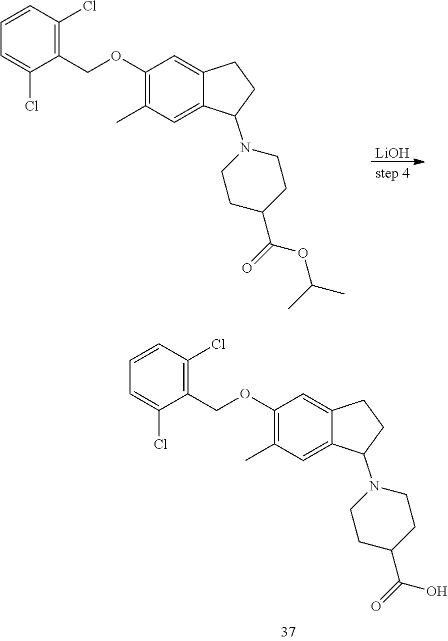 [SMILES: Cc1cc2c(cc1OCc1c(Cl)cccc1Cl)CCC2N1CCC(C(=O)OC(C)C)CC1 ; "LiOH" ; Cc1cc2c(cc1OCc1c(Cl)cccc1Cl)CCC2N1CCC(C(=O)O)CC1]

To a solution of isopropyl 1-(5-((2,6-dichlorobenzyl)oxy)-6-methyl-2,3-dihydro-1H-inden-1-yl)piperidine-4-carboxylate (100 mg, 0.210 mmol, 1.00 equiv) in THF (1.0 mL) and water (1.0 mL) was added KOH (35 mg, 0.630 mmol, 3.00 equiv). The reaction was stirred overnight at 70° C. LCMS showed the reaction was complete. The mixture was acidified to pH 4-5 with 1 M HCl, and concentrated under reduced pressure. The residue was purified by prep-HPLC (Column: YMC-Actus Triart C18, 30*250, 5 m, Mobile Phase A: water (10 mM NH₄HCO_{3+0.1}% NH₃·H₂O), Mobile Phase B: ACN; Flow rate: 60 mL/min; Gradient: 22% B to 38% B in 10 min, hold at 38% B for 3 min; 254/210 nm; RT: 10.25 min) to give 1-(5-((2,6-dichloro-benzyl)oxy)-6-methyl-2,3-dihydro-1H-inden-1-yl)piperidine-4-carboxylic acid (21.4 mg, 22%) as an off-white solid.

LCMS (ESI, m/z): 434 [M+H]⁺. Analytic Conditions: EVO C18, 3.0*50 mm, 2.6 m; Mobile Phase A: water (5 mM NH₄HCO₃), Mobile Phase B: ACN; Flow rate: 1.20 mL/min; Gradient: 10% B to 95% B in 2.0 min, hold at 95% B for 0.60 min, 95% B to 10% B in 0.15 min; 210 nm; RT: 1.154 min.

¹H NMR (400 MHz, DMSO-d₆) δ 7.51-7.49 (m, 2H), 7.42-7.38 (m, 1H), 6.95 (s, 2H), 5.12 (s, 2H), 4.12 (t, J=6.8 Hz, 1H), 2.81-2.62 (m, 4H), 2.19-2.13 (m, 1H), 2.10-2.02 (m, 2H), 1.99 (s, 3H), 1.95-1.87 (m, 2H), 1.74-1.65 (m, 2H), 1.55-1.45 (m, 1H), 1.42-1.33 (m, 1H).

### Example S38. Synthesis of 1-(5-((2,6-dichlorobenzyl)oxy)-3-methyl-2,3-dihydro-1H-inden-1-yl)piperidine-4-carboxylic acid (38)

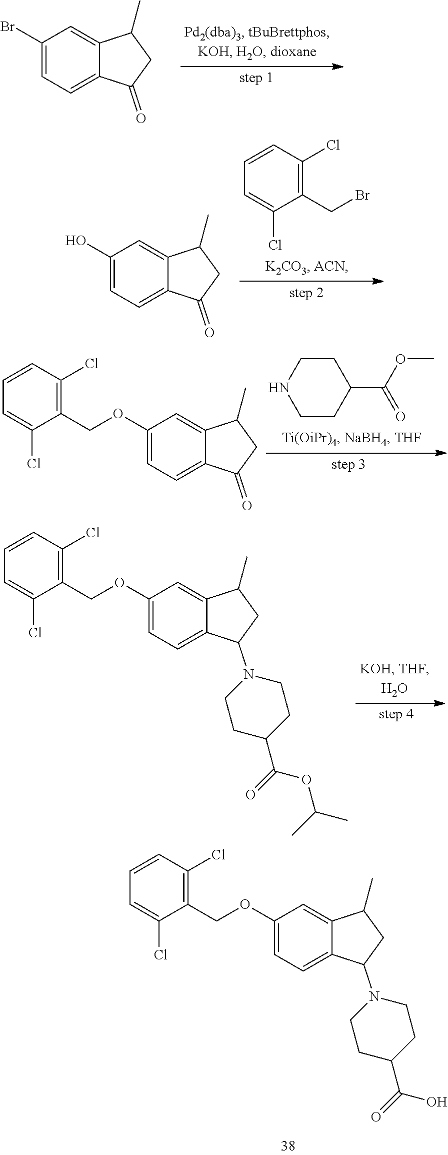

### Synthesis of 5-hydroxy-3-methyl-2,3-dihydro-1H-inden-1-one

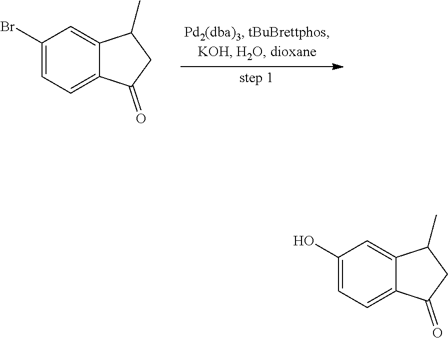

To a stirred solution of 5-bromo-3-methyl-indan-1-one (500 mg, 2.22 mmol, 1.00 equiv) in 1,4-dioxane (2.0 mL) and water (0.4 mL) was added KOH (373 mg, 6.66 mmol, 3.00 equiv), Pd₂(dba)₃ (203 mg, 0.22 mmol, 0.10 equiv) and t-BuBrettPhos (215 mg, 0.440 mmol, 0.20 equiv). The reaction was stirred for 12 h at 80° C. under nitrogen atmosphere. LCMS showed the reaction was complete. The reaction was acidified to pH 4-5 with 1 M HCl. The resulting mixture was concentrated under reduced pressure. The residue was purified by flash column chromatography on C18 silica (5 mM NH₄HCO₃/ACN, 80/20) to give 5-hydroxy-3-methyl-2,3-dihydro-1H-inden-1-one (350 mg, 97%) as a yellow solid. LCMS (ESI, m/z): 163 [M+H]⁺.

### Synthesis of 5-((2,6-dichlorobenzyl)oxy)-3-methyl-2,3-dihydro-1H-inden-1-one

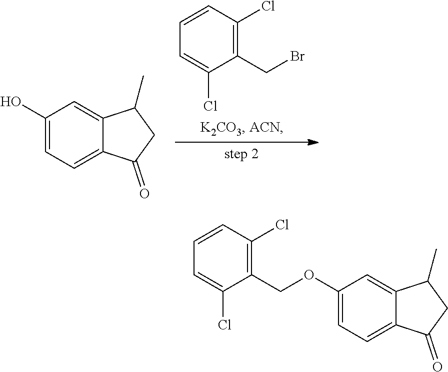

To a stirred solution of 5-hydroxy-3-methyl-2,3-dihydro-1H-inden-1-one (250 mg, 1.54 mmol, 1.00 equiv) in MeCN (5.0 mL) was added 2-(bromomethyl)-1,3-dichloro-benzene (554 mg, 2.31 mmol, 1.50 equiv) and K₂CO₃ (638 mg, 4.62 mmol, 3.00 equiv). The reaction was stirred for 4 h at 60° C. LCMS showed the reaction was complete. The reaction mixture was concentrated under reduced pressure and the residue was purified by column chromatography (EA/PE=1/1) to give 5-((2,6-dichlorobenzyl)oxy)-3-methyl-2,3-dihydro-1H-inden-1-one (200 mg, 40%) as a yellow solid. LCMS (ESI, m/z): 321 [M+H]⁺.

### Synthesis of isopropyl 1-(5-((2,6-dichlorobenzyl)oxy)-3-methyl-2,3-dihydro-1H-inden-1-yl)piperidine-4-carboxylate

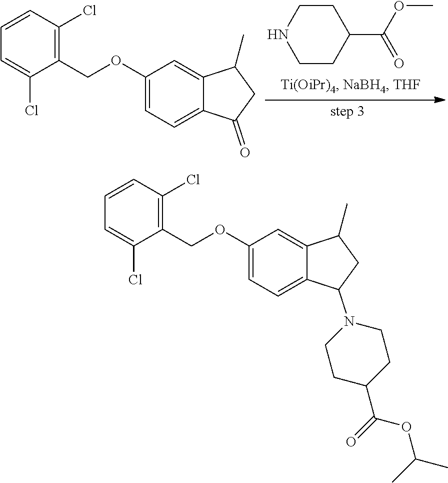

To a stirred solution of 5-((2,6-dichlorobenzyl)oxy)-3-methyl-2,3-dihydro-1H-inden-1-one (200 mg, 0.62 mmol, 1.00 equiv) in THF (5.0 mL) were added Ti(OiPr)₄ (0.5 mL, 1.87 mmol, 3.00 equiv) and methyl piperidine-4-carboxylate (89 mg, 0.62 mmol, 1.00 equiv). The reaction was stirred for 12 h at 60° C. LCMS indicated the formation of imine intermediate. The reaction was cooled to room temperature and NaBH₄ (70 mg, 1.87 mmol, 3.00 equiv) was added. The resulting mixture was stirred for 0.5 h. LCMS showed the reaction was complete. The reaction was quenched with 10 mL of water and filtered. The filtrate was extracted with ethyl acetate (20 mL*3). The combined organic layers were dried over anhydrous Na₂SO₄ and concentrated under vacuum. The residue was purified by flash chromatography on C18 silica (eluted with water (5 mM NH₄HCO₃/ACN, 30/70) to give isopropyl 1-(5-((2,6-dichloro-benzyl)-oxy)-3-methyl-2,3-dihydro-1H-inden-1-yl)piperidine-4-carboxylate (200 mg, 67%) as a yellow solid. LCMS (ESI, m/z): 476 [M+H]⁺.

### Synthesis of 1-(5-((2,6-dichlorobenzyl)oxy)-3-methyl-2,3-dihydro-1H-inden-1-yl)-piperidine-4-carboxylic acid

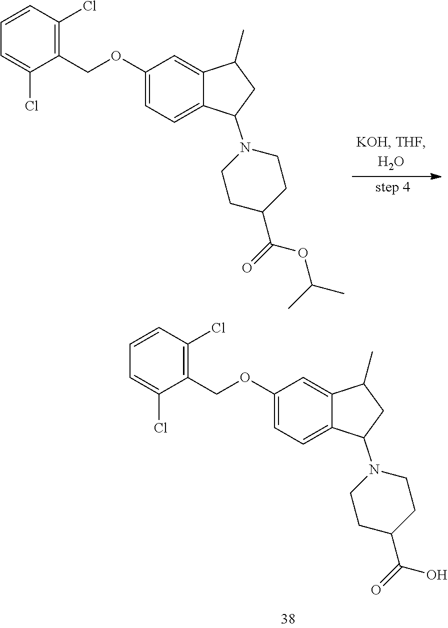 [SMILES: CC(C)OC(=O)C1CCN(C2CC(C)c3cc(OCc4c(Cl)cccc4Cl)ccc32)CC1 ; CC1CC(N2CCC(C(=O)O)CC2)c2ccc(OCc3c(Cl)cccc3Cl)cc21]

To a solution of isopropyl 1-(5-((2,6-dichlorobenzyl)oxy)-3-methyl-2,3-dihydro-1H-inden-1-yl)piperidine-4-carboxylate (200 mg, 0.42 mmol, 1.00 equiv) in THF (2.0 mL) and water (2.0 mL) was added KOH (70 mg, 1.26 mmol, 3.00 equiv). The reaction was stirred overnight at room temperature. LCMS showed the reaction was complete. The mixture was acidified to pH 4-5 with 1 M HCl, and concentrated under reduced pressure. The residue was purified by prep-HPLC (Column: XBridge C18 OBD Prep Column, 5 μm, 19*250 mm; Mobile Phase A: water (10 mM NH₄HCO₃+0.1% NH₃·H₂O), Mobile Phase B: ACN; Flow rate: 25 mL/min; Gradient: 30% B to 50% B in 7 min; 254/210 nm; RT: 6.42 min) to afford 1-(5-((2,6-dichlorobenzyl)oxy)-3-methyl-2,3-dihydro-1H-inden-1-yl)piperidine-4-carboxylic acid (28.8 mg, 15%) as an off-white solid.

LCMS (ESI, m/z): 434 [M+H]⁺. Analytic Conditions: column: Shim-pack XR-ODS, 3.0*50 mm, 2.2 m; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 210 nm; RT: 1.590 min.

¹H NMR (300 MHz, DMSO-d₆) δ 12.12 (br, 1H), 7.58-7.55 (m, 2H), 7.49-7.44 (m, 1H), 7.18-7.10 (m, 1H), 6.92-6.87 (m, 2H), 5.20 (s, 2H), 4.24-4.18 (m, 1H), 3.23-3.10 (m, 1H), 2.99-2.91 (m, 1H), 2.85-2.73 (m, 1H), 2.37-2.25 (m, 1H), 2.23-2.04 (m, 3H), 1.84-1.75 (m, 2H), 1.63-1.44 (m, 3H), 1.28-1.19 (m, 3H).

### Example S39. Synthesis of 1-(5-((2,6-dichlorobenzyl)oxy)-3-methyl-2,3-dihydro-1H-inden-1-yl)piperidine-4-carboxylic acid (39)

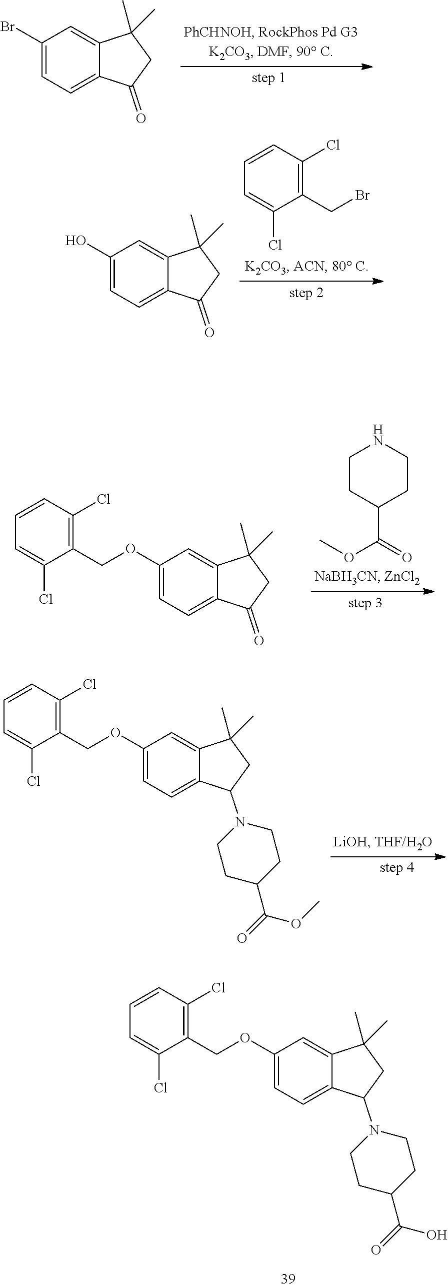

### Step 1: Synthesis of 5-hydroxy-3,3-dimethyl-2,3-dihydro-1H-inden-1-one

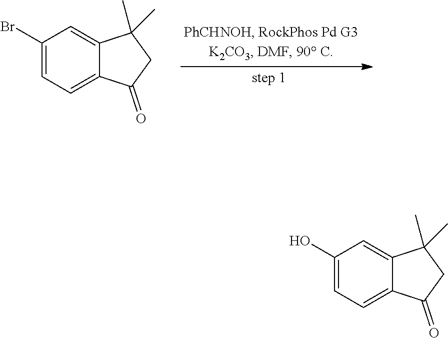

A mixture of 5-bromo-3,3-dimethyl-indan-1-one (500 mg, 2.09 mmol, 1.00 equiv), PhCHNOH (1518.13 mg, 3.14 mmol, 1.50 equiv), RockPhos G3 Pd (191.49 mg, 0.21 mmol, 0.10 equiv) and K₂CO₃ (865.71 mg, 6.270 mmol, 3.00 equiv) in DMF (5.0 mL) was stirred at 90° C. for 12 h. LCMS showed the reaction was complete. The reaction was acidified to pH 4-5 with 1 M HCl. The resulting mixture was concentrated under reduced pressure. The residue was purified by flash chromatography on silica gel (PE/EA, 1/1) to give 5-hydroxy-3,3-dimethyl-indan-1-one (300 mg, 81%) as a light yellow oil. LCMS (ESI, m/z): 177 [M+H]⁺.

### Synthesis of 5-((2,6-dichlorobenzyl)oxy)-3,3-dimethyl-2,3-dihydro-1H-inden-1-one

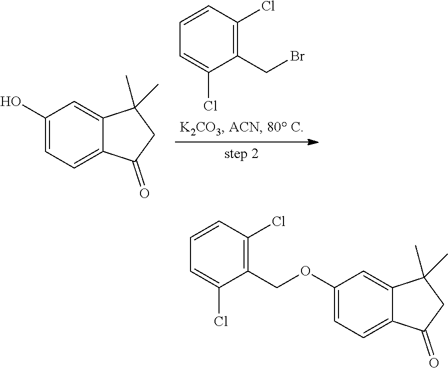

A mixture of 5-hydroxy-3,3-dimethyl-indan-1-one (300 mg, 1.70 mmol, 1.00 equiv), 2-(bromomethyl)-1,3-dichloro-benzene (408 mg, 1.70 mmol, 1.00 equiv) and K₂CO₃ (704 mg, 5.10 mmol, 3.00 equiv) in MeCN (6.0 mL) was stirred at 80° C. for 12 h. LCMS showed the reaction was complete. The reaction mixture was concentrated under reduced pressure. The residue was purified by flash chromatography on silica gel (PE/EA, 2/1) to give 5-((2,6-di-chlorobenzyl)oxy)-3,3-dimethyl-2,3-dihydro-1H-inden-1-one (200 mg, 35%) as a light yellow oil. LCMS (ESI, m/z): 335 [M+H]⁺.

### Synthesis of methyl 1-(5-((2,6-dichlorobenzyl)oxy)-3,3-dimethyl-2,3-dihydro-1H-inden-1-yl)piperidine-4-carboxylate

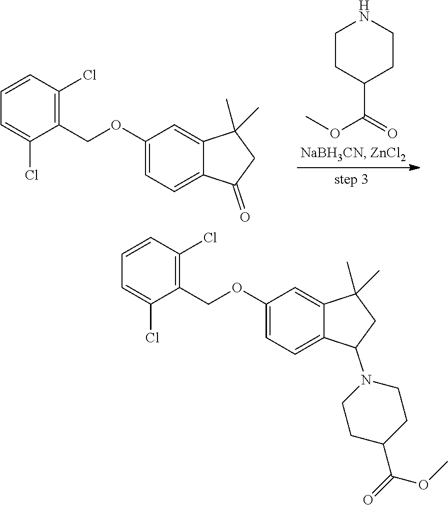

A mixture of 5-((2,6-dichlorobenzyl)oxy)-3,3-dimethyl-2,3-dihydro-1H-inden-1-one (200 mg, 0.60 mmol, 1.00 equiv), methyl piperidine-4-carboxylate (170.85 mg, 1.20 mmol, 2.00 equiv), zinc chloride (1.9 M in 2-Me-THF, 0.64 mL, 1.20 mmol, 2.00 equiv) and NaBH₃CN (152.74 mg, 2.400 mmol, 4.00 equiv) in methanol (5.0 mL) was stirred at 60° C. for 12 h. LCMS showed the reaction was complete. The reaction was quenched by the addition of 50 mL of water and filtered. The filtrate was extracted with ethyl acetate (20 mL*3). The combined organic layers were dried over anhydrous Na₂SO₄ and concentrated under vacuum. The residue was purified by flash chromatography on silica gel (PE/EA, 3/1) to give methyl 1-(5-((2,6-dichlorobenzyl)oxy)-3,3-dimethyl-2,3-dihydro-1H-inden-1-yl)piperidine-4-carboxylate (100 mg, 36%) as a light yellow oil. LCMS (ESI, m/z): 462 [M+H]⁺.

### Synthesis of 1-(5-((2,6-dichlorobenzyl)oxy)-3,3-dimethyl-2,3-dihydro-1H-inden-1-yl)-piperidine-4-carboxylic acid

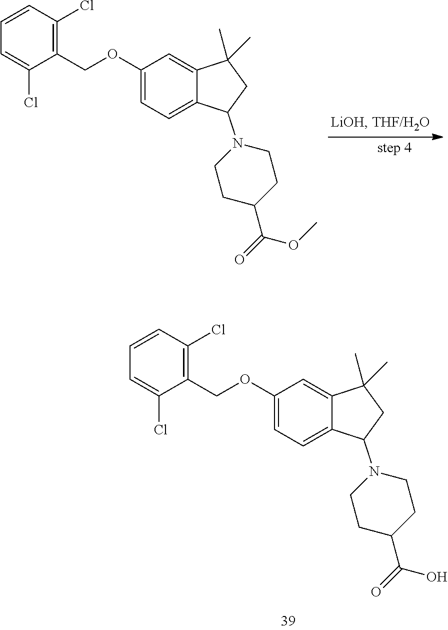 [SMILES: COC(=O)C1CCN(C2CC(C)(C)c3cc(OCc4c(Cl)cccc4Cl)ccc32)CC1 ; CC1(C)CC(N2CCC(C(=O)O)CC2)c2ccc(OCc3c(Cl)cccc3Cl)cc21]

A mixture of methyl 1-(5-((2,6-dichlorobenzyl)oxy)-3,3-dimethyl-2,3-dihydro-1H-inden-1-yl)piperidine-4-carboxylate (100 mg, 0.22 mmol, 1.00 equiv) and LiOH H₂O (45.41 mg, 1.08 mmol, 5.00 equiv) in THE (2.0 mL) and water (2.0 mL) was stirred at room temperature for 1 h. LCMS showed the reaction was complete. The mixture was acidified to pH 4-5 with 1 M HCl and was filtered. The solid was collected to give 1-(5-((2,6-dichlorobenzyl)oxy)-3,3-dimethyl-2,3-dihydro-1H-inden-1-yl)piperidine-4-carboxylic acid (54.4 mg, 56%) as a white solid.

LCMS (ESI, m/z): 448 [M+H]⁺. Analytic Conditions: EVO C18, 3.0*50 mm, 2.6 m; Mobile Phase A: water (5 mM NH₄HCO₃), Mobile Phase B: ACN; Flow rate: 1.20 mL/min; Gradient: 10% B to 95% B in 2.0 min, hold at 95% B for 0.60 min, 95% B to 10% B in 0.15 min; 220 nm; RT: 1.255 min.

¹H NMR (400 MHz, DMSO-d₆) δ 12.52 (s, 1H), 11.08 (s, 1H), 7.90-7.85 (m, 1H), 7.59-7.57 (m, 2H), 7.51-7.47 (m, 1H), 7.06-7.00 (m, 2H), 5.26 (s, 2H), 5.12-5.07 (m, 1H), 3.49-3.46 (m, 1H), 3.07-2.95 (m, 3H), 2.63-2.56 (m, 1H), 2.26-2.14 (m, 3H), 2.08-2.00 (m, 2H), 1.92-1.89 (m, 1H), 1.41 (m, 3H), 1.19 (s, 3H).

### Biological Examples

### Example B1. Cell Membrane Preparations

CHO cells expressing recombinant S1P5 receptors were cultured in 500 cm² culture trays and, once confluent, rinsed and detached with cell-lifting buffer (10 mM HEPES, 154 mM NaCl, 6.85 mM EDTA, pH 7.4). Cells were then pelleted by centrifugation, resuspended, and homogenized in membrane preparation buffer (10 mM HEPES and 10 mM EDTA, pH 7.4) using a Polytron PT 1200E homogenizer (Kinematica, Luzern, Switzerland). Cellular proteins were pelleted by centrifugation at 48,000×g at 4° C. for 30 minutes. The resulting supernatant was discarded, and the pellet was re-suspended again in membrane preparation buffer, homogenized for a second time, and then centrifuged again as described above. The final cellular protein pellet was suspended in ice cold resuspension buffer (10 mM HEPES and 0.1 mM EDTA, pH 7.4), divided into aliquots, and stored at −80° C. until use.

### Example B2. GTPγS Binding Assay

Functional binding assays for [³⁵S]-GTPγS were performed in 96-well non-binding surface plates with a final volume of 200 μL. The test compounds were serially diluted in DMSO and added to assay plates using a Tecan D300E digital printer with a total volume of 0.4 L. The control sphingosine-1-phosphate (S1P) was prepared separately by preparing a 400 μM stock solution from a 100 nmol pellet of S1P in 10 mM Na₂CO₃ with 2% O-cyclodextrin. The serial dilution of S1P was done using complete assay buffer (20 mM HEPES, 10 mM MgCl₂, 100 mM NaCl, 1 mM EDTA, 0.1% fatty acid free bovine serum albumin (BSA), and 30 μg/mL saponin, pH 7.4) and transferred to wells already containing 0.4 μL DMSO. All the wells were then loaded to a total volume of 40 μL of complete assay buffer, except the non-specific binding (NSB) wells. For NSB wells, 40 μL/well of 50 μM GTPγS (Sigma Aldrich, cat #G8634, St. Louis, MO) was added to wells containing 0.4 μL of DMSO. The assay was started by the addition of 120 μL/well of CHO-SlP receptor membrane solution containing 40 μg/mL of membrane protein, 16.67 μM guanosine diphosphate (GDP; Sigma Aldrich, cat #G7127, St. Louis, MO), and 2.5 mg/mL of WGA PVT SPA beads in complete buffer. Assay plates were then sealed and incubated at room temperature with gentle agitation for 30 minutes. Next, 40 L/well of 1 nM of [³⁵S]-GTPγS (PerkinElmer, cat #NEG030X25OUC, Waltham, MA) in basic assay buffer (20 mM HEPES, 10 mM MgCl₂, 100 mM NaCl, and 1 mM EDTA, pH7.4) was added to the assay plates to yield a final concentration of 200 pM and the plates were further incubated for 40 minutes at room temperature with gentle agitation. The assay was terminated by centrifugation of the plates at 1000 rpm for 3 minutes using an Eppendorf 5810R centrifuge (Eppendorf, Hamburg, Germany) and G protein bound radioactivity was quantitated using a MicroBeta2 microplate scintillation counter (PerkinElmer, Waltham, MA). As G protein bound radioactivity directly correlates to receptor activation and coupling to the G protein, this assay is a measure of S1P5 agonism. Results are shown in Table 2.

TABLE 2
S1P5 GTPγS Binding of Exemplary Compounds.

| Compound No. | S1P5 GTPγS binding |
|---|---|
| 1 | ++++ |
| 2 | +++ |
| 3 | ++++ |
| 4 | +++ |
| 5 | ++++ |
| 6 | +++ |
| 7 | +++ |
| 8 | +++ |
| 9 | ++++ |
| 10 | +++ |
| 11 | +++ |
| 12 | +++ |
| 13 | +++ |
| 14 | +++ |
| 15 | +++ |
| 16 | +++ |
| 17 | ++++ |
| 18 | +++ |
| 19 | +++ |
| 20 | +++ |
| 21 | ++ |
| 22 | ++ |
| 23 | ++++ |
| 24a | +++ |
| 24b | ++++ |
| 25a | ++++ |
| 25b | ++++ |
| 26a | +++ |
| 26b | ++ |
| 27 | ++++ |
| 28 | +++ |
| 29 | +++ |
| 30 | +++ |
| 31 | +++ |
| 32 | ++++ |
| 33 | ++++ |
| 34 | +++++ |
| 35 | +++++ |
| 36 | +++++ |
| 37 | ++++ |
| 38 | ++++ |
| 39 | +++ |

ND = not determined
+++++ indicates binding ≤1nM
++++ indicates binding between greater than 1 nM and ≤10 nM
+++ indicates binding between greater than 10 nM and ≤100 nM
++ indicates binding between greater than 100 nM and ≤1,000 nM
+ indicates binding between greater than 1,000 nM and ≤10,000 nM

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 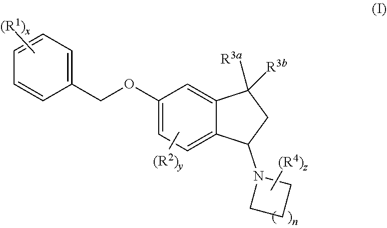
or a pharmaceutically acceptable salt thereof, wherein:
each R¹ is independently halo, —CN, C₁-C₆ alkyl, C₁-C₆ haloalkyl, C₁-C₆ alkoxy, or C₃-C₆ cycloalkyl;
each R² is independently halo, C₁-C₆ alkyl, or C₁-C₆ haloalkyl;
R^{3a} and R^{3b} are independently H, C₁-C₆ alkyl, halo, or C₁-C₆ haloalkyl;
x is 1-5;
y is 0-3;
z is 1-5;
n is 1, 2, or 3; and
each R⁴ is independently —CO₂H, halo, C₁-C₆ haloalkyl, or C₁-C₆ alkyl,
or two R⁴ groups are taken together with the carbon atoms to which they are attached to form a fused, bridged, or spiro C₃-C₅ cycloalkyl optionally substituted by —CO₂H,
provided that at least one R⁴ group is —CO₂H or contains a —CO₂H moiety.

2. The compound of claim 1, or a pharmaceutically acceptable salt thereof, wherein:
each R¹ is independently halo, —CN, C₁-C₃ alkyl, C₁-C₃ haloalkyl, C₁-C₃ alkoxy, or C₃-C₆ cycloalkyl.

3. The compound of claim 1 or 2, or a pharmaceutically acceptable salt thereof, wherein:
each R¹ is independently F, Cl, I, —CN, —CH₃, —CF₃, —OCH(CH₃)₂, or cyclopropyl.

4. The compound of any one of claims 1-3, or a pharmaceutically acceptable salt thereof, wherein:
x is 1, 2, or 3.

5. The compound of any one of claims 1-4, or a pharmaceutically acceptable salt thereof, wherein:

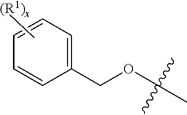

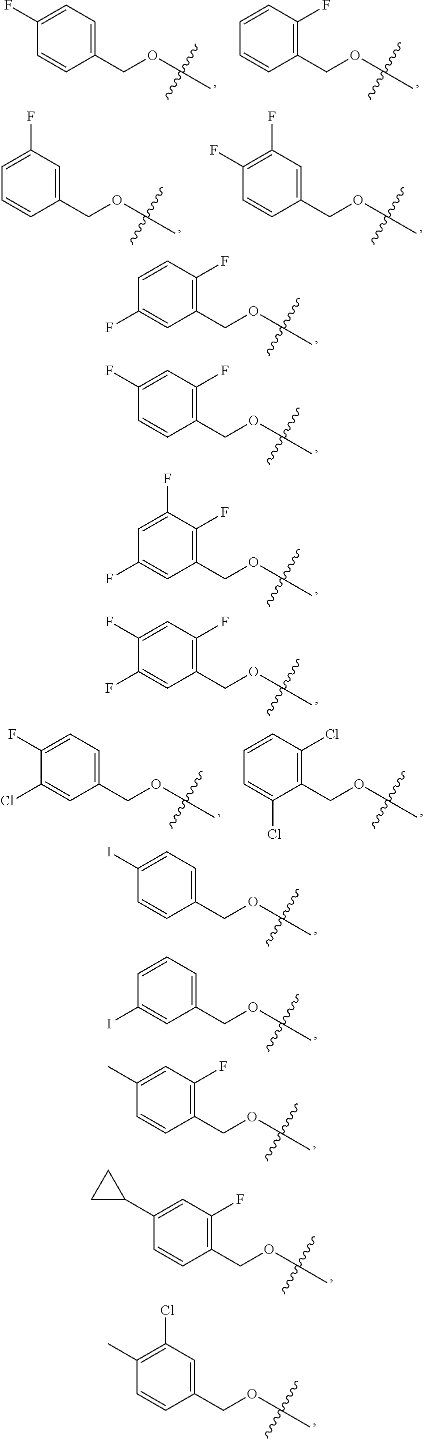

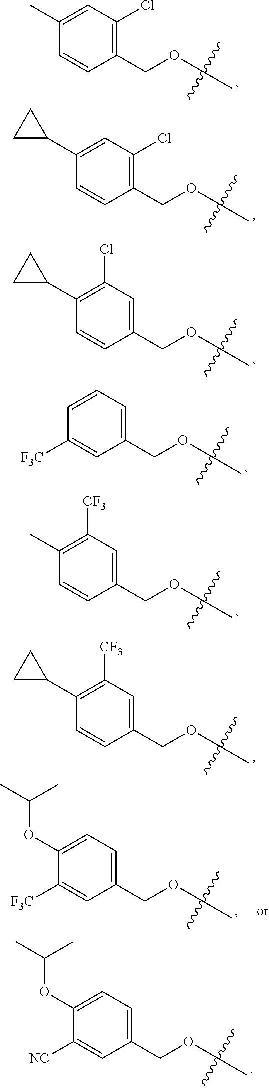

6. The compound of any one of claims 1-5, or a pharmaceutically acceptable salt thereof, wherein:
each R² is independently halo, C₁-C₃ alkyl, or C₁-C₃ haloalkyl.

7. The compound of claim 6, or a pharmaceutically acceptable salt thereof, wherein:
each R² is independently F or —CH₃.

8. The compound of any one of claims 1-7, or a pharmaceutically acceptable salt thereof, wherein:
y is 1.

9. The compound of any one of claims 1-5, or a pharmaceutically acceptable salt thereof, wherein:
y is 0.

10. The compound of any one of claims 1-9, or a pharmaceutically acceptable salt thereof, wherein:

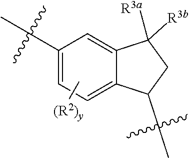

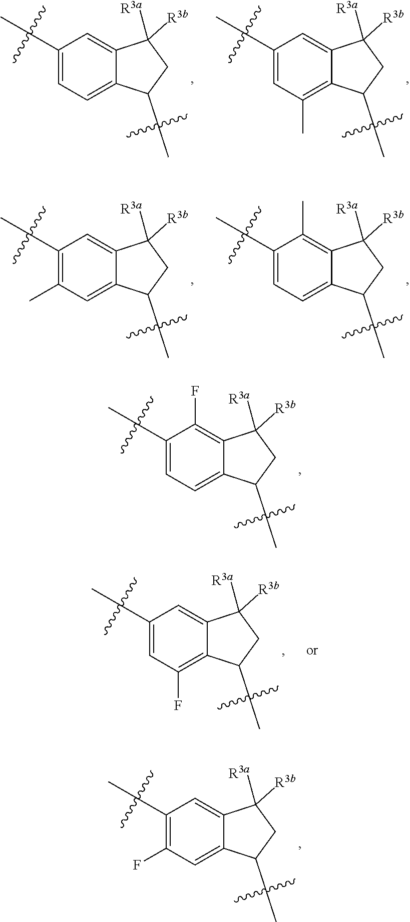

11. The compound of any one of claims 1-10, or a pharmaceutically acceptable salt thereof, wherein:
R^{3a} and R^{3b} are independently H, C₁-C₃ alkyl, halo, or C₁-C₃ haloalkyl.

12. The compound of claim 11, or a pharmaceutically acceptable salt thereof, wherein:
R^{3a} and R^{3b} are independently H or —CH₃.

13. The compound of any one of claims 1-12, or a pharmaceutically acceptable salt thereof, wherein:

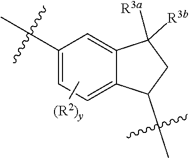

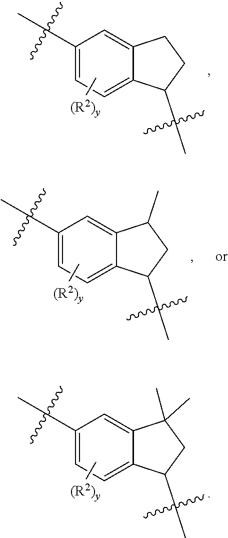

14. The compound of any one of claims 1-13, or a pharmaceutically acceptable salt thereof, wherein:

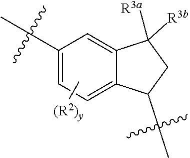

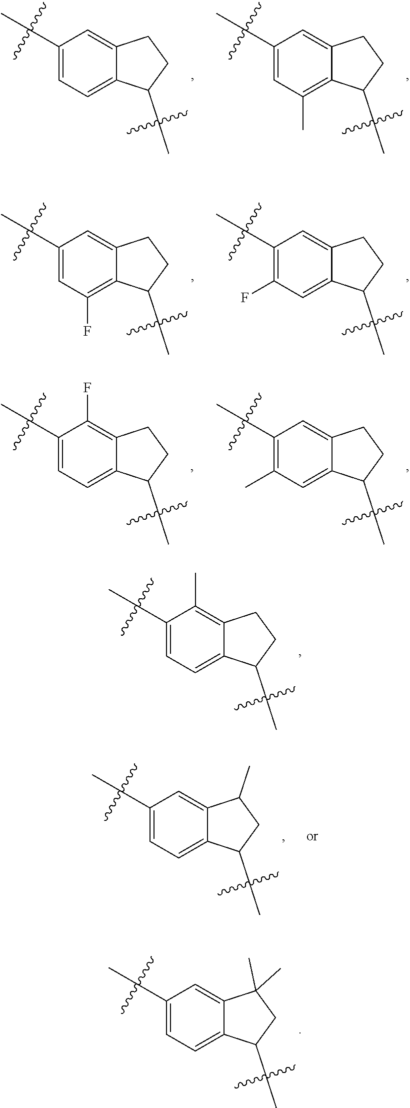

15. The compound of any one of claims 1-14, or a pharmaceutically acceptable salt thereof, wherein:
n is 1.

16. The compound of any one of claims 1-14, or a pharmaceutically acceptable salt thereof, wherein:
n is 2.

17. The compound of any one of claims 1-14, or a pharmaceutically acceptable salt thereof, wherein:
n is 3.

18. The compound of any one of claims 1-17, or a pharmaceutically acceptable salt thereof, wherein:
each R⁴ is independently —CO₂H, halo, C₁-C₃ haloalkyl, or C₁-C₃ alkyl, provided that at least one R⁴ group is —CO₂H.

19. The compound of any one of claims 1-17, or a pharmaceutically acceptable salt thereof, wherein:
two R⁴ groups are taken together with the carbon atoms to which they are attached to form a fused or spiro C₃-C₅ cycloalkyl substituted by —CO₂H.

20. The compound of any one of claims 1-19, or a pharmaceutically acceptable salt thereof, wherein:
z is 1-3.

21. The compound of any one of claims 1-20, or a pharmaceutically acceptable salt thereof, wherein:

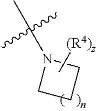

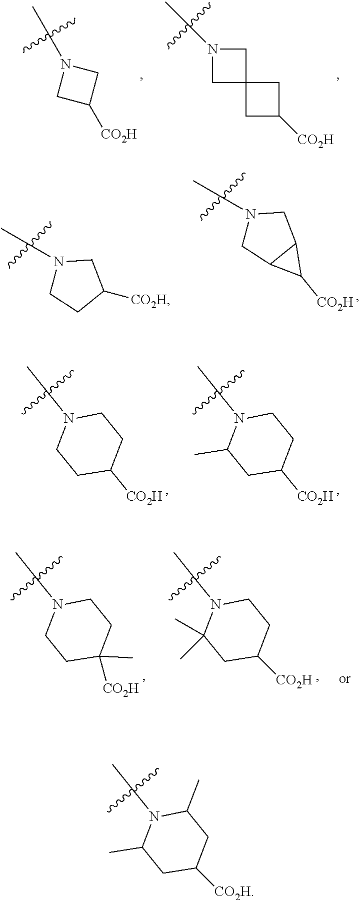

22. A compound selected from the compounds of Table 1 or a pharmaceutically acceptable salt thereof.

23. A pharmaceutical composition comprising the compound of any one of claims 1-22, or a pharmaceutically acceptable salt thereof, and a pharmaceutically acceptable excipient.

24. A method of modulating sphingosine 1-phosphate receptor 5 (S1P5) comprising contacting S1P5 with an effective amount of the compound of any one of claims 1-22, or a pharmaceutically acceptable salt thereof, or the pharmaceutical composition of claim 23.

25. A method of treating a neurological disease in a subject in need thereof, comprising administering to the subject an effective amount of the compound of any one of claims 1-22, or a pharmaceutically acceptable salt thereof, or the pharmaceutical composition of claim 23.

26. The method of claim 25, wherein the neurological disease is Alzheimer's disease or multiple sclerosis.
